SEQUENCE LISTING (90 sequences)

SEQ ID NO: 1 - DNA, 26 bp, Artificial Sequence
  Description of Artificial Sequence Synthetic polynucleotide
taactataacggtcctaaggtagcga

SEQ ID NO: 2 - DNA, 2820 bp, Artificial Sequence
  Description of Artificial Sequence Synthetic polynucleotide
ggccggcctgactaactaaactagtgcgattaacattagcacagagagcgttttgccgagtcccatgtcgtcggcaagcacgccgccgttgggccggccgtttttttctctgttaatcatccactgtatgccttttttctgatgcgccaatagattgggcgtttgatgttcaaagttgggatcgtcgttgtcgggcgcttggtcaaaaaattcttgcaattgcaatttgtaattatccattgtggcgacggctattctcttcgattaaactatggttattgcgcgctcgtaatctaagatatcacatttaacacggacattatctaatcaacgtgattgacgacgagttcatttattgtatcttcaataatttgttcgtaaactggtaatgttgttacttgtatcgcaccaagttttctaacatacacacgaacatgtttgtagcttgtttctgaaaaattttgcacaaaatacggcaacccgtccgcgtacatgttagcttgtagcgtgcgcgtgtacacgaccaatttgagggccacgacgtttttaaaaagcgtgtaattcatgtacgtgtaacgcgactagttgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcaggccggcc

SEQ ID NO: 3 - DNA, 2405 bp, Artificial Sequence
  Description of Artificial Sequence Synthetic polynucleotide
ggccggcctgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcaggccggatggataattacaaattgcaattgcaagaattttttgaccaagcgcccgacaacgacgatcccaactttgaacatcaaacgcccaatctattggcgcatcagaaaaaaggcatacagtggatgattaacagagaaaaaaacggccggcc

SEQ ID NO: 4 - DNA, 15 bp, Artificial Sequence
  Description of Artificial Sequence Synthetic polynucleotide
atgaacaaagattaa

SEQ ID NO: 5 - DNA, 18 bp, Artificial Sequence
  Description of Artificial Sequence Synthetic polynucleotide
atgcctcaatgctactag

SEQ ID NO: 6 - DNA, 1128 bp, Artificial Sequence
  Description of Artificial Sequence Synthetic polynucleotide
atgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 7 - protein, 375 aa, Artificial Sequence
  Description of Artificial Sequence Synthetic polypeptide
MTQPKKKRKVPGQVTMKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPSTTIKQDFRLLGQTSVDRLLQLSQGQAVKGNQLLPVSLVKRKTTLAPNTQTASPRALADSLMQLARQVSRLESGQ

SEQ ID NO: 8 - DNA, 1220 bp, Artificial Sequence
  Description of Artificial Sequence Synthetic polynucleotide
atcttggagataattaaattgataaccatctcgcaaataaataagtattttactgttttcgtaacagttttgtaataaaaaaacctataaatatgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 9 - DNA, 1128 bp, Artificial Sequence
  Description of Artificial Sequence Synthetic polynucleotide
atgacccagcccaaaaaaaagagaaaagtccctggtcaggtaactatgaagccggtcaccttgtacgacgtcgctgaatacgcaggtgtgtcctatcaaacagtgagtcgtgtggtcaaccaggccagccacgttagtgcaaaaacccgtgagaaagtggaggcggcaatggccgagcttaattacataccaaaccgtgttgcacaacagcttgcaggcaagcaatccctgctcattggcgttgcaactagtagcctggctcttcatgcgccttcccagattgtggccgctattaagagtagagcagatcagttgggagcatccgttgtcgtcagcatggttgaaaggagtggcgttgaggcttgcaaagctgcggtccataacctgctggcacaaagagtgagcggactcatcatcaactacccactggatgatcaagacgcgattgccgtcgaggctgcatgtacgaacgttccagctctttttcttgacgtgtcagaccaaacacccataaattctattatttttagtcacgaggacggtacccgcttgggtgttgaacacttggtagctttgggacatcagcagatcgcattgttggctggtccacttagtagcgtgagtgcaagacttagactggcaggttggcacaagtacttgacccgtaatcaaatacaacctatagctgagcgcgaaggtgactggagcgctatgagcggtttccagcaaacgatgcagatgctgaacgagggaattgtacccaccgctatgttggtcgctaacgaccaaatggccttgggcgccatgagagctattactgaaagcggcttgcgcgtgggcgcagatatctctgtcgtaggttacgatgatacggaagactctagctgctatatccccccaagcacgacgatcaagcaggatttccgtctccttggacagaccagtgtagacaggctccttcaactgagccagggccaagctgtgaagggcaaccaactcttgccagtgtcgttggtgaagcgcaagaccaccctggctcccaacacgcaaactgcttctcccagggctttggctgattccctgatgcagctcgcccgccaagtcagtagacttgaatcaggccagtga

SEQ ID NO: 10 - DNA, 1125 bp, Artificial Sequence
  Description of Artificial Sequence Synthetic polynucleotide
atggcacagcccaaaaagaagcgtaaggtgcccggccaggtcacgatgaaacctgtcaccctttatgacgttgccgaatatgcgggtgtttcgtatcagacagtttcacgcgtagtgaaccaagcttctcacgtcagcgccaagaccagggaaaaggtagaggctgccatggcagaactgaattatatacccaacagagtcgcacaacagcttgcaggaaaacagagtctcttgatcggtgttgccacatcttcgttggcacttcacgctcctagtcaaatcgttgctgctatcaagagtagggcagatcaacttggtgcctcggtagttgtgagtatggtagagcgctccggcgtcgaagcttgtaaggccgctgtgcataatctcctcgcacagcgcgttagcggtcttataattaattacccacttgatgaccaggacgctatagctgtagaagcggcatgtactaacgtgccagcgctgtttttggacgtatccgaccaaacacccattaactccattattttctcacacgaggatggcacgaggctcggcgtggagcatcttgtagcattgggtcaccaacaaatcgcccttcttgctggcccattgtcttcagtctctgctcgccttcgtcttgcgggctggcataagtatcttacgcgcaaccaaatacaacccattgcagaacgtgagggcgacttctcggccatgtcgggatttcaacaaactatgcaaatgctgaacgagggaatcgttcctaccgcaatgcttgtagcaaacgatcaaatggccttgggagccatgcgcgctattaccgaaagcggattgagagttggtgctgacattagcgtggtaggctacgacgacactgaggactctagttgttatattcctcccttgactacgataaagcaggactttaggctccttggtcagacatctgtggacagactgctccagttgagccagggacaagcggttaagggcaatcagctcctccccgtttcccttgtgaaaaggaagactacgctggcccccaatacgcaaactgcctcaccgcgtgctctcgcggattctctcatgcagctggccaggcaagtaagcaggcttgaatcaggccag

SEQ ID NO: 11 - protein, 375 aa, Artificial Sequence
  Description of Artificial Sequence Synthetic polypeptide
MAQPKKKRKVPGQVTMKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDFSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 12 - DNA, 1133 bp, Artificial Sequence
  Description of Artificial Sequence Synthetic polynucleotide
ccgccaccatggcacagcccaaaaagaagcgtaaggtgcccggccaggtcacgatgaaacctgtcaccctttatgacgttgccgaatatgcgggtgtttcgtatcagacagtttcacgcgtagtgaaccaagcttctcacgtcagcgccaagaccagggaaaaggtagaggctgccatggcagaactgaattatatacccaacagagtcgcacaacagcttgcaggaaaacagagtctcttgatcggtgttgccacatcttcgttggcacttcacgctcctagtcaaatcgttgctgctatcaagagtagggcagatcaacttggtgcctcggtagttgtgagtatggtagagcgctccggcgtcgaagcttgtaaggccgctgtgcataatctcctcgcacagcgcgttagcggtcttataattaattacccacttgatgaccaggacgctatagctgtagaagcggcatgtactaacgtgccagcgctgtttttggacgtatccgaccaaacacccattaactccattattttctcacacgaggatggcacgaggctcggcgtggagcatcttgtagcattgggtcaccaacaaatcgcccttcttgctggcccattgtcttcagtctctgctcgccttcgtcttgcgggctggcataagtatcttacgcgcaaccaaatacaacccattgcagaacgtgagggcgacttctcggccatgtcgggatttcaacaaactatgcaaatgctgaacgagggaatcgttcctaccgcaatgcttgtagcaaacgatcaaatggccttgggagccatgcgcgctattaccgaaagcggattgagagttggtgctgacattagcgtggtaggctacgacgacactgaggactctagttgttatattcctcccttgactacgataaagcaggactttaggctccttggtcagacatctgtggacagactgctccagttgagccagggacaagcggttaagggcaatcagctcctccccgtttcccttgtgaaaaggaagactacgctggcccccaatacgcaaactgcctcaccgcgtgctctcgcggattctctcatgcagctggccaggcaagtaagcaggcttgaatcaggccag

SEQ ID NO: 13 - DNA, 1403 bp, Artificial Sequence
  Description of Artificial Sequence Synthetic polynucleotide
aaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaaggctagcgtatacatcttggagataattaaattgataaccatctcgcaaataaataagtattttactgttttcgtaacagttttgtaataaaaaaacctataaatccgccaccatggcacagcccaaaaagaagcgtaaggtgcccggccaggtcacgatgaaacctgtcaccctttatgacgttgccgaatatgcgggtgtttcgtatcagacagtttcacgcgtagtgaaccaagcttctcacgtcagcgccaagaccagggaaaaggtagaggctgccatggcagaactgaattatatacccaacagagtcgcacaacagcttgcaggaaaacagagtctcttgatcggtgttgccacatcttcgttggcacttcacgctcctagtcaaatcgttgctgctatcaagagtagggcagatcaacttggtgcctcggtagttgtgagtatggtagagcgctccggcgtcgaagcttgtaaggccgctgtgcataatctcctcgcacagcgcgttagcggtcttataattaattacccacttgatgaccaggacgctatagctgtagaagcggcatgtactaacgtgccagcgctgtttttggacgtatccgaccaaacacccattaactccattattttctcacacgaggatggcacgaggctcggcgtggagcatcttgtagcattgggtcaccaacaaatcgcccttcttgctggcccattgtcttcagtctctgctcgccttcgtcttgcgggctggcataagtatcttacgcgcaaccaaatacaacccattgcagaacgtgagggcgacttctcggccatgtcgggatttcaacaaactatgcaaatgctgaacgagggaatcgttcctaccgcaatgcttgtagcaaacgatcaaatggccttgggagccatgcgcgctattaccgaaagcggattgagagttggtgctgacattagcgtggtaggctacgacgacactgaggactctagttgttatattcctcccttgactacgataaagcaggactttaggctccttggtcagacatctgtggacagactgctccagttgagccagggacaagcggttaagggcaatcagctcctccccgtttcccttgtgaaaaggaagactacgctggcccccaatacgcaaactgcctcaccgcgtgctctcgcggattctctcatgcagctggccaggcaagtaagcaggcttgaatcaggccag

SEQ ID NO: 14 - DNA, 20 bp, Artificial Sequence
  Description of Artificial Sequence Synthetic polynucleotide
gattgtgagcgctcacaatt

SEQ ID NO: 15 - DNA, 1379 bp, Artificial Sequence
  Description of Artificial Sequence Synthetic polynucleotide
cctagggctagcatcttggagataattaaaatgataaccatctcgcaaataaataagtattttactgttttcgtaacagttttgtaataaaaaaacctataaatatgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaaacttgtttattgcagcttataatggttacaaataaagcaatagcatcacaaatttcacaaataaagcatttttttcactgcattctagttgtggtttgtccaaactcatcaatgtatcttatcatgtctggatcactagtcctagg

SEQ ID NO: 16 - DNA, 26 bp, Artificial Sequence
  Description of Artificial Sequence Synthetic polynucleotide
gcgtataattgcacactaacatgttg

SEQ ID NO: 17 - DNA, 19 bp, Artificial Sequence
  Description of Artificial Sequence Synthetic polynucleotide
gctggaagtgcgcagtcgc

SEQ ID NO: 18 - DNA, 2705 bp, Artificial Sequence
  Description of Artificial Sequence Synthetic polynucleotide
tcgctaccttaggaccgttatagttatgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcatcgctaccttaggaccgttatagtta

SEQ ID NO: 19 - DNA, 28 bp, Artificial Sequence
  Description of Artificial Sequence Synthetic polynucleotide
agtctgcattttacacgcgttgttaatc

SEQ ID NO: 20 - DNA, 24 bp, Artificial Sequence
  Description of Artificial Sequence Synthetic polynucleotide
tatacgatccagagcgctacgctc

SEQ ID NO: 21 - DNA, 23 bp, Artificial Sequence
  Description of Artificial Sequence Synthetic polynucleotide
ccaagtcagcgtggagatcgagt

SEQ ID NO: 22 - DNA, 22 bp, Artificial Sequence
  Description of Artificial Sequence Synthetic polynucleotide
gccattgggaatcgcaatgcca

SEQ ID NO: 23 - DNA, 2585 bp, Artificial Sequence
  Description of Artificial Sequence Synthetic polynucleotide
ggccggcctgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtcgcgttacacgtacatgaattacacgctttttaaaaacgtcgtggccctcaaattggtcgtgtacacgcgcacgctacaagctaacatgtacgcggacgggttgccgtattttgtgcaaaatttttcagaaacaagctacaaacatgttcgtgtgtatgttagaaaacttggtgcgatacaagtaacaacattaccagtttacgaacaaattattgaagatacaataaatgaactcgtcgtcaatcacgttgattagataatgtccgtgttaaatgtgatatcttagattacgagcgcgcaataaccatagtttaatcgaagagaatagccgtcgccacaatggataattacaaattgcaattgcaagaattttttgaccaagcgcccgacaacgacgatcccaactttgaacatcaaacgcccaatctattggcgcatcagaaaaaaggcatacagtggatgattaacagagaaaaaaacggccggcc

SEQ ID NO: 24 - DNA, 20 bp, Artificial Sequence
  Description of Artificial Sequence Synthetic polynucleotide
agtcccatgtcgtcggcaag

SEQ ID NO: 25 - DNA, 20 bp, Artificial Sequence
  Description of Artificial Sequence Synthetic polynucleotide
tctttgtgcgtgtcagacga

SEQ ID NO: 26 - DNA, 1780 bp, Artificial Sequence
  Description of Artificial Sequence Synthetic polynucleotide
gacctttaattcaacccaacacaatatattatagttaaataagaattattatcaaatcatttgtatattaattaaaatactatactgtaaattacattttatttacaatcactcgacgaagacttgatcaccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 27 - DNA, 1398 bp, Artificial Sequence
  Description of Artificial Sequence Synthetic polynucleotide
aaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaaggctagcgtatacatcttggagataattaaattgataaccatctcgcaaataaataagtattttactgttttcgtaacagttttgtaataaaaaaacctataaatatgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 28 - DNA, 1639 bp, Artificial Sequence
  Description of Artificial Sequence Synthetic polynucleotide
acatcctaggtacaatcaaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaaggctagcgtatacatcttggagataattaaattgataaccatctcgcaaataaataagtattttactgttttcgtaacagttttgtaataaaaaaacctataaatatgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaaacttgtttattgcagcttataatggttacaaataaagcaatagcatcacaaatttcacaaataaagcatttttttcactgcattctagttgtggtttgtccaaactcatcaatgtatcttatcatgtctggatcactagtcctaggatcggatcccgggcccgtcgactgcagaggcctgcatgcaagcttggcgtaatcatggtcatagctgtttcctgtgt

SEQ ID NO: 29 - DNA, 23 bp, Artificial Sequence
  Description of Artificial Sequence Synthetic polynucleotide
gcgagatttaatcgccgcgacaa

SEQ ID NO: 30 - DNA, 29 bp, Artificial Sequence
  Description of Artificial Sequence Synthetic polynucleotide
aaaggcgtatcagaaactgtgctacattc

SEQ ID NO: 31 - DNA, 5834 bp, Artificial Sequence
  Description of Artificial Sequence Synthetic polynucleotide
gatccagacatgataagatacattgatgagtttggacaaaccacaactagaatgcagtgaaaaaaatgctttatttgtgaaatttgtgatgctattgctttatttgtaaccattataagctgcaataaacaagtttcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatagtcacttgcccgggaaccttcctcttcttcttaggttgcgtcatatttataggtttttttattacaaaactgttacgaaaacagtaaaatacttatttatttgcgagatggttatcaatttaattatctccaagatgtatacgctagccttgtaggtcttgtagtgttcctgtgaagagcacagtgcaactgatttaccagaacactgtaggcccttatataccgatgcttatcaacgcgttgggtgcccacgtgactttatcaacgtacactcgggtgacccaatcaacgtgcgccttatcttgcgataatttgattgtacctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagcttgtcgagactgcaggctctagagtcgacctgcaggcatgcaagcttgattcactagttttattgttcaaagatgcagtcatccaaatccacattgaccagatcgcaggcagtgcaagcgtctggcacctttcccatgatatgatgaatgtagcacagtttctgatacgcctttttgacgacagaaacgggttgagattctgacacgggaaagcactctaaacagtctttctgtccgtgagtgaagcagatatttgaattctgattcattctctcgcattgtctgcagggaaacagcatcagattcatgcccacgtgacgagaacatttgttttggtacctgtctgcgtagttgatcgaagcttccgcgtctgacgtcgatggctgcgcaactgactcgcgcacccgtttgggctcacttatatctgcgtcactgggggcgggtcttttcttggctccaccctttttgacgtagaattcatgctccacctcaaccacgtgatcctttgcccaccggaaaaagtctttgacttcctgcttggtgaccttcccaaagtcatgatccagacggcgggtgagttcaaatttgaacatccggtcttgcaacggctgctggtgttcgaaggtcgttgagttcccgtcaatcacggcgcacatgttggtgttggaggtgacgatcacgggagtcgggtctatctgggccgaggacttgcatttctggtccacgcgcaccttgcttcctccgagaatggctttggccgactccacgaccttggcggtcatcttcccctcctcccaccagatcaccatcttgtcgacacagtcgttgaagggaaagttctcattggtccagtttacgcacccgtagaagggcacagtgtgggctatggcctccgcgatgttggtcttcccggtagttgcaggcccaaacagccagatggtgttcctcttgccgaactttttcgtggcccatcccagaaagacggaagccgcatattggggatcgtacccgtttagttccaaaattttataaatccgattgctggaaatgtcctccacgggctgctggcccaccaggtagtcgggggcggttttagtcaggctcataatctttcccgcattgtccaaggcagccttgatttgggaccgcgagttggaggccgcattgaaggagatgtatgaggcctggtcctcctggatccactgcttctccgaggtaatccccttgtccacgagccacccgaccagctccatgtacctggctgaagtttttgatctgatcaccggcgcgtcagaattgggattctgattctctttgttctgctcctgcgtctgcgacacgtgcgtcagatgctgcgccaccaaccgtttacgctccgtgagattcaaacaggcgcttaaatactgttctaaattagtccacgcccactggagctcaggctgggttttggggagcaagtaattggggatgtagcactcgtccaccaccttgttcccgcctccggcgccgtttctggtctttgtgaccgcgaaccagtttggcaaagtcggctcgatcccgcggtaaattctctgaatcagtttttcgcgaatctgactcaggaaacgtcccaaaactaaggatttcaccccggtggtttccacgagcacgtgtaagtggaagtagctctctcccttctcaaattgcacaaagaaaagggcctccggggccttactcacacggcgccactccgtcagaaagtcgcgctgcagcttctcggccacggtcaggggtgcctgctcaatcagattcagatccaagtcagaatctggcggcaactcccactccttctcggccacccagttcacaaagctgtcagaaatgccgggcagatgctcgtcaaggtcgctggggaccttaatcacaatctcgtaaaaccccgccagggcggcagatccgcgcccgatggtgggacggtatgaataatccggaatatttataggtttttttattacaaaactgttacgaaaacagtaaaatacttatttatttgcgagatggttatcattttaattatctccatgatctattaatattccggagtatacggacctttaattcaacccaacacaatatattatagttaaataagaattattatcaaatcatttgtatattaattaaaatactatactgtaaattacattttatttacaatcactcgacgaagacttgatcaccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcagctgatgcatagcatgcggtaccgggagatgggggaggctaactgaaacacggaaggagacaataccggaaggaacccgcgctatgacggcaataaaaagacagaataaaacgcacgggtgttgggtcgtttgttc

SEQ ID NO: 32 - DNA, 2401 bp, Artificial Sequence
  Description of Artificial Sequence Synthetic polynucleotide
tgtgttgggttgaattaaaggtccgtaaatagggccggccgttaacatcatggagataattaaaatgataaccatctcgcaaataaataagtattttactgttttcgtaacagttttgtaataaaaaaacctataaatattccggattattcataccgtcccaccatcgggcgcggttaaccttaagaataaaaatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaactcgaggaatcaagcttgcatgcctgcaggtcgactctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctag

SEQ ID NO: 33 - DNA, 2070 bp, Artificial Sequence
  Description of Artificial Sequence Synthetic polynucleotide
atcatggagataattaaaatgataaccatctcgcaaataaataagtattttactgttttcgtaacagttttgtaataaaaaaacctataaatcgcggatctgccgccaagcttacttacttacttagcttacttacttacttagcttacttacttacttagcttacttacttaattaacctacaacatgaacaaagattaaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 34 - DNA, 2070 bp, Artificial Sequence
  Description of Artificial Sequence Synthetic polynucleotide
atcatggagataattaaaatgataaccatctcgcaaataaataagtattttactgttttcgtaacagttttgtaataaaaaaacctataaatcgcggatctgccgccaagcttacttacttacttagcttacttacttacttagcttacttacttacttaacctacaacatgaacaaagattaaattagcttacttacttaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 35 - DNA, 2070 bp, Artificial Sequence
  Description of Artificial Sequence Synthetic polynucleotide
atcatggagataattaaaatgataaccatctcgcaaataaataagtattttactgttttcgtaacagttttgtaataaaaaaacctataaatcgcggatctgccgccaagcttacttacttacttagcttacttacttacttaacctacaacatgaacaaagattaaattagcttacttacttacttagcttacttacttaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 36 - DNA, 2070 bp, Artificial Sequence
  Description of Artificial Sequence Synthetic polynucleotide
atcatggagataattaaaatgataaccatctcgcaaataaataagtattttactgttttcgtaacagttttgtaataaaaaaacctataaatcgcggatctgccgccaagcttacttacttacttaacctacaacatgaacaaagattaaattagcttacttacttacttagcttacttacttacttagcttacttacttaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 37 - DNA, 2070 bp, Artificial Sequence
  Description of Artificial Sequence Synthetic polynucleotide
atcatggagataattaaaatgataaccatctcgcaaataaataagtattttactgttttcgtaacagttttgtaataaaaaaacctataaatcgcggatctgccgccaaacctacaacatgaacaaagattaaattagcttacttacttacttagcttacttacttacttagcttacttacttacttagcttacttacttaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 38 - DNA, 1432 bp, Artificial Sequence
  Description of Artificial Sequence Synthetic polynucleotide
agacacggtaaccgccgctaactacgctagcgtatcatggagataattaaaatgataaccatctcgcaaataaataagtattttactgttttcgtaacagttttgtaataaaaaaacctataaatattccggattattcataccgtcccaccatcgggcgcggttaaccttaagaataaaaatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaacaatcaaattatcgcaagctagcttgattgaaatgttcaaagaccagtttgacaat

SEQ ID NO: 39 - DNA, 595 bp, Artificial Sequence
  Description of Artificial Sequence Synthetic polynucleotide
ggatgattaacagagaaaaaaacggccggtgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccgcgcgcaccggcaagatagcccgggcccaccggacacgtcaatcgtagattacacaaaatgttcatgtgcacgttgcaattctattaataatggtatttaataataatgtagtattaattatttaaccgtagagaatagctaacgttgcaatggacaactataagctccaacttcaggagttcttcgaccaggccccagataatgatgatcctaattttgagcaccagaccccgaatctgctcgctcaccagaagaagggtattcaatggatgatcaatagggagaagaatggtcggcccaacggcggcgt

SEQ ID NO: 40 - DNA, 228 bp, Artificial Sequence
  Description of Artificial Sequence Synthetic polynucleotide
gattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaatt

SEQ ID NO: 41 - DNA, 527 bp, Artificial Sequence
  Description of Artificial Sequence Synthetic polynucleotide
aggaattcggtacctcgctaccttagtgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccgctacccatccgcacgtttcgctaccttaggaccgttatagttaccgccacgtagaaccagggagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcattaggaccgttatagttaggtacccacga

SEQ ID NO: 42 - DNA, 21 bp, Artificial Sequence
  Description of Artificial Sequence Synthetic polynucleotide
gtctggcgcaacgccaacacg

SEQ ID NO: 43 - DNA, 24 bp, Artificial Sequence
  Description of Artificial Sequence Synthetic polynucleotide
gcacgcccgtcttgcagtttttga

SEQ ID NO: 44 - DNA, 24 bp, Artificial Sequence
  Description of Artificial Sequence Synthetic polynucleotide
gtaaaccctgccggtttgcgcttc

SEQ ID NO: 45 - DNA, 24 bp, Artificial Sequence
  Description of Artificial Sequence Synthetic polynucleotide
cggcaccagtctgagcgacatcac

SEQ ID NO: 46 - DNA, 54 bp, Artificial Sequence
  Description of Artificial Sequence Synthetic polynucleotide
ccgctcaagcagtgatcagatccagacatgataagatacattgatgagtttgga

SEQ ID NO: 47 - DNA, 45 bp, Artificial Sequence
  Description of Artificial Sequence Synthetic polynucleotide
aaggcgcgccttaacaggatacagtctacgtcgagtgattgtaaa

SEQ ID NO: 48 - DNA, 2578 bp, Artificial Sequence
  Description of Artificial Sequence Synthetic polynucleotide
aaggcgcgccttaacaggatacagtctacgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatcgcggatctgccgccaagcttacttacttacttagcttacttacttacttagcttacttacttacttagcttacttacttaattaacctacaacatgaacaaagattaaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaactcgaggaatcaagcttgcatgcctgcaggtcgactctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgcctggatctgatcactgcttgagcgg

SEQ ID NO: 49 - DNA, 1322 bp, Artificial Sequence
  Description of Artificial Sequence Synthetic polynucleotide
tgttaaggcgcgcctttggcaggtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatggcgcgccgctagcaaccgcctctaaccgcgtgtaccgggagatgggggaggctaactgaaacacggaaggagacaataccggaaggaacccgcgctatgacggcaataaaaagacagaataaaacgcacgggtgttgggtcgtttgttccctgccatagccactacgggtac

SEQ ID NO: 50 - DNA, 24 bp, Artificial Sequence
  Description of Artificial Sequence Synthetic polynucleotide
agattgtcgcacctgattgcccga

SEQ ID NO: 51 - DNA, 24 bp, Artificial Sequence
  Description of Artificial Sequence Synthetic polynucleotide
agaacggagtaacctcggtgtgcg

SEQ ID NO: 52 - DNA, 70 bp, Artificial Sequence
  Description of Artificial Sequence Synthetic polynucleotide
agacacggtaaccgccgctaactacgctagcgtatcatggagataattaaaatgataaccatctcgcaaa

SEQ ID NO: 53 - DNA, 76 bp, Artificial Sequence
  Description of Artificial Sequence Synthetic polynucleotide
attgtcaaactggtctttgaacatttcaatcaagctagcttgcgataatttgattgtttattgttcaaagatgcag

SEQ ID NO: 54 - DNA, 27 bp, Artificial Sequence
  Description of Artificial Sequence Synthetic polynucleotide
tcacaacgttacggtcgtcaagcccaa

SEQ ID NO: 55 - DNA, 23 bp, Artificial Sequence
  Description of Artificial Sequence Synthetic polynucleotide
ttgaattacgggcgccggatcgt

SEQ ID NO: 56 - DNA, 869 bp, Artificial Sequence
  Description of Artificial Sequence Synthetic polynucleotide
tagggataacagggtaatcctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttagggataacagggtaat

SEQ ID NO: 57 - DNA, 68 bp, Artificial Sequence
  Description of Artificial Sequence Synthetic polynucleotide
aattcatttgcaagctatacgttaaatcaatagggataacagggtaatcctgggccaacttttggcga

SEQ ID NO: 58 - DNA, 80 bp, Artificial Sequence
  Description of Artificial Sequence Synthetic polynucleotide
ctaataaatatttcattatatattgttgtttattttaagtccattaccctgttatccctaaccaggcgtttaagggcacc

SEQ ID NO: 59 - DNA, 23 bp, Artificial Sequence
  Description of Artificial Sequence Synthetic polynucleotide
gttaaatcaaacggacgttatgg

SEQ ID NO: 60 - DNA, 23 bp, Artificial Sequence
  Description of Artificial Sequence Synthetic polynucleotide
ttaagtcctatataacatggcgg

SEQ ID NO: 61 - DNA, 55 bp, Artificial Sequence
  Description of Artificial Sequence Synthetic polynucleotide
gcaagctatacgttaaatcaatagggataaaaattatcgcaagataaggcgcacg

SEQ ID NO: 62 - DNA, 60 bp, Artificial Sequence
  Description of Artificial Sequence Synthetic polynucleotide
attttaagtccattaccctgttattcattactggcctgattcaagcctgcttacttgcct

SEQ ID NO: 63 - DNA, 2232 bp, Artificial Sequence
  Description of Artificial Sequence Synthetic polynucleotide
ctggctgccgacggttatctacccgattggctcgaggacaaccttagtgaaggaattcgcgagtggtgggctcttaaacctggagcccctcaacccaaggcaaatcaacaacatcaagacaacgcccgaggtcttgtccttccgggttacaaataccttggacccggcaacggactcgacaagggcgagccggtcaacgcagcagacgcggcggccctcgaacacgacaaggcctacgaccagcagctcaaggccggagacaacccgtacctcaagtacaaccacgccgacgccgagttccaggagcgcctcaaagaagatacgtcttttggtggcaacctcggccgagcagtcttccaggccaaaaagaggcttcttgaacctcttggtctcgttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgacggcactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 64 - DNA, 45 bp, Artificial Sequence
  Description of Artificial Sequence Synthetic polynucleotide
ccgctacccatccgcacgttggatggctgccgacggttatctacc

SEQ ID NO: 65 - DNA, 52 bp, Artificial Sequence
  Description of Artificial Sequence Synthetic polynucleotide
ccctggttctacgtggcggtttacagattacgagtcaggtatctggtgccaa

SEQ ID NO: 66 - DNA, 2274 bp, Artificial Sequence
  Description of Artificial Sequence Synthetic polynucleotide
ccgctacccatccgcacgttggatggctgccgacggttatctacccgattggctcgaggacaaccttagtgaaggaattcgcgagtggtgggctcttaaacctggagcccctcaacccaaggcaaatcaacaacatcaagacaacgcccgaggtcttgtccttccgggttacaaataccttggacccggcaacggactcgacaagggcgagccggtcaacgcagcagacgcggcggccctcgaacacgacaaggcctacgaccagcagctcaaggccggagacaacccgtacctcaagtacaaccacgccgacgccgagttccaggagcgcctcaaagaagatacgtcttttggtggcaacctcggccgagcagtcttccaggccaaaaagaggcttcttgaacctcttggtctcgttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgacggcactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaaaccgccacgtagaaccaggg

SEQ ID NO: 67 - DNA, 21 bp, Artificial Sequence
  Description of Artificial Sequence Synthetic polynucleotide
gctccctggttctacgtggcg

SEQ ID NO: 68 - DNA, 25 bp, Artificial Sequence
  Description of Artificial Sequence Synthetic polynucleotide
gactgcaggaattcggtacctcgct

SEQ ID NO: 69 - DNA, 2451 bp, Artificial Sequence
  Description of Artificial Sequence Synthetic polynucleotide
gattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccgctacccatccgcacgttggatggctgccgacggttatctacccgattggctcgaggacaaccttagtgaaggaattcgcgagtggtgggctcttaaacctggagcccctcaacccaaggcaaatcaacaacatcaagacaacgcccgaggtcttgtccttccgggttacaaataccttggacccggcaacggactcgacaagggcgagccggtcaacgcagcagacgcggcggccctcgaacacgacaaggcctacgaccagcagctcaaggccggagacaacccgtacctcaagtacaaccacgccgacgccgagttccaggagcgcctcaaagaagatacgtcttttggtggcaacctcggccgagcagtcttccaggccaaaaagaggcttcttgaacctcttggtctcgttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgacggcactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgcccca

SEQ ID NO: 70 - DNA, 51 bp, Artificial Sequence
  Description of Artificial Sequence Synthetic polynucleotide
ccgcgcgcaccggcaagatacgtatggctcctggaaagaagaggcctgtag

SEQ ID NO: 71 - DNA, 52 bp, Artificial Sequence
  Description of Artificial Sequence Synthetic polynucleotide
cgattgacgtgtccggtgggttacagattacgagtcaggtatctggtgccaa

SEQ ID NO: 72 - DNA, 1864 bp, Artificial Sequence
  Description of Artificial Sequence Synthetic polynucleotide
ccgcgcgcaccggcaagatacgtatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgacggcactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaacccaccggacacgtcaatcg

SEQ ID NO: 73 - DNA, 2041 bp, Artificial Sequence
  Description of Artificial Sequence Synthetic polynucleotide
gattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccgcgcgcaccggcaagatacgtatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgacggcactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgcccca

SEQ ID NO: 74 - DNA, 51 bp, Artificial Sequence
  Description of Artificial Sequence Synthetic polynucleotide
caatcactcgacgtagactgtatcctgttaagatggcttcaggtggtggcg

SEQ ID NO: 75 - DNA, 56 bp, Artificial Sequence
  Description of Artificial Sequence Synthetic polynucleotide
acgcggttagaggcggttgctagcttacagattacgagtcaggtatctggtgccaa

SEQ ID NO: 76 - DNA, 1626 bp, Artificial Sequence
  Description of Artificial Sequence Synthetic polynucleotide
caatcactcgacgtagactgtatcctgttaagatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgacggcactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgcccca

SEQ ID NO: 77 - DNA, 20 bp, Artificial Sequence
  Description of Artificial Sequence Synthetic polynucleotide
ggcctccggggccttactca

SEQ ID NO: 78 - DNA, 20 bp, Artificial Sequence
  Description of Artificial Sequence Synthetic polynucleotide
tagcgcgggttccttccggt

SEQ ID NO: 79 - DNA, 1731 bp, Artificial Sequence
  Description of Artificial Sequence Synthetic polynucleotide
gacctttaattcaacccaacacaatatattatagttaaataagaattattatcaaatcatttgtatattaattaaaatactatactgtaaattacattttatttacaatcactcgacgtagactgtatcctgttaagatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgacggcactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgcccca

SEQ ID NO: 80 - DNA, 853 bp, Artificial Sequence
  Description of Artificial Sequence Synthetic polynucleotide
ccctggttctacgtggcggcctaggttgacagctagctcagtcctaggtataatgctagctactagagaaagaggagaaatactagatggaaaagaaaattacgggatataccacagttgacatctcacaatggcaccgcaaagaacacttcgaagcctttcaaagtgtcgcacaatgcacgtataaccagaccgttcaacttgacatcaccgcttttctgaaaacggtaaaaaaaaacaagcacaaattctatccggccttcattcacatcttggcccgccttatgaacgcccacccggagtttcgtatggctatgaaagatggtgagttggtcatttgggactctgtacatccatgctacacggtctttcacgaacagactgaaacttttagttcactgtggtcagaatatcatgatgactttcgccaatttttacatatttatagccaagatgtcgcatgctatggagaaaacttagcttacttccccaaggggtttattgagaatatgttctttgtctcggctaacccctgggttagctttacgtcgttcgaccttaatgtggcgaatatggataatttttttgcccctgtgtttacgatgggtaagtattacacccagggtgataaagtattgatgcctttggcgatccaagtccatcatgcggtctgtgacggatttcatgtaggtcgtatgttgaacgagcttcaacaatattgtgacgagtggcaagggggcgcctaataagaatcctccggcaaaaaagggcaaggtgtcaccaccctgccctttttctttaaaaccgaaaagattacttcgcgttatcctaggaacgtgcggatgggtagcgg

SEQ ID NO: 81 - DNA, 2232 bp, Artificial Sequence
  Description of Artificial Sequence Synthetic polynucleotide
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtccgcttaatggtgccgtccatctttatgctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 82 - protein, 743 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSPLNGAVHLYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 83 - DNA, 2232 bp, Artificial Sequence
  Description of Artificial Sequence Synthetic polynucleotide
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacagattgttatgaattcgttgaaggctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 84 - protein, 743 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQIVMNSLKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 85 - DNA, 56 bp, Artificial Sequence
  Description of Artificial Sequence Synthetic polynucleotide
cgctacccatccgcacgttccgtatggctgccgacggttatctacccgattggctc

SEQ ID NO: 86 - DNA, 48 bp, Artificial Sequence
  Description of Artificial Sequence Synthetic polynucleotide
cctggttctacgtggcggcttacagattacgagtcaggtatctggtgc

SEQ ID NO: 87 - DNA, 44 bp, Artificial Sequence
  Description of Artificial Sequence Synthetic polynucleotide
cgcaccggcaagatagccccgtatggctcctggaaagaagaggc

SEQ ID NO: 88 - DNA, 45 bp, Artificial Sequence
  Description of Artificial Sequence Synthetic polynucleotide
cgtgtccggtgggcccttacagattacgagtcaggtatctggtgc

SEQ ID NO: 89 - DNA, 47 bp, Artificial Sequence
  Description of Artificial Sequence Synthetic polynucleotide
acgtagactgtatcctgttaaggaagatggcttcaggtggtggcgca

SEQ ID NO: 90 - DNA, 47 bp, Artificial Sequence
  Description of Artificial Sequence Synthetic polynucleotide
agaggcggttgctagcggttacagattacgagtcaggtatctggtgc

(19) United States
(12) Patent Application Publication
(10) Pub. No.: US 2024/0141378 A1
(43) Pub. Date: May 2, 2024

(54) CONTROLLED EXPRESSION OF VIRAL PROTEINS

(71) Applicant: Voyager Therapeutics, Inc., Cambridge, MA (US)

(72) Inventors: Jeffrey Morley SLACK, Cambridge, MA (US); Christopher NGUYEN, Cambridge, MA (US); Peter SLADE, Milton, MA (US)

(21) Appl. No.: 18/279,819

(22) PCT Filed: Mar. 3, 2022
(86) PCT No.: PCT/US2022/018787

§ 371 Date: Aug. 31, 2023

Related U.S. Application Data
(60) Provisional application No. 63/155,922, filed on Mar. 3, 2021.
(60) Provisional application No. 63/155,921, filed on Mar. 3, 2021.

(51) Int. Cl. C12N 15/86

(52) U.S. Cl. CPC C12N 15/86; C12N 2710/14144; C12N 2750/14122; C12N 2750/14143; C12N 2750/14151; C12N 2800/105; C12N 2830/002

(57) ABSTRACT

The present disclosure describes methods and systems for use in the production of adeno-associated virus (AAV) particles, including recombinant adeno-associated virus (rAAV) particles. The production process and system use Baculoviral Expression Vectors (BEVs) and/or Baculoviral Infected Insect Cells (BIICs) in the production of AAV particles (e.g., rAAVs) which allow for the controlled expression of AAV structural (e.g., capsid) proteins, such as VP1, VP2, and VPS and the controlled expression of AAV nonstructural (e.g., replication) proteins, such as Rep78 and Rep52.

### RELATED APPLICATIONS

This application claims priority to U.S. Provisional Application 63/155,921 filed on Mar. 3, 2021, and U.S. Provisional Application 63/155,922 filed on Mar. 3, 2021, the entire contents of which are hereby incorporated by reference.

### REFERENCE TO THE SEQUENCE LISTING

The present application is being filed along with a Sequence Listing in electronic format. The Sequence Listing is provided as a file entitled VTJ-1550PC_SL.txt, created on Mar. 3, 2022, which is 129,262 bytes in size. The information in the electronic format of the sequence listing is incorporated herein by reference in its entirety.

### FIELD

The present disclosure describes methods and systems for use in the production of adeno-associated virus (AAV) particles, including recombinant adeno-associated virus (rAAV) particles. In certain embodiments, the production process and system use Spodoptera frugiperda insect cells (such as Sf9 or Sf21) as viral production cells (VPCs). The production process and system use Baculoviral Expression Vectors (BEVs) and/or Baculoviral Infected Insect Cells (BIICs) in the production of AAV particles (e.g., rAAVs) which allow for the controlled expression of AAV structural (e.g., capsid) proteins, such as VP1, VP2, and VP3 and the controlled expression of AAV nonstructural (e.g., replication) proteins, such as Rep78 and Rep52.

### BACKGROUND

AAVs have emerged as one of the most widely studied and utilized viral vectors for gene transfer to mammalian cells. See, e.g., Tratschin et al., Mol. Cell Biol., 5(11):3251-3260 (1985) and Grimm et al., Hum. Gene Ther., 10(15):2445-2450 (1999), the contents of which are each incorporated herein by reference in their entireties. Adeno-associated virus (rAAV) based gene therapy is a rapidly advancing technology involving therapeutic transgenes which are engineered with flanking inverted terminal repeats (ITRs) and then packaged into rAAV virion capsids. These rAAV capsids can target specific tissue cells and deliver the transgene payloads into the cells where they become semipermanent episomes expressing the therapeutic transgene products. Adeno-associated viral (AAV) vectors are promising candidates for therapeutic gene delivery and have proven safe and efficacious in clinical trials.

The key to this technology is the ability to produce rAAV capsids at large scale and with high potency such that many patients can be economically treated for targeted diseases. In recent years, rAAVs have been harnessed gene therapy vectors with several therapeutics being authorized for clinical use in the United States (Glybera™, Luxturna™, Zolgensma™). There have been 149 clinical trials of different rAAV drug products (Kuzmin et al., Nat Rev Drug Discov., 20(3):173-174 (2021)) and genetically modified capsid serotypes with improved tissue specificities and potencies are on the horizon for a new generation rAAV therapeutics (Deverman et al., Nat Biotechnol., 34(2):204-9 (2016); Nonnenmacher et al., Mol Ther Methods Clin Dev., 20:366-378 (2020)).

The baculovirus expression vector (BEV)/Sf9 insect cell platform has great potential to produce rAAV gene therapy products. Baculovirus expression vector systems (BEVS) are widely used to produce abundant recombinant proteins in cultured insect cells. This abundance is achieved by expressing the gene of interest (GOI) in recombinant BEVs under control of hyper-expressed polh or p10 promoters. BEVS have been successfully used to produce therapeutics, such as vaccines, e.g., Cervarix™ (HPV vaccine against cervical cancer), FluBlok® (an influenza subunit vaccine), and Covovax™ (SARS-CoV-2 vaccine).

However, challenges still exist translating the mammalian origin rAAV genes to equivalence in the insect cell line-based platform. There remains a need for improved baculovirus expression systems, particularly for the expression of AAV structural (e.g., capsid) proteins and AAV capsids, AAV nonstructural (e.g., replication) proteins, and corresponding AAV vectors (e.g., rAAV particles).

### SUMMARY

Provided herein are viral expression constructs (e.g., baculovirus expression constructs) comprising variant viral genomes (e.g., variant baculovirus genomes), as well as methods for efficient production of the same. Such viral expression constructs (e.g., baculovirus expression constructs provided herein are engineered for use in controlling the expression of AAV nonstructural (e.g., replication) proteins, such as Rep78 and Rep52, and/or the controlled expression of AAV structural proteins (e.g., capsid proteins), such as VP1, VP2 and VP3, during the production of recombinant adeno-associated viral (rAAV) particles.

In one aspect, provided herein is an AAV expression construct comprising (i) at least two Rep-coding regions, each comprising a nucleotide sequence encoding a Rep protein independently chosen from Rep52, Rep40, Rep68, or Rep78 protein, (ii) at least two VP coding regions comprising a nucleotide sequence encoding a VP protein chosen independently chosen from a VP1 protein, a VP2 protein, a VP3 protein, or a combination thereof, (iii) a transcriptional regulator binding sequence (e.g., a lac repressor sequence) and (iv) at least one regulator binding sequence (e.g., a lacO sequence), wherein the at least one regulator binding sequence is operably linked to the VP1 and/or VP2 sequence, and wherein AAV expression construct comprises a variant baculovirus genome.

In another aspect, provided herein is a cell (e.g., host cell, such as an insect cell) comprising a baculovirus expression construct described herein. The cell can be, e.g., a bacterial cell (e.g., E. coli), a mammalian cell (e.g., HEK293), or an insect cell (e.g., Sf9, Sf21).

In another aspect, provided herein is an AAV expression construct and/or AAV payload construct comprising the variant baculovirus genomes described herein, as well as AAV viral production systems comprising the same.

In another aspect, provided herein is a method of producing a recombinant AAV (rAAV) particle in an AAV viral production cell, as well as rAAV particles produced using the method, wherein the method comprises: (i) providing an AAV viral production system described herein, (ii) transfecting the AAV viral production system into an AAV viral production cell; (iii) exposing the AAV viral production cell to conditions which allow the AAV viral production cell to process the AAV expression construct and the AAV payload construct into rAAV particles; and, optionally, (iv) collecting the rAAV particles from the AAV viral production cell, e.g., an insect cell such as a Sf9 cell or a Sf21 cell.

In yet another aspect, provided herein are baculoviruses produced using the AAV expression constructs described herein, variant baculovirus genomes described herein, baculovirus expression constructs described herein, and cells described herein.

In yet another aspect, provided herein are compositions (e.g., pharmaceutical compositions) and kits comprising, e.g., the AAV expression constructs described herein, variant baculovirus genomes described herein, baculovirus expression constructs described herein, or AAV particles described herein.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

E1. An AAV expression construct comprising:
(i) at least two Rep-coding regions, each comprising a nucleotide sequence encoding a Rep protein independently chosen from Rep52, Rep40, Rep68, or Rep78 protein,
(ii) at least two VP coding regions comprising a nucleotide sequence encoding a VP protein chosen independently chosen from a VP1 protein, a VP2 protein, a VP3 protein, or a combination thereof,
(iii) at least one transcriptional regulator element coding sequence (e.g., a lac repressor sequence); and
(iv) at least one regulator binding sequence (e.g., a lacO sequence), wherein the at least one regulator binding sequence is operably linked to the VP1 and/or VP2 sequence, and wherein AAV expression construct comprises a variant baculovirus genome,
wherein the at least two Rep-coding regions and/or the at least two VP coding regions each comprise a different nucleotide sequence and/or is present in different location;
wherein the AAV expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from gta, egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.
   E2. The AAV expression construct of embodiment E1, wherein the variant baculovirus genome comprises a nucleotide sequence or a portion thereof from a baculovirus genome selected from Autographa californica multiple nucleopolyhedrovirus (AcMNPV) (e.g., an AcMNPV strain E2, C6, or HR3), Bombyx mori nucleopolyhedrovirus (BmNPV), Anticarsia gemmatalis nucleopolyhedrovirus (AgMNPV), Orgyia pseudotsugata nucleopolyhedrovirus (OpMNPV), or Thysanoplusia orichalcea nucleopolyhedrovirus (ThorMNPV).
   E3. The AAV expression construct of any one of embodiments E1-E2, wherein the variant baculovirus genome comprises a nucleotide sequence or a portion thereof from the AcMNPV (e.g., AcMNPV E2) baculovirus genome.
   E4. The AAV expression construct of any one of embodiments E1-E3, wherein the disruption results in inactivation of the non-essential gene (e.g., auxiliary and/or per os infectivity factor gene) or the regulatory region of the non-essential gene (e.g., promoter modification or insertion of heterologous DNA adjacent to non-essential gene).
   E5. The AAV expression construct of any one of embodiments E1-E4, wherein the disruption of the at least two non-essential genes is or comprises an insertion, deletion, substitution, or mutation (e.g., frame-shift mutation).
   E6. The AAV expression construct of any one of embodiments E1-E5, wherein the disruption of one or both of the at least two non-essential genes is present in the regulatory region of the non-essential gene (e.g., a promoter modification or insertion of heterologous DNA adjacent to non-essential gene).
   E7. The AAV expression construct of any one of embodiments E1-E6, wherein the variant baculovirus genome comprises a disruption of at least three, four, five, six, seven, eight, nine, or ten non-essential genes (e.g., auxiliary and/or per os infectivity factor genes), wherein the at least three, four, five, six, seven, eight, nine, or ten non-essential genes are independently chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
   E8. The AAV expression construct of any one of embodiments E1-E7, wherein the at least two non-essential genes comprise:
(i) v-cath and egt;
(ii) v-cath, egt, and SOD;
(iii) v-cath, gta, egt and SOD;
(iv) v-cath, gta, egt, SOD and p74;
(v) chiaA, v-cath, gta, egt, SOD and p74;
(vi) chiA, v-cath, egt, p26, p10, and p74;
(vii) chiA, v-cath, egt, p26, p10, p74, and SOD; or
(viii) chiA, v-cath, egt, p26, p10, p74, SOD, AcORF-91, and AcORF-108.
   E9. The AAV expression construct of any one of embodiments E1-E8, wherein the disruption comprises a deletion of a chiA gene, a v-cath gene, a p26 gene, a p10 gene, and/or a p74 gene, or a portion thereof.
   E10. The AAV expression construct of any one of embodiments E1-E9, wherein the disruption comprises an insertion of a heterologous sequence in the non-essential gene or adjacent region.
   E11. The AAV expression construct of any one of embodiments E1-E10, wherein the disruption comprises a one or more mutations in the non-essential gene or adjacent region.
   E12. The AAV expression construct of any one of embodiments E1-E11, wherein one or both of the at least two non-essential genes are present near (e.g., downstream or upstream) of a homologous repeat region hr1, hr2, hr3, hr4 or hr5, optionally hr5.
   E13. The AAV expression construct of any one of embodiments E1-E12, wherein the at least two Rep-coding regions each comprise a different nucleotide sequence and is present in different locations in the variant baculovirus genome.
   E14. The AAV expression construct of any one of embodiments E1-E13, wherein the at least two Rep-coding regions comprise a first Rep-coding region and a second Rep-coding region.
   E15. The AAV expression construct of embodiment E14, wherein the first Rep-coding region comprises a first a first open reading frame (ORF) comprising a start codon and a nucleotide sequence encoding a Rep78 protein and the second Rep-coding region comprises a second ORF comprising a start codon and a nucleotide sequence encoding a Rep52 protein.
   E16. The AAV expression construct of embodiments E14-E15, wherein the first Rep-coding region, the second Rep-coding region or both comprises an ATG start codon (e.g., a canonical start codon).
   E17. The AAV expression construct of embodiments E14-E15, wherein the first Rep-coding region, the second Rep-coding region or both comprises an ACG start codon, a CTG start codon, a TTG start codon, or a GTG start codon (e.g., a non-canonical start codon).
   E18. The AAV expression construct of any one of the preceding embodiments, wherein the first Rep-coding region comprises a nucleotide sequence encoding Rep78.
   E19. The AAV expression construct of any one of the preceding embodiments, wherein the first Rep-coding region comprises a nucleotide sequence encoding primarily a Rep78 protein, e.g., at least 50%, 60%, 70%, 80%, 90% or more Rep78 protein relative to a Rep52 protein.
   E20. The AAV expression construct of any one of the preceding embodiments, wherein, the first Rep-coding region comprises a nucleotide sequence encoding Rep78 only.
   E21. The AAV expression construct of any one of the preceding embodiments, wherein, the first Rep-coding region comprises a nucleotide sequence encoding Rep78 but not Rep52.
   E22. The AAV expression construct of any one of embodiments E14-E21, wherein the second Rep-coding comprises a nucleotide sequence encoding a Rep52 protein.
   E23. The AAV expression construct of any one of embodiments E14-E22, wherein the second Rep-coding comprises a nucleotide sequence encoding primarily a Rep52 protein, e.g., at least 50%, 60%, 70%, 80%, 90% or more Rep52 protein relative to a Rep78 protein.
   E24. The AAV expression construct of any one of embodiments E14-E23, wherein the second Rep-coding comprises a nucleotide sequence encoding a Rep52 protein only.
   E25. The AAV expression construct of any one of embodiments E14-E22, wherein the second Rep-coding comprises a nucleotide sequence encoding a Rep52 protein but not a Rep78 protein.
   E26. The AAV expression construct of any one of embodiments E14-E25, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) the second Rep-coding region comprises a nucleotide sequence encoding a Rep52 protein but not a Rep78 protein.
   E27. The AAV expression construct of any one of embodiments E14-E26, wherein the first Rep-coding region comprises the nucleotide sequence of SEQ ID NO: 143, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 143; or a nucleotide sequence having at least 10, 20, 50, 100, 150, 200, 250, 300, 350, 400, or 450 but no more than 500 modifications (e.g., substitutions) relative to SEQ ID NO: 143.
   E28. The AAV expression construct of any one of embodiments E14-E27, wherein the first Rep-coding region encodes the amino acid sequence of SEQ ID NO: 144; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 144; or an amino acid sequence comprising at least 1, 2, 3, 4, 5, 10, 15, or 20 but no more than 30 modifications (e.g., substitutions (e.g., conservative substitutions), insertions, or deletions) relative to the amino acid sequence of SEQ ID NO: 144.
   E29. The AAV expression construct of any one of embodiments E14-E28, wherein the second Rep-coding region comprises the nucleotide sequence of SEQ ID NO: 145, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 145; or a nucleotide sequence having at least 10, 20, 50, 100, 150, 200, 250, 300, 350, 400, or 450 but no more than 500 modifications (e.g., substitutions) relative to SEQ ID NO: 145.
   E30. The AAV expression construct of any one of embodiments E14-E29, wherein the second Rep-coding region encodes the amino acid sequence of SEQ ID NO: 146; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 146; or an amino acid sequence comprising at least 1, 2, 3, 4, 5, 10, 15, or 20 but no more than 30 modifications (e.g., substitutions (e.g., conservative substitutions), insertions, or deletions) relative to SEQ ID NO: 146.
   E31. The AAV expression construct of any one of the preceding embodiments, wherein, the first Rep-coding region, second Rep-coding region or both are codon optimized for an insect cell, (e.g., an sf9 or an sf21 cell).
   E32. The AAV expression construct of any one of the preceding embodiments, wherein the nucleotide sequence of the first Rep-coding region is operably linked to a first promoter.
   E33. The AAV expression construct of any one of embodiments E14-E32, wherein the nucleotide sequence of the second Rep-coding region is operably linked to a second promoter.
   E34. The AAV expression construct of E32 or E33, wherein the first promoter, second promoter, or both is selected from a polyhedrin (polh) promoter, a p10 promoter, a conotoxin (ctx) promoter, a gp64 promoter an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof
   E35. The AAV expression construct of any one of embodiment E32-E34, wherein the first promoter and the second promoter are the same.
   E36. The AAV expression construct of any one of embodiment E32-E34, wherein the first promoter and the second promoter are different.
   E37. The AAV expression construct of any one of embodiment E32-E35, the first promoter and the second promoter are each a polh promoter.
   E38. The AAV expression construct of embodiment E37, wherein the polh promoter comprises the nucleotide sequence of SEQ ID NO: 175; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 175; or a nucleotide sequence comprising at least one, two, three, four, five, six, or seven, but no more than ten modifications (e.g., substitutions) relative to SEQ ID NO: 175.
   E39. The AAV expression construct of any one of the preceding embodiments, wherein the first Rep-coding region or the second Rep-coding region comprises an expression-modifier sequence which decreases transcription initiation of the first Rep-coding region.
   E40. The AAV expression construct of E39, wherein the first Rep-coding region comprises between 3-100 nucleotides between the expression-modifier sequence and the start codon of the first Rep ORF; optionally between 3-25 nucleotides, between 3-10 nucleotides, or 3 nucleotides between the expression-modifier sequence and the start codon of the first Rep ORF.
   E41. The AAV expression construct of E39, wherein the expression-modifier sequence comprises a minicistron sequence.
   E42. The AAV expression construct of any one of embodiments E39-E41, the first Rep-coding region comprises a minicistron sequence, optionally wherein the minicistron sequence is present at the 5′ end of the first Rep-coding region.
   E43. The AAV expression construct of E42, wherein the minicistron insertion sequence is from a baculovirus gene, optionally a baculovirus gp64 gene.
   E44. The AAV expression construct of E43, wherein the minicistron insertion sequence comprises SEQ ID NO: 4 or 5; a nucleotide sequence at least 90%, 95%, 96%, 97%, 98%, or 99% identical to SEQ ID NO: 4 or 5; a nucleotide sequence comprising one, two, or three modifications (e.g., substitutions), but no more than four modifications (e.g., substitutions) relative to SEQ ID NO: 4 or 5; or a nucleotide sequence comprising one, two, or three, but no more than four different nucleotides relative to SEQ ID NO: 4 or 5.
   E45. The AAV expression construct of any one of embodiments E14-E44, which comprises in in 5′ to 3′ order: a polh promoter, a minicistron sequence, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein).
   E46. The AAV expression construct of any one of embodiments E14-E38, which comprises in 5′ to 3′ order: a polh promoter, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein).
   E47. The AAV expression construct of any one of embodiments E14-E46, which comprises in 5′ to 3′ order: a polh promoter and the second Rep-coding region comprising a nucleotide sequence encoding a Rep52 protein but not a Rep78 protein.
   E48. The AAV expression construct of any one of embodiments E14-47, which comprises:
(i) in 5′ to 3′ order: a polh promoter, a minicistron sequence, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) in 5′ to 3′ order: a polh promoter and the second Rep-coding region comprising a nucleotide sequence encoding a Rep52 protein but not a Rep78 protein.
   E49. The AAV expression construct of any one of embodiments E14-E48, wherein:
(i) in 5′ to 3′ order: a polh promoter and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) in 5′ to 3′ order: a polh promoter and the second Rep-coding region comprising a nucleotide sequence encoding a Rep52 protein but not a Rep78 protein.
   E50. The AAV expression construct of any one of embodiments E14-E49, wherein:
(i) the first Rep-coding region is present in first location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94; and
(ii) the second Rep-coding region is present in a second location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94; wherein the first locus and the second locus are different.
   E51. The AAV expression construct of any one of embodiments E14-E50, wherein the first Rep-coding region is present in SOD locus and the second Rep-coding region is present in the egt locus.
   E52. The AAV expression construct of ant one of embodiments E14-E50, wherein the first Rep-coding region is present in Tn7/polh locus and the second Rep-coding region is present in the egt locus.
   E53. The AAV expression construct of any one of embodiments E14-E50, wherein the first Rep-coding region is present in the SOD locus of the variant baculovirus genome and is operably linked to a polh promoter, and the second Rep-coding region is present in the egt locus of the variant baculovirus genome and is operably linked to a polh promoter.
   E54. The AAV expression construct of any one of embodiments E14-E51 or E53, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the SOD locus of the variant baculovirus genome; and
(ii) the second Rep-coding region comprises a nucleotide sequence encoding a Rep52 protein but not a Rep78 protein, wherein the second Rep-coding region is present in the egt locus of the variant baculovirus genome.
   E55. The AAV expression construct of any one of embodiments E14-E51 or E53-E54, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the SOD locus of the variant baculovirus genome and is operably linked to a polh promoter; and
(ii) the second Rep-coding region comprises a nucleotide sequence encoding a Rep52 protein but not a Rep78 protein, wherein the second Rep-coding region is present in the egt locus of the variant baculovirus genome and is operably linked to a polh promoter.
   E56. The AAV expression construct of any one of embodiments E14-E51 or E53-E55, which comprises:
(i) in 5′ to 3′ order a polh promoter, a minicistron sequence, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the SOD locus of the variant baculovirus genome; and
(ii) in 5′ to 3′ order: a polh promoter and the second Rep-coding region comprising a nucleotide sequence encoding a Rep52 protein but not a Rep78 protein, wherein the second Rep-coding region is present in the egt locus of the variant baculovirus genome.
   E57. The AAV expression construct of any one of embodiments E14-E51 or E53-E55, which comprises:
(i) in 5′ to 3′ order: a polh promoter and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the SOD locus of the variant baculovirus genome; and
(ii) in 5′ to 3′ order: a polh promoter and the second Rep-coding region comprising a nucleotide sequence encoding a Rep52 protein but not a Rep78 protein, wherein the second Rep-coding region is present in the egt locus of the variant baculovirus genome.
   E58. The AAV expression construct of any one of embodiments E1-E58, wherein the at least two VP-coding regions comprise a first VP-coding region and a second VP-coding region.
   E59. The AAV expression construct of any one of embodiments E1-E57, which further comprises a third VP-coding region.
   E60. The AAV expression construct of any one of the preceding embodiments, wherein the first VP-coding region comprises a nucleotide sequence encoding a VP1 protein.
   E61. The AAV expression construct of any one of embodiments E1-E60, wherein the first VP-coding region comprises a nucleotide sequence encoding primarily a VP1 protein, e.g., at least 50%, 60%, 70%, 80%, 90% or more VP1 protein relative to a VP2 protein and/or a VP3 protein.
   E62. The AAV expression construct of any one of embodiments E1-E61, wherein the first VP-coding region comprises a nucleotide sequence encoding a VP1 protein only.
   E63. The AAV expression construct of any one of embodiments E1-E61, wherein the first VP-coding region comprises a nucleotide sequence encoding a VP1 protein, but not a VP2 protein or a VP3 protein.
   E64. The AAV expression construct of any one of embodiments E1-E63, wherein the second VP-coding region comprises a nucleotide sequence encoding a VP2 protein.
   E65. The AAV expression contract of any one of embodiments E1-E64, wherein the second VP-coding region comprises a nucleotide sequence encoding:
(i) a VP2 protein and a VP3 protein;
(ii) primarily a VP2 protein, e.g., at least about 50%, 60%, 70%, 80%, 90% or more VP2 protein relative to a VP1 protein and/or a VP3 protein;
(iii) a VP2 protein only;
(iv) a VP2 protein, but not a VP1 protein or a VP3 protein.
   E66. The AAV expression construct of any one of embodiments E1-E65, comprising a third VP-coding region comprising a nucleotide sequence encoding
(i) primarily a VP3 protein;
(ii) a VP3 protein, but not a VP1 protein or a VP2 protein;
(iii) a VP3 protein only.
   E67. The AAV expression construct of any one of embodiments E1-E66, wherein the VP-coding regions encode an AAV1 capsid protein, an AAV2 capsid protein, an AAV3 capsid protein, an AAV4 capsid protein, an AAVS capsid protein, an AAV6 capsid protein, an AAV8 capsid protein, an AAV9 capsid protein, an AAVrh10 capsid protein or a variant of any of the aforesaid capsid proteins.
   E68. The AAV expression construct of any one of embodiments E1-E67, wherein the VP-coding regions encode an AAV5 capsid protein or variant thereof, or an AAV9 capsid protein or variant thereof.
   E69. The AAV expression construct of any one of embodiments 1-66, wherein the VP-coding region encodes a VP1 protein comprising the amino acid sequence of any of SEQ ID NOs: 149, 150, 153, 155, 156, 82, 161, 164, 84, 168, 171, or 174, or an amino acid sequence at least 70%, 75%, 80%, 85%, 90%, 95%, 96%, 97%, 98%, or 99% identical to any of the aforesaid amino acid sequences.
   E70. The AAV expression construct of any one of embodiments 1-67, wherein the VP-coding region encodes a VP2 protein comprising amino acids 138-736 or SEQ ID NOs: 171, 149, or 150; amino acids 138-743 of SEQ ID NOs: 153, 155, 156, 82, 161, 164, 84; or amino acids 137-724 of SEQ ID NO: 174.
   E71. The AAV expression construct of any one of embodiments 1-68, wherein the VP-coding region encodes a VP3 protein comprising amino acids 203-736 of SEQ ID NOs: 171, 149, or 150; amino acids 203-743 of SEQ ID NOs: 153, 155, 156, 82, 161, 164, 84; or amino acids 193-724 of SEQ ID NO: 174.
   E72. The AAV expression construct of any one of the preceding embodiments, wherein one, two, or all of the first VP-coding region, the second VP-coding region, and the third VP-coding region is codon optimized for an insect cell, e.g., a Spodoptera frugiperda insect cell.
   E72. The AAV expression construct of any one of the preceding embodiments, wherein the first VP-coding region is operably linked to a promoter.
   E73. The AAV expression construct of any one of the preceding embodiments, wherein the second VP-coding region is operably linked to a promoter.
   E74. The AAV expression construct of any one of the preceding embodiments, wherein the third VP-coding region is operably linked to a promoter.
   E75. The AAV expression construct of any one of embodiments E72-E74, wherein the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof.
   E76. The AAV expression construct of any one of embodiments E72-E75, wherein the promoter is chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   E77. The AAV expression construct of any one of embodiments E72-E75, wherein the promoter of the first VP-coding region comprises is selected from: polh, ΔIE-1, p10, Δp10, and variations or derivatives thereof.
   E78. The AAV expression construction of embodiment, E77 wherein the promoter of the first VP-coding region comprises a p10 promoter sequence.
   E79. The AAV expression construct of any one of embodiments E72-E75, wherein the promoter of the second VP-coding region comprises is selected from: polh, ΔIE-1, p10, Δp10, and variations or derivatives thereof.
   E80. The AAV expression construction of embodiment, E79 wherein the promoter of the second VP-coding region comprises a p10 promoter sequence.
   E81. The AAV expression construct of any one of embodiments E72-E75, wherein the promoter of the second VP-coding region comprises is selected from: polh, ΔIE-1, p10, Δp10, and variations or derivatives thereof.
   E82. The AAV expression construction of embodiment, E81, wherein the promoter of the second VP-coding region comprises a p10 promoter sequence.
   E83. The AAV expression construct of any one of embodiments E77-E82, wherein the p10 promoter comprises the nucleotide sequence of SEQ ID NO: 176; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 200; or a nucleotide sequence comprising at least one, two, three, four, five, six, or seven, but no more than ten modifications (e.g., substitutions) relative to SEQ ID NO: 176.
   E84. The AAV expression construct of any one of the preceding embodiments, wherein the at least one transcriptional regulator element coding region comprising a first regulator element ORF which comprises a start codon and a nucleotide sequence encoding one or more transcriptional regulator elements.
   E85. The AAV expression construct of any one of the preceding claims, wherein the at least one regulator element is a Lac repressor (LacR) protein.
   E86. The AAV expression construct of any one of the preceding claims, wherein the at least one regulator element is an engineered Lac repressor protein (eLacr).
   E87. The AAV expression construct of any one of the E85-E86, wherein the LacR coding region is codon optimized for a Spodoptera frugiperda insect cell.
   E88. The AAV expression construct of E86, wherein the engineered LacR protein is encoded by a polynucleotide comprising a nucleotide sequence selected from SEQ ID NO: 6 or a nucleotide sequence which is at least 75%, at least 80%, at least 85%, at least 90%, at least 95%, at least 97%, or at least 99% identical to SEQ ID NO: 6.
   E89. The AAV expression construct of E86, wherein the engineered LacR protein is encoded by a polynucleotide comprising a nucleotide sequence selected from SEQ ID NO: 9 or a nucleotide sequence which is at least 75%, at least 80%, at least 85%, at least 90%, at least 95%, at least 97%, or at least 99% identical to SEQ ID NO: 9.
   E90. The AAV expression construct of E86, wherein the engineered LacR protein is encoded by a polynucleotide comprising a nucleotide sequence selected from SEQ ID NO: 10 or a nucleotide sequence which is at least 75%, at least 80%, at least 85%, at least 90%, at least 95%, at least 97%, or at least 99% identical to SEQ ID NO: 10.
   E91. The AAV expression construct of any one of the preceding embodiments, wherein, the at least one regulator binding sequence is a Lac Operator (LacO) sequence.
   E92. The AAV expression construct of embodiment E91, comprising at least one LacO sequence operably linked to the VP1 coding sequence.
   E92. The AAV expression construct of embodiments E91-92, comprising two LacO sequences operably linked to the VP1 coding sequence.
   E93. The AAV expression construct of E91-E92, comprising three LacO sequences operably linked to the VP1 coding sequence.
   E94. The AAV expression construct of any one of embodiments E91-E93, comprising at least one LacO sequence operably linked to the VP2 coding sequence.
   E95. The AAV expression construct of any one of embodiments E91-E94, comprising two LacO sequences operably linked to the VP2 coding sequence.
   E96. The AAV expression construct of any one of embodiments E91-E94, comprising three LacO sequences operably linked to the VP2 coding sequence.
   E97. The AAV expression vector of any one of embodiments E91-E96 wherein at least one regulator binding sequence is a LacO sequence comprising a nucleotide sequence selected from SEQ ID NO: 14 or a nucleotide sequence which is at least 75%, at least 80%, at least 85%, at least 90%, at least 95%, or at least 99% identical to SEQ ID NO: 14.
   E98. The AAV expression construct of any one of embodiments E91-E97, wherein the VP1-coding region comprises a promoter sequence, and at least one LacO sequence within 5-100 nucleotides from at least one end of the promoter of the VP1-coding region.
   E99. The AAV expression construct of embodiment E98, wherein the VP1-coding region comprises a LacO sequence which is 5-100 nucleotides upstream of the 5′ end of the promoter, and a LacO sequence which is 5-100 nucleotides downstream of the 3′ end of the promoter.
   E100. The AAV expression construct of embodiments E98-E99, wherein the promoter of the VP1 coding region is p10.
   E101. The AAV expression construct of any one of embodiments E91-E100, wherein VP1-coding region comprises at least one LacO-p10-LacO expression control sequence which comprises the nucleotide sequence of SEQ ID NO: 40 or a nucleotide sequence which is at least 75%, at least 80%, at least 85%, at least 90%, at least 93%, at least 95%, at least 97%, or at least 99% identical to SEQ ID NO: 40.
   E102. The AAV expression construct of any one of embodiments E91-E101, wherein the VP1-coding region comprises a LacO sequence at the 3′ end of the coding sequence.
   E103. The AAV expression construct of any one of the preceding embodiments, wherein the VP2-coding region comprises a promoter sequence, and at least one LacO sequence within 5-100 nucleotides from at least one end of the promoter of the VP2-coding region.
   E104. The AAV expression construct of any one of embodiments E91-E103, wherein the VP2-coding region comprises a LacO sequence which is 5-100 nucleotides upstream of the 5′ end of the promoter, and a LacO sequence which is 5-100 nucleotides downstream of the 3′ end of the promoter.
   E105. The AAV expression construct of any one of embodiments E91-E104, wherein the VP2-coding region comprises a p10 promoter sequence, a first LacO sequence which is 5-100 nucleotides upstream of the 5′ end of the p10 promoter, and a second LacO sequence which is 5-100 nucleotides downstream of the 5′ end of the p10 promoter.
   E106. The AAV expression construct of any one of embodiment E91-E105, wherein VP2-coding region comprises at least one LacO-p10-LacO expression control sequence which comprises the nucleotide sequence of SEQ ID NO: 40 or a nucleotide sequence which is at least 75%, at least 80%, at least 85%, at least 90%, at least 93%, at least 95%, at least 97%, or at least 99% identical to SEQ ID NO: 40.
   E107. The AAV expression construct of any one of embodiments E91-E106, wherein the VP2-coding region comprises a LacO sequence at the 3′ end of the coding sequence.
   E108. The AAV expression construct of any one of embodiments E1-E107, wherein
(i) the first Rep-coding region is located in a first location of the baculovirus genome, and the second Rep-coding region is located in a second location of the baculovirus genome which is different from the first location of the baculovirus genome;
(ii) the first VP-coding region is located in a third location of the baculovirus genome, and the second VP-coding region is located in a fourth location of the baculovirus genome which is different from the third location of the baculovirus genome, and the third VP-coding region is located in a fifth location of the baculovirus genome which is different from the third location and the fourth location of the baculovirus genome;
(iii) the regulator element coding region is located in a sixth location of the baculovirus genome.
   E109. The AAV expression construct of embodiment E108, wherein the first location, the second location, the third location, the fourth location, the fifth location, and/or the sixth location of the baculovirus genome are selected from: egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94 gene locus.
   E110. The AAV expression construct of any one of embodiments E1-E109, wherein the first VP-coding region comprises a nucleotide sequence encoding VP1 and is located in the ChiA/v-cath gene locus of the baculovirus genome.
   E111. The AAV expression construct of any one of embodiments E1-E109, wherein the first VP-coding region comprises a nucleotide sequence encoding VP1 and is located in the gta gene locus of the baculovirus genome.
   E112. The AAV expression construct of any one of embodiments E1-E111, wherein the second VP-coding region comprises a nucleotide sequence encoding VP2 and is located in the ChiA/v-cath gene locus of the baculovirus genome.
   E113. The AAV expression construct of any one of embodiments E1-E111, wherein the second VP-coding region comprises a nucleotide sequence encoding VP2 and is located in the gta gene locus of the baculovirus genome.
   E114. The AAV expression construct of any one of embodiments E1-E113, wherein the third VP-coding region comprises a nucleotide sequence encoding VP3 and is located in the Tn7/polh gene locus of the baculovirus genome.
   E115. The AAV expression construct of any one of embodiments E1-E113, the third VP-coding region comprises a nucleotide sequence encoding VP3 and is located in the SOD gene locus of the baculovirus genome.
   E116. The AAV expression construct of any one of embodiments E1-E115, wherein the regulator element coding region comprises a nucleotide sequence encoding one or more regulator elements and is located in the p74 gene locus of the baculovirus genome.
   E117. The AAV expression construct of any one of embodiments E1-E115, wherein the regulator element coding region encodes the LacR of embodiments E85-E90.
   E118. The AAV expression construct of embodiment E117, wherein the LacR coding region comprises at least one promoter sequence selected from: polh, ΔIE-1, p10, Δp10, gp64 and variations or derivatives thereof.
   E119. The AAV expression construct of any one of embodiments E116-118, wherein the LacR coding sequence comprises a gp64/polh promoter.
   E120. The AAV expression construct of any one of embodiments E116-119, is wherein the LacR coding sequence present near (e.g., downstream or upstream) a homologous repeat region (hr1, hr2, hr3, hr4 hr5).
   E121. The AAV expression construct of embodiment E120, wherein the homologous repeat region is hr5.
   E122. A recombinant baculovirus genome comprising:
(i) a Rep78-coding region comprising a polh promoter located in the Tn7/polh gene locus of the baculovirus genome;
(ii) a Rep52-coding region comprising a polh promoter located in the egt gene locus of the baculovirus genome;
(iii) a VP1-coding region comprising a p10 promoter and at least one LacO sequence 5′ to the p10 promoter located in the ChiA/v-cath gene locus of the baculovirus genome;
(iv) a VP2-coding region comprising a p10 promoter located in the gta gene locus of the baculovirus genome;
(v) a VP3-coding region comprising a p10 promoter located in the Tn7/polh gene locus of the baculovirus genome; and
(vi) a LacR-coding region comprising a gp64/pol10 promoter located in the p74 gene locus of the baculovirus genome downstream of homologous repeat region hr5.
   E123. The AAV expression construct of embodiment E122, wherein the VP1 coding region comprises a 5′ to 3′ a LacO-p10-LacO promoter sequence and a VP1 coding region.
   E124. The AAV expression construct of embodiments E122-E123, wherein the VP1 coding region comprises a LacO sequence at the 3′ end of the nucleotide sequence encoding VP1.
   E125. The AAV expression construct of E122-E124, wherein the VP2 coding region comprises at least LacO sequence 5′ of the p10 promoter.
   E126. The AAV expression construct of any one of embodiments E122-E125, wherein the VP2 coding region comprises a LacO-p10-LacO promoter sequence.
   E127. The AAV expression construct of any one of embodiments E122-E126, wherein the VP2 coding region comprises a LacO sequence at the 3′ end of the nucleotide sequence encoding VP2.
   E128. An AAV expression construct comprising:
(i) a Rep78-coding region comprising a polh promoter located in the SOD gene locus of the baculovirus genome;
(ii) a Rep52-coding region comprising a polh promoter, located in the egt gene locus of the baculovirus genome;
(iii) a VP1-coding region comprising a p10 promoter and at least one LacO sequence 5′ to the p10 promoter located in the gta gene locus of the baculovirus genome;
(iv) a VP2-coding region comprising a p10 promoter located in the ChiA/v-cath gene locus of the baculovirus genome;
(v) a VP3-coding region comprising a p10 promoter located in the SOD gene locus of the baculovirus genome; and
(vi) a LacR-coding region comprising a gp64/polh promoter located in the p74 gene locus of the baculovirus genome downstream of homologous repeat region hr5.
   E129. The AAV expression construct of embodiment E128, wherein the VP1 coding region comprises a LacO-p10-LacO promoter sequence.
   E130. The AAV expression construct of embodiments E128-E129, wherein the VP1 coding region comprises a LacO sequence at the 3′ end of the nucleotide sequence encoding VP1.
   E131. The AAV expression construct of embodiments E128-130, wherein the VP2 coding region comprises at least LacO sequence 5′ of the p10 promoter.
   E132. The AAV expression construct of any one of embodiments E128-131, wherein the VP2 coding region comprises a LacO-p10-LacO promoter sequence.
   E133. The AAV expression construct any one of embodiments E128-132, wherein the VP2 coding region comprises a LacO sequence at the 3′ end of the nucleotide sequence encoding VP2.
   E134. An AAV expression construct comprising:
(i) a Rep78-coding region comprising a polh promoter located in the SOD gene locus of the baculovirus genome;
(ii) a Rep52-coding region comprising a polh promoter, located in the egt gene locus of the baculovirus genome;
(iii) a first VP-coding region located in the gta gene locus of the baculovirus genome, wherein the first VP-coding region comprises a nucleotide sequence encoding VP1-coding region, a p10 promoter and at least one LacO sequence 5′ to the p10 promoter;
(iv) a second VP-coding region located in the SOD locus of the baculovirus genome, wherein the second VP-coding region comprises a nucleotide sequence encoding VP2 and VP3, and a p10 promoter; and
(vi) a LacR-coding region comprising a gp64/polh promoter located in the p74 gene locus of the baculovirus genome downstream of the homologous repeat region hr5.
   E135. The AAV expression construct of embodiment E134, wherein the first VP-coding region comprises a LacO-p10-LacO promoter sequence.
   E136. The AAV expression construct of embodiments E134-E135, wherein the first VP-coding region comprises a LacO sequence at the 3′ end of the nucleotide sequence encoding VP1.
   E137. An AAV viral production system comprising an AAV expression construct of any one of embodiments E1-E136, and an AAV payload construct which comprises a transgene payload.
   E138. The AAV viral production system of embodiment E137, comprising an AAV viral production cell which comprises the AAV expression construct and the AAV payload construct.
   E139. The AAV viral production system of embodiment E138, wherein the cell is an insect cell, for example, an Sf9 cell or an Sf21 cell.
   E140. A method of producing one, two, three, four, or all of a Rep78 protein, a Rep52 protein, a VP1 protein, a VP protein, and/or a VP3 protein, the method comprising:
(i) providing a cell comprising the AAV expression construct of any one of embodiments E1-E139;
(ii) incubating the cell under conditions suitable to produce the one, two, three, four, or all of the Rep78 protein, the Rep52 protein, the VP1 protein, the VP protein, and/or the VP3 protein.
   E141. The method of embodiment E140, further comprising, prior to step (i), introducing the AAV expression construct into the cell.
   E142. A method of producing an AAV particle, the method comprising:
(i) providing a cell comprising the AAV expression construct of any one of embodiments E1-E139 and an AAV payload construct;
(ii) incubating the cell under conditions suitable to produce the AAV particle; thereby producing the AAV particle.
   E143. The method of embodiment E142, wherein the AAV payload expression construct comprises a payload coding region comprising a nucleotide sequence encoding a payload wherein the AAV payload expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.
   E144. The method of embodiments E142-143, wherein the payload coding region is present in a location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
   E145. The method of any one of embodiments E142-E144, wherein the payload coding region comprises a start codon and a nucleotide sequence encoding the payload.
   E146. The method of any one of embodiments E142-E145, wherein the encoded payload comprises a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E147. The method of any one of embodiments E140-E146, wherein an inducer element (e.g., IPTG) is introduced at a concentration between about 1.0 μM to about 20 μM, between about 1.0 μM to about 5.0 μM, between about 2.0 μM to about 3.0 μM, between about 5.0 μM to about 15.0 μM, or at a concentration of about 10.0 μM.
   E148. A cell comprising the AAV expression construct of any one of embodiments E1-E136.
   E149. A cell comprising the AAV production system of any one of embodiments E137-139.
   E150. The cell of embodiments E148-E149, wherein the cell is an insect cell (e.g., an Sf9 cell or an Sf21).
   E151. A composition comprising the AAV expression construct of any one of embodiments E1-E136, and a carrier.
   E152. An AAV particle made by the method of any one of embodiments E142-E147.
   E153. A kit comprising the AAV expression construct of any one of embodiments E1-E136.

### Other Embodiments

O1. An AAV expression construct comprising: a first Rep-coding region comprising a first Rep open reading frame (ORF) which comprises a start codon and a nucleotide sequence encoding one or more AAV Rep78 proteins; a second Rep-coding region comprising a second Rep ORF which comprises a start codon and a nucleotide sequence encoding one or more AAV Rep52 proteins; a first VP-coding region comprising a first VP ORF which comprises a start codon and a nucleotide sequence encoding one or more AAV VP1 proteins; a second VP-coding region comprising a second VP ORF which comprises a start codon and a nucleotide sequence encoding one or more AAV VP2 proteins; and a third VP-coding region comprising a third VP ORF which comprises a start codon and a nucleotide sequence encoding one or more AAV VP3 proteins.
O2. The AAV expression construct of embodiment O1, wherein the first Rep-coding region comprises a nucleotide sequence encoding Rep78 only.
O3. The AAV expression construct of any one of embodiments O1-2, wherein at least a portion of the first Rep-coding region is codon optimized from a reference Rep-coding nucleotide sequence; optionally wherein the first Rep-coding region is codon optimized for an insect cell; optionally a Spodoptera frugiperda insect cell.
O4. The AAV expression construct of any one of embodiments O1-3, wherein the first Rep-coding region comprises one or more expression control regions which comprise one or more promoter sequences; optionally wherein the expression control region of the first Rep-coding region comprises at least one promoter sequence selected from: polH, ΔIE-1, p10, Δp10, and variations or derivatives thereof; optionally wherein the expression control region of the first Rep-coding region comprises at least one polH promoter sequence.
O5. The AAV expression construct of any one of embodiments O1-4, wherein the first Rep-coding region comprises a polH promoter sequence, and wherein the first Rep ORF comprises a nucleotide sequence encoding Rep78 only.
O6. The AAV expression construct of any one of embodiments O1-5, wherein the first Rep-coding region comprises one or more expression-modifier sequences 5′ of the first Rep ORF, and wherein the one or more expression-modifier sequences decreases transcription and/or translation initiation at the start codon of the first Rep ORF.
O7. The AAV expression construct of embodiment O6, wherein the first Rep-coding region comprises between 3-100 nucleotides between the expression-modifier sequence and the start codon of the first Rep ORF; optionally between 3-25 nucleotides, between 3-10 nucleotides, or 3 nucleotides between the expression-modifier sequence and the start codon of the first Rep ORF.
O8. The AAV expression construct of any one of embodiments O1-7, wherein the second Rep-coding region comprises a nucleotide sequence encoding Rep52 only.
O9. The AAV expression construct of any one of embodiments O1-8, wherein at least a portion of the second Rep-coding region is codon optimized from a reference Rep-coding nucleotide sequence; optionally wherein the second Rep-coding region is codon optimized for an insect cell; optionally a Spodoptera frugiperda insect cell.
O10. The AAV expression construct of any one of embodiments O1-9, wherein the second Rep-coding region comprises one or more expression control regions which comprise one or more promoter sequences; optionally wherein the expression control region of the second Rep-coding region comprises at least one promoter sequence selected from: polH, ΔIE-1, p10, Δp10, and variations or derivatives thereof; optionally wherein the expression control region of the second Rep-coding region comprises at least one polH promoter sequence.
O11. The AAV expression construct of any one of embodiments O1-10, wherein the second Rep-coding region comprises a polH promoter sequence, and wherein the second Rep ORF comprises a nucleotide sequence encoding Rep52 only.
O12. The AAV expression construct of any one of embodiments O1-11, wherein the second Rep-coding region comprises one or more expression-modifier sequences 5′ of the second Rep ORF, and wherein the one or more expression-modifier sequences decreases transcription and/or translation initiation at the start codon of the second Rep ORF.
O13. The AAV expression construct of embodiment O12, wherein the second Rep-coding region comprises between 3-100 nucleotides between the expression-modifier sequence and the start codon of the second Rep ORF; optionally between 3-25 nucleotides, between 3-10 nucleotides, or 3 nucleotides between the expression-modifier sequence and the start codon of the second Rep ORF.
O14. The AAV expression construct of any one of embodiments O6-7 or O12-13, wherein the one or more expression-modifier sequences comprises a minicistron sequence.
O15. The AAV expression construct of embodiment O14, wherein the minicistron insertion sequence is from a baculovirus gene; optionally a baculovirus gp64 gene.
O16. The AAV expression construct of embodiment O14, wherein the minicistron insertion sequence comprises SEQ ID NO: 4, or a sequence which is at least 90% identical, at least 93% identical, at least 95% identical, at least 97% identical, or at least 99% identical with SEQ ID NO: 4.
O17. The AAV expression construct of embodiment O14, wherein the minicistron insertion sequence comprises SEQ ID NO: 5, or a sequence which is at least 90% identical, at least 93% identical, at least 95% identical, at least 97% identical, or at least 99% identical with SEQ ID NO: 5.
O18. The AAV expression construct of any one of embodiments O1-17, wherein the first VP-coding region comprises a nucleotide sequence encoding VP1 only.
O19. The AAV expression construct of any one of embodiments O1-18, wherein at least a portion of the first VP-coding region is codon optimized from a reference VP-coding nucleotide sequence; optionally wherein the first VP-coding region is codon optimized for an insect cell; optionally a Spodoptera frugiperda insect cell.
O20. The AAV expression construct of any one of embodiments O1-19, wherein the first VP-coding region comprises one or more expression control regions which comprise one or more promoter sequences; optionally wherein the expression control region of the first VP-coding region comprises at least one promoter sequence selected from: polH, ΔIE-1, p10, Δp10, and variations or derivatives thereof; optionally wherein the expression control region of the first VP-coding region comprises at least one p10 promoter sequence.
O21. The AAV expression construct of any one of embodiments O1-20, wherein the first VP-coding region comprises a p10 promoter sequence, and wherein the first VP ORF comprises a nucleotide sequence encoding VP1 only.
O22. The AAV expression construct of any one of embodiments O1-21, wherein the second VP-coding region comprises a nucleotide sequence encoding VP2 only.
O23. The AAV expression construct of any one of embodiments O1-22, wherein at least a portion of the second VP-coding region is codon optimized from a reference VP-coding nucleotide sequence; optionally wherein the second VP-coding region is codon optimized for an insect cell; optionally a Spodoptera frugiperda insect cell.
O24. The AAV expression construct of any one of embodiments O1-23, wherein the second VP-coding region comprises one or more expression control regions which comprise one or more promoter sequences; optionally wherein the expression control region of the second VP-coding region comprises at least one promoter sequence selected from: polH, ΔIE-1, p10, Δp10, and variations or derivatives thereof; optionally wherein the expression control region of the second VP-coding region comprises at least one p10 promoter sequence.
O25. The AAV expression construct of any one of embodiments O1-24, wherein the second VP-coding region comprises a p10 promoter sequence, and wherein the second VP ORF comprises a nucleotide sequence encoding VP2 only.
O26. The AAV expression construct of any one of embodiments O1-25, wherein the third VP-coding region comprises a nucleotide sequence encoding VP3 only.
O27. The AAV expression construct of any one of embodiments O1-26, wherein at least a portion of the third VP-coding region is codon optimized from a reference VP-coding nucleotide sequence; optionally wherein the third VP-coding region is codon optimized for an insect cell; optionally a Spodoptera frugiperda insect cell.
O28. The AAV expression construct of any one of embodiments O1-27, wherein the third VP-coding region comprises one or more expression control regions which comprise one or more promoter sequences; optionally wherein the expression control region of the third VP-coding region comprises at least one promoter sequence selected from: polH, ΔIE-1, p10, Δp10, and variations or derivatives thereof; optionally wherein the expression control region of the third VP-coding region comprises at least one p10 promoter sequence.
O29. The AAV expression construct of any one of embodiments O1-28, wherein the third VP-coding region comprises a p10 promoter sequence, and wherein the third VP ORF comprises a nucleotide sequence encoding VP3 only.
O30. The AAV expression construct of any one of embodiments O1-29, further comprising at least one sequence encoding at least one element of a transcriptional regulatory system, wherein the transcriptional regulatory system comprises: (i) one or more regulator elements, (ii) one or more regulator binding sequences; and (iii) one or more inducer elements.
O31. The AAV expression construct of embodiment O30, wherein the AAV expression construct comprises regulator coding region comprising a first regulator ORF which comprises a start codon and a nucleotide sequence encoding one or more regulator elements; optionally wherein the one or more regulator elements have a high affinity for binding to the one or more regulator binding sequences.
O32. The AAV expression construct of embodiment O31, wherein the AAV expression construct comprises at least two regulator binding sequences; and wherein the one or more regulator elements have a high affinity for binding to two regulator binding sequences simultaneously; optionally wherein the simultaneous binding of the regulator element to the two regulator binding sequences results in the formation of a loop structure in a nucleotide sequence between the two regulator binding sequences.
O33. The AAV expression construct of any one of embodiments O31-32, wherein the one or more inducer elements of the transcriptional regulatory system can bind to the one or more regulator elements, thereby reducing the affinity of the regulator element for binding to the one or more regulator binding sequences; optionally wherein the binding of the inducer element to the regulator element causes a conformational change in the regulator element, thereby reducing the affinity of the regulator element for binding to the one or more regulator binding sequences.
O34. The AAV expression construct of any one of embodiments O31-33, wherein at least one regulator element is a Lac repressor (LacR) protein; optionally an engineered Lac repressor protein (eLacr).
O35. The AAV expression construct of embodiment O34, wherein the engineered LacR protein is encoded by a polynucleotide comprising a nucleotide sequence selected from SEQ ID NO: 6 or a nucleotide sequence which is at least 75%, at least 80%, at least 85%, at least 90%, at least 95%, at least 97%, or at least 99% identical to SEQ ID NO: 6.
O36. The AAV expression construct of embodiment O34, wherein the engineered LacR protein is encoded by a polynucleotide comprising a nucleotide sequence selected from SEQ ID NO: 9 or a nucleotide sequence which is at least 75%, at least 80%, at least 85%, at least 90%, at least 95%, at least 97%, or at least 99% identical to SEQ ID NO: 9.
O37. The AAV expression construct of embodiment O34, wherein the engineered LacR protein is encoded by a polynucleotide comprising a nucleotide sequence selected from SEQ ID NO: 10 or a nucleotide sequence which is at least 75%, at least 80%, at least 85%, at least 90%, at least 95%, at least 97%, or at least 99% identical to SEQ ID NO: 10.
O38. The AAV expression construct of any one of embodiments O31-37, wherein at least one regulator binding sequence is a Lac Operator (LacO) sequence.
O39. The AAV expression construct of embodiment O38, wherein at least one regulator binding sequence is a LacO sequence which comprises a nucleotide sequence selected from SEQ ID NO: 14 or a nucleotide sequence which is at least 75%, at least 80%, at least 85%, at least 90%, at least 95%, or at least 99% identical to SEQ ID NO: 14.
O40. The AAV expression construct of any one of embodiments O31-39, wherein at least one inducer element is selected from lactose, allolactose and isopropyl-β-D-thiogalactose (IPTG); optionally wherein at least one inducer element is IPTG.
O41. The AAV expression construct of any one of embodiments O31-40, wherein the first VP-coding region comprises a promoter sequence, and at least one regulator binding sequence within 5-100 nucleotides from at least one end of the promoter of the first VP-coding region; optionally wherein the first VP-coding region comprises a promoter sequence, a regulator binding sequence which is 5-100 nucleotides upstream of the 5′ end of the promoter, and a regulator binding sequence which is 5-100 nucleotides downstream of the 3′ end of the promoter.
O42. The AAV expression construct of embodiment O41, wherein the first VP-coding region comprises a p10 promoter sequence, a first LacO regulator binding sequence which is 5-100 nucleotides upstream of the 5′ end of the p10 promoter, and a second LacO regulator binding sequence which is 5-100 nucleotides downstream of the 5′ end of the p10 promoter; optionally wherein the first VP ORF of the first VP-coding region comprises a nucleotide sequence encoding VP1 only.
O43. The AAV expression construct of any one of embodiments O31-42, wherein the second VP-coding region comprises a promoter sequence, and at least one regulator binding sequence within 5-100 nucleotides from at least one end of the promoter of the second VP-coding region; optionally wherein the second VP-coding region comprises a promoter sequence, a regulator binding sequence which is 5-100 nucleotides upstream of the 5′ end of the promoter, and a regulator binding sequence which is 5-100 nucleotides downstream of the 3′ end of the promoter.
O44. The AAV expression construct of embodiment O43, wherein the second VP-coding region comprises a p10 promoter sequence, a first LacO regulator binding sequence which is 5-100 nucleotides upstream of the 5′ end of the p10 promoter, and a second LacO regulator binding sequence which is 5-100 nucleotides downstream of the 5′ end of the p10 promoter; optionally wherein the second VP ORF of the second VP-coding region comprises a nucleotide sequence encoding VP2 only.
O45. The AAV expression construct of any one of embodiments O41-44, comprising at least one LacO-p10-LacO expression control sequence, wherein the LacO-p10-LacO expression control sequence comprises the nucleotide sequence of SEQ ID NO: 40, or a nucleotide sequence which is at least 75%, at least 80%, at least 85%, at least 90%, at least 93%, at least 95%, at least 97%, or at least 99% identical to SEQ ID NO: 40.
O46. The AAV expression construct of any one of embodiments O41-44, wherein the first VP-coding region comprises at least one LacO-p10-LacO expression control sequence which comprises the nucleotide sequence of SEQ ID NO: 40 or a nucleotide sequence which is at least 75%, at least 80%, at least 85%, at least 90%, at least 93%, at least 95%, at least 97%, or at least 99% identical to SEQ ID NO: 40; and/or wherein the second VP-coding region comprises at least one LacO-p10-LacO expression control sequence which comprises the nucleotide sequence of SEQ ID NO: 40 or a nucleotide sequence which is at least 75%, at least 80%, at least 85%, at least 90%, at least 93%, at least 95%, at least 97%, or at least 99% identical to SEQ ID NO: 40.
O47. The AAV expression construct of any one of embodiments O1-46, wherein the AAV expression construct comprises a recombinant baculovirus genome (i.e., bacmid).
O48. The AAV expression construct of embodiment O47, wherein the first Rep-coding region is located in a first location of the baculovirus genome, wherein the second Rep-coding region is located in a second location of the baculovirus genome which is different from the first location of the baculovirus genome, wherein the first VP-coding region is located in a third location of the baculovirus genome, wherein the second VP-coding region is located in a fourth location of the baculovirus genome which is different from the third location of the baculovirus genome, and wherein the third VP-coding region is located in a fifth location of the baculovirus genome which is different from the third location and the fourth location of the baculovirus genome.
O49. The AAV expression construct of any one of embodiments O31-46, wherein the AAV expression construct comprises a recombinant baculovirus genome (i.e., bacmid), wherein the first Rep-coding region is located in a first location of the baculovirus genome, wherein the second Rep-coding region is located in a second location of the baculovirus genome which is different from the first location of the baculovirus genome, wherein the first VP-coding region is located in a third location of the baculovirus genome, wherein the second VP-coding region is located in a fourth location of the baculovirus genome which is different from the third location of the baculovirus genome, wherein the third VP-coding region is located in a fifth location of the baculovirus genome which is different from the third location and the fourth location of the baculovirus genome, and wherein the Oregulator coding region is located in a sixth location of the baculovirus genome.
O50. The AAV expression construct of any one of embodiments O48-49, wherein the first location, the second location, the third location, the fourth location, the fifth location, and the sixth location of the baculovirus genome are selected from: chiA/v-catch gene locus, egt gene locus, gta gene locus, p74 gene locus, and Tn7/polH gene locus.
O51. The AAV expression construct of any one of embodiments O48-50, wherein the first Rep-coding region comprises a nucleotide sequence encoding Rep78 only and is located in the Tn7/polH gene locus of the baculovirus genome; and/or wherein the second Rep-coding region comprises a nucleotide sequence encoding Rep52 only and is located in the egt gene locus of the baculovirus genome; and/or wherein the first VP-coding region comprises a nucleotide sequence encoding VP1 only and is located in the ChiA/v-cath gene locus of the baculovirus genome; and/or wherein the second VP-coding region comprises a nucleotide sequence encoding VP2 only and is located in the gta gene locus of the baculovirus genome; and/or wherein the third VP-coding region comprises a nucleotide sequence encoding VP3 only and is located in the Tn7/polH gene locus of the baculovirus genome; and/or wherein the regulator coding region comprises a nucleotide sequence encoding one or more regulator elements and is located in the p74 gene locus of the baculovirus genome.
O52. An AAV viral production system comprising an AAV expression construct and an AAV payload construct which comprises a transgene payload; wherein the AAV expression construct as an AAV expression construct of any one of embodiments O1-51.
O53. The AAV viral production system of embodiment O52, wherein the AAV viral production system comprises an AAV viral production cell which comprises the AAV expression construct and the AAV payload construct; optionally wherein the AAV viral production cell is an insect cell; optionally a Sf9 cell or a Sf21 cell.
O54. A method of expressing AAV Rep78, Rep52, VP1, VP2, and/or VP3 proteins in an AAV viral production cell, the method comprising: (i) providing an AAV expression construct of any one of embodiments O1-51; (ii) transfecting the AAV expression construct into an AAV viral production cell; (iii) and exposing the AAV viral production cell to conditions which allow the AAV viral production cell to process the Rep-coding regions into corresponding AAV Rep78 and/or Rep52 proteins and/or to process the VP-coding regions into corresponding AAV VP1, VP2 and/or VP3 proteins; optionally wherein the AAV viral production cell is an insect cell; optionally a Sf9 cell or a Sf21 cell.
O55. An Rep78, Rep52, VP1, VP2, and/or VP3 AAV protein produced by the method of embodiment O54.
O56. A method of producing recombinant adeno-associated virus (rAAV) particle in an AAV viral production cell, the method comprising: (i) providing an AAV viral production system of embodiment O52 or embodiment O53; (ii) transfecting the AAV viral production system into at least one AAV viral production cell within a bioreactor; (iii) exposing the at least one AAV viral production cell to conditions within the bioreactor which allow the AAV viral production cell to process the AAV expression construct and the AAV payload construct into rAAV particles; and, optionally, (iv) collecting the rAAV particles from the at least one AAV viral production cell; optionally wherein the AAV viral production cell is an insect cell; optionally a Sf9 cell or a Sf21 cell.
O57. The method of embodiment O56, wherein the AAV viral production system comprises at least one payloadBIIC comprising the AAV payload construct which comprises the transgene payload, and at least one expressionBIIC comprising the AAV expression construct; and wherein the payloadBIIC and the expressionBIIC are introduced into the bioreactor at a ratio (v/v) of between about 1:1 to 1:12; optionally between about 1:1 to 1:6; optionally between about 1:3 to 1:6; optionally about 1:4. 058. The method of embodiment O56 or embodiment O57, wherein an inducer element (e.g., IPTG) is introduced into the bioreactor at a concentration between about 1.0 μM to about 20 PM; optionally between about 5.0 μM to about 15.0 μM; optionally about 10.0 μM.
O59. A recombinant adeno-associated virus (rAAV) particle produced by the method of any one of embodiments O56-58.
O60. A pharmaceutical composition comprising the rAAV particle of embodiment O59 and a pharmaceutically acceptable excipient

### BRIEF DESCRIPTION OF THE FIGURES

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIGS. 1A-1G present graphical representations of constructs for Lac inducible Cap baculoviruses. FIG. 1A shows the common RepCap Baculovirus construct design. FIG. 1B is a graphical representation of LacRepCap Baculovirus constructs. The RepCap Baculovirus had all foreign genes inserted into a mini-AttTn7 transposition site while the LacRepCap Baculovirus had additional foreign genes ligated into an engineered I-CeuI REN site in the v-cath (VC) gene locus, an FseI REN site in the gta (global transactivator locus) and an AvrII REN site in the egt (ecdysosteroid UDP-glycosyltransferase) locus. In the circular 141 kbp Baculovirus used, minniAttTn7, I-CeuI, FseI and AvrII were separated by 31 kbp, 72 kbp, 23 kbp and 15 kbp respectively. FIG. 1C shows the common overlapping VP1, VP2, VP3 ORFs expressed by very late (VL) p10 promoter in the RepCap Baculovirus construct. VP1 and VP2 had non-canonical CTG and ACG start codons. FIG. 2 D shows the LacRepCap Baculovirus having VP1, VP2 and VP3 ORFs separately cloned with ATG start codons and p10 promoters. The p10 promoters of VP1 and VP2 were flanked by LacO spaced 188 bp apart. FIG. 2E shows the baculovirus having overlapping Rep78/Rep52 ORFs expressed by VL polyhedrin (polh) promoters. Rep78 had a non-canonical CTG start codon Rep52 an ATG start codon. The LacRepCap Baculovirus had two copies of LacR in the virus with polh promoters. The copy of LacR in the Tn7 locus also had an early (E) and late (L) OpMNPV gp64 promoter upstream of the VL polh promoter. The intent was to ensure early and abundant expression of LacR to ensure repression of VP1 and VP2 was consistent and continuous. FIG. 2F shows the LacR gene in the egt locus expressed from a polh promoter. FIG. 2G shows the second LacR gene designed with a hybrid early/late/very late baculovirus promoter to increase LacR abundance prior to the burst of very late gene promoter transcription.

FIG. 2A presents a graphical representation of a transcriptional regulatory system of the present disclosure which includes an inducible regulator element (e.g., homotetrameric LacR protein) bound to a regulator binding region (e.g., LacO nucleotide sequence) on each side of a p10 promoter, thereby constraining the p10 promoter into a transcriptional-repressing loop.

FIG. 2B presents a graphical representation of a transcriptional regulatory system of the present disclosure which includes an inducer element (e.g., IPTG) bound to an inducible regulator element (e.g., homotetrameric LacR protein), thereby preventing the regulator element from binding to the regulator binding regions (e.g., LacO), such that transcription from the p10 protein can proceed.

FIG. 3 presents a gel column showing separation of a polh-NLS-LacR insert from a polh-NLS-LacR-pUC57 plasmid.

FIG. 4A and FIG. 4B present gel columns showing the results of Colony PCR screening for LacR insertion into AvrII-cut bacmids.

FIG. 5 presents gel columns showing the results of REN digestion analysis for orientation of LacR insertion into AvrII-cut bacmids.

FIG. 6 presents a gel column showing separation of a LacO-p10-LacO-VP1 insert from LacO-p10-LacO-VP1-pUC57 plasmids and pUC57 fragments.

FIG. 7A, FIG. 7B and FIG. 7C present gel columns showing the results of Colony PCR screening for LacO-p10-LacO-VP1 insertion into the I-CeuI-cut 639 Bacmids.

FIG. 8A and FIG. 8B present gel columns showing the results of Western Blot analysis for LacR repression in Colony 1095.

FIG. 9A presents a gel column showing the results of Colony PCR screening for FseI-LacO-p10-LacO-VP2-AClef12-ACgta-FseI insertion into FseI-cut 1095 Bacmids. FIG. 9B presents a gel column showing the results of JS61-JS91 (715 bp), JS124-JS92 (1199 bp) and JS124-JS91 (3262 bp) PCR screening of Colonies 1186 and 1191.

FIG. 10 presents a gel column showing the results of Colony PCR screening for opgp64-polh-NLS-LacR insertion into AvrII-cut 1249 Plasmids.

FIG. 11 presents a gel column showing the results of JS95-JS42 (1037 bp), JS124-JS92 (1199 bp) and JS17-JS92 (1092 bp) PCR screening of Colony 1260.

FIG. 12 presents a graphical representation of certain components in Bacmid 1260.

FIG. 13A presents a Western blot analysis for AAV Cap proteins (VP1, VP2, and VP3) for AA654/AA656 co-infection into sf9 cells under various BIIC Ratio and IPTG study concentrations. FIG. 13B presents crude cell lysate qPCR titers for ITR-SEAP-GFP-ITR for AA654/AA656 co-infection into sf9 cells under various BIIC Ratio and IPTG study concentrations.

FIG. 14A presents a Western blot analysis for AAV Cap proteins (VP1, VP2, and VP3) for AA654/AA656 co-infection into sf9 cells under various BIIC Ratio and IPTG study concentrations. Titers generated at ITR-SEAP-GFP:LacRepCap co-infection ratios 1:1 (open triangle), 1:3 (black diamond) and 1:6 (open circle) are shown. Cells were lysed and AAV capsids were purified by sucrose cushion ultracentrifugation. These AAV samples used to transduce HEK 293T cells and the alkaline phosphatase activity was measured after 4 days. FIG. 14B presents clarified cell lysate qPCR titers for ITR-SEAP-GFP-ITR for AA654/AA656 co-infection into sf9 cells under various BIIC Ratio and IPTG study concentrations.

FIG. 15 presents results related to AA654/AA656 co-infection into sf9 cells under various BIIC Ratio and IPTG study concentrations, including alkaline phosphatase activity (i.e., SEAP potency) testing of resulting AAV particles transduced onto 293 HEK cells relative to AAV sample genome titer for ITR-SEAP-GFP-ITR from the AA654/AA656 co-infection. ITR-SEAP-GFP:LacRepCap co-infection ratios 1:1 (open triangle), 1:3 (black diamond) and 1:6 (open circle) are shown. The graph is split scaled to emphasize the potency values from capsids produced at IPTG concentrations between 0 uM and 10 uM.

FIGS. 16A-16B presents graphical representations of LacR regulation of LacO-p10-VP1 expression. FIG. 16A is graphical representation of the LacR-LacOVP1 baculovirus construct with a polh promoter LacR in the egt locus and lacO-p10 promoter VP1 in the VC locus. Sf9 cells were infected at 10 moi with the recombinant LacR-LacOVP1 baculovirus construct. FIG. 16B is a plot of the relative abundances of ECL proteins (quantified using ImageJ software).

FIGS. 17A-17C present graphical representations of the time course of Cap and Lac expression in baculovirus infected cells. Sf9 cells were infected at 10 MOI with LacRepCap baculovirus. Cells were collected at different hours post infection from 0 hpi to 50 hpi. Cell proteins were fractionated by SDS-PAGE and then Western blot probed with anti-capsid monoclonal antibody and anti-lac repressor antibody (FIG. 17A). FIG. 17B is a plot of the ECL signals from the Western detected proteins (quantified using ImageJ software). FIG. 17C is a bar graph of the calculated capsid VP ratios.

FIGS. 18A-18D present graphical representations of the IPTG induction of VP1 and VP2 expression. Sf9 cells were infected with LacRepCap baculovirus or RepCap baculovirus in presence of different concentrations of IPTG. Cells were collected at 72 hpi and their proteins were fractionated by SDS-PAGE and Western blot probed with anti-Capsid monoclonal antibody. FIGS. 18A and 18B show the relative capsid abundances in LacRepCap and RepCap infected cells, respectively, as observed by Western ECL signals (quantified using ImageJ software). FIG. 18C shows the capsid abundances for LacRepCap baculovirus infected cells graphed relative to IPTG concentration. FIG. 18D is a bar graph of the calculated capsid ratios for the LacRepCap baculovirus and the RepCap baculovirus.

FIGS. 19A-19C present graphical representations of the optimization of ITR and LacRepCap co-infection ratios to maximize AAV titers. Sf9 cells were co-infected with an ITR SEAP-GFP baculovirus and LacRepCap baculoviruses at different co-infection ratios in the presence of 0 uM and 200 uM IPTG. Cell lysate protein samples were fractionated by SDS-PAGE and Western blot probed with anti-capsid antibody (FIG. 19A). FIG. 19B is a bar graph of the crude cell lysate titers for ITR-SEAP-GFP-ITR determined by Q-PCR. FIG. 19C shows bar graphs of the estimated capsid ratios under different concentrations IPTG.

FIGS. 20A-20F present graphical representations of effect of IPTG on total AAV Capsid protein abundance in cells and in purified AAV capsids. Sf9 cells were co-infected with ITR and LacRepCap baculoviruses at co-infection ratios of 1:1, 1:3 and 1:6 in the presence of different concentrations of IPTG. Protein samples were obtained from cell lysates and from sucrose cushion ultracentrifugation pellet fractions of cell lysates. All samples were fractionated by SDS-PAGE and Western blot probed with anti-capsid monoclonal antibody. FIGS. 20A and 20B are images of the Western blots of the cell lysates and sucrose cushion ultracentrifugation pellet fractions of cell lysates, respectively. Immunolabelled capsid proteins were detected by ECL and quantified by using ImageJ software. Total capsid protein abundances in cell lysates (FIG. 20C) and sucrose cushion pellets (FIG. 20D) were plotted relative to IPTG concentration for each co-infection group; 1:1 (open triangle), 1:3 (black diamond) and 1:6 (open circle). FIGS. 20E and 20F are bar graphs of the estimated capsid ratios for the 1:6 co-infection for the cell lysate samples and sucrose cushion samples, respectively.

FIGS. 21A-21C present graphical representations of the comparison of capsid ratios before and after affinity purification. Sf9 cells were cultured at 800 ml scale and co-infected with ITR-SEAP-GFP and LacRepCap baculoviruses at a 1:6 co-infection ratio in the presence of 0 uM, 2 uM, 10 uM and 50 uM IPTG. Capsids were affinity purified and then further sucrose cushion purified. FIG. 21A shows the analysis of the total capsid proteins in cell lysates as analyzed by Western blot and quantified from ECL images using ImageJ software. FIGS. 21B and 20C show the analysis of the affinity purified capsids and affinity/sucrose cushion purified capsids, respectively, as analyzed by CE-SDS.

FIGS. 22A-22C present graphical representations of the percent full of affinity purified capsids, and the potency of affinity purified compared to affinity/sucrose cushion purified capsids on HEK 293 cells. Affinity purified and affinity/sucrose cushion purified AAV capsids derived from ITR-SEAP-GFP/LacRepCap baculovirus co-infection of Sf9 cells were used to transduce HEK 293T cells. The alkaline phosphatase activity was measured after 4 days. Potency was measured as alkaline phosphatase activity relative to AAV viral genome (vg) added to HEK 293T cells and is plotted against IPTG concentration for affinity purified AAV (FIG. 22A) and for affinity/sucrose cushion purified AAV (FIG. 22B). Affinity purified capsids were also subjected to SEC-MALS analysis and the percent full (ITR-SEAP-GFP genomes) in capsids relative to IPTG concentration is shown (FIG. 22C).

FIG. 23 presents a graphical representation of certain components in Bacmids AA879, AA886, and AA887.

FIG. 24 presents a Western blot analysis for AAV Rep proteins (Rep78 and Rep52), AAV Cap proteins (VP1, VP2, and VP3), and LacI proteins from Bacmids AA879, AA886, and AA887 under various IPTG study concentrations.

FIG. 25 presents a Western blot analysis for AAV Rep proteins (Rep78 and Rep52), AAV Cap proteins (VP1, VP2, and VP3), and LacI proteins from Bacmid AA887 under various IPTG study concentrations.

FIG. 26 presents a gel column analysis of AAV Cap proteins (VP1, VP2, and VP3) for Bacmids AA900 to AA905.

FIG. 27 presents a graphical representation of certain components in Bacmid AA904.

FIG. 28A presents a Coomassie Stain gel column analysis of AAV Cap proteins (VP1, VP2, and VP3) for AAV particles produced using Bacmids AA904 and AA935. FIG. 28B presents results related to AA904/AA935 co-infection into sf9 cells under various IPTG study concentrations, including alkaline phosphatase activity (i.e., SEAP potency) testing of resulting AAV particles transduced onto 293 HEK cells relative Coomassie Stain gel column results for AAV VP3 proteins.

FIG. 29 presents a graphical representation of certain components in the bacmids of the present application.

FIGS. 30A-30B present graphical representations of the passage stability study on AAV titer measured by Q-PCR. FIG. 30A is a bar graph of the AAV titer after 6 passages under IPTG concentrations of 0 uM and 1000 uM IPTG. FIG. 30B presents the Western blot analysis of the AAV Rep proteins (Rep78 and Rep52), and AAV Cap proteins (VP1, VP2, and VP3) under IPTG concentrations of 0 uM and 1000 uM.

FIGS. 31A-31B present graphical representation of the VP1 to VP3 capsid protein ration expression of bacmid constructs comprising two LacOs (FIG. 31A) in comparison to constructs with three LacOs (FIG. 31B).

FIGS. 32A-32B present graphical representation of the ratio of VP1, VP2 and VP3 capsid proteins expressed under differing concentration of IPTG from cell lysates (FIG. 32A) and from sucrose cushion purified capsids (FIG. 32B). The VP ratios were compared to two prior art constructs (Constructs A and B).

FIG. 33 is a graphical representation of the Q-PCR determined AAV titers from capsids expressed under differing concentration of IPTG from cell lysates. The AAV titers were compared to two prior art constructs (Constructs A and B).

FIG. 34 is a graphical representation of the Q-PCR determined AAV titers from capsids expressed under differing concentration of IPTG from sucrose cushion purified capsids. The AAV titers were compared to two prior art constructs (Constructs A and B).

### DETAILED DESCRIPTION

### I. Adenovirus-Associated Viruses

### Overview

Adeno-associated viruses (AAVs) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species. Dependovirus require co-infection of another DNA virus type such as an adenovirus, herpesvirus, or papillomavirus (Blacklow et al., J Exp Med., 125(5): 755-65 (1967); Buller et al., J Virol., 40(1):241-7 (1981) Ogston et al., J Virol., 74(8):3494-504 (2000), which are hereby incorporated by reference in their entirety). The parvoviruses and other members of the Parvoviridae family are generally described in Kenneth I. Berns, “Parvoviridae: The Viruses and Their Replication,” Chapter 69 in Fields Virology (3d Ed. 1996), which is hereby incorporated by reference in its entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the content of which is incorporated herein by reference in its entirety as related to AAV9/hu.14, insofar as it does not conflict with the present disclosure) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

rAAV gene therapeutics generally involve expressing AAV replicase (Rep) and capsid (Cap) genes in presence of a therapeutic transgene which is flanked by AAV inverted terminal repeat sequences (ITRs). The result is a recombinant rAAV (rAAV) with single stranded DNA transgene packaged into a rAAV capsid. The rAAV is inoculated into the patient where it infects (transduces) targeted cell types. The AAV capsid ensures tissue specificity and transgene delivery into the cell nucleus where it is converted into a stable double stranded DNA episome from which the transgene encoded therapeutic gene product is expressed.

An early method to produce rAAV therapeutics involved triple transfecting an ITR transgene plasmid, a RepCap plasmid and a helper plasmid into immortalized human embryonic kidney (HEK) 293T cells (Xiao et al., J Virol., 72(3):2224-32 (1998), which is hereby incorporated by reference in its entirety). The Large T antigen transformed HEK 293T cell line was useful for experimental rAAV therapeutic production but is prohibited from human therapeutics. Triple transfection was not intended for large scale production of rAAV therapeutics and is plagued by limited cell culture production capacity, high cost for plasmid DNA manufacture and the potential to introduce endotoxins or undesired human pathogens into the drug product (Wright, J F., Gene Ther., 15(11):840-8 (2008), which is hereby incorporated by reference in its entirety).

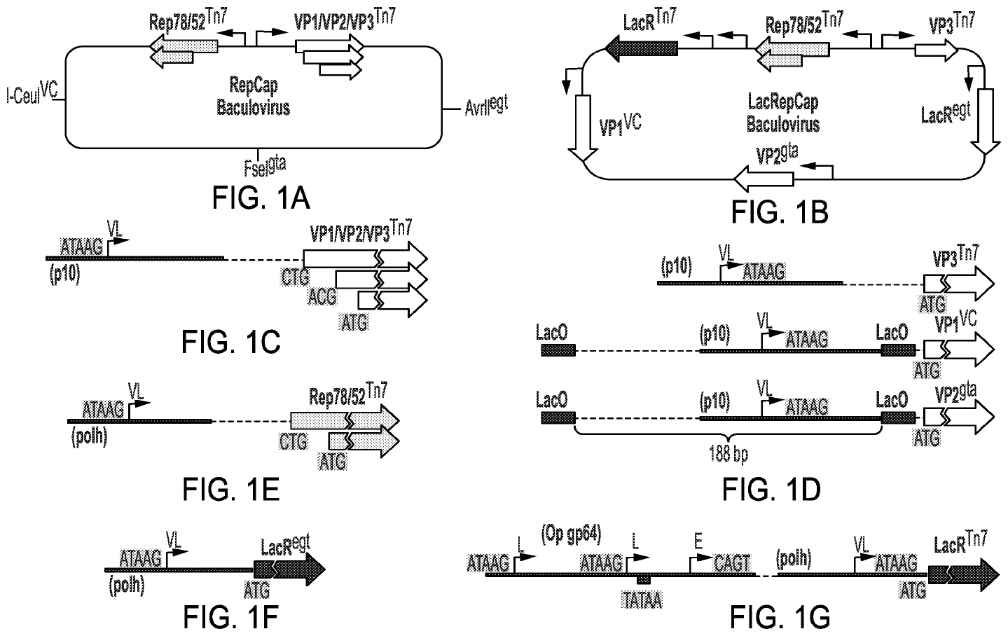

The application of BEV systems to produce rAAV therapeutics was first described by Urabe and later improved by Smith (Urabe et al., Hum Gene Ther., 13(16):1935-43 (2002); Smith et al., Mol Ther., 17(11):1888-96 (2009), which are hereby incorporated by reference in their entirety). The BEV system is ideal for larger scale production of rAAV therapeutics with unlimited cell culture production capacity, economically amplified BEV inoculums and absence of endotoxins or human potential pathogens. The Smith method to produce rAAV in the BEV system involves co-infection of a RepCap BEV with an ITR transgene BEV (Smith et al., Mol Ther., 17(11):1888-96 (2009), which is hereby incorporated by reference in its entirety). The RepCap expressing BEV is engineered with Rep and Cap genes pointing in opposite directions and expressed from very late polh and p10 baculovirus promoters (FIG. 1A). Rep78 and Rep52 proteins are translated from a common ORF with Rep78 having a non-canonical non-ATG translational start codon ensuring downstream translation the Rep52. The Cap gene encodes for VP1 (81 kDa), VP2 (67 kDa), and VP3 (61 kDa) Cap proteins on a common ORF (FIG. 1). The Cap gene also includes an out of frame ORF for a 21 kDa assembly-activating protein (AAP). The AAP is essential for capsid assembly in multiple rAAV serotypes (Sonntag et al., Proc Natl Acad Sci USA., 107(22):10220-5 (2010); Maurer et al., Cell Rep., 23(6):1817-1830 (2018), which are hereby incorporated by reference in their entirety). In the BEV system, VP1, VP2 and AAP protein ORFs have non-canonical, non-ATG translational start codons. Abundances of translated VP1, VP2 and AAP proteins are less than VP3 which has a canonical ATG translational start codon.

A challenge of producing potent rAAV capsids using the BEV system has been optimizing VP capsid ratios. The ideal VP1:VP2:VP3 ratios for rAAV capsids are undefined have been reported as 1:1:20 (Rose et al., J Virol., 8(5):766-70 (1971), which is hereby incorporated by reference in its entirety), 1:1:10 (Urabe et al., Hum Gene Ther., 13(16):1935-43 (2002); Venkatakrishnan et al., J Virol., 87(9):4974-84 (2013), which are hereby incorporated by reference in their entirety) and 1:1:8 (Johnson et al., J. Virol., 8(6):860-63 (1971); Kronenberg et al., EMBO Rep., 2(11):997-1002 (2001), which are hereby incorporated by reference in their entirety). Varying the ratio of VPs affects the titers and potency of assembled rAAV capsids (Bosma et al., Gene Ther., 25(6):415-424 (2018), which is hereby incorporated by reference in its entirety). Methods for changing VP ratios produced using BEVs have most involved the modulation of the translational context of VP1 (Kondratov et al., Mol Ther., 25(12):2661-2675 (2017); Bosma et al., Gene Ther., 25(6):415-424 (2018), which are hereby incorporated by reference in their entirety).

Accordingly, provided herein are baculovirus expression constructs (BEV) which, instead of expressing the AAV proteins (e.g., capsid proteins and/or replication proteins) from a common ORF, the protein ORFs are separated to be expressed independently in a single BEV construct. The ORFs of one or more of the VPs are be placed under a regulator element (e.g., an E. coli lac repressor (LacR)) providing inducible regulation in the context of the BEV expression system. For example, the expression of VP1 and/or VP2 can be decreased using regulator elements allowing for expression of VP1, VP2 and VP3 in a controlled ratio (e.g., 1:1:10) from a single BEV expression system. The same method can also be applied to the expression of AAV nonstructural proteins (e.g., replication proteins, such as Rep78 and Rep52).

Also provided herein are improved methods for expressing AAV capsid proteins, such as VP1, VP2 and VP3, and/or AAV nonstructural (e.g., replication) proteins, such as Rep78 and Rep52, during the production of recombinant adeno-associated viral (rAAV) particles. The method allows for the controlled expression of the different proteins from a single BEV expression system.

### II. Viral Constructs for Expression of Viral Proteins

The viral production system of the present disclosure comprises one or more viral expression constructs which can be transfected/transduced into a viral production cell (e.g., Sf9). In certain embodiments, a viral expression construct or a payload construct of the present disclosure can be a bacmid, also known as a baculovirus plasmid or recombinant baculovirus genome. In certain embodiments, a viral expression construct of the present disclosure can be a baculovirus expression vector (BEV). In certain embodiments, a viral expression construct of the present disclosure can be a BIIC which includes a BEV. As used herein, the term “expressionBac” or “Rep/Cap Bac” refers to a bacmid (such as a BEV) comprising a viral expression construct and/or viral expression region. Viral production cells (e.g., Sf9 cells) may be transfected with expressionBacs and/or with BIICs comprising expressionBacs.

In certain embodiments, the viral expression region comprises a protein-coding nucleotide sequence and at least one expression control sequence for expression in a viral production cell. In certain embodiments, the viral expression region comprises a protein-coding nucleotide sequence operably linked to least one expression control sequence for expression in a viral production cell. In certain embodiments, the viral expression construct contains parvoviral genes under control of one or more promoters. Parvoviral genes can comprise nucleotide sequences encoding non-structural AAV replication proteins, such as Rep genes which encode Rep52, Rep40, Rep68, or Rep78 proteins, e.g., a combination of Rep78 and Rep52. Parvoviral genes can comprise nucleotide sequences encoding structural AAV proteins, such as Cap genes which encode VP1, VP2, and VP3 proteins.

The viral production system of the present disclosure is not limited by the viral expression vector used to introduce the parvoviral functions into the virus replication cell. The presence of the viral expression construct in the virus replication cell need not be permanent. The viral expression constructs can be introduced by any means known, for example by chemical treatment of the cells, electroporation, or infection.

Viral expression constructs of the present disclosure may comprise any compound or formulation, biological or chemical, which facilitates transformation, transfection, or transduction of a cell with a nucleic acid. Exemplary biological viral expression constructs comprise plasmids, linear nucleic acid molecules, and recombinant viruses comprising baculovirus. Exemplary chemical vectors comprise lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and, Philiport and Scluber, eds. Liposomes as tools in Basic Research and Industry. CRC Press, Ann Arbor, Mich. (1995), the contents of which are each incorporated herein by reference in their entireties as related to viral expression constructs and uses thereof, insofar as they do not conflict with the present disclosure.

In certain embodiments, the viral expression construct is an AAV expression construct which comprises one or more nucleotide sequences encoding non-structural AAV replication proteins, structural AAV capsid proteins, or a combination thereof. In certain embodiments, the viral expression region is an AAV expression region of an expression construct which comprises one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may encode the components of a Parvoviral capsid with incorporated Gly-Ala repeat region, which may function as an immune evasion sequence, as described in US Patent Application 20110171262, the content of which is incorporated herein by reference in its entirety as related to Parvoviral capsid proteins, insofar as it does not conflict with the present disclosure.

In certain embodiments of the present disclosure, a viral expression construct may be used for the production of AAV particles in insect cells. In certain embodiments, modifications may be made to the wild type AAV sequences of the capsid and/or rep genes, for example to improve attributes of the viral particle, such as increased infectivity or specificity, or to enhance production yields from insect cells.

### VP-Coding Region

The present disclosure refers to structural capsid proteins (including VP1, VP2, and VP3) which are encoded by capsid (Cap) genes. These capsid proteins form an outer protein structural shell (i.e., capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins including the viral capsid may be produced, some of which may include a Met1/AA1 amino acid (Met+/AA+) and some of which may lack a Met1/AA1 amino acid as a result of Met/AA− clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in their entirety.

According to the present disclosure, references to capsid proteins is not limited to either clipped (Met−/AA−) or unclipped (Met+/AA+) and may, in context, refer to independent capsid proteins, viral capsids included of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also include VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which includes or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

In certain embodiments, a viral expression construct can comprise a VP-coding region; a VP-coding region is a nucleotide sequence which comprises a VP nucleotide sequence encoding VP1, VP2, VP3, or a combination thereof. In certain embodiments, a viral expression construct can comprise a VP1-coding region; a VP1-coding region is a nucleotide sequence which comprises a VP1 nucleotide sequence encoding a VP1 protein. In certain embodiments, a viral expression construct can comprise a VP2-coding region; a VP2-coding region is a nucleotide sequence which comprises a VP2 nucleotide sequence encoding a VP2 protein. In certain embodiments, a viral expression construct can comprise a VP3-coding region; a VP3-coding region is a nucleotide sequence which comprises a VP3 nucleotide sequence encoding a VP3 protein.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, the viral expression construct comprises a first VP-coding region which comprises a nucleotide sequence encoding one or more AAV capsid proteins selected from VP1, VP2, and VP3. In certain embodiments, the first VP-coding region comprises a nucleotide sequence encoding one or more AAV capsid proteins selected from VP2 and VP3. In certain embodiments, the first VP-coding region comprises a nucleotide sequence encoding VP1, VP2, and VP3 AAV capsid proteins. In certain embodiments, the first VP-coding region comprises a nucleotide sequence encoding VP2 and VP3 AAV capsid proteins. In certain embodiments, the first VP-coding region comprises a nucleotide sequence encoding only VP2 and VP3 AAV capsid proteins. In certain embodiments, the first VP-coding region comprises a nucleotide sequence encoding VP2 and VP3 AAV capsid proteins, but not VP1.

In certain embodiments, the nucleic acid construct comprises a second VP-coding region which comprises a nucleotide sequence encoding one or more AAV capsid proteins selected from VP1, VP2, and VP3. In certain embodiments, the second VP-coding region comprises a nucleotide sequence encoding VP1 AAV capsid proteins. In certain embodiments, the second VP-coding region comprises a nucleotide sequence encoding only VP1 AAV capsid proteins. In certain embodiments, the second VP-coding region comprises a nucleotide sequence encoding VP1 AAV capsid proteins, but not VP2 or VP3.

In certain embodiments, the viral expression construct is an engineered nucleic acid construct. In certain embodiments, the viral expression construct comprises a first nucleotide sequence which comprises the first VP-coding region and the second VP-coding region. In certain embodiments, the first nucleotide sequence comprises a first open reading frame (ORF) which comprises the first VP-coding region, and a second open reading frame (ORF) which comprises the second VP-coding region.

In certain embodiments, the viral expression construct comprises a first nucleotide sequence which comprises the first VP-coding region and a second nucleotide sequence which comprises the second VP-coding region. In certain embodiments, the first nucleotide sequence comprises a first open reading frame (ORF) which comprises the first VP-coding region, and the second nucleotide sequence comprises a second open reading frame (ORF) which comprises the second VP-coding region. In certain embodiments, the first open reading frame is different from the second open reading frame.

In certain embodiments, the viral expression construct comprises a first VP-coding region which comprises a nucleotide sequence encoding one or more AAV capsid proteins selected from VP1, VP2, and VP3; and a second VP-coding region which comprises a nucleotide sequence encoding one or more AAV capsid proteins selected from VP1, VP2, and VP3. In certain embodiments, the first VP-coding region comprises a nucleotide sequence encoding VP1, VP2, and VP3 AAV capsid proteins; and the second VP-coding region comprises a nucleotide sequence encoding only VP1 AAV capsid proteins. In certain embodiments, the first VP-coding region comprises a nucleotide sequence encoding VP1, VP2, and VP3 AAV capsid proteins; and the second VP-coding region comprises a nucleotide sequence encoding VP1 AAV capsid proteins, but not VP2 or VP3. In certain embodiments, the first VP-coding region comprises a nucleotide sequence encoding only VP2 and VP3 AAV capsid proteins; and the second VP-coding region comprises a nucleotide sequence encoding only VP1 AAV capsid proteins. In certain embodiments, the first VP-coding region comprises a nucleotide sequence encoding VP2 and VP3 AAV capsid proteins, but not VP1; and the second VP-coding region which comprises a nucleotide sequence encoding VP1 AAV capsid proteins, but not VP2 or VP3.

In certain embodiments, the first VP-coding region encodes AAV capsid proteins of an AAV serotype, e.g., AAV2, AAV9 or AAVPHPN. In certain embodiments, the second VP-coding region encodes AAV capsid proteins of an AAV serotype, e.g., AAV2, AAV9 or AAVPHPN. In certain embodiments, the AAV serotype of the first VP-coding region is the same as the AAV serotype of the second VP-coding region. In certain embodiments, the AAV serotype of the first VP-coding region is different from the AAV serotype of the second VP-coding region. In certain embodiments, a VP-coding region can be codon optimized for an insect cell. In certain embodiments, a VP-coding region can be codon optimized for a Spodoptera frugiperda cell.

In certain embodiments, the viral expression construct comprises: (i) a first nucleotide sequence which comprises a first expression control region comprising a first promoter sequence, and a first VP-coding region which comprises a nucleotide sequence encoding one or more AAV capsid proteins selected from VP1, VP2, and VP3; and (ii) a second nucleotide sequence which comprises a second expression control region comprising a second promoter sequence, and a second VP-coding region which comprises a nucleotide sequence encoding VP1 AAV capsid proteins, but not VP2 or VP3. In certain embodiments, the viral expression construct comprises: (i) a first nucleotide sequence which comprises a first expression control region comprising a first promoter sequence, and a first VP-coding region which comprises a nucleotide sequence encoding VP2 and VP3 AAV capsid proteins, but not VP1; and (ii) a second nucleotide sequence which comprises a second expression control region comprising a second promoter sequence, and a second VP-coding region which comprises a nucleotide sequence encoding VP1 AAV capsid proteins, but not VP2 or VP3. In certain embodiments, the nucleotide sequence of the second VP-coding region is codon optimized. In certain embodiments, the nucleotide sequence of the second VP-coding region is codon optimized for an insect cell, or more specifically for a Spodoptera frugiperda cell. In certain embodiments, the nucleotide sequence of the second VP-coding region is codon optimized codon optimized to have a nucleotide homology with the reference nucleotide sequence of less than 100%, less than 90%, or less than 80%.

In certain embodiments, the viral expression construct comprises: (i) a first nucleotide sequence which comprises a first expression control region comprising a first promoter sequence, a first start codon region which comprises a first start codon, a first VP-coding region which comprises a nucleotide sequence encoding one or more AAV capsid proteins selected from VP1, VP2, and VP3, and a first stop codon region which comprises a first stop codon; and (ii) a second nucleotide sequence which comprises a second expression control region comprising a second promoter sequence, a second start codon region which comprises a second start codon, a second VP-coding region which comprises a nucleotide sequence encoding VP1 AAV capsid proteins, but not VP2 or VP3, and a second stop codon region which comprises a second stop codon. In certain embodiments, the nucleic acid construct comprises: (i) a first nucleotide sequence which comprises a first expression control region comprising a first promoter sequence, a first start codon region which comprises a first start codon, a first VP-coding region which comprises a nucleotide sequence encoding VP2 and VP3 AAV capsid proteins, but not VP1, and a first stop codon region which comprises a first stop codon; and (ii) a second nucleotide sequence which comprises a second expression control region comprising a second promoter sequence, a second start codon region which comprises a second start codon, a second VP-coding region which comprises a nucleotide sequence encoding VP1 AAV capsid proteins, but not VP2 or VP3, and a second stop codon region which comprises a second stop codon. In certain embodiments, the first start codon is ATG, the second start codon is ATG, or both the first and second start codons are ATG.

In certain embodiments, a nucleotide sequence encoding a VP1 capsid protein can be codon optimized. In certain embodiments, a nucleotide sequence encoding a VP1 capsid protein can be codon optimized for an insect cell. In certain embodiments, a nucleotide sequence encoding a VP2 capsid protein can be codon optimized. In certain embodiments, a nucleotide sequence encoding a VP2 capsid protein can be codon optimized for an insect cell. In certain embodiments, a nucleotide sequence encoding a VP3 capsid protein can be codon optimized. In certain embodiments, a nucleotide sequence encoding a VP3 capsid protein can be codon optimized for an insect cell.

In certain embodiments, a nucleotide sequence encoding a VP1 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a nucleotide sequence encoding a VP2 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a nucleotide sequence encoding a VP3 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

Structural VP proteins, VP1, VP2, and VP3 of a viral expression construct can be encoded in a single open reading frame regulated by utilization of both alternative splice acceptor and non-canonical translational initiation codons. VP1, VP2, and VP3 can be transcribed and translated from a single transcript in which both in-frame and/or out-of-frame start codons are engineered to control the VP1:VP2:VP3 ratio produced by the nucleotide transcript.

In certain embodiments, VP1 can be produced from a sequence which encodes for VP1 only. As used herein, the terms “only for VP1” or “VP1 only” refer to a nucleotide sequence or transcript which encodes primarily for VP1 capsid protein relative to non-VP1 capsid proteins (e.g., VP2 capsid proteins or VP3 capsid proteins). In some embodiments, the nucleotide sequence or transcript: (i) lacks a necessary element within the VP1 sequence such that transcription or translation of VP2 and VP3, as a full or partial sequence, from the VP1 sequence is reduced or inhibited (e.g., a deletion or mutation in one or more start codons within the VP1 sequence upstream of the VP2 or VP3 sequence); (ii) comprises an exogenous nucleic acid sequence or structure (e.g., one or more additional codons) within the VP1 sequence which prevents transcription or translation of VP2 and VP3 from the same sequence; and/or (iii) comprises a start codon for VP1 (e.g., ATG), such that VP1 is the primary VP protein produced by the nucleotide transcript.

In certain embodiments, VP2 can be produced from a sequence which encodes for VP2 only. As used herein, the terms “only for VP2” or “VP2 only” refer to a nucleotide sequence or transcript which encodes primarily for VP2 capsid protein relative to non-VP2 capsid proteins (e.g., VP1 capsid proteins or VP3 capsid proteins). In some embodiments, the nucleotide sequence or transcript: (i) is a truncated variant of a full VP capsid sequence (e.g., full VP1 capsid sequence) which encodes only VP2 capsid proteins; (ii) lacks a necessary element within the VP2 sequence such that transcription or translation of VP3, as a full or partial sequence, from the VP2 sequence is reduced or inhibited (e.g., a deletion or mutation in one or more start codons within the VP2 sequence upstream of the VP3 sequence); (iii) comprises an exogenous nucleic acid sequence or structure (e.g., one or more additional codons) within the VP2 sequence which prevents transcription or translation of VP3 from the same sequence; and/or (iv) comprises a start codon for VP2 (e.g., ATG), such that VP2 is the primary VP protein produced by the nucleotide transcript.

In certain embodiments, VP1 and VP2 can be produced from a sequence which encodes for VP1 and VP2 only. As used herein, the terms “only for VP1 and VP2” or “VP1 and VP2 only” refer to a nucleotide sequence or transcript which encodes primarily for VP1 and VP2 capsid proteins relative to non-VP1/VP2 capsid proteins (e.g., VP3 capsid proteins). In some embodiments, the nucleotide sequence or transcript: (i) lacks a necessary element within the VP1 and/or VP2 sequence such that transcription or translation of VP3, as a full or partial sequence, from the VP1/VP2 sequence is reduced or inhibited (e.g., a deletion or mutation in one or more start codons within the VP1/VP2 sequence upstream of the VP3 sequence); (ii) comprises an exogenous nucleic acid sequence or structure (e.g., one or more additional codons) within the VP1/VP2 sequence which prevents transcription or translation of VP3 from the same sequence; (iii) comprises start codons for VP1 (e.g., ATG) and/or VP2 (e.g., ATG), such that VP1 and VP2 are the primary VP proteins produced by the nucleotide transcript; and/or (iv) comprises VP1-only nucleotide transcript and a VP2-only nucleotide transcript connected by a linker, such as an IRES region.

In certain embodiments, VP3 can be produced from a sequence which encodes for VP3 only. As used herein, the terms “only for VP3” or “VP3 only” refers to a nucleotide sequence or transcript which encodes only VP3 capsid proteins relative to non-VP3 capsid proteins (e.g., VP1 capsid proteins or VP2 capsid proteins). In some embodiments, the nucleotide sequence or transcript: (i) is a truncated variant of a full VP capsid sequence (e.g., full VP1 capsid sequence) which encodes only VP3 capsid proteins; and/or (ii) comprise a start codon for VP3 (e.g., ATG), such that VP3 is the only VP protein produced from the nucleotide transcript.

In certain embodiments, the viral expression construct may contain a nucleotide sequence which comprises a start codon region, such as a sequence encoding AAV capsid proteins which comprise one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG). In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the content of which is incorporated herein by reference in its entirety as related to AAV capsid proteins and the production thereof, insofar as it does not conflict with the present disclosure.

In some embodiments, the VP-coding region encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein or a variant of any of the aforesaid capsid proteins. In some embodiments, the VP-coding region encodes an AAV5 capsid protein or variant thereof, or an AAV9 capsid protein or variant thereof. In some embodiments, the VP-coding region encodes a capsid protein as provided in WO2021230987, WO2019028306, WO2019222329, WO2020077165, WO2020028751, WO2020223280, WO2019222444, WO2019222441, or WO2017100671, the contents of which are hereby incorporated by reference in their entirety. In some embodiments, the VP-coding region encodes a capsid protein encoded by or comprising a sequence as provided in Table 14, or a sequence substantially identical (e.g., having at least about 70%, 75%, 80%, 85%, 90%, 92%, 95%, 97%, 98%, or 99% sequence identity) to any of the aforesaid sequences.

TABLE 14
Exemplary full length capsid sequences

| Description | SEQ ID NO: | Sequence |
|---|---|---|
| AAV9 | 147 | ATGGCTGCCGATGGTTATCTTCCAGATTGGCTCGAGGACAACCTTAGTGAAGGAATTCG |
| (DNA v1) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCITAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |
| AAV9 | 148 | ACGGCTGCCGACGGTTATCTACCCGATTGGCTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |
| AAV9 | 149 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9.v1 | 150 | MAADGYLPDWLEDNLSEGIREWWALKPGAPQPKANQQHQDNARGLVLPGYKYLGPGNGL |
| | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9.v2 | 151 | ATGGCTGCCGATGGTTATCTTCCAGATTGGCTCGAGGACAACCTTAGTGAAGGAATTCG |
| (DNA v1) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCIGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGATGGGACTTTG |
| | | GCGGTGCCTTTTAAGGCACAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCGGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGCTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v2 | 152 | ACGGCTGCCGACGGTTATCTACCCGATTGGCTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCIGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGITACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCITAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGATGGGACTTTG |
| | | GCGGTGCCTTTTAAGGCACAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCGGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGCTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v2 | 153 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v3 | 154 | ACGGCTGCCGACGGTTATCTACCCGATTGGCTCGAGGACAACCTTAGTGAAGGAATTCG |
| (DNA) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAAIGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAGGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGATGGGACTTTG |
| | | GCGGTGCCTTTTAAGGCACAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCGGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGCTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v3 | 155 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v4 | 156 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v5 | 81 | atggctgccgatggttatcttccagattggctcgaggacaaccttagtgaaggaattcg |
| (CG085) | | cgagtggtgggctttgaaacctggagcccctcaacccaaggcaaatcaacaacatcaag |
| (DNA v1) | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtccgcttaatggt |
| | | gccgtccatctttatgctcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v5 | 157 | ACGGCTGCCGACGGTTATCTACCCGATTGGCTCGAGGACAACCTTAGTGAAGGAATTCG |
| (CG085) | | CGAGTGGTGGGCTTTGAAACCTGGAGCCCCTCAACCCAAGGCAAATCAACAACATCAAG |
| (DNA v2) | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTCCGCTTAATGGT |
| | | GCCGTCCATCTTTATGCTCAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGGTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v5 | 82 | MAADGYLPDWLEDNLSEGIREWWALKPGAPQPKANQQHQDNARGLVLPGYKYLGPGNGL |
| (CG085) | | DKGEPVNAADAAALEHDKAYDQQLKAGDNPYLKYNHADAEFQERLKEDTSFGGNLGRAV |
| (amino acid) | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSPLNG |
| | | AVHLYAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v6 | 158 | atggctgccgatggttatcttccagattggctcgaggacaaccttagtgaaggaattcg |
| (DNA v1) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacaggctcgt |
| | | gattctccgaagggttggcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v6 | 159 | ACGGCTGCCGACGGTTATCTACCCGATTGGCTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGICAGACIGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCIGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCACAGGCTCGT |
| | | GATTCTCCGAAGGGTTGGCAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGGTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| | 160 | atggctgccgatggttatcttccagattggctcgaggacaaccttagtgaaggaattcg |
| AAV9.v6 | | cgagtggtgggctttgaaacctggagcccctcaacccaaggcaaatcaacaacatcaag |
| (DNA v3) | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacaggctcgt |
| | | gattctccgaagggttggcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcggatcctccaacggcctt |
| | | caacaaggacaagctgaactctttcatcacccagtattctactggccaagtcagcgtgg |
| | | agatcgagtgggagctgcagaaggaaaacagcaagcgctggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v6 | 161 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAR |
| | | DSPKGWQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v7 | 162 | atggctgccgatggttatcttccagattggctcgaggacaaccttagtgaaggaattcg |
| (DNA v1) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacaggcttat |
| | | tctacggatgtgaggatgcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v7 | 163 | ACGGCTGCCGACGGTTATCTACCCGattggctcgaggacaaccttagtgaaggaattcg |
| (DNA v2) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatottcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttoccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacaggcttat |
| | | tctacggatgtgaggatgcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v7 | 164 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAY |
| | | STDVRMQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v8 | 83 | atggctgccgatggttatcttccagattggctcgaggacaaccttagtgaaggaattcg |
| (CG088) | | cgagtggtgggctttgaaacctggagcccctcaacccaaggcaaatcaacaacatcaag |
| (DNA v1) | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacagattgtt |
| | | atgaattcgttgaaggctcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v8 | 165 | ACGGCTGCCGACGGTTATCTACCCGattggcTCGAGGACAACCTTAGTGAAGGAATTCG |
| (CG088) | | CGAGTGGTGGGCTTTGAAACCTGGAGCCCCTCAACCCAAGGCAAATCAACAACATCAAG |
| (DNA v2) | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCICAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCITAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGgccacaaaccaccagagtGCACAGATTGTT |
| | | ATGAATTCGTTGAAGGCTCAGGCGCAGaccggctggGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGgTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v8 | 84 | MAADGYLPDWLEDNLSEGIREWWALKPGAPQPKANQQHQDNARGLVLPGYKYLGPGNGL |
| (CG088)(am | | DKGEPVNAADAAALEHDKAYDQQLKAGDNPYLKYNHADAEFQERLKEDTSFGGNLGRAV |
| ino acid) | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQIV |
| | | MNSLKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v9 | 166 | atggctgccgatggttatcttccagattggctcgaggacaaccttagtgaaggaattcg |
| (DNA v1) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacaggctcgg |
| | | gagagtcctcgtgggctgcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v9 | 167 | ACGGCTGCCGACGGTTATCTACCCGATTGGCTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCITAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGITTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCACAGGCTCGG |
| | | GAGAGTCCTCGTGGGCTGCAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGGTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v9 | 168 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAR |
| | | ESPRGLQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV1 | 169 | atggctgccgatggttatcttccagattggctcgaggacaacctctctgagggcattcg |
| (DNA v1) | | cgagtggtgggacttgaaacctggagccccgaagcccaaagccaaccagcaaaagcagg |
| | | acgacggccggggtctggtgcttcctggctacaagtacctcggacccttcaacggactc |
| | | gacaagggggagcccgtcaacgcggcggacgcagcggccctcgagcacgacaaggccta |
| | | cgaccagcagctcaaagcgggtgacaatccgtacctgcggtataaccacgccgacgccg |
| | | agtttcaggagcgtctgcaagaagatacgtcttttgggggcaacctcgggcgagcagtc |
| | | ttccaggccaagaagcgggttctcgaacctctcggtctggttgaggaaggcgctaagac |
| | | ggctcctggaaagaaacgtccggtagagcagtcgccacaagagccagactcctcctcgg |
| | | gcatcggcaagacaggccagcagcccgctaaaaagagactcaattttggtcagactggc |
| | | gactcagagtcagtccccgatccacaacctctcggagaacctccagcaacccccgctgc |
| | | tgtgggacctactacaatggcttcaggcggtggcgcaccaatggcagacaataacgaag |
| | | gcgccgacggagtgggtaatgcctcaggaaattggcattgcgattccacatggctgggc |
| | | gacagagtcatcaccaccagcacccgcacctgggccttgcccacctacaataaccacct |
| | | ctacaagcaaatctccagtgcttcaacgggggccagcaacgacaaccactacttcggct |
| | | acagcaccccctgggggtattttgatttcaacagattccactgccacttttcaccacgt |
| | | gactggcagcgactcatcaacaacaattggggattccggcccaagagactcaacttcaa |
| | | actcttcaacatccaagtcaaggaggtcacgacgaatgatggcgtcacaaccatcgcta |
| | | ataaccttaccagcacggttcaagtcttctcggactcggagtaccagcttccgtacgtc |
| | | ctcggctctgcgcaccagggctgcctccctccgttcccggcggacgtgttcatgattcc |
| | | gcaatacggctacctgacgctcaacaatggcagccaagccgtgggacgttcatcctttt |
| | | actgcctggaatatttcccttctcagatgctgagaacgggcaacaactttaccttcagc |
| | | tacacctttgaggaagtgcctttccacagcagctacgcgcacagccagagcctggaccg |
| | | gctgatgaatcctctcatcgaccaatacctgtattacctgaacagaactcaaaatcagt |
| | | ccggaagtgcccaaaacaaggacttgctgtttagccgtgggtctccagctggcatgtct |
| | | gttcagcccaaaaactggctacctggaccctgttatcggcagcagcgcgtttctaaaac |
| | | aaaaacagacaacaacaacagcaattttacctggactggtgcttcaaaatataacctca |
| | | atgggcgtgaatccatcatcaaccctggcactgctatggcctcacacaaagacgacgaa |
| | | gacaagttctttcccatgagcggtgtcatgatttttggaaaagagagcgccggagcttc |
| | | aaacactgcattggacaatgtcatgattacagacgaagaggaaattaaagccactaacc |
| | | ctgtggccaccgaaagatttgggaccgtggcagtcaatttccagagcagcagcacagac |
| | | cctgcgaccggagatgtgcatgctatgggagcattacctggcatggtgtggcaagatag |
| | | agacgtgtacctgcagggtcccatttgggccaaaattcctcacacagatggacactttc |
| | | acccgtctcctcttatgggcggctttggactcaagaacccgcctcctcagatcctcatc |
| | | aaaaacacgcctgttcctgcgaatcctccggcggagttttcagctacaaagtttgcttc |
| | | attcatcacccaatactccacaggacaagtgagtgtggaaattgaatgggagctgcaga |
| | | aagaaaacagcaagcgctggaatcccgaagtgcagtacacatccaattatgcaaaatct |
| | | gccaacgttgattttactgtggacaacaatggactttatactgagcctcgccccattgg |
| | | cacccgttaccttacccgtcccctgtaa |
| AAV1 | 170 | ACGGCTGCCGACGGTTATCTACCCGATTGGCTCGAGGACAACCTCTCTGAGGGCATTCG |
| (DNA v2) | | CGAGTGGTGGGACTTGAAACCTGGAGCCCCGAAGCCCAAAGCCAACCAGCAAAAGCAGG |
| | | ACGACGGCCGGGGTCTGGTGCTTCCTGGCTACAAGTACCTCGGACCCTTCAACGGACTC |
| | | GACAAGGGGGAGCCCGTCAACGCGGCGGACGCAGCGGCCCTCGAGCACGACAAGGCCTA |
| | | CGACCAGCAGCTCAAAGCGGGTGACAATCCGTACCTGCGGTATAACCACGCCGACGCCG |
| | | AGTTTCAGGAGCGTCTGCAAGAAGATACGTCTTTTGGGGGCAACCTCGGGCGAGCAGTC |
| | | TTCCAGGCCAAGAAGCGGGTTCTCGAACCTCTCGGTCTGGTTGAGGAAGGCGCTAAGAC |
| | | GGCTCCTGGAAAGAAACGTCCGGTAGAGCAGTCGCCACAAGAGCCAGACTCCTCCTCGG |
| | | GCATCGGCAAGACAGGCCAGCAGCCCGCTAAAAAGAGACTCAATTTTGGTCAGACTGGC |
| | | GACTCAGAGTCAGTCCCCGATCCACAACCTCTCGGAGAACCTCCAGCAACCCCCGCTGC |
| | | TGTGGGACCTACTACAATGGCTTCAGGCGGTGGCGCACCAATGGCAGACAATAACGAAG |
| | | GCGCCGACGGAGTGGGTAATGCCTCAGGAAATTGGCATTGCGATTCCACATGGCTGGGC |
| | | GACAGAGTCATCACCACCAGCACCCGCACCTGGGCCTTGCCCACCTACAATAACCACCT |
| | | CTACAAGCAAATCTCCAGTGCTTCAACGGGGGCCAGCAACGACAACCACTACTTCGGCT |
| | | ACAGCACCCCCTGGGGGTATTTTGATTTCAACAGATTCCACTGCCACTTTTCACCACGT |
| | | GACTGGCAGCGACTCATCAACAACAATTGGGGATTCCGGCCCAAGAGACTCAACTTCAA |
| | | ACTCTTCAACATCCAAGTCAAGGAGGTCACGACGAATGATGGCGTCACAACCATCGCTA |
| | | ATAACCTTACCAGCACGGTTCAAGTCTTCTCGGACTCGGAGTACCAGCTTCCGTACGTC |
| | | CTCGGCTCTGCGCACCAGGGCTGCCTCCCTCCGTTCCCGGCGGACGTGTTCATGATTCC |
| | | GCAATACGGCTACCTGACGCTCAACAATGGCAGCCAAGCCGTGGGACGTTCATCCTTTT |
| | | ACTGCCTGGAATATTTCCCTTCTCAGATGCTGAGAACGGGCAACAACTTTACCTTCAGC |
| | | TACACCTTTGAGGAAGTGCCTTTCCACAGCAGCTACGCGCACAGCCAGAGCCTGGACCG |
| | | GCTGATGAATCCTCTCATCGACCAATACCTGTATTACCTGAACAGAACTCAAAATCAGT |
| | | CCGGAAGTGCCCAAAACAAGGACTTGCTGTTTAGCCGTGGGTCTCCAGCIGGCATGTCT |
| | | GTTCAGCCCAAAAACTGGCTACCTGGACCCTGTTATCGGCAGCAGCGCGTTTCTAAAAC |
| | | AAAAACAGACAACAACAACAGCAATTTTACCTGGACTGGTGCTTCAAAATATAACCTCA |
| | | ATGGGCGTGAATCCATCATCAACCCTGGCACTGCTATGGCCTCACACAAAGACGACGAA |
| | | GACAAGTTCTTTCCCATGAGCGGTGTCATGATTTTTGGAAAAGAGAGCGCCGGAGCTTC |
| | | AAACACTGCATTGGACAATGTCATGATTACAGACGAAGAGGAAATTAAAGCCACTAACC |
| | | CTGTGGCCACCGAAAGATTTGGGACCGTGGCAGTCAATTTCCAGAGCAGCAGCACAGAC |
| | | CCTGCGACCGGAGATGTGCATGCTATGGGAGCATTACCTGGCATGGTGTGGCAAGATAG |
| | | AGACGTGTACCTGCAGGGTCCCATTTGGGCCAAAATTCCTCACACAGATGGACACTTTC |
| | | ACCCGTCTCCTCTTATGGGCGGCTTTGGACTCAAGAACCCGCCTCCTCAGATCCTCATC |
| | | AAAAACACGCCTGTTCCTGCGAATCCTCCGGCGGAGTTTTCAGCTACAAAGTTTGCTTC |
| | | ATTCATCACCCAATACTCCACAGGACAAGTGAGTGTGGAAATTGAATGGGAGCTGCAGA |
| | | AAGAAAACAGCAAGCGCTGGAATCCCGAAGTGCAGTACACATCCAATTATGCAAAATCT |
| | | GCCAACGTTGATTTTACTGTGGACAACAATGGACTTTATACTGAGCCTCGCCCCATTGG |
| | | CACCCGTTACCTTACCCGTCCCCTGTAA |
| AAV1 | 171 | MAADGYLPDWLEDNLSEGIREWWDLKPGAPKPKANQQKQDDGRGLVLPGYKYLGPENGL |
| (amino acid) | | DKGEPVNAADAAALEHDKAYDQQLKAGDNPYLRYNHADAEFQERLQEDTSFGGNLGRAV |
| | | FQAKKRVLEPLGLVEEGAKTAPGKKRPVEQSPQEPDSSSGIGKTGQQPAKKRLNFGQTG |
| | | DSESVPDPQPLGEPPATPAAVGPTTMASGGGAPMADNNEGADGVGNASGNWHCDSTWLG |
| | | DRVITTSTRTWALPTYNNHLYKQISSASTGASNDNHYFGYSTPWGYFDFNRFHCHFSPR |
| | | DWQRLINNNWGFRPKRLNFKLFNIQVKEVTTNDGVTTIANNLTSTVQVFSDSEYQLPYV |
| | | LGSAHQGCLPPFPADVFMIPQYGYLTLNNGSQAVGRSSFYCLEYFPSQMLRTGNNFTFS |
| | | YTFEEVPFHSSYAHSQSLDRLMNPLIDQYLYYLNRTQNQSGSAQNKDLLFSRGSPAGMS |
| | | VQPKNWLPGPCYRQQRVSKTKTDNNNSNFTWIGASKYNLNGRESIINPGTAMASHKDDE |
| | | DKFFPMSGVMIFGKESAGASNTALDNVMITDEEEIKATNPVATERFGTVAVNFQSSSTD |
| | | PATGDVHAMGALPGMVWQDRDVYLQGPIWAKIPHTDGHFHPSPLMGGFGLKNPPPQILI |
| | | KNTPVPANPPAEFSATKFASFITQYSTGQVSVEIEWELQKENSKRWNPEVQYTSNYAKS |
| | | ANVDFTVDNNGLYTEPRPIGTRYLTRPL |
| AAV5 | 172 | atgtcttttgttgatcaccctccagattggttggaagaagttggtgaaggtcttcgcga |
| (DNA v1) | | gtttttgggccttgaagcgggcccaccgaaaccaaaacccaatcagcagcatcaagatc |
| | | aagcccgtggtcttgtgctgcctggttataactatctcggacccggaaacggtctcgat |
| | | cgaggagagcctgtcaacagggcagacgaggtcgcgcgagagcacgacatctcgtacaa |
| | | cgagcagcttgaggcgggagacaacccctacctcaagtacaaccacgcggacgccgagt |
| | | ttcaggagaagctcgccgacgacacatccttcgggggaaacctcggaaaggcagtcttt |
| | | caggccaagaaaagggttctcgaaccttttggcctggttgaagagggtgctaagacggc |
| | | ccctaccggaaagcggatagacgaccactttccaaaaagaaagaaggctcggaccgaag |
| | | aggactccaagccttccacctcgtcagacgccgaagctggacccagcggatcccagcag |
| | | ctgcaaatcccagcccaaccagcctcaagtttgggagctgatacaatgtctgcgggagg |
| | | tggcggcccattgggcgacaataaccaaggtgccgatggagtgggcaatgcctcgggag |
| | | attggcattgcgattccacgtggatgggggacagagtcgtcaccaagtccacccgaacc |
| | | tgggtgctgcccagctacaacaaccaccagtaccgagagatcaaaagcggctccgtcga |
| | | cggaagcaacgccaacgcctactttggatacagcaccccctgggggtactttgacttta |
| | | accgcttccacagccactggagcccccgagactggcaaagactcatcaacaactactgg |
| | | ggcttcagaccccggtccctcagagtcaaaatcttcaacattcaagtcaaagaggtcac |
| | | ggtgcaggactccaccaccaccatcgccaacaacctcacctccaccgtccaagtgttta |
| | | cggacgacgactaccagctgccctacgtcgtcggcaacgggaccgagggatgcctgccg |
| | | gccttccctccgcaggtctttacgctgccgcagtacggttacgcgacgctgaaccgcga |
| | | caacacagaaaatcccaccgagaggagcagcttcttctgcctagagtactttcccagca |
| | | agatgctgagaacgggcaacaactttgagtttacctacaactttgaggaggtgcccttc |
| | | cactccagcttcgctcccagtcagaacctgttcaagctggccaacccgctggtggacca |
| | | gtacttgtaccgcttcgtgagcacaaataacactggcggagtccagttcaacaagaacc |
| | | tggccgggagatacgccaacacctacaaaaactggttcccggggcccatgggccgaacc |
| | | cagggctggaacctgggctccggggtcaaccgcgccagtgtcagcgccttcgccacgac |
| | | caataggatggagctcgagggcgcgagttaccaggtgcccccgcagccgaacggcatga |
| | | ccaacaacctccagggcagcaacacctatgccctggagaacactatgatcttcaacagc |
| | | cagccggcgaacccgggcaccaccgccacgtacctcgagggcaacatgctcatcaccag |
| | | cgagagcgagacgcagccggtgaaccgcgtggcgtacaacgtcggcgggcagatggcca |
| | | ccaacaaccagagctcTACTACTGCCCCCGCGACCGGCACGTACAACCTCCAGGAAATC |
| | | GTGCCCGGCAGCGTGTGGATGGAGAGGGACGTGTACCTCCAAGGACCCATCTGGGCCAA |
| | | GATCCCAGAGACGGGGGCGCACTTTCACCCCTCTCCGGCCATGGGCGGATTCGGACTCA |
| | | AACACCCACCGCCCATGATGCTCATCAAGAACACGCCTGTGCCCGGAAATATCACCAGC |
| | | TTCTCGGACGTGCCCGTCAGCAGCTTCATCACCCAGTACAGCACCGGGCAGGTCACCGT |
| | | GGAGATGGAGTGGGAGCTCAAGAAGGAAAACTCCAAGAGGTGGAACCCAGAGATCCAGT |
| | | ACACAAACAACTACAACGACCCCCAGTTTGTGGACTTTGCCCCGGACAGCACCGGGGAA |
| | | TACAGAACCACCAGACCTATCGGAACCCGATACCTTACCCGACCCCTTTAA |
| AAV5 | 173 | AcGagTTTTGTTGATCACCCaCCCGATTGGTTGGAAGAAGTTGGTGAAGGTCTTCGCGA |
| (DNA v2) | | GTTTTTGGGCCTTGAAGCGGGCCCACCGAAACCAAAACCCAATCAGCAGCATCAAGATC |
| | | AAGCCCGTGGTCTTGTGCTGCCTGGTTATAACTATCTCGGACCCGGAAACGGTCTCGAT |
| | | CGAGGAGAGCCTGTCAACAGGGCAGACGAGGTCGCGCGAGAGCACGACATCTCGTACAA |
| | | CGAGCAGCTTGAGGCGGGAGACAACCCCTACCTCAAGTACAACCACGCGGACGCCGAGT |
| | | TTCAGGAGAAGCTCGCCGACGACACATCCTTCGGGGGAAACCTCGGAAAGGCAGTCTTT |
| | | CAGGCCAAGAAAAGGGTTCTCGAACCTTTTGGCCTGGTTGAAGAGGGTGCTAAGACGGC |
| | | CCCTACCGGAAAGCGGATAGACGACCACTTTCCAAAAAGAAAGAAGGCCCGGACCGAAG |
| | | AGGACTCCAAGCCTTCCACCTCGTCAGACGCCGAAGCTGGACCCAGCGGATCCCAGCAG |
| | | CTGCAAATCCCAGCCCAACCAGCCTCAAGTTTGGGAGCTGATACAATGTCTGCGGGAGG |
| | | TGGCGGCCCATTGGGCGACAATAACCAAGGTGCCGATGGAGTGGGCAATGCCTCGGGAG |
| | | ATTGGCATTGCGATTCCACGTGGATGGGGGACAGAGTCGTCACCAAGTCCACCCGAACC |
| | | TGGGTGCTGCCCAGCTACAACAACCACCAGTACCGAGAGATCAAAAGCGGCTCCGTCGA |
| | | CGGAAGCAACGCCAACGCCTACTTTGGATACAGCACCCCCTGGGGGTACTTTGACTTTA |
| | | ACCGCTTCCACAGCCACTGGAGCCCCCGAGACTGGCAAAGACTCATCAACAACTACTGG |
| | | GGCTTCAGACCCCGGTCCCTCAGAGTCAAAATCTTCAACATTCAAGTCAAAGAGGTCAC |
| | | GGTGCAGGACTCCACCACCACCATCGCCAACAACCTCACCTCCACCGTCCAAGTGTTTA |
| | | CGGACGACGACTACCAGCTGCCCTACGTCGTCGGCAACGGGACCGAGGGATGCCTGCCG |
| | | GCCTTCCCTCCGCAGGTCTTTACGCTGCCGCAGTACGGTTACGCGACGCTGAACCGCGA |
| | | CAACACAGAAAATCCCACCGAGAGGAGCAGCTTCTTCTGCCTAGAGTACTTTCCCAGCA |
| | | AGATGCTGAGAACGGGCAACAACTTTGAGTTTACCTACAACTTTGAGGAGGTGCCCTTC |
| | | CACTCCAGCTTCGCTCCCAGTCAGAACCTCTTCAAGCTGGCCAACCCGCTGGTGGACCA |
| | | GTACTTGTACCGCTTCGTGAGCACAAATAACACTGGCGGAGTCCAGTTCAACAAGAACC |
| | | TGGCCGGGAGATACGCCAACACCIACAAAAACTGGTTCCCGGGGCCCATGGGCCGAACC |
| | | CAGGGCTGGAACCTGGGCTCCGGGGTCAACCGCGCCAGTGTCAGCGCCTTCGCCACGAC |
| | | CAATAGGATGGAGCTCGAGGGCGCGAGTTACCAGGTGCCCCCGCAGCCGAACGGCATGA |
| | | CCAACAACCTCCAGGGCAGCAACACCTATGCCCTGGAGAACACTATGATCTTCAACAGC |
| | | CAGCCGGCGAACCCGGGCACCACCGCCACGTACCTCGAGGGCAACATGCTCATCACCAG |
| | | CGAGAGCGAGACGCAGCCGGTGAACCGCGTGGCGTACAACGTCGGCGGGCAGATGGCCA |
| | | CCAACAACCAGAGCTCCACCACTGCCCCCGCGACCGGCACGTACAACCTCCAGGAAATC |
| | | GTGCCCGGCAGCGTGTGGATGGAGAGGGACGTGTACCTCCAAGGACCCATCTGGGCCAA |
| | | GATCCCAGAGACGGGGGCGCACTTTCACCCCTCTCCGGCCATGGGCGGATTCGGACTCA |
| | | AACACCCACCGCCCATGATGCTCATCAAGAACACGCCTGTGCCCGGAAATATCACCAGC |
| | | TTCTCGGACGTGCCCGTCAGCAGCTTCATCACCCAGTACAGCACCGGGCAGGTCACCGT |
| | | GGAGATGGAGTGGGAGCTCAAGAAGGAAAACTCCAAGAGGIGGAACCCAGAGATCCAGT |
| | | ACACAAACAACTACAACGACCCCCAGTTTGTGGACTTTGCCCCGGACAGCACCGGGGAA |
| | | TACAGAACCACCAGACCTATCGGAACCCGATACCTTACCCGACCCCTTTAA |
| AAV5 | 174 | MSFVDHPPDWLEEVGEGLREFLGLEAGPPKPKPNQQHQDQARGLVLPGYNYLGPGNGLD |
| (amino acid) | | RGEPVNRADEVAREHDISYNEQLEAGDNPYLKYNHADAEFQEKLADDTSFGGNLGKAVE |
| | | QAKKRVLEPFGLVEEGAKTAPTGKRIDDHFPKRKKARTEEDSKPSTSSDAEAGPSGSQQ |
| | | LQIPAQPASSLGADTMSAGGGGPLGDNNQGADGVGNASGDWHCDSTWMGDRVVTKSTRT |
| | | WVLPSYNNHQYREIKSGSVDGSNANAYFGYSTPWGYFDFNRFHSHWSPRDWQRLINNYW |
| | | GFRPRSLRVKIFNIQVKEVTVQDSTTTIANNLTSTVQVFTDDDYQLPYVVGNGTEGCLP |
| | | AFPPQVFTLPQYGYATLNRDNTENPTERSSFFCLEYFPSKMLRTGNNFEFTYNFEEVPF |
| | | HSSFAPSQNLFKLANPLVDQYLYRFVSTNNTGGVQFNKNLAGRYANTYKNWFPGPMGRT |
| | | QGWNLGSGVNRASVSAFATTNRMELEGASYQVPPQPNGMTNNLQGSNTYALENTMIENS |
| | | QPANPGTTATYLEGNMLITSESETQPVNRVAYNVGGQMATNNQSSTTAPATGTYNLQEI |
| | | VPGSVWMERDVYLQGPIWAKIPETGAHFHPSPAMGGFGLKHPPPMMLIKNTPVPGNITS |
| | | FSDVPVSSFITQYSTGQVTVEMEWELKKENSKRWNPEIQYTNNYNDPQFVDFAPDSTGE |
| | | YRTTRPIGTRYLTRPL |

In some embodiments, any of the nucleotide sequences provided in Table 14 can comprises an ATG start codon (e.g., a non-canonical start codon). In some embodiments, any of the sequence a non-canonical start codon, e.g., ACG, CTG, TTG, and GTG. In some embodiments, any of the nucleotide sequences in Table 14 does not comprise a stop codon.

In some embodiments, the VP-coding region encodes a VP1 protein comprising the amino acid sequence of any of SEQ ID NOs: 149, 150, 153, 155, 156, 82, 161, 164, 84, 168, 171, or 174, or an amino acid sequence at least 70%, 75%, 80%, 85%, 90%, 95%, 96%, 97%, 98%, or 99% identical to any of the aforesaid amino acid sequences.

In some embodiments, the VP-coding region encodes a VP2 protein comprising amino acids 138-736 or SEQ ID NOs: 171, 149, or 150; amino acids 138-743 of SEQ ID NOs: 153, 155, 156, 82, 161, 164, 84; or amino acids 137-724 of SEQ ID NO: 174.

In some embodiments, the VP-coding region encodes a VP3 protein comprising amino acids 203-736 of SEQ ID NOs: 171, 149, or 150; amino acids 203-743 of SEQ ID NOs: 153, 155, 156, 82, 161, 164, 84; or amino acids 193-724 of SEQ ID NO: 174.

### Rep-Coding Region

In certain embodiments, a viral expression construct can comprise a Rep52-coding region. A Rep52-coding region is a nucleotide sequence which comprises a Rep52 nucleotide sequence encoding a Rep52 protein. In certain embodiments, a viral expression construct can comprise a Rep78-coding region. A Rep78-coding region is a nucleotide sequence which comprises a Rep78 nucleotide sequence encoding a Rep78 protein. In certain embodiments, a viral expression construct can comprise a Rep40-coding region. A Rep40-coding region is a nucleotide sequence which comprises a Rep40 nucleotide sequence encoding a Rep40 protein. In certain embodiments, a viral expression construct can comprise a Rep68-coding region. A Rep68-coding region is a nucleotide sequence which comprises a Rep68 nucleotide sequence encoding a Rep68 protein.

In certain embodiments, Rep78 can be produced from a sequence which encodes for Rep78 only. As used herein, the terms “only for Rep78” or “Rep78 only” refer to a nucleotide sequence or transcript which encodes primarily for Rep78 protein relative to non-Rep78 replication proteins (e.g., Rep52 replication proteins). In some embodiments, the nucleotide sequence or transcript: (i) lacks a necessary element within the Rep78 sequence such that transcription or translation of Rep52, as a full or partial sequence, from the Rep78 sequence is reduced or inhibited (e.g., a deletion or mutation in one or more start codons within the Rep78 sequence upstream of the Rep52 sequence); (ii) comprises an exogenous nucleic acid sequence or structure (e.g., one or more additional codons) within the Rep78 sequence which prevents transcription or translation of Rep52 from the same sequence; and/or (iii) comprises a start codon for Rep78 (e.g., ATG), such that Rep78 is the primary Rep protein produced by the nucleotide transcript.

In certain embodiments, Rep52 can be produced from a sequence which encodes for Rep52 only. As used herein, the terms “only for Rep52” or “Rep52 only” refer to a nucleotide sequence or transcript which encodes primarily for Rep52 protein relative to non-Rep52 replication proteins (e.g., Rep78 replication proteins). In some embodiments, the nucleotide sequence or transcript: (i) is a truncated variant of a full Rep sequence (e.g., full Rep78 sequence) which encodes only Rep52 proteins; and/or (ii) comprises a start codon for Rep52 (e.g., ATG), such that Rep52 is the primary Rep protein produced by the nucleotide transcript.

In certain embodiments, the viral expression construct comprises a first nucleotide sequence which comprises: a Rep52-coding region which comprises a Rep52 sequence encoding a Rep52 protein, a Rep78-coding region which comprises a Rep78 sequence encoding a Rep78 protein, or a combination thereof. In certain embodiments, the first nucleotide sequence comprises both a Rep52-coding region and a Rep78-coding region. In certain embodiments, the first nucleotide sequence comprises a single open reading frame, consists essentially of a single open reading frame, or consists of a single open reading frame. In certain embodiments, the first nucleotide sequence comprises a first open reading frame which comprises a Rep52-coding region, and a second open reading frame which comprises a Rep78-coding region and which is different from the first open reading frame.

In some embodiments, a Rep-coding region in an AAV expression construct described herein comprises a nucleotide sequence in Table 13, or encodes a Rep protein comprising an amino acid sequence as provided in Table 13, or a sequence substantially identical (e.g., having at least 70%, 75%, 80%, 85%, 90%, 95%, 96%, 97%, 98%, or 99%) thereto. In some embodiments, the nucleotide sequence encoding the Rep52 comprises nucleotides 673-1866 of SEQ ID NO: 143. In some embodiments, the encoded Rep52 protein comprises amino acids 225-621 of SEQ ID NO: 144.

TABLE 13
Exemplary full length Rep sequences

| Description | SEQ ID NO: | Sequence |
|---|---|---|
| Rep78 | 143 | ATGGCGGGGTTTTACGAGATTGTGATTAAGGTCCCCAGCGACCTTGACGAGCATCTGCC |
| (DNA) | | CGGCATTTCTGACAGCTTTGTGAACTGGGTGGCCGAGAAGGAGTGGGAGTTGCCGCCAG |
| | | ATTCTGACTTGGATCTGAATCTGATTGAGCAGGCACCCCTGACCGTGGCCGAGAAGCTG |
| | | CAGCGCGACTTTCTGACGGAGTGGCGCCGTGTGAGTAAGGCCCCGGAGGCCCTTTTCTT |
| | | TGTGCAATTTGAGAAGGGAGAGAGCTACTTCCACTTACACGTGCTCGTGGAAACCACCG |
| | | GGGTGAAATCCTTAGTTTTGGGACGTTTCCTGAGTCAGATTCGCGAAAAACTGATTCAG |
| | | AGAATTTACCGCGGGATCGAGCCGACTTTGCCAAACTGGTTCGCGGTCACAAAGACCAG |
| | | AAACGGCGCCGGAGGCGGGAACAAGGTGGTGGACGAGTGCTACATCCCCAATTACTTGC |
| | | TCCCCAAAACCCAGCCTGAGCTCCAGTGGGCGTGGACTAATTTAGAACAGTATTTAAGC |
| | | GCCTGTTTGAATCTCACGGAGCGTAAACGGTTGGTGGCGCAGCATCTGACGCACGTGTC |
| | | GCAGACGCAGGAGCAGAACAAAGAGAATCAGAATCCCAATTCTGACGCGCCGGTGATCA |
| | | GATCAAAAACTTCAGCCAGaTACATGGAGCTGGTCGGGTGGCTCGTGGACAAGGGGATT |
| | | ACCTCGGAGAAGCAGTGGATCCAGGAGGACCAGGCCTCATACATCTCCTTCAATGCGGC |
| | | CTCCAACTCGCGGTCCCAAATCAAGGCTGCCTTGGACAATGCGGGAAAGATTATGAGCC |
| | | TGACTAAAACCGCCCCCGACTACCTGGTGGGCCAGCAGCCCGTGGAGGACATTTCCAGC |
| | | AATCGGATTTATAAAATTTTGGAACTAAACGGGTACGATCCCCAATATGCGGCTTCCGT |
| | | CTTTCTGGGATGGGCCACGAAAAAGTTCGGCAAGAGGAACACCATCTGGCTGTTTGGGC |
| | | CTGCAACTACCGGGAAGACCAACATCGCGGAGGCCATAGCCCACACTGTGCCCTTCTAC |
| | | GGGTGCGTAAACTGGACCAATGAGAACTTTCCCTTCAACGACTGTGTCGACAAGATGGT |
| | | GATCTGGTGGGAGGAGGGGAAGATGACCGCCAAGGTCGTGGAGTCGGCCAAAGCCATTC |
| | | TCGGAGGAAGCAAGGTGCGCGTGGACCAGAAATGCAAGTCCTCGGCCCAGATAGACCCG |
| | | ACTCCCGTGATCGTCACCTCCAACACCAACATGTGCGCCGTGATTGACGGGAACTCAAC |
| | | GACCTTCGAACACCAGCAGCCGTTGCAAGACCGGATGTTCAAATTTGAACTCACCCGCC |
| | | GTCTGGATCATGACTTTGGGAAGGTCACCAAGCAGGAAGTCAAAGACTTTTTCCGGTGG |
| | | GCAAAGGATCACGTGGTTGAGGTGGAGCATGAATTCTACGTCAAAAAGGGTGGAGCCAA |
| | | GAAAAGACCCGCCCCCAGTGACGCAGATATAAGTGAGCCCAAACGGGTGCGCGAGTCAG |
| | | TTGCGCAGCCATCGACGTCAGACGCGGAAGCTTCGATCAACTACGCAGACAGGTACCAA |
| | | AACAAATGTTCTCGTCACGTGGGCATGAATCTGATGCTGTTTCCCTGCAGACAATGCGA |
| | | GAGAATGAATCAGAATTCAAATATCTGCTTCACTCACGGACAGAAAGACTGTTTAGAGT |
| | | GCTTTCCCGTGTCAGAATCTCAACCCGTTTCTGTCGTCAAAAAGGCGTATCAGAAACTG |
| | | TGCTACATTCATCATATCATGGGAAAGGTGCCAGACGCTTGCACTGCCTGCGATCTGGT |
| | | CAATGTGGATTTGGATGACTGCATCTTTGAACAATAA |
| Rep78 | 144 | MAGFYEIVIKVPSDLDEHLPGISDSFVNWVAEKEWELPPDSDLDLNLIEQAPLTVAEKL |
| (amino acid) | | QRDFLTEWRRVSKAPEALFFVQFEKGESYFHLHVLVETTGVKSLVLGRFLSQIREKLIQ |
| | | RIYRGIEPTLPNWFAVTKTRNGAGGGNKVVDECYIPNYLLPKTQPELQWAWTNLEQYLS |
| | | ACLNLTERKRLVAQHLTHVSQTQEQNKENQNPNSDAPVIRSKTSARYMELVGWLVDKGI |
| | | TSEKQWIQEDQASYISFNAASNSRSQIKAALDNAGKIMSLTKTAPDYLVGQQPVEDISS |
| | | NRIYKILELNGYDPQYAASVFLGWATKKFGKRNTIWLFGPATTGKINIAEAIAHTVPFY |
| | | GCVNWTNENFPFNDCVDKMVIWWEEGKMTAKVVESAKAILGGSKVRVDQKCKSSAQIDP |
| | | TPVIVTSNTNMCAVIDGNSTTFEHQQPLQDRMFKFELTRRLDHDFGKVTKQEVKDFFRW |
| | | AKDHVVEVEHEFYVKKGGAKKRPAPSDADISEPKRVRESVAQPSTSDAEASINYADRYQ |
| | | NKCSRHVGMNLMLFPCRQCERMNQNSNICFTHGQKDCLECFPVSESQPVSVVKKAYQKL |
| | | CYIHHIMGKVPDACTACDLVNVDLDDCIFEQ |
| Rep52 | 145 | ATGGAGCTGGTCGGGTGGCTCGTGGACAAGGGGATTACCTCGGAGAAGCAGTGGATCCA |
| (DNA) | | GGAGGACCAGGCCTCATACATCTCCTTCAATGCGGCCTCCAACTCGCGGTCCCAAATCA |
| | | AGGCTGCCTTGGACAATGCGGGAAAGATTATGAGCCTGACTAAAACCGCCCCCGACTAC |
| | | CTGGTGGGCCAGCAGCCCGTGGAGGACATTTCCAGCAATCGGATTTATAAAATTTTGGA |
| | | ACTAAACGGGTACGATCCCCAATATGCGGCTTCCGTCTTTCTGGGATGGGCCACGAAAA |
| | | AGTTCGGCAAGAGGAACACCATCTGGCTGTTTGGGCCTGCAACTACCGGGAAGACCAAC |
| | | ATCGCGGAGGCCATAGCCCACACTGTGCCCTTCTACGGGTGCGTAAACTGGACCAATGA |
| | | GAACTTTCCCTTCAACGACTGTGTCGACAAGATGGTGATCTGGTGGGAGGAGGGGAAGA |
| | | TGACCGCCAAGGTCGTGGAGTCGGCCAAAGCCATTCTCGGAGGAAGCAAGGTGCGCGTG |
| | | GACCAGAAATGCAAGTCCTCGGCCCAGATAGACCCGACTCCCGTGATCGTCACCTCCAA |
| | | CACCAACATGTGCGCCGTGATTGACGGGAACTCAACGACCTTCGAACACCAGCAGCCGT |
| | | TGCAAGACCGGATGTTCAAATTTGAACTCACCCGCCGTCIGGATCATGACTTTGGGAAG |
| | | GTCACCAAGCAGGAAGTCAAAGACTTTTTCCGGTGGGCAAAGGATCACGTGGTTGAGGT |
| | | GGAGCATGAATTCTACGTCAAAAAGGGTGGAGCCAAGAAAAGACCCGCCCCCAGTGACG |
| | | CAGATATAAGTGAGCCCAAACGGGTGCGCGAGTCAGTTGCGCAGCCATCGACGTCAGAC |
| | | GCGGAAGCTTCGATCAACTACGCAGACAGgtaccaaaacaaatgttctcgtcacgtggg |
| | | catgaatctgatgctgtttccctgcagacaatgcgagagaatgaatcagaattcaaata |
| | | tctgcttcactcacggacagaaagactgtttagagtgctttcccgtgtcagaatctcaa |
| | | cccgtttctgtcgtcaaaaaggcgtatcagaaactgtgctacattcatcatatcatggg |
| | | aaaggtgccagacgcttgcactgcctgcgatctggtcaatgtggatttggatgactgca |
| | | tctttgaacaataa |
| Rep52 | 146 | MELVGWLVDKGITSEKQWIQEDQASYISFNAASNSRSQIKAALDNAGKIMSLIKTAPDY |
| (amino acid) | | LVGQQPVEDISSNRIYKILELNGYDPQYAASVELGWATKKFGKRNTIWLFGPATTGKIN |
| | | IAEAIAHTVPFYGCVNWTNENFPFNDCVDKMVIWWEEGKMTAKVVESAKAILGGSKVRV |
| | | DQKCKSSAQIDPTPVIVTSNTNMCAVIDGNSTTFEHQQPLQDRMFKFELTRRLDHDFGK |
| | | VTKQEVKDFFRWAKDHVVEVEHEFYVKKGGAKKRPAPSDADISEPKRVRESVAQPSTSD |
| | | AEASINYADRYQNKCSRHVGMNLMLFPCRQCERMNQNSNICFTHGQKDCLECFPVSESQ |
| | | PVSVVKKAYQKLCYIHHIMGKVPDACTACDLVNVDLDDCIFEQ |

In some embodiments, the Rep-coding region comprises the nucleotide sequence of SEQ ID NO: 143, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 143; or a nucleotide sequence having at least 10, 20, 50, 100, 150, 200, 250, 300, 350, 400, or 450 but no more than 500 modifications (e.g., substitutions) relative to SEQ ID NO: 143.

In some embodiments, the first Rep-coding region encodes the amino acid sequence of SEQ ID NO: 144; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 144; or an amino acid sequence comprising at least 1, 2, 3, 4, 5, 10, 15, or 20 but no more than 30 modifications (e.g., substitutions (e.g., conservative substitutions), insertions, or deletions) relative to the amino acid sequence of SEQ ID NO: 144.

In some embodiments, the second Rep-coding region comprises the nucleotide sequence of SEQ ID NO: 145, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 145; or a nucleotide sequence having at least 10, 20, 50, 100, 150, 200, 250, 300, 350, 400, or 450 but no more than 500 modifications (e.g., substitutions) relative to SEQ ID NO: 145.

In some embodiments, the second Rep-coding region encodes the amino acid sequence of SEQ ID NO: 146; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 146; or an amino acid sequence comprising at least 1, 2, 3, 4, 5, 10, 15, or 20 but no more than 30 modifications (e.g., substitutions (e.g., conservative substitutions), insertions, or deletions) relative to SEQ ID NO: 146.

In certain embodiments, the viral expression construct comprises a Rep78-coding region in a first transcriptional cassette (e.g., ORF). In certain embodiments, the viral expression construct comprises a Rep52-coding region in a second transcriptional cassette (e.g., ORF), which is separate from the first (i.e., Rep78-coding) transcriptional cassette. In certain embodiments, the first (i.e., Rep78-coding) transcriptional cassette is at a first location of a baculovirus vector, and the second (i.e., Rep52-coding) transcriptional cassette is at a second location of the baculovirus vector. In certain embodiments, the first location and the second location are distal from each other (e.g., at least 5000 bp apart). In certain embodiments, the first location and the second location are at least 2000 bp apart, at least 2500 bp apart, at least 3000 bp apart, at least 3500 bp apart, at least 4000 bp apart, at least 4500 bp apart, at least 5000 bp apart, at least 5500 bp apart, at least 6000 bp apart, at least 6500 bp apart, at least 7000 bp apart, at least 7500 bp apart, at least 8000 bp apart, at least 8500 bp apart, at least 9000 bp apart, at least 9500 bp apart, at least 10000 bp apart, at least 10500 bp apart, at least 11000 bp apart, at least 11500 bp apart, at least 12000 bp apart, at least 12500 bp apart, at least 13000 bp apart, at least 13500 bp apart, at least 14000 bp apart, at least 14500 bp apart, at least 15000 bp apart, at least 15500 bp apart, at least 16000 bp apart, at least 16500 bp apart, at least 17000 bp apart, at least 17500 bp apart, at least 18000 bp apart, at least 18500 bp apart, at least 19000 bp apart, at least 19500 bp apart, at least 20000 bp apart, at least 20500 bp apart, at least 21000 bp apart, at least 21500 bp apart, at least 22000 bp apart, at least 22500 bp apart, at least 23000 bp apart, at least 23500 bp apart, at least 24000 bp apart, at least 24500 bp apart, or at least 25000 bp apart, within the baculovirus vector. In certain embodiments, the first (i.e., Rep78-coding) transcriptional cassette is in the polh gene location of the baculovirus vector. In certain embodiments, the first (i.e., Rep78-coding) transcriptional cassette is in the egt gene location of the baculovirus vector. In certain embodiments, the second (i.e., Rep52-coding) transcriptional cassette is in the polh gene location of the baculovirus vector. In certain embodiments, the second (i.e., Rep52-coding) transcriptional cassette is in the egt gene location of the baculovirus vector. In certain embodiments, the first (i.e., Rep78-coding) transcriptional cassette is in the polh gene location of the baculovirus vector, and the second (i.e., Rep52-coding) transcriptional cassette is in the egt gene location of the baculovirus vector. In certain embodiments, the first (i.e., Rep78-coding) transcriptional cassette is in the egt gene location of the baculovirus vector, and the second (i.e., Rep52-coding) transcriptional cassette is in the polh gene location of the baculovirus vector.

In certain embodiments, non-structural proteins, Rep52 and Rep78, of a viral expression construct can be encoded in a single open reading frame regulated by utilization of both alternative splice acceptor and non-canonical translational initiation codons.

Both Rep78 and Rep52 can be translated from a single transcript: Rep78 translation initiates at a first start codon (AUG or non-AUG) and Rep52 translation initiates from a Rep52 start codon (e.g., AUG) within the Rep78 sequence. Rep78 and Rep52 can also be translated from separate transcripts with independent start codons. The Rep52 initiation codons within the Rep78 sequence can be mutated, modified, or removed, such that processing of the modified Rep78 sequence will not produce Rep52 proteins.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG and GTG, that effects partial exon skipping upon expression in insect cells, as described in U.S. Pat. No. 8,512,981, the content of which is incorporated herein by reference in its entirety as related to the promotion of less abundant expression of Rep78 as compared to Rep52 to promote high vector yields, insofar as it does not conflict with the present disclosure.

In certain embodiments, the viral expression construct may be a plasmid vector or a baculoviral construct for the expression in insect cells that contains repeating codons with differential codon biases, for example to achieve improved ratios of Rep proteins, e.g., Rep78 and Rep52 thereby improving large scale (commercial) production of viral expression construct and/or payload construct vectors in insect cells, as taught in U.S. Pat. No. 8,697,417, the content of which is incorporated herein by reference in its entirety as related to AAV replication proteins and the production thereof, insofar as it does not conflict with the present disclosure.

In certain embodiment, improved ratios of rep proteins may be achieved using the method and constructs described in U.S. Pat. No. 8,642,314, the content of which is incorporated herein by reference in its entirety as related to AAV replications proteins and the production thereof, insofar as it does not conflict with the present disclosure.

In certain embodiments, the viral expression construct may encode mutant parvoviral Rep polypeptides which have one or more improved properties as compared with their corresponding wild type Rep polypeptide, such as the preparation of higher virus titers for large scale production. Alternatively, they may be able to allow the production of better-quality viral particles or sustain more stable production of virus. In a non-limiting example, the viral expression construct may encode mutant Rep polypeptides with a mutated nuclear localization sequence or zinc finger domain, as described in Patent Application US 20130023034, the content of which is incorporated herein by reference in its entirety as related to AAV replications proteins and the production thereof, insofar as it does not conflict with the present disclosure.

In certain embodiments, the nucleic acid construct comprises a first nucleotide sequence, and a second nucleotide sequence which is separate from the first nucleotide sequence within the nucleic acid construct. In certain embodiments, the nucleic acid construct comprises a first nucleotide sequence which comprises a Rep52-coding region, and a separate second nucleotide sequence which comprises a Rep78-coding region. In certain embodiments, the nucleic acid construct comprises a first nucleotide sequence and a separate second nucleotide sequence; wherein the first nucleotide sequence comprises a Rep52-coding region and a 2A sequence region; and wherein the second nucleotide sequence comprises a Rep78-coding region and a 2A sequence region.

In certain embodiments, a first nucleotide sequence comprises a Rep52-coding region and 2A sequence region. In certain embodiments, a first nucleotide sequence comprises a Rep78-coding region and 2A sequence region. In certain embodiments, a first nucleotide sequence comprises a Rep52-coding region, a Rep78-coding region, and 2A sequence region. In certain embodiments, a first nucleotide sequence comprises a 2A sequence region located between a Rep52-coding region and a Rep78-coding region on the nucleotide sequence. In certain embodiments, a first nucleotide comprises, in order from the 5′-end to the 3′-end, a Rep52-coding region, a 2A sequence region, and a Rep78-coding region. In certain embodiments, a first nucleotide comprises, in order from the 5′-end to the 3′-end, a Rep78-coding region, a 2A sequence region, and a Rep52-coding region.

For example, in certain embodiments, a first nucleotide sequence comprises a start codon region, a Rep52-coding region, 2A sequence region, and a stop codon region. In certain embodiments, a first nucleotide sequence comprises a start codon region, a Rep78-coding region, 2A sequence region, and a stop codon region. In certain embodiments, a first nucleotide sequence comprises a start codon region, a Rep52-coding region, a 2A sequence region, a Rep78-coding region, and a stop codon region. In certain embodiments, a first nucleotide comprises, in order from the 5′-end to the 3′-end, a start codon region, a Rep52-coding region, a 2A sequence region, a Rep78-coding region, and a stop codon region. In certain embodiments, a first nucleotide comprises, in order from the 5′-end to the 3′-end, a start codon region, a Rep78-coding region, a 2A sequence region, a Rep52-coding region, and a stop codon region.

In certain embodiments, the viral expression construct comprises one or more essential-gene regions which comprises an essential-gene nucleotide sequence encoding an essential protein for the nucleic acid construct. In certain embodiments, the essential-gene nucleotide sequence is a baculoviral sequence encoding an essential baculoviral protein. In certain embodiments, the essential baculoviral protein is a baculoviral envelope protein or a baculoviral capsid protein. For example, in certain embodiments, the nucleic acid construct comprises a first nucleotide sequence and a separate second nucleotide sequence; wherein the first nucleotide sequence comprises a Rep52-coding region and a first essential-gene region; and wherein the second nucleotide sequence comprises a Rep78-coding region and a second essential-gene region. In certain embodiments, the nucleic acid construct comprises a first nucleotide sequence and a separate second nucleotide sequence; wherein the first nucleotide sequence comprises a Rep52-coding region, a 2A sequence region, and a first essential-gene region; and wherein the second nucleotide sequence comprises a Rep78-coding region, a 2A sequence region, and a second essential-gene region. In certain embodiments, the nucleic acid construct comprises a first nucleotide sequence and a separate second nucleotide sequence; wherein the first nucleotide sequence comprises, in order, a Rep52-coding region, a 2A sequence region, and a first essential-gene region; and wherein the second nucleotide sequence comprises, in order, a Rep78-coding region, a 2A sequence region, and a second essential-gene region.

In certain embodiments, the essential baculoviral protein is a GP64 baculoviral envelope protein. In certain embodiments, the essential baculoviral protein is a VP39 baculoviral capsid protein.

In certain embodiments, a first nucleotide sequence comprises a Rep52-coding region, a Rep78-coding region, and an IRES sequence region. In certain embodiments, a first nucleotide sequence comprises an IRES sequence region located between a Rep52-coding region and a Rep78-coding region on the nucleotide sequence. In certain embodiments, a first nucleotide comprises, in order from the 5′-end to the 3′-end, a Rep52-coding region, an IRES sequence region, and a Rep78-coding region. In certain embodiments, a first nucleotide comprises, in order from the 5′-end to the 3′-end, a Rep78-coding region, an IRES sequence region, and a Rep52-coding region.

In certain embodiments, the first nucleotide sequence comprises a first open reading frame which comprises a Rep52-coding region, a second open reading frame which comprises a Rep78-coding region, and an IRES sequence region located between the first open reading frame and the second open reading frame. In certain embodiments, a first nucleotide sequence comprises, in order from the 5′-end to the 3′-end, a first open reading frame which comprises a Rep52-coding region, an IRES sequence region, and a second open reading frame which comprises a Rep78-coding region. In certain embodiments, a first nucleotide sequence comprises, in order from the 5′-end to the 3′-end, a first open reading frame which comprises a Rep78-coding region, an IRES sequence region, and a second open reading frame which comprises a Rep52-coding region.

In certain embodiments, a first nucleotide sequence comprises, in order from the 5′-end to the 3′-end: a first open reading frame which comprises a first start codon region, a Rep52-coding region, and a first stop codon region; an IRES sequence region; and a second open reading frame which comprises a second start codon region, a Rep78-coding region, and a second stop codon region. In certain embodiments, a first nucleotide sequence comprises, in order from the 5′-end to the 3′-end: a first open reading frame which comprises a first start codon region, a Rep78-coding region, and a first stop codon region; an IRES sequence region; and a second open reading frame which comprises a second start codon region, a Rep52-coding region, and a second stop codon region.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the IE-1 promoter, the ΔIE-1 promoter, has about 20% of the transcriptional activity of that IE-1 promoter. A promoter substantially homologous to the ΔIE-1 promoter may be used. In respect to promoters, a homology of at least 50%, 60%, 70%, 80%, 90% or more, is considered to be a substantially homologous promoter.

### Expression Control Regions

A viral expression construct (e.g., expressionBac) of the present disclosure can comprise one or more expression control region encoded by expression control sequences. In certain embodiments, the expression control sequences are for expression in a viral production cell, such as an insect cell. In certain embodiments, the expression control sequences are operably linked to a protein-coding nucleotide sequence. In certain embodiments, the expression control sequences are operably linked to a VP coding nucleotide sequence or a Rep coding nucleotide sequence.

Herein, the terms “coding nucleotide sequence”, “protein-encoding gene” or “protein-coding nucleotide sequence” refer to a nucleotide sequence that encodes or is translated into a protein product, such as VP proteins or Rep proteins. Being operably linked indicates that the expression control sequence is positioned relative to the coding sequence such that it can promote the expression of the encoded gene product.

“Expression control sequence” refers to a nucleic acid sequence that regulates the expression of a nucleotide sequence to which it is operably linked. Thus, an expression control sequence can include promoters, enhancers, untranslated regions (UTRs), internal ribosome entry sites (IRES), transcription terminators, a start codon in front of a protein-encoding gene, splicing signal for introns, and stop codons. The term “expression control sequence” is intended to include, at a minimum, a sequence whose presence are designed to influence expression, and can also include additional advantageous components. For example, leader sequences and fusion partner sequences are expression control sequences. The term can also comprise the design of the nucleic acid sequence such that undesirable, potential initiation codons in and out of frame, are removed from the sequence. It can also comprise the design of the nucleic acid sequence such that undesirable potential splice sites are removed. It comprises sequences or polyadenylation sequences (pA) which direct the addition of a polyA tail, i.e., a string of adenine residues at the 3′-end of an mRNA, sequences referred to as polyA sequences. It also can be designed to enhance mRNA stability. Expression control sequences which affect the transcription and translation stability, e.g., promoters, as well as sequences which effect the translation, e.g., Kozak sequences, are known in insect cells. Expression control sequences can be of such nature as to modulate the nucleotide sequence to which it is operably linked such that lower expression levels or higher expression levels are achieved.

In certain embodiments, the expression control sequence can comprise one or more promoters. Promoters can comprise, but are not limited to, baculovirus major late promoters, insect virus promoters, non-insect virus promoters, vertebrate virus promoters, nuclear gene promoters, chimeric promoters from one or more species comprising virus and non-virus elements, and/or synthetic promoters. In certain embodiments, a promoter can be Ctx, Op-E1, E1, ΔEI, EI-1, pH, PIO, polh (polyhedron), Δpolh, Dmhsp70, Hr1, Hsp70, 4×Hsp27 EcRE+minimal Hsp70, IE, IE-1, ΔIE-1, ΔIE, p10, Δp10 (modified variations or derivatives of p10), p5, p19, p35, p40, p6.9, and variations or derivatives thereof. In certain embodiments, the promoter is a Ctx promoter. In certain embodiments, the promoter is a p10 promoter. In certain embodiments, the promoter is a polh promoter. In certain embodiments, a promoter can be selected from tissue-specific promoters, cell-type-specific promoters, cell-cycle-specific promoters, and variations or derivatives thereof. In certain embodiments, a promoter can be a CMV promoter, an alpha 1-antitrypsin (al-AT) promoter, a thyroid hormone-binding globulin promoter, a thyroxine-binding globulin (LPS) promoter, an HCR-ApoCII hybrid promoter, an HCR-hAAT hybrid promoter, an albumin promoter, an apolipoprotein E promoter, an α1-AT+EaIb promoter, a tumor-selective E2F promoter, a mononuclear blood IL-2 promoter, and variations or derivatives thereof. In certain embodiments, the promoter is a low-expression promoter sequence. In certain embodiments, the promoter is an enhanced-expression promoter sequence. In certain embodiments, the promoter can comprise Rep or Cap promoters as described in US Patent Application 20110136227, the content of which is incorporated herein by reference in its entirety as related to expression promoters, insofar as it does not conflict with the present disclosure.

In some embodiments, the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof. In some embodiments, the promoter is chosen from a polh promoter, a p10 promoter, a Ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof. In some embodiments, the promoter is a p10 promoter. In some embodiments, the promoter comprises a nucleotide sequence provided in Table 15 or a nucleotide sequence at least 70%, 75%, 80%, 85%, 90%, 95%, 96%, 97%, 98%, or 99% identical thereto. In some embodiments, the promoter comprises the nucleotide sequence of SEQ ID NO: 176, or a nucleotide sequence at least 70%, 75%, 80%, 85%, 90%, 95%, 96%, 97%, 98%, or 99% identical thereto. In some embodiments, the promoter is a p10 promoter and comprises the nucleotide sequence of SEQ ID NO: 176, or a nucleotide sequence at least 70%, 75%, 80%, 85%, 90%, 95%, 96%, 97%, 98%, or 99% identical thereto. In some embodiments, the promoter is a polh promoter comprises the nucleotide sequence of SEQ ID NO: 175; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 175; or a nucleotide sequence comprising at least one, two, three, four, five, six, or seven, but no more than ten modifications (e.g., substitutions) relative to SEQ ID NO: 175.

TABLE 15
Exemplary Promoter Sequences

| | SEQ | |
|---|---|---|
| | ID | |
| Description | NO | Sequence |
| polh | 175 | ATCATGGAGATAATTAAAATGATAACCATCTCG |
| Promoter | | CAAATAAATAAGTATTTTACTGTTTTCGTAACA |
| | | GTTTTGTAATAAAAAAACCTATAAAT |
| p10 | 176 | GACCTTTAATTCAACCCAACACAATATATTATA |
| promoter | | GTTAAATAAGAATTATTATCAAATCATTTGTAT |
| | | ATTAATTAAAATACTATACTGTAAATTACATTT |
| | | TA |
| CTX | 177 | tgaaactaacttacaagTtggctagtttgttaa |
| promoter | | aatacgcgctgcgcttgactcgggaatacaaag |
| | | aaaacattattccacactttgatcacttgactc |
| | | gattgcgcgatttaatcgacggcTtgattaaaa |
| | | gcgaggatgtacaacgttttaatcgcactaatc |
| | | gcaatgatttaatttcggcttgcTtgcaaatca |
| | | acgttcggacgtacTtgcccaacgccacgatag |
| | | atTtgcgcaaacaacccaactgtatatattttc |
| | | gaatttgccaatattgccacttggaggccgacg |
| | | tgccttcgcccgacgatcattcggtgtacagat |
| | | acttgtgcgtcgcgtgcggcacgccgctggtca |
| | | tcgaccacccgctcgacgtgttcggccacacgg |
| | | aggaaggcgtcaacgaactgctcgaggtgcagc |
| | | gagtcaacgcgggcggggagttgtaggcgtcat |
| | | aactatttattaaATAAGataatttaaaaaatc |
| | | gccgttaat |
| gp64 | 178 | AAATTATCGCAAGATAAGGCGCACGTTGATTGG |
| promoter | | GTCACCCGAGTGTACGTTGATAAAGTCACGTGG |
| | | GCACCCAACGCGTTGATAAGCATCGGTATATAA |
| | | GGGCCTACAGTGTTCTGGTAAATCAGTTGCACT |
| | | GTGCTCTTCACAGGAACACTACAAGACCTACAA |
| | | G |

In some embodiments, the AAV expression construct comprises a ctx promoter. In some embodiments, the CTX promoter comprises a sequence as provided in Table 16, or a sequence substantially identical (e.g., having at least about 70%, 75%, 80%, 85%, 90%, 92%, 95%, 97%, 98%, or 99% sequence identity) thereto.

TABLE 16
Exemplary CTX promoter sequences

| | SEQ | |
|---|---|---|
| | ID | |
| Description | NO: | Sequence |
| CTX | 177 | tgaaactaacttacaagTtggctagtttgtta |
| promoter | | aaatacgcgctgcgcttgactcgggaatacaa |
| | | agaaaacattattccacactttgatcacttga |
| | | ctcgattgcgcgatttaatcgacggcTtgatt |
| | | aaaagcgaggatgtacaacgttttaatcgcac |
| | | taatcgcaatgatttaatttcggcttgcTtgc |
| | | aaatcaacgttcggacgtacTtgcccaacgcc |
| | | acgatagatTtgcgcaaacaacccaactgtat |
| | | atattttcgaatttgccaatattgccacttgg |
| | | aggccgacgtgccttcgcccgacgatcattcg |
| | | gtgtacagatacttgtgcgtcgcgtgcggcac |
| | | gccgctggtcatcgaccacccgctcgacgtgt |
| | | tcggccacacggaggaaggcgtcaacgaactg |
| | | ctcgaggtgcagcgagtcaacgcgggcgggga |
| | | gttgtaggcgtcataactatttattaaATAAG |
| | | ataatttaaaaaatcgccgttaat |
| CTX | 179 | ATAAGataatttaaaaaatcgccgttaat |
| promoter | | |
| CTX | 180 | gggcggggagttgtaggcgtcataactattta |
| promoter | | ttaaATAAGataatttaaaaaatcgccgttaa |
| | | t |

In certain embodiments, the expression control sequence can comprise one or more expression-modifier sequences, such as a minicistron insertion sequence. In certain embodiments, the expression control sequence can comprise one or more expression modifiers (e.g., minicistron insertion) which is upstream and functionally adjacent/near a start codon (e.g., VP1 start codon, Rep78 start codon). Without being bound by theory, insertion of an expression modifier (e.g., minicistron) upstream and functionally adjacent/near a start codon can result in scanning ribosomes being less competent to recognize, bind, and/or initiate translation at the target ORF start codon (i.e., Rep78 ATG start codon). This can result in decreased translation initiation at a target ORF start codon (i.e., Rep78 ATG start codon), and correspondingly result in increased initiation at downstream ORF start codons (i.e., Rep52 start codon within bicistronic sequence).

In certain embodiments, the expression modifier (e.g., minicistron insertion) is upstream of a target ORF start codon (i.e., Rep78 ATG start codon). In certain embodiments, the expression control sequence comprises one or more nucleotides between the expression modifier (e.g., minicistron insertion) and the target ORF start codon (i.e., Rep78 ATG start codon). In certain embodiments, the expression control sequence comprises between 1-100 nucleotides between the expression modifier and the target ORF start codon. In certain embodiments, the expression control sequence comprises between 3-100 nucleotides between the expression modifier and the target ORF start codon. In certain embodiments, the expression control sequence comprises between 3-75 nucleotides between the expression modifier and the target ORF start codon. In certain embodiments, the expression control sequence comprises between 3-50 nucleotides between the expression modifier and the target ORF start codon. In certain embodiments, the expression control sequence comprises between 3-25 nucleotides between the expression modifier and the target ORF start codon. In certain embodiments, the expression control sequence comprises between 3-15 nucleotides between the expression modifier and the target ORF start codon. In certain embodiments, the expression control sequence comprises between 3-10 nucleotides between the expression modifier and the target ORF start codon. In certain embodiments, the expression control sequence comprises between 3-6 nucleotides between the expression modifier and the target ORF start codon. In certain embodiments, the expression control sequence comprises 3 nucleotides between the expression modifier and the target ORF start codon.

In certain embodiments, the expression modifier is a minicistron insertion sequence (i.e., small open reading frame). In certain embodiments, the minicistron insertion sequence is from a baculovirus gene. In certain embodiments, the minicistron insertion sequence is from a baculovirus gp64 gene. In certain embodiments, the minicistron insertion sequence comprises SEQ ID NO: 4. In certain embodiments, the minicistron insertion sequence comprises SEQ ID NO: 5.

In certain embodiments, a viral expression construct can comprise the same promoter in all nucleotide sequences. In certain embodiments, a viral expression construct can comprise the same promoter in two or more nucleotide sequences. In certain embodiments, a viral expression construct can comprise a different promoter in two or more nucleotide sequences. In certain embodiments, a viral expression construct can comprise a different promoter in all nucleotide sequences.

In certain embodiments the viral expression construct encodes elements to improve expression in certain cell types. In a further embodiment, the expression construct may comprise polh and/or ΔIE-1 insect transcriptional promoters, CMV mammalian transcriptional promoter, and/or p10 insect specific promoters for expression of a desired gene in a mammalian or insect cell.

More than one expression control sequence can be operably linked to a given nucleotide sequence. For example, a promoter sequence, a translation initiation sequence, and a stop codon can be operably linked to a nucleotide sequence.

In certain embodiments, the viral expression construct can comprise one or more expression control sequence between protein-coding nucleotide sequences. In certain embodiments, an expression control region can comprise an IRES sequence region which comprises an IRES nucleotide sequence encoding an internal ribosome entry sight (IRES). The internal ribosome entry sight (IRES) can be selected from the group consisting or: FMDV-IRES from Foot-and-Mouth-Disease virus, EMCV-IRES from Encephalomyocarditis virus, and combinations thereof.

In certain embodiments, the viral expression construct is as described in PCT/US2019/054600 and/or U.S. Provisional Patent Application No. 62/741,855 the contents of which are each incorporated by reference in their entireties.

In certain embodiments, the viral expression construct may contain a nucleotide sequence which comprises a start codon region, such as a sequence encoding AAV capsid proteins which comprise one or more start codon regions. In certain embodiments, the start codon region can be within an expression control sequence.

In certain embodiments, the translational start site of eukaryotic mRNA can be controlled in part by a nucleotide sequence referred to as a Kozak sequence as described in Kozak, M Cell. 1986 Jan. 31; 44(2):283-92 and Kozak, M. J Cell Biol. 1989 February; 108(2):229-41 the contents of each of which are herein incorporated by reference in their entirety as related to Kozak sequences and uses thereof. Both naturally occurring and synthetic translational start sites of the Kozak form can be used in the production of polypeptides by molecular genetic techniques, Kozak, M. Mamm Genome. 1996 August; 7(8):563-74 the contents of which are herein incorporated by reference in their entirety as related to Kozak sequences and uses thereof. Splice sites are sequences on an mRNA which facilitate the removal of parts of the mRNA sequences after the transcription (formation) of the mRNA. Typically, the splicing occurs in the nucleus, prior to mRNA transport into a cell's cytoplasm.

In certain embodiments, the viral expression construct may contain a nucleotide sequence which comprises a stop codon region, such as a sequence encoding AAV capsid proteins which comprise one or more stop codon regions. In certain embodiments, the stop codon region can be within an expression control sequence.

In certain embodiments, the viral expression construct comprises one or more start codon regions which include a start codon. In certain embodiments, the viral expression construct comprises one or more stop codon regions which include a stop codon. In certain embodiments, the viral expression construct comprises one or more start codon regions and one or more stop codon regions. In certain embodiments, the start codon region and/or stop codon region can be within an expression control sequence.

In certain embodiments, the viral expression construct comprises one or more expression control regions which comprise an expression control sequence. In certain embodiments, the expression control region comprises one or more promoter sequences. In certain embodiments, the expression control region comprises one or more promoter sequences selected from the group consisting of: baculovirus major late promoters, insect virus promoters, non-insect virus promoters, vertebrate virus promoters, nuclear gene promoters, chimeric promoters from one or more species including virus and non-virus elements, synthetic promoters, and variations or derivatives thereof. In certain embodiments, the expression control region comprises one or more promoter sequences selected from the group consisting of: Ctx promoter, polh insect transcriptional promoters, ΔIE-1 insect transcriptional promoters, p10 insect specific promoters, Δp10 insect specific promoters (variations or derivatives of p10), CMV mammalian transcriptional promoter, and variations or derivatives thereof. In certain embodiments, the expression control region comprises one or more low-expression promoter sequences. In certain embodiments, the expression control region comprises one or more enhanced-expression promoter sequences.

In certain embodiments, an expression control region can comprise a 2A sequence region which comprises a 2A nucleotide sequence encoding a viral 2A peptide. The sequence allows for co-translation of multiple polypeptides within a single open reading frame (ORF). As the ORF is translated, glycine and proline residues with the 2A sequence prevent the formation of a normal peptide bond, which results in ribosomal “skipping” and “self-cleavage” within the polypeptide chain. The viral 2A peptide can be selected from the group consisting of: F2A from Foot-and-Mouth-Disease virus, T2A from Thosea asigna virus, E2A from Equine rhinitis A virus, P2A from porcine teschovirus-1, BmCPV2A from cytoplasmic polyhedrosis virus, BmIFV 2A from B. mori flacherie virus, and combinations thereof.

In some embodiments, the first and/or second nucleotide sequence comprises a start codon and/or stop codon and/or internal ribosome entry site (IRES). In certain embodiments, the IRES nucleotide sequence encodes an internal ribosome entry site (IRES) selected from the group consisting of: FMDV-IRES from Foot-and-Mouth-Disease virus, EMCV-IRES from Encephalomyocarditis virus, and combinations thereof.

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or which is 1:1:10 (VP1:VP2:VP3).

In certain embodiments, AAV particles are produced in viral production cells (such as mammalian or insect cells) wherein all three VP proteins are expressed at a stoichiometry approaching, about or which is 2:2:10 (VP1:VP2:VP3).

In certain embodiments, AAV particles are produced in viral production cells (such as mammalian or insect cells) wherein all three VP proteins are expressed at a stoichiometry approaching, about or which is 2:0:10 (VP1:VP2:VP3).

In certain embodiments, AAV particles are produced in viral production cells (such as mammalian or insect cells) wherein all three VP proteins are expressed at a stoichiometry approaching, about or which is 1-2:0-2:10 (VP1:VP2:VP3).

In certain embodiments, AAV particles are produced in viral production cells (such as mammalian or insect cells) wherein all three VP proteins are expressed at a stoichiometry approaching, about or which is 1-2:1-2:10 (VP1:VP2:VP3).

In certain embodiments, AAV particles are produced in viral production cells (such as mammalian or insect cells) wherein all three VP proteins are expressed at a stoichiometry approaching, about or which is 2-3:0-3:10 (VP1:VP2:VP3).

In certain embodiments, AAV particles are produced in viral production cells (such as mammalian or insect cells) wherein all three VP proteins are expressed at a stoichiometry approaching, about or which is 2-3:2-3:10 (VP1:VP2:VP3).

In certain embodiments, AAV particles are produced in viral production cells (such as mammalian or insect cells) wherein all three VP proteins are expressed at a stoichiometry approaching, about or which is 3:3:10 (VP1:VP2:VP3).

In certain embodiments, AAV particles are produced in viral production cells (such as mammalian or insect cells) wherein all three VP proteins are expressed at a stoichiometry approaching, about or which is 3-5:0-5:10 (VP1:VP2:VP3).

In certain embodiments, AAV particles are produced in viral production cells (such as mammalian or insect cells) wherein all three VP proteins are expressed at a stoichiometry approaching, about or which is 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

### Transcriptional Regulatory Systems

The present disclosure presents transcriptional regulatory systems which can be used to regulate the expression of a protein-coding nucleotide sequence. The present disclosure presents viral expression constructs which include a transcriptional regulatory system which can be used to regulate the expression of a protein-coding nucleotide sequence. The present disclosure presents expression control regions which include a transcriptional regulatory system which can be used to regulate the expression of a protein-coding nucleotide sequence (i.e., regulatable expression control region).

In certain embodiments, the transcriptional regulatory system is functional in increasing the expression of a protein-coding nucleotide sequence. In certain embodiments, the transcriptional regulatory system is functional in decreasing or silencing the expression of a protein-coding nucleotide sequence. In certain embodiments, the transcriptional regulatory system is functional in increasing, decreasing or silencing the expression of a nucleotide sequence encoding one or more structural AAV capsid proteins (e.g., VP1, VP2, VP3, or a combination thereof). In certain embodiments, the transcriptional regulatory system is functional in increasing, decreasing or silencing the expression of a nucleotide sequence encoding one or more non-structural AAV replication proteins (e.g., Rep78, Rep52, or a combination thereof). In certain embodiments, the transcriptional regulatory system is functional in increasing, decreasing or silencing the expression of a nucleotide sequence encoding one or more payload polypeptides.

In certain embodiments, the transcriptional regulatory system includes at least one regulator element and at least one regulator binding region. In certain embodiments, the regulator element can bind to the regulator binding region. In certain embodiments, the regulator element has a high affinity for binding to the regulator binding region. In certain embodiments, the regulator element is an inducible regulator element. In certain embodiments, the transcriptional regulatory system includes at least one regulator element, at least one regulator binding region, and at least one inducer element. In certain embodiments, the inducer element can reduce the affinity of the regulator element for binding to the regulator binding region. In certain embodiments, the regulator element has a high affinity for binding to the regulator binding region when the inducer element is not present or present at low concentrations, and a low affinity for binding to the regulator binding region when the inducer element is present or present at high concentrations. In certain embodiments, the inducer element binds to regulator element and causes a conformational change in the regulator element to reduce binding affinity to the regulator binding region.

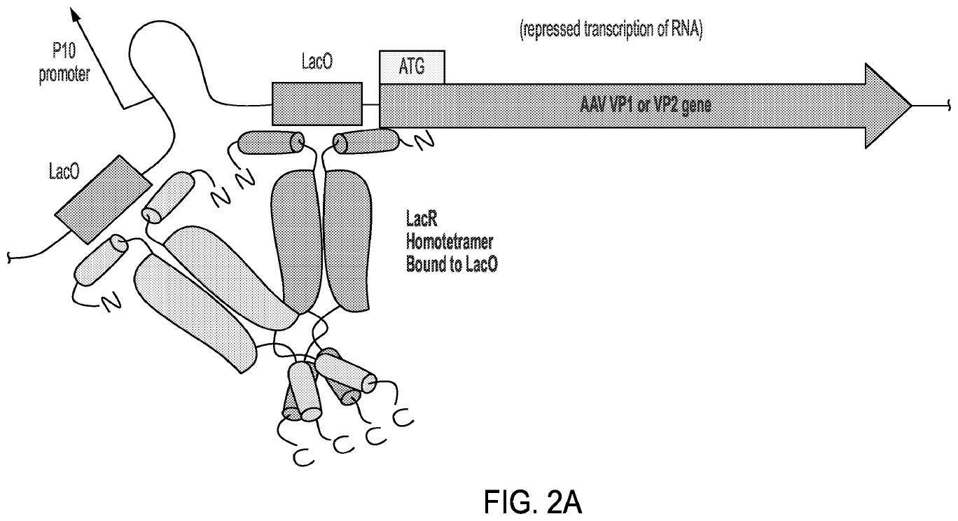

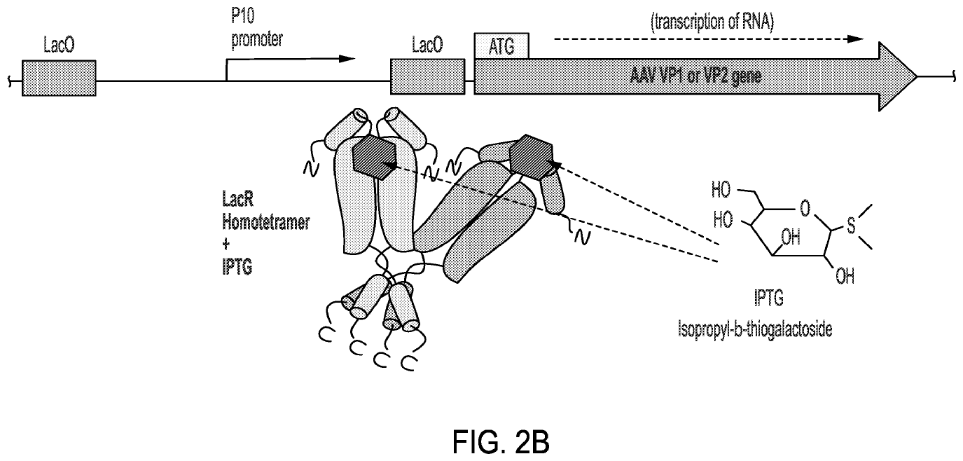

In certain embodiments, the regulator element is a Lac repressor (LacR) protein, the regulator binding region is a Lac Operator (LacO) nucleotide sequence, and the inducer element is a LacR inducer element selected from Lactose, Allolactose and isopropyl-β-D-thiogalactose (IPTG). As shown in FIG. 2A, the LacR protein is a homotetrameric protein which binds to one or more Lac Operator (LacO) nucleotide sequences. The tetrameric LacR protein typically binds to two LacO sequences simultaneously (such as one LacO sequence on each side of a promoter) and constrains the promoter (e.g., p10 promoter) into a loop when acting on the LacO sequences. When this happens, transcription initiation of the promoter is reduced or fully repressed. As shown in FIG. 2B, binding of LacR to LacO can controlled by the presence of an inducer element, such as the sugar allolactose. When allolactose binds to LacR, it causes LacR to conformationally change and to not bind to LacO nucleotide sequences. The synthetic analog of allolactose is isopropyl β-d-1-thiogalactopyranoside (IPTG). In certain embodiments, IPTG is preferred to allolactose because it is not metabolized and thus maintains stable induction of LacR after being added to cell cultures.

In certain embodiments, the regulator element is a Lac repressor (LacR) protein. LacR is typically a 360 amino acid protein with a molecular weight of 38 kDa which is typically encoded by the LacI gene. In certain embodiments, the regulator element is a Lac repressor (LacR) protein encoded by a LacR nucleotide sequence (i.e., LacI gene). In certain embodiments the LacR protein can be wt E. coli LacR from the LacI gene. In certain embodiments the LacR protein is an engineered LacR protein for expression in viral production cells, such as insect cells. Modifications to the LacI gene (and corresponding engineering LacR protein) can include: changing the translation initiation codon to ATG or a Kozak sequence (or modified Kozak sequence) which includes ATG; and the addition of an SV40 nuclear localization signal (NLS) to the N-terminus of LacR. In certain embodiments, the engineered LacR protein is encoded by a sequence which includes an NLS sequence, a linker sequence, and a modified LacI gene which includes a modified Kozak sequence and an ATG start codon. In certain embodiments, the engineered LacR protein is encoded by SEQ ID NO: 6. In certain embodiments, the engineered LacR protein is encoded by nucleotide sequence which has at least 70%, at least 75%, at least 80%, at least 85%, at least 90%, at least 95%, or at least 99% identity to SEQ ID NO: 6. In certain embodiments, the engineered LacR protein comprises SEQ ID NO: 7. In certain embodiments, the engineered LacR protein comprises an amino acid sequence which has at least 70%, at least 75%, at least 80%, at least 85%, at least 90%, at least 95%, or at least 99% identity to SEQ ID NO: 7. In certain embodiments, the transcriptional regulatory system comprises a nucleotide sequence comprises a polh promoter driving a NLS-LacR sequence. In certain embodiments, the transcriptional regulatory system comprises a nucleotide sequence comprising SEQ ID NO: 8. In certain embodiments, the transcriptional regulatory system comprises a nucleotide sequence which has at least 70%, at least 75%, at least 80%, at least 85%, at least 90%, at least 95%, or at least 99% identity to SEQ ID NO: 8.

In certain embodiments, the engineered LacR protein is codon optimized. In certain embodiments, the engineered LacR protein is codon optimized for insect cells. In certain embodiments, the engineered LacR protein is codon optimized for Spodoptera frugiperda insect cells. In certain embodiments, the engineered LacR protein is encoded by SEQ ID NO: 9. In certain embodiments, the engineered LacR protein is encoded by nucleotide sequence which has at least 70%, at least 75%, at least 80%, at least 85%, at least 90%, at least 95%, or at least 99% identity to SEQ ID NO: 9. In certain embodiments, the engineered LacR protein comprises a W220F mutation. In certain embodiments, the engineered LacR protein is encoded by SEQ ID NO: 10. In certain embodiments, the engineered LacR protein is encoded by nucleotide sequence which has at least 70%, at least 75%, at least 80%, at least 85%, at least 90%, at least 95%, or at least 99% identity to SEQ ID NO: 10. In certain embodiments, the engineered LacR protein comprises SEQ ID NO: 11. In certain embodiments, the engineered LacR protein comprises an amino acid sequence which has at least 70%, at least 75%, at least 80%, at least 85%, at least 90%, at least 95%, or at least 99% identity to SEQ ID NO: 11.

In certain embodiments, the transcriptional regulatory system comprises a nucleotide sequence comprises a hybrid gp64-polh promoter (without ATGs in the polh) driving a codon-optimized LacR sequence that includes an optimal Kozak. In certain embodiments, the transcriptional regulatory system comprises a nucleotide sequence comprising SEQ ID NO: 12. In certain embodiments, the transcriptional regulatory system comprises a nucleotide sequence which has at least 70%, at least 75%, at least 80%, at least 85%, at least 90%, at least 95%, or at least 99% identity to SEQ ID NO: 12. In certain embodiments, the transcriptional regulatory system comprises a nucleotide sequence comprising SEQ ID NO: 13. In certain embodiments, the transcriptional regulatory system comprises a nucleotide sequence which has at least 70%, at least 75%, at least 80%, at least 85%, at least 90%, at least 95%, or at least 99% identity to SEQ ID NO: 13.

In certain embodiments, the transcriptional regulatory system comprises a nucleotide sequence comprises a hybrid gp64-polh promoter (without ATGs in the polh) driving a codon-optimized NLS-LacR sequence that includes an optimal Kozak. In certain embodiments, the transcriptional regulatory system comprises a nucleotide sequence comprising SEQ ID NO: 12. In certain embodiments, the transcriptional regulatory system comprises a nucleotide sequence which has at least 70%, at least 75%, at least 80%, at least 85%, at least 90%, at least 95%, or at least 99% identity to SEQ ID NO: 12. In certain embodiments, the transcriptional regulatory system comprises a nucleotide sequence comprising SEQ ID NO: 13. In certain embodiments, the transcriptional regulatory system comprises a nucleotide sequence which has at least 70%, at least 75%, at least 80%, at least 85%, at least 90%, at least 95%, or at least 99% identity to SEQ ID NO: 13.

In certain embodiments, the regulator binding region is a Lac Operator (LacO) nucleotide sequence (usually a 35 bp semipalindromic DNA element). In certain embodiments, the inducer element is a LacR inducer element, such as Lactose, Allolactose (intermediate metabolite of lactose), or isopropyl-β-D-thiogalactose (IPTG) (allolactose analogue). In certain embodiments, the LacR inducer element (e.g., IPTG) binds to LacR and causes a conformational change in LacR to reduce binding affinity to LacO.

In certain embodiments, the regulator element is a Tet repressor (TetR) protein or a tetracycline-controlled transactivator protein (tTA) (composed of TetR fused to strong transactivating domain of VP16 from Herpes simplex virus). In certain embodiments, the regulator element is a TetR protein encoded by a TetR nucleotide sequence. In certain embodiments, the regulator element is a tTA fusion protein encoded by a tTA nucleotide sequence. In certain embodiments, the regulator binding region is a Tet Operator (tetO) nucleotide sequence (usually a 19 bp DNA element) or a Tet Response Element (TRE) (which includes a series of two or more (e.g., seven) repeating tetO units). In certain embodiments, the inducer element is a TetR/tTA inducer element, such as tetracycline (Tet) or a tetracycline analog such as doxycycline (Dox). In certain embodiments, the regulator element includes a TetR protein or a tTA fusion protein, the regulator binding region includes at least one tetO nucleotide sequence (such as a TRE region which includes 2-7 repeating tetO units), and the inducer element is a TetR/tTA inducer element selected from tetracycline (Tet) or doxycycline (Dox). In certain embodiments, the TetR/tTA inducer element (e.g., Tet or Dox) binds to the TetR protein or TetR component of the tTA fusion protein, and causes conformational change in the TetR polypeptide to reduce binding affinity to tetO.

In certain embodiments, the transcriptional regulatory system can include one or more components as described in U.S. Pat. No. 6,133,027 (the contents of which are herein incorporated by reference in its entirety), including specific regulator element, regulator binding regions, and inducer elements.

In certain embodiments, the transcriptional regulatory system includes at least one regulator binding region (i.e., regulator binding sequence) within the expression control region of a viral expression construct. In certain embodiments, the expression control region includes a promoter and at least one regulator binding region. In certain embodiments, the regulator binding region is 5-150 or 5-100 nucleotides from the promoter. In certain embodiments, the regulator binding region is between 5-10, 10-15, 15-20, 20-25, 25-30, 30-35, 35-40, 40-45, 45-50, 50-55, 55-60, 60-65, 65-70, 70-80, 80-90, 90-100, 100-110, 110-120, 120-130, 130-140 or 140-150 nucleotides from the promoter. In certain embodiments, the regulator binding region is placed in a region known to be not essential for promoter function. In certain embodiments, the regulator binding region is a Lac Operator (LacO) nucleotide sequence. In certain embodiments, the Lac Operator (LacO) nucleotide sequence is SEQ ID NO: 14. In certain embodiments, the Lac Operator (LacO) nucleotide sequence is a nucleotide sequence which has at least 75%, at least 80%, at least 85%, at least 90% or at least 95% identity to SEQ ID NO: 14. In certain embodiments, the regulator binding region includes at least one tetO nucleotide sequence (such as a TRE region which includes 2-7 repeating tetO units). In certain embodiments, the promoter is a p10 promoter. In certain embodiments, the promoter is a polh promoter. In certain embodiments, the regulator binding region is a Lac Operator (LacO) nucleotide sequence and the promoter is a p10 promoter.

In certain embodiments, the expression control region includes a promoter and 2-7 regulator binding regions. In certain embodiments, the expression control region includes a promoter and two regulator binding regions. In certain embodiments, the expression control region includes a promoter, and upstream regulator binding region which is upstream of the promoter, and a downstream regulator binding region which is downstream from the promoter. In certain embodiments, the two regulator binding regions have a space interval of 100-300 nucleotides between them (measured from the center nucleotide of each regulator binding region). In certain embodiments, the two regulator binding regions have a space interval of 150-300, 150-250, 150-225, or 150-210 nucleotides between them (measured from the center nucleotide of each regulator binding region). In certain embodiments, the two regulator binding regions have a space interval of 100-105, 105-110, 110-115, 115-120, 120-125, 125-130, 130-135, 135-140, 140-145, 145-150, 150-155, 155-160, 160-165, 165-170, 170-175, 175-180, 180-185, 185-190,190-195, 195-200, 200-205, 200-210, 200-215, 205-210, 205-215, 210-215, 215-220, 220-225, 225-230, 230-235, 235-240, 240-245, 245-250, 250-255, 255-260, 260-265, 265-270, 270-275, 275-280, 280-285, 285-290, 290-295 or 295-300 nucleotides from the promoter. In certain embodiments, the two regulator binding regions have a space interval of 112 nucleotides. In certain embodiments, the two regulator binding regions have a space interval of 148 nucleotides. In certain embodiments, the two regulator binding regions have a space interval of 152 nucleotides. In certain embodiments, the two regulator binding regions have a space interval of 200 nucleotides. In certain embodiments, the two regulator binding regions have a space interval of 208 nucleotides.

In certain embodiments, the expression control region includes a promoter and two Lac Operator (LacO) nucleotide sequences. In certain embodiments, the expression control region includes a promoter, an upstream LacO nucleotide sequences which is upstream of the promoter, and a downstream LacO nucleotide sequences which is downstream of the promoter. In certain embodiments, the promoter is a p10 promoter. In certain embodiments, the promoter is a polh promoter. In certain embodiments, the expression control region includes a p10 promoter, an upstream LacO and downstream LacO, wherein the upstream LacO and downstream LacO have a space interval of 200-215 nucleotides (measured from the center nucleotide of each LacO sequence).

In certain embodiments, the transcriptional regulatory system includes at least one regulator element. In certain embodiments, the transcriptional regulatory system includes at least one regulator element. In certain embodiments, the regulator element is a Lac repressor (LacR) protein. In certain embodiments, the regulator element is a Tet repressor (TetR) protein. In certain embodiments, the regulator element is a tetracycline-controlled transactivator protein (tTA) composed of TetR fused to strong transactivating domain of VP16 from Herpes simplex virus.

In certain embodiments, the regulator element is a polypeptide that binds to one or more regulator binding sequences. In certain embodiments, the regulator element is a polypeptide that binds to two regulator binding sequences. In certain embodiments, the regulator element is a polypeptide that binds to 1-7 regulator binding sequences. In certain embodiments, the regulator element is a polypeptide that binds to one or more LacO sequences. In certain embodiments, the regulator element is a polypeptide that binds to two LacO sequences. In certain embodiments, the regulator element is a LacR protein that binds to one or more (e.g., two) LacO sequences. In certain embodiments, the regulator element is a polypeptide that binds to one or more tetO nucleotide sequence (such as a TRE region which includes 2-7 repeating tetO units). In certain embodiments, the regulator element is a TetR protein or tTA fusion protein that binds to one or more tetO nucleotide sequences.

In certain embodiments, the transcriptional regulatory system includes a promoter, at least one regulator binding region within 100 nucleotides from the promoter and at least one regulator element that binds to the regulator binding region. In certain embodiments, the regulator element is functional in decreasing and/or silencing transcription from the promoter when the regulator element is bound to the regulator binding region within 100 nucleotides from the promoter. In certain embodiments, the regulator element is functional in decreasing and/or silencing the expression of a protein-coding nucleotide sequence from the promoter when the regulator element is bound to the regulator binding region within 100 nucleotides from the promoter. In certain embodiments, the regulator element is functional in decreasing or silencing the expression of a protein-coding nucleotide sequence from the promoter by interfering with RNA polymerase activity at the promoter, thereby inhibiting or reducing transcriptional elongation from the promoter. In certain embodiments, the transcriptional regulatory system includes a p10 promoter, at least one LacO sequence within 100 nucleotides from the p10 promoter, and at least one LacR protein that binds to the LacO sequence. In certain embodiments, the LacR protein is functional in decreasing or silencing the expression of a protein-coding nucleotide sequence from the p10 promoter when the LacR protein is bound to the LacO sequence within 100 nucleotides from the promoter. In certain embodiments, the LacR protein is functional in decreasing or silencing the expression of a protein-coding nucleotide sequence from the p10 promoter when the LacR protein is bound to the LacO sequence by interfering with RNA polymerase activity at the p10 promoter, thereby inhibiting or reducing transcriptional elongation from the p10 promoter.

In certain embodiments, the expression control region includes a promoter, at least two regulator binding regions (i.e., regulator binding sequences) that are within 100 nucleotides from each end of the promoter region and with a space interval of 200-215 nucleotides (measured from the center nucleotide of each regulator binding sequence), and at least one regulator element that binds to the regulator binding region. In certain embodiments, the regulator element is functional in decreasing transcription from the promoter when the regulator element is bound to the regulator binding region within 100 nucleotides from the promoter. In certain embodiments, the regulator element is functional in silencing transcription from the promoter when the regulator element is bound to the regulator binding region within 100 nucleotides from the promoter. In certain embodiments, the regulator element is functional in decreasing or silencing the expression of a protein-coding nucleotide sequence from the promoter when the regulator element is bound to the regulator binding region within 100 nucleotides from the promoter. In certain embodiments, the regulator element is functional in decreasing or silencing the expression of a protein-coding nucleotide sequence from the promoter by interfering with RNA polymerase activity at the promoter, thereby inhibiting or reducing transcriptional elongation from the promoter. In certain embodiments, the transcriptional regulatory system includes a p10 promoter, at least one LacO sequence within 100 nucleotides upstream from the p10 promoter, at least one LacO sequence within 100 nucleotides downstream from the p10 promoter, and at least one LacR protein that simultaneously binds to both the upstream LacO sequence and the downstream LacO sequence. In certain embodiments, the LacR protein is functional in decreasing or silencing the expression of a protein-coding nucleotide sequence from the p10 promoter when the LacR protein is bound to both the upstream LacO sequence and downstream LacO sequence. In certain embodiments, the simultaneous binding of the LacR protein to both the upstream LacO sequence and the downstream LacO sequence results in the formation of a loop structure around the p10 promoter. In certain embodiments, the formation of the loop structure interferes with RNA polymerase activity at the p10 promoter, thereby inhibiting or reducing transcriptional elongation from the p10 promoter.

The present disclosure presents a viral expression construct which includes a nucleotide sequence which encodes a regulator element. In certain embodiments, the viral expression construct includes: (i) a first region or open reading frame (ORF) which includes a protein-coding nucleotide sequence operably linked to an expression control sequence, wherein the expression control sequence includes a promoter and at least one regulator binding region within 100 nucleotides from the promoter; and (ii) a second region or ORF which includes a nucleotide sequence which encodes a regulator element; and wherein the regulator element encoded by the nucleotide sequence in the second region/ORF has a binding affinity for the at least one regulator binding region within the expression control sequence of the first region/ORF. In certain embodiments, the regulator element from the second region/ORF is functional in decreasing or silencing the expression of the protein-coding nucleotide sequence from the promoter in the first region/ORF when the regulator element is bound to the regulator binding region within the expression control sequence of the first region/ORF.

In certain embodiments, the viral expression construct includes a LacI gene (or engineered variation thereof) which encodes a LacR protein (e.g., wt LacR protein or engineered LacR protein). In certain embodiments, the viral expression construct includes: (i) a first region or ORF which includes a protein-coding nucleotide sequence operably linked to an expression control sequence, wherein the expression control sequence includes a p10 promoter and at least one LacO sequence within 100 nucleotides from the promoter; and (ii) a second region or ORF which includes a nucleotide sequence which encodes a LacR protein (e.g., wt LacR protein or engineered LacR protein); and wherein the LacR protein encoded by the nucleotide sequence in the second region/ORF has a binding affinity for the at least one LacO sequence within the expression control sequence of the first region/ORF. In certain embodiments, the LacR protein encoded in the second region/ORF is functional in decreasing or silencing the expression of the protein-coding nucleotide sequence from the p10 promoter in the first region/ORF when the LacR protein is bound to the LacO sequence within the expression control sequence of the first region/ORF. In certain embodiments, the first region/ORF includes at least one LacO sequence within 100 nucleotides upstream from the p10 promoter and at least one LacO sequence within 100 nucleotides downstream from the p10 promoter, wherein the LacR protein can simultaneously bind to both the upstream LacO sequence and the downstream LacO sequence. In certain embodiments, the upstream LacO sequence and downstream LacO sequence have a space interval of 200-215 nucleotides (measured from the center nucleotide of each regulator binding sequence).

In certain embodiments, the viral expression construct includes: (i) a first region or ORF which includes a protein-coding nucleotide sequence operably linked to an expression control sequence, wherein the protein-coding nucleotide sequence includes a nucleotide sequence encoding one or more structural AAV capsid proteins (e.g., VP1, VP2, VP3, or a combination thereof), and wherein the expression control sequence includes a p10 promoter and at least one LacO sequence within 100 nucleotides from the promoter; and (ii) a second region or ORF which includes a nucleotide sequence which encodes a LacR protein (e.g., wt LacR protein or engineered LacR protein); and wherein the LacR protein encoded by the nucleotide sequence in the second region/ORF has a binding affinity for the at least one LacO sequence within the expression control sequence of the first region/ORF. In certain embodiments, the LacR protein encoded in the second region/ORF is functional in decreasing or silencing the expression of the structural AAV capsid proteins from the p10 promoter in the first region/ORF when the LacR protein is bound to the LacO sequence within the expression control sequence of the first ORF. In certain embodiments, the first ORF includes at least one LacO sequence within 100 nucleotides upstream from the p10 promoter and at least one LacO sequence within 100 nucleotides downstream from the p10 promoter, wherein the LacR protein can simultaneously bind to both the upstream LacO sequence and the downstream LacO sequence. In certain embodiments, the upstream LacO sequence and downstream LacO sequence have a space interval of 200-215 nucleotides (measured from the center nucleotide of each regulator binding sequence). In certain embodiments, the protein-coding nucleotide sequence encodes VP1, VP2, and VP3. In certain embodiments, the protein-coding nucleotide sequence encodes VP1 only. In certain embodiments, the protein-coding nucleotide sequence encodes VP2 only. In certain embodiments, the protein-coding nucleotide sequence encodes VP3 only.

In certain embodiments, the viral expression construct includes: (i) a first region or ORF which includes a protein-coding nucleotide sequence operably linked to an expression control sequence, wherein the protein-coding nucleotide sequence includes a nucleotide sequence encoding one or more non-structural AAV replication proteins (e.g., Rep78, Rep52, or a combination thereof), and wherein the expression control sequence includes a p10 promoter and at least one LacO sequence within 100 nucleotides from the promoter; and (ii) a second region or ORF which includes a nucleotide sequence which encodes a LacR protein (e.g., wt LacR protein or engineered LacR protein); and wherein the LacR protein encoded by the nucleotide sequence in the second region/ORF has a binding affinity for the at least one LacO sequence within the expression control sequence of the first region/ORF. In certain embodiments, the LacR protein encoded in the second region/ORF is functional in decreasing or silencing the expression of the non-structural AAV replication proteins from the p10 promoter in the first region/ORF when the LacR protein is bound to the LacO sequence within the expression control sequence of the first region/ORF. In certain embodiments, the first ORF includes at least one LacO sequence within 100 nucleotides upstream from the p10 promoter and at least one LacO sequence within 100 nucleotides downstream from the p10 promoter, wherein the LacR protein can simultaneously bind to both the upstream LacO sequence and the downstream LacO sequence. In certain embodiments, the upstream LacO sequence and downstream LacO sequence have a space interval of 200-215 nucleotides (measured from the center nucleotide of each regulator binding sequence). In certain embodiments, the protein-coding nucleotide sequence encodes Rep78 and Rep52. In certain embodiments, the protein-coding nucleotide sequence encodes Rep78 only. In certain embodiments, the protein-coding nucleotide sequence encodes Rep52 only.

In certain embodiments, the transcriptional regulatory system includes at least one inducer element which reduces the affinity of the regulator element for binding to the regulator binding region. In certain embodiments, the inducer element is a LacR inducer element. In certain embodiments, the LacR inducer element binds to LacR and causes a conformational change in LacR to reduce binding affinity to LacO. In certain embodiments, the LacR inducer element is Lactose. In certain embodiments, the LacR inducer element is Allolactose (intermediate metabolite of lactose). In certain embodiments, the LacR inducer element is isopropyl-β-D-thiogalactose (IPTG) (allolactose analogue). In certain embodiments, the inducer element is a TetR/tTA inducer element. In certain embodiments, the TetR/tTA inducer element binds to TetR (or the TetR component of tTA) and causes a conformational change in TetR to reduce binding affinity to TetO. In certain embodiments, the TetR/tTA inducer element is tetracycline (Tet). In certain embodiments, the TetR/tTA inducer element is a tetracycline analog. In certain embodiments, the TetR/tTA inducer element is doxycycline (Dox).

In certain embodiments, the inducer element is present at a target concentration of the inducer element. In certain embodiments, the inducer element is present at a concentration of about 0.0 μM, about 0.5 μM, about 1.0 μM, about 1.5 μM, about 2.0 μM, about 2.5 μM, about 3.0 μM, about 3.5 μM, about 4.0 μM, about 4.5 μM, about 5.0 μM, about 5.5 μM, about 6.0 μM, about 6.5 μM, about 7.0 μM, about 7.5 μM, about 8.0 μM, about 8.5 μM, about 9.0 μM, about 9.5 μM, about 10.0 μM, about 10.5 μM, about 11.0 μM, about 11.5 μM, about 12.0 μM, about 12.5 μM, about 13.0 μM, about 13.5 μM, about 14.0 μM, about 14.5 μM, about 15.0 μM, about 15.5 μM, about 16.0 μM, about 16.5 μM, about 17.0 μM, about 17.5 μM, about 18.0 μM, about 18.5 μM, about 19.0 μM, about 19.5 μM, about 20.0 μM, about 20.5 μM, about 21.0 μM, about 21.5 μM, about 22.0 μM, about 22.5 μM, about 23.0 μM, about 23.5 μM, about 24.0 μM, about 24.5 μM, about 25.0 μM, about 25.5 μM, about 26.0 μM, about 26.5 μM, about 27.0 μM, about 27.5 μM, about 28.0 μM, about 28.5 μM, about 29.0 μM, about 29.5 μM, or about 30 μM.

In certain embodiments, the inducer element is present at a concentration of about 0.0 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 105 μM, about 110 μM, about 115 μM, about 120 μM, about 125 μM, about 130 μM, about 135 μM, about 140 μM, about 145 μM, about 150 μM, about 155 μM, about 160 μM, about 165 μM, about 170 μM, about 175 μM, about 180 μM, about 185 μM, about 190 μM, about 195 μM, about 200 μM, about 205 μM, about 210 μM, about 215 μM, about 220 μM, about 225 μM, about 230 μM, about 235 μM, about 240 μM, about 245 μM, about 250 μM, about 255 μM, about 260 μM, about 265 μM, about 270 μM, about 275 μM, about 280 μM, about 285 μM, about 290 μM, about 295 μM, or about 300 μM.

In certain embodiments, the inducer element is present at a concentration between about 1.0 μM to about 200 μM, between about 1.0 μM to about 100 μM, between about 1.0 μM to about 50 μM, between about 1.0 μM to about 40 μM, between about 1.0 μM to about 35 μM, between about 10 μM to about 35 μM, between about 10 μM to about 25 μM, between about 12.5 μM to about 22.5 μM, between about 13 μM to about 17 μM, between about 5 μM to about 15 μM, between about 8 μM to about 12 μM, between about 1.0 μM to about 5.0 μM, between about 1.0 μM to about 3.0 μM, between about 2.0 μM to about 3.0 μM, between about 5.0 μM to about 15.0 μM, between about 5.0 μM to about 12.0 μM, or between about 5.0 μM to about 10.0 μM. In certain embodiments, the inducer element is present at a concentration of about 10 μM.

In certain embodiments, the transcriptional regulatory system includes a controlled amount or concentration of the inducer element. In certain embodiments, the amount of the inducer element included within the transcriptional regulatory system is proportional to the effect the inducer element has on the binding affinity between the regulator element and the regulator binding sequence. In certain embodiments, controlling the concentration of the inducer element within the transcriptional regulatory system allows for corresponding control of the expression of a protein-coding nucleotide sequence from the promoter.

In certain embodiments, the inducer element is not present or present at low concentrations. As a result, the regulator element has a high affinity for binding to the regulator binding region and expression of a protein-coding nucleotide sequence from the promoter is decreased or silenced. In certain embodiments, the inducer element is present or present at high concentrations. As a result, the regulator element has a low affinity for binding to the regulator binding region and expression of a protein-coding nucleotide sequence from the promoter is not decreased or minimally decreased. In certain embodiments, the concentration of the regulator element present in the transcriptional regulatory system is proportional to the affinity of the regulator element for binding to the regulator binding region. In certain embodiments, the concentration of the regulator element present in the transcriptional regulatory system is proportional to the level of decreased expression of a protein-coding nucleotide sequence resulting from the binding of regulator elements to regulator binding regions. In certain embodiments, the concentration of the regulator element present in the transcriptional regulatory system is proportional to amount of protein material produced by the expression of the protein-coding nucleotide sequence from a promoter.

In certain embodiments, the transcriptional regulatory system is operable with a nucleotide sequence encoding one or more structural AAV capsid proteins (e.g., VP1, VP2, VP3, or a combination thereof), such that the concentration of the regulator element present in the transcriptional regulatory system is proportional to amount of the AAV capsid protein material produced by the expression of the protein-coding nucleotide sequence from a promoter. In certain embodiments, the transcriptional regulatory system is operable with a nucleotide sequence encoding VP1 only. In certain embodiments, the transcriptional regulatory system is operable with a nucleotide sequence encoding VP2 only. In certain embodiments, the transcriptional regulatory system is operable with a nucleotide sequence encoding VP3 only. In certain embodiments, a transcriptional regulatory system is engineered to provide a VP protein ratio (VP1:VP2:VP3) of about 1-2:1-2:10 when the viral expression construct is processed by a viral production cell. In certain embodiments, the transcriptional regulatory system is engineered to include a concentration of a regulator element which results in a VP protein ratio (VP1:VP2:VP3) of about 1-2:1-2:10 when the viral expression construct is processed by a viral production cell.

In certain embodiments, the transcriptional regulatory system is operable with a nucleotide sequence encoding one or more non-structural AAV replication proteins (e.g., Rep78, Rep52, or a combination thereof), such that the concentration of the regulator element present in the transcriptional regulatory system is proportional to amount of the AAV replication protein material produced by the expression of the protein-coding nucleotide sequence from a promoter. In certain embodiments, the transcriptional regulatory system is operable with a nucleotide sequence encoding Rep78 only. In certain embodiments, the transcriptional regulatory system is operable with a nucleotide sequence encoding Rep52 only. In certain embodiments, a transcriptional regulatory system is engineered to provide a ratio of p5 Rep proteins (Rep78 and Rep68) to p19 Rep proteins (Rep52 and Rep40) of about 1:1-10 when the viral expression construct is processed by a viral production cell. In certain embodiments, the transcriptional regulatory system is engineered to include a concentration of a regulator element which results in a ratio of p5 Rep proteins (Rep78 and Rep68) to p19 Rep proteins (Rep52 and Rep40) of about 1:1-10 when the viral expression construct is processed by a viral production cell.

In certain embodiments, the transcriptional regulatory system can include one or more regulatable elements presented in WO2016137949 or WO2017075335, the contents of each of which are herein incorporated by reference in their entireties.

### Polynucleotide Insertion

In certain embodiments, a viral expression construct or a payload construct of the present disclosure (e.g., bacmid) can include a polynucleotide incorporated into the bacmid by standard molecular biology techniques (e.g., transposon donor/acceptor system) known and performed by a person skilled in the art. In certain embodiments, the polynucleotide incorporated into the bacmid can include an expression control sequence operably linked to a protein-coding nucleotide sequence. In certain embodiments, the polynucleotide incorporated into the bacmid can include an expression control sequence which includes a promoter, such as p10 or polh, and which is operably linked to a nucleotide sequence which encodes a structural AAV capsid protein (e.g., VP1, VP2, VP3 or a combination thereof). In certain embodiments, the polynucleotide incorporated into the bacmid can include an expression control sequence which includes a promoter, such as p10 or polh, and which is operably linked to a nucleotide sequence which encodes a non-structural AAV capsid protein (e.g., Rep78, Rep52, or a combination thereof).

In certain embodiments, the polynucleotide insert can be incorporated into the bacmid using the Gibson Assembly method, as described in Gibson et al. (2009) Nat. Methods 6, 343-345, and Gibson et al. (2010) Science 329, 52-56; the contents of which are each incorporated herein by reference in their entireties as related to the use of Gibson Assembly method for incorporating polynucleotide inserts into a bacmid. In certain embodiments, the polynucleotide insert can include one or more Gibson Assembly sequences at the 5′ end of the insert, at the 3′ end of the insert, or at both the 5′ end and 3′ end of the insert; such that the one or more Gibson Assembly sequences allow for the incorporation of the polynucleotide insert into a target location of bacmid. In certain embodiments, the Gibson Assembly method can include the use of NEBuilder Hifi optimized enzyme mix.

In certain embodiments, the polynucleotide insert can be incorporated into the bacmid at the location of a baculoviral gene. In certain embodiments, the polynucleotide insert can be incorporated into the bacmid at the location of a non-essential baculoviral gene. In certain embodiments, the polynucleotide insert can be incorporated into the bacmid by replacing a baculoviral gene or a portion of the baculoviral gene with the polynucleotide insert. In certain embodiments, the polynucleotide insert can be incorporated into the bacmid by replacing a baculoviral gene or a portion of the baculoviral gene with a fusion-polynucleotide which includes the polynucleotide insert and the baculoviral gene (or portion thereof) being replaced.

In certain embodiments, the polynucleotide can be incorporated into the bacmid at the location of a restriction endonuclease (REN) cleavage site (i.e., REN access point) associated with a baculoviral gene. In certain embodiments, the polynucleotide can be incorporated into the bacmid using one or more endonucleases (e.g., homing endonucleases). See, for example, Lihoradova et al., J Virol Methods, 140(1-2):59-65 (2007), the content of which is incorporated herein by reference in its entirety as related to the direct cloning of foreign DNA into baculovirus genomes, and insofar as it does not conflict with the present disclosure.

In certain embodiments, the REN access point in the bacmid is FseI (corresponding with the global transactivator (gta) baculovirus gene) (ggccggcc). In certain embodiments, the REN access point in the bacmid is SdaI (corresponding with the DNA polymerase baculovirus gene) (cctgcagg). In certain embodiments, the REN access point in the bacmid is MauBI (corresponding with the lef-4 baculovirus gene) (cgcgcgcg). In certain embodiments, the REN access point in the bacmid is SbfI (corresponding with the gp64/gp67 baculovirus gene) (cctgcagg). In certain embodiments, the REN access point in the bacmid is I-CeuI (corresponding with the v-cath baculovirus gene) (SEQ ID NO: 1). In certain embodiments, the REN access point in the bacmid is AvrII (corresponding with the ecdysteroid UDP-glucosyltransferase (egt) baculovirus gene) (cctagg). In certain embodiments, the REN access point in the bacmid is NheI (gctagc). In certain embodiments, the REN access point in the bacmid is SpeI (actagt). In certain embodiments, the REN access point in the bacmid is BstZ17I (gtatac). In certain embodiments, the REN access point in the bacmid is NcoI (ccatgg). In certain embodiments, the REN access point in the bacmid is MluI (acgcgt).

Polynucleotides can be incorporated into these REN access points by: (i) providing a polynucleotide insert which has been engineered to include a target REN cleavage sequence (e.g., a polynucleotide insert engineered to include FseI REN sequences at both ends of the polynucleotide); (ii) proving a bacmid which includes the target REN access point for polynucleotide insertion (e.g., a variant of the AcMNPV bacmid bMON14272 which includes an FseI cleavage site (ii) digesting the REN-engineered polynucleotide with the appropriate REN enzyme (e.g., using FseI enzyme to digesting the REN-engineering polynucleotide which includes the FseI regions at both ends, to produce a polynucleotide-FseI insert); (iii) digesting the bacmid with the same REN enzyme to produce a single-cut bacmid at the REN access point (e.g., using FseI enzyme to produce a single-cut bacmid at the FseI location); and (iv) ligating the polynucleotide insert into the single-cut bacmid using an appropriate ligation enzyme, such as T4 ligase enzyme. The result is engineered bacmid DNA which includes the engineered polynucleotide insert at the target REN access point.

The insertion process can be repeated one or more times to incorporate other engineered polynucleotide inserts into the same bacmid at different REN access points (e.g., insertion of a first engineered polynucleotide insert at the AvrII REN access point in the egt, followed by insertion of a second engineered polynucleotide insert at the I-CeuI REN access point in the cath gene, and followed by insertion of a third engineered polynucleotide insert at the FseI REN access point in the gta gene).

In certain embodiments, the polynucleotide insert can be incorporated into the bacmid by splitting a baculoviral gene with the polynucleotide insert (i.e., the polynucleotide insert is incorporated into the middle of the gene, separating a 5′-portion of the gene from a 3-portion of the bacmid gene). In certain embodiments, the polynucleotide insert can be incorporated into the bacmid by splitting a baculoviral gene with the fusion-polynucleotide which includes the polynucleotide insert and a portion of the baculoviral gene which was split. In certain embodiments, the 3′ end of the fusion-polynucleotide includes the 5′-portion of the gene that was split, such that the 5′-portion of the gene in the fusion-polynucleotide and the 3′-portion of the gene remaining in the bacmid form a full or functional portion of the baculoviral gene. In certain embodiments, the 5′ end of the fusion-polynucleotide includes the 3′-portion of the gene that was split, such that the 3′-portion of the gene in the fusion-polynucleotide and the 5′-portion of the gene remaining in the bacmid form a full or functional portion of the baculoviral gene. In certain embodiments, fusion-polynucleotides are engineered and produced to include components from the gta gene ORF (full/partial Ac-lef12 promoter, full/partial Ac-gta gene). Non-limiting examples of fusion polynucleotides of the present disclosure include the polynucleotides of SEQ ID NO: 2 and SEQ ID NO: 3.

In certain embodiments, restriction endonuclease (REN) cleavage can be used to remove one or more wild-type genes from a bacmid. In certain embodiments, restriction endonuclease (REN) cleavage can be used to remove one or more engineered polynucleotide insert which has been previously inserted into the bacmid. In certain embodiments, restriction endonuclease (REN) cleavage can be used to replace one or more engineered polynucleotide inserts with a different engineered polynucleotide insert which includes the same REN cleavage sequences (e.g., an engineered polynucleotide insert at the FseI REN access point can be replaced with a different engineered polynucleotide insert which includes FseI REN cleavage sequences).

In certain embodiments, one or more polynucleotide inserts comprising a VP-coding region can be incorporated into the bacmid at one or more REN access points of the bacmid genome. In certain embodiments, a polynucleotide inserts comprising a VP1-only coding region (e.g., atgVP1 sequence) can be incorporated into the bacmid at one or more REN access points of the bacmid genome (e.g., chiA gene locus). In certain embodiments, a polynucleotide inserts comprising a VP2-only coding region (e.g., atgVP2 sequence) can be incorporated into the bacmid at one or more REN access points of the bacmid genome (e.g., gta gene locus). In certain embodiments, a polynucleotide inserts comprising a VP3-only coding region (e.g., atgVP3 sequence) can be incorporated into the bacmid at one or more REN access points of the bacmid genome (e.g., polh gene locus). In certain embodiments, a first polynucleotide insert comprising a VP1-only coding region (e.g., atgVP1 sequence) can be incorporated into the bacmid at a first REN access point of the bacmid genome (e.g., chiA gene locus), and/or a second polynucleotide insert comprising a VP2-only coding region (e.g., atgVP2 sequence) can be incorporated into the bacmid at a second REN access point of the bacmid genome (e.g., gta gene locus), and/or a third polynucleotide insert comprising a VP3-only coding region (e.g., atgVP3 sequence) can be incorporated into the bacmid at a third REN access point of the bacmid genome (e.g., polh gene locus).

In certain embodiments, two or more polynucleotide inserts comprising a VP-coding region can be incorporated into the bacmid independently through multiple incorporation steps (e.g., separate Gibson assembly mixtures). In certain embodiments, two or more polynucleotide inserts comprising a VP-coding region can be incorporated into the bacmid in a single incorporation step (e.g., single Gibson assembly mixtures). In certain embodiments, a VP1-only coding region (e.g., atgVP1 sequence), and/or a VP2-only coding region (e.g., atgVP2 sequence), and/or a VP3-only coding region (e.g., atgVP3 sequence) can be incorporated into the bacmid independently through multiple incorporation steps (e.g., separate Gibson assembly mixtures). In certain embodiments, a VP1-only coding region (e.g., atgVP1 sequence), and/or a VP2-only coding region (e.g., atgVP2 sequence), and/or a VP3-only coding region (e.g., atgVP3 sequence) can be incorporated into the bacmid in a single incorporation step (e.g., single Gibson assembly mixtures).

In certain embodiments, one or more polynucleotide inserts comprising a Rep-coding region can be incorporated into the bacmid at one or more REN access points of the bacmid genome. In certain embodiments, a polynucleotide insert comprising a Rep78-only coding region (e.g., atgRep78 sequence) can be incorporated into the bacmid at one or more REN access points of the bacmid genome. In certain embodiments, a polynucleotide insert comprising a Rep52-only coding region (e.g., atgRep52 sequence) can be incorporated into the bacmid at one or more REN access points of the bacmid genome. In certain embodiments, a first polynucleotide insert comprising a Rep78-only coding region (e.g., atgRep78 sequence) can be incorporated into the bacmid at a first REN access point of the bacmid genome, and/or a second polynucleotide insert comprising a Rep52-only coding region (e.g., atgRep52 sequence) can be incorporated into the bacmid at a second REN access point of the bacmid genome.

In certain embodiments, one or more polynucleotide inserts comprising a VP-coding region or a Rep-coding region can be incorporated into the bacmid at one or more REN access points of the bacmid genome. In certain embodiments, a first polynucleotide insert comprising a VP1-only coding region (e.g., atgVP1 sequence) can be incorporated into the bacmid at a first REN access point of the bacmid genome (e.g., chiA gene locus), and/or a second polynucleotide insert comprising a VP2-only coding region (e.g., atgVP2 sequence) can be incorporated into the bacmid at a second REN access point of the bacmid genome (e.g., gta gene locus), and/or a third polynucleotide insert comprising a VP3-only coding region (e.g., atgVP3 sequence) can be incorporated into the bacmid at a third REN access point of the bacmid genome (e.g., Tn7/polh gene locus), and/or a fourth polynucleotide insert comprising a Rep78-only coding region (e.g., atgRep78 sequence) can be incorporated into the bacmid at a fourth REN access point of the bacmid genome (e.g., Tn7/polh gene locus), and/or a fifth polynucleotide insert comprising a Rep52-only coding region (e.g., atgRep52 sequence) can be incorporated into the bacmid at a fifth REN access point of the bacmid genome (e.g., egt gene locus).

In certain embodiments, one or more polynucleotide inserts comprising a VP-coding region, a Rep-coding region, or sequence encoding a regulator element of a transcriptional regulatory system (e.g., NLS-LacR sequence encoding a LacR protein) can be incorporated into the bacmid at one or more REN access points of the bacmid genome. In certain embodiments, a first polynucleotide insert comprising a VP1-only coding region (e.g., atgVP1 sequence) can be incorporated into the bacmid at a first REN access point of the bacmid genome (e.g., chiA gene locus), and/or a second polynucleotide insert comprising a VP2-only coding region (e.g., atgVP2 sequence) can be incorporated into the bacmid at a second REN access point of the bacmid genome (e.g., gta gene locus), and/or a third polynucleotide insert comprising a VP3-only coding region (e.g., atgVP3 sequence) can be incorporated into the bacmid at a third REN access point of the bacmid genome (e.g., Tn7/polh gene locus), and/or a fourth polynucleotide insert comprising a Rep78-only coding region (e.g., atgRep78 sequence) can be incorporated into the bacmid at a fourth REN access point of the bacmid genome (e.g., Tn7/polh gene locus), and/or a fifth polynucleotide insert comprising a Rep52-only coding region (e.g., atgRep52 sequence) can be incorporated into the bacmid at a fifth REN access point of the bacmid genome (e.g., egt gene locus), and/or a sixth polynucleotide insert comprising a sequence encoding a regulator element of a transcriptional regulatory system (e.g., NLS-LacR sequence encoding a LacR protein) can be incorporated into the bacmid at a sixth REN access point of the bacmid genome (e.g., p74 gene locus).

### Cells and Vectors

Cells for the production of the viral proteins from the expression construct may be selected from any biological organism, comprising prokaryotic (e.g., bacterial) cells, and eukaryotic cells, comprising, insect cells, yeast cells and mammalian cells. In certain embodiments, the AAV expression constructs of the present disclosure may be produced in a viral production cell that comprises an insect cell.

Growing conditions for insect cells in culture, and production of heterologous products in insect cells in culture are well-known in the art, see U.S. Pat. No. 6,204,059, the content of which is incorporated herein by reference in its entirety as related to the growth and use of insect cells in viral production, insofar as it does not conflict with the present disclosure.

Any insect cell which allows for replication of parvovirus and which can be maintained in culture can be used in accordance with the present disclosure. AAV viral production cells commonly used for production of recombinant AAV particles comprise, but is not limited to, Spodoptera frugiperda, comprising, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, N J (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of which are each incorporated herein by reference in their entireties as related to the use of insect cells in viral production, insofar as they do not conflict with the present disclosure.

In one embodiment, the AAV particles are made using the methods described in WO2015/191508, the content of which is incorporated herein by reference in its entirety, insofar as it does not conflict with the present disclosure.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the content of which is incorporated herein by reference in its entirety, insofar as it does not conflict with the present disclosure.

Expansion, culturing, transfection, infection and storage of insect cells can be carried out in any cell culture media, cell transfection media or storage media known in the art, including Hyclone SFX Insect Cell Culture Media, Expression System ESF AF Insect Cell Culture Medium, ThermoFisher Sf900II media, ThermoFisher Sf900III media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus Production Systems

In certain embodiments, processes of the present disclosure can comprise production of AAV particles or viral vectors in a baculoviral system using a viral expression construct and a payload construct vector. In certain embodiments, the baculoviral system comprises Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated into the bacmid by standard molecular biology techniques (e.g., transposon donor/acceptor system) known and performed by a person skilled in the art. Transfection of separate viral replication cell populations produces two or more groups (e.g., two, three) of baculoviruses (BEVs), one or more group which can comprise the viral expression construct (e.g., the baculovirus is an “Expression BEV” or “expressionBac”), and one or more group which can comprise the payload construct (e.g., the baculovirus is a “Payload BEV” or “payloadBac”). The baculoviruses may be used to infect a viral production cell for production of AAV particles or viral vector.

In certain embodiments, the process comprises transfection of a single viral replication cell population to produce a single baculovirus (BEV) group which comprises both the viral expression construct and the payload construct. These baculoviruses may be used to infect a viral production cell for production of AAV particles or viral vector.

In certain embodiments, BEVs are produced using a Bacmid Transfection agent, such as Promega FuGENE HD, WFI water, or ThermoFisher Cellfectin II Reagent. In certain embodiments, BEVs are produced and expanded in viral production cells, such as an insect cell.

In certain embodiments, the method utilizes seed cultures of viral production cells that comprise one or more BEVs, comprising baculovirus infected insect cells (BIICs). The seed BIICs have been transfected/transduced/infected with an Expression BEV which comprises a viral expression construct, and also a Payload BEV which comprises a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN₂ vapor.

Baculoviruses are made of several essential proteins which are essential for the function and replication of the Baculovirus, such as replication proteins, envelope proteins and capsid proteins. The Baculovirus genome thus comprises several essential-gene nucleotide sequences encoding the essential proteins. As a non-limiting example, the genome can comprise an essential-gene region which comprises an essential-gene nucleotide sequence encoding an essential protein for the Baculovirus construct. The essential protein can comprise: GP64 baculovirus envelope protein, VP39 baculovirus capsid protein, or other similar essential proteins for the Baculovirus construct.

Baculovirus expression vectors (BEV) for producing AAV particles in insect cells, comprising but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the content of which is incorporated herein by reference in its entirety as related to the production and use of BEVs and viral particles, insofar as it does not conflict with the present disclosure.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture Wasilko D J et al. Protein Expr Purif. 2009 June; 65(2):122-32, the content of which is incorporated herein by reference in its entirety as related to the production and use of BEVs and viral particles, insofar as it does not conflict with the present disclosure.

A genetically stable baculovirus may be used to produce a source of the one or more of the components for producing AAV particles in invertebrate cells. In certain embodiments, defective baculovirus expression vectors may be maintained episomally in insect cells. In such an embodiment the corresponding bacmid vector is engineered with replication control elements, comprising but not limited to promoters, enhancers, and/or cell-cycle regulated replication elements.

The expression construct (e.g., baculovirus expression constructs described herein) or combination of baculovirus expression constructs described herein may be used, for example, to produce adeno-associated virus (AAV) particles.

In some embodiments, one or more of the baculovirus genomes described herein, and/or baculovirus expression construct described herein, comprise a nucleotide sequence encoding one or more adeno-associated virus (AAV) genes.

The AAV production of the present disclosure comprises processes and methods for producing AAV particles and viral vectors which can contact a target cell to deliver a payload construct, e.g., a recombinant viral construct, which comprises a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

In some embodiments, the AAV genes needed to produce an AAV particle are provided in one or more of baculovirus genomes described herein, and/or baculovirus expression construct described herein. For example, in some embodiments, the nucleic acid sequences encoding one or more Rep proteins may be present in one or more baculovirus expression constructs described herein, and the nucleic acid sequences encoding the VP capsid proteins and payload may be present in one or more separate baculovirus expression constructs described herein.

In some embodiments, one or more of baculovirus genome described herein, and/or baculovirus expression construct described herein encodes an AAV Rep protein. In some embodiments, one or more of the baculovirus genome described herein, and/or baculovirus expression construct described herein encodes Rep40, Rep52, Rep68, Rep78, or a combination thereof. In some embodiments, one or more baculovirus genome described herein, and/or baculovirus expression construct described herein encodes a Rep52 protein and/or a Rep78 protein.

In some embodiments, one or more of the baculovirus genome described herein, and/or baculovirus expression construct described herein encodes an AAV capsid protein, e.g., a VP1 protein, a VP2 protein, a VP3 protein, or a combination thereof.

In some embodiments, one or more of the baculovirus genome described herein, and/or baculovirus expression construct described herein encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein, or a variant thereof. In some embodiments, one or more of the baculovirus genome described herein, and/or baculovirus expression construct described herein encodes an AAV5 capsid protein or variant thereof, or an AAV9 capsid protein or variant thereof.

In certain embodiments, baculoviruses may be engineered with a marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In certain embodiments, stable viral producing cells permissive for baculovirus infection are engineered with at least one stable integrated copy of any of the elements necessary for AAV replication and vector production comprising, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In certain embodiments, the Baculovirus expression vectors (BEV) are based on the Autographa californica multicapsid nucleopolyhedrosis virus (AcMNPV baculovirus, e.g., strain E2) or BmNPV baculovirus. In certain embodiments, a bacmid of the present disclosure is based on (i.e., engineered variant of) an AcMNPV bacmid such as bmon14272, vAce25ko or vAclef11KO.

In certain embodiments, the Baculovirus expression vectors (BEV) is a BEV in which the baculoviral v-cath proteinase gene has been mutated, partially deleted, or fully deleted (“v-cath modified BEV”). In certain embodiments, the BEVs lack the v-cath gene or comprise a mutationally inactivated version of the v-cath gene (“v-cath inactivated BEV”). In certain embodiments, the BEVs lack the v-cath gene. In certain embodiments, the BEVs comprise a mutationally inactivated version of the v-cath gene. In certain embodiments, the Baculovirus expression vectors (BEV) is a BEV in which the baculoviral chiA chitinase gene has been mutated, partially deleted, or fully deleted (“chiA modified BEV”). In certain embodiments, the BEVs lack the chiA gene or comprise a mutationally inactivated version of the chiA gene (“chiA inactivated BEV”). In certain embodiments, the BEVs lack the chiA gene. In certain embodiments, the BEVs comprise a mutationally inactivated version of the chiA gene. In certain embodiments, the Baculovirus expression vectors (BEV) is a BEV in which the baculoviral v-cath proteinase gene and/or the baculoviral chiA chitinase gene have been mutated, partially deleted, or fully deleted (“v-cath modified BEV”). In certain embodiments, the v-cath and/or chiA genes are mutated/deleted by homologous recombination. In certain embodiments, the v-cath and/or chiA genes are mutated/deleted by homologous recombination with regions mapping to the chiA C terminus and gp64 C terminus derived from AcMNPV strain C6 (rather than parental strain E2). In certain embodiments, the v-cath and/or chiA genes are mutated/deleted by homologous recombination, which results in several point mutations relative to strain E2 (i.e., in the vestigial chiA C-terminus). In certain embodiments, the v-cath and/or chiA genes are mutated/deleted by replacement with a 26-bp recognition site of homing endonuclease I-CeuI. In certain embodiments, the chiA gene is mutated/deleted such that a portion of the chiA C terminus is left to retain the promoter region of essential baculovirus gene lef7.

In certain embodiments, the v-cath and/or chiA genes are mutated/deleted by replacement with an AscI-flanked LacZa cassette (e.g., AscI-flanked codon-optimized LacZa cassette). In certain embodiments, the AscI-flanked LacZa cassette is inserted functionally downstream from a p10 promoter in the v-cath locus. In certain embodiments, the AscI-flanked LacZa cassette allows for blue/white colony phenotyping in colony screening steps. In certain embodiments, the AscI-flanked LacZa cassette can be digested with AscI, thereby resulting in DNA ends which are compatible with Gibson assembly of Pac-excised sequence inserts (e.g., PacI-excised transgene inserts from transgene plasmid constructs, or VP1/VP2/VP3 expression constructs).

In certain embodiments, the Baculovirus expression vectors (BEV) is a BEV in which baculovirus gene p26 is deleted or mutationally inactivated. In certain embodiments, the Baculovirus expression vectors (BEV) is a BEV in which baculovirus gene p10 is deleted or mutationally inactivated. In certain embodiments, the Baculovirus expression vectors (BEV) is a BEV in which baculovirus gene p74 is deleted or mutationally inactivated. In certain embodiments, the Baculovirus expression vectors (BEV) is a BEV in which baculovirus genes p26, p10, and/or p74 are deleted or mutationally inactivated. See, e.g., Hitchman et al, Cell biology and toxicology 26.1 (2010): 57-68; which is incorporated herein by reference in its entirety as related to the deletion, replacement, and/or mutational inactivation of p26, p10, and/or p74 genes in a baculovirus vector. In certain embodiments, the Baculovirus expression vectors (BEV) is a BEV in which baculovirus genes p26, p10, and/or p74 are deleted and replaced with an I-SceI-flanked chloramphenicol-resistance cassette. In certain embodiments, the chloramphenicol-resistance cassette is removed to provide a single I-SceI cut site.

In certain embodiments, the Baculovirus expression vectors (BEV) is a BEV in which an AscI-flanked LacZa cassette (e.g., AscI-flanked codon-optimized LacZa cassette) is inserted between the kanamycin resistance cassette and the mini-F replicon (e.g., polyhedrin locus) of the baculovirus vector (e.g., by replacing the native LacZa cassette, such as the native LacZa cassette in bMON14272). In certain embodiments, the AscI-flanked LacZa cassette allows for blue/white colony phenotyping in colony screening steps. In certain embodiments, the AscI-flanked LacZa cassette can be digested with AscI, thereby resulting in DNA ends which are compatible with Gibson assembly of PacI-excised sequence inserts (e.g., PacI-excised transgene inserts from transgene plasmid constructs). In certain embodiments, the AscI-flanked LacZa cassette is removed from the polyhedrin locus, and replaced with a single SrfI cut site.

In certain embodiments, the Baculovirus expression vectors (BEV) is a BEV in which the SrfI site located in the ccdB ORF of the bacterial mini-F replicon is silently mutated (i.e., no amino acid change). In certain embodiments, the Baculovirus expression vectors (BEV) is a BEV in which AscI sites in the ac-arif-1 and ac-pkip-1 genes are silently mutated (i.e., no amino acid changes).

Viral production bacmids of the present disclosure can comprise deletion of certain baculoviral genes or loci.

The present disclosure presents methods for producing a baculovirus infected insect cell (BIIC), e.g., expression BIICs and/or payload BIICs. In certain embodiments, the present disclosure presents methods for producing a baculovirus infected insect cell (BIIC) which comprises the following steps: (a) introducing a volume of cell culture medium into a bioreactor; (b) introducing at least one viral production cell (VPC) into the bioreactor and expanding the number of VPCs in the bioreactor to a target VPC cell density; (c) introduction at least one Baculoviral Expression Vector (BEV) into the bioreactor, wherein the BEV comprises an AAV viral expression construct or an AAV payload construct; (d) incubating the mixture of VPCs and BEVs in the bioreactor under conditions which allow at least one BEV to infect at least one VPC to produce a baculovirus infected insect cell (BIIC); (e) incubating the bioreactor under conditions which allow the number of BIICs in the bioreactor to reach a target BIIC cell density; and (f) harvesting the BIICs from the bioreactor. In certain embodiments, the bioreactor has a volume of at least 5 L, 10 L, 20 L, 50 L, 100 L, or 200 L. In certain embodiments, the volume of cell culture medium (i.e., working volume) in the bioreactor is at least 5 L, 10 L, 20 L, 50 L, 100 L, or 200 L.

In certain embodiments, the VPC density at BEV introduction is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at BEV introduction is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the target VPC cell density at BEV introduction is 1.5-4.0×10⁶ cells/mL. In certain embodiments, the target VPC cell density at BEV introduction is 2.0-3.5×10⁶ cells/mL.

In certain embodiments, the BEVs are introduced into the bioreactor at a target Multiplicity of Infection (MOI) of BEVs to VPCs. In certain embodiments, the BEV MOI is 0.0005-0.003, or more specifically 0.001-0.002.

In certain embodiments, the BIICs are harvested from the bioreactor at a specific BIIC cell density. In certain embodiments, the BIICs harvested from the bioreactor have a specific BIIC cell density. In certain embodiments, the BIIC cell density at harvesting is 6.0-18.0×10⁶ cells/mL, 8.0-16.5×10⁶ cells/mL, 10.0-16.5×10⁶ cells/mL.

In certain embodiments, BIICs (expression BIICs, payload BIICs) are used to transfect viral production cells, e.g., Sf9 cells. In some embodiments, baculoviruses comprising bacmids such as BEVs (expressionBacs, payloadBacs) are used to transfect viral production cells, e.g., Sf9 cells.

### Other

In certain embodiments expression hosts comprise, but are not limited to, bacterial species within the genera Escherichia, Bacillus, Pseudomonas, or Salmonella.

In certain embodiments, a host cell which comprises AAV rep and cap genes stably integrated within the cell's chromosomes, may be used for AAV particle production. In a non-limiting example, a host cell which has stably integrated in its chromosome at least two copies of an AAV rep gene and AAV cap gene may be used to produce the AAV particle according to the methods and constructs described in U.S. Pat. No. 7,238,526, the content of which is incorporated herein by reference in its entirety as related to the production of viral particles, insofar as it does not conflict with the present disclosure.

In certain embodiments, the AAV particle can be produced in a host cell stably transformed with a molecule comprising the nucleic acid sequences which permit the regulated expression of a rare restriction enzyme in the host cell, as described in US20030092161 and EP1183380, the contents of which are each incorporated herein by reference in their entireties as related to the production of viral particles, insofar as they do not conflict with the present disclosure.

In certain embodiments, production methods and cell lines to produce the AAV particle may comprise, but are not limited to those taught in PCT/US1996/010245, PCT/US1997/015716, PCT/US1997/015691, PCT/US1998/019479, PCT/US1998/019463, PCT/US2000/000415, PCT/US2000/040872, PCT/US2004/016614, PCT/US2007/010055, PCT/US1999/005870, PCT/US2000/004755, U.S. patent application Ser. Nos. 08/549,489, 08/462,014, 09/659,203, 10/246,447, 10/465,302, U.S. Pat. Nos. 6,281,010, 6,270,996, 6,261,551, 5,756,283, 6,428,988, 6,274,354, 6,943,019, 6,482,634, (Assigned to NIH: U.S. Pat. Nos. 7,238,526, 6,475,769), U.S. Pat. No. 6,365,394 (Assigned to NIH), U.S. Pat. Nos. 7,491,508, 7,291,498, 7,022,519, 6,485,966, 6,953,690, 6,258,595, EP2018421, EP1064393, EP1163354, EP835321, EP931158, EP950111, EP1015619, EP1183380, EP2018421, EP1226264, EP1636370, EP1163354, EP1064393, US20030032613, US20020102714, US20030073232, US20030040101 (Assigned to NIH), US20060003451, US20020090717, US20030092161, US20070231303, US20060211115, US20090275107, US2007004042, US20030119191, US20020019050, the contents of which are each incorporated herein by reference in their entireties, insofar as they do not conflict with the present disclosure.

### III. Payload Expression Constructs

Also provided herein are viral expression constructs for expression of transgenes utilizing viral genomes.

### AAV Viral Genomes

The wild-type AAV viral genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions comprising, but not limited to, acting as an origin for DNA replication by functioning as primers for the endogenous DNA polymerase complex of the host viral replication cell.

For use as a biological tool, the wild-type AAV viral genome can be modified to replace the rep/cap sequences with a nucleic acid sequence comprising a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. See Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entireties as related to AAV variants and equivalents, insofar as they do not conflict with the present disclosure.

In certain embodiments, AAV particles, viral genomes and/or payloads of the present disclosure, and the methods of their use, may be as described in WO2017189963, the content of which is incorporated herein by reference in its entirety as related to AAV particles, viral genomes and/or payloads, insofar as it does not conflict with the present disclosure.

AAV particles of the present disclosure may be formulated in any of the gene therapy formulations of the disclosure comprising any variations of such formulations apparent to those skilled in the art. The reference to “AAV particles”, “AAV particle formulations” and “formulated AAV particles” in the present application refers to the AAV particles which may be formulated and those which are formulated without limiting either.

In certain embodiments, AAV particles of the present disclosure are recombinant AAV (rAAV) viral particles which are replication defective, lacking sequences encoding functional Rep and Cap proteins within their viral genome. These defective AAV particles may lack most or all parental coding sequences and essentially carry only one or two AAV ITR sequences and the nucleic acid of interest (i.e., payload) for delivery to a cell, a tissue, an organ or an organism.

In certain embodiments, the viral genome of the AAV particles of the present disclosure comprises at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements comprise sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV particles of the present disclosure may be produced recombinantly and may be based on adeno-associated virus (AAV) parent or reference sequences. As used herein, a “vector” is any molecule or moiety which transports, transduces or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV viral genomes contain DNA strands which anneal together to form double stranded DNA. By skipping second strand synthesis, scAAVs allow for rapid expression in the cell.

In certain embodiments, the AAV viral genome of the present disclosure is a scAAV. In certain embodiments, the AAV viral genome of the present disclosure is a ssAAV.

Methods for producing and/or modifying AAV particles are disclosed in the art, such as pseudotyped AAV particles (PCT Patent Publication Nos. WO200028004; WO200123001; WO2004112727; WO 2005005610 and WO 2005072364, the contents of each of which are incorporated herein by reference in their entireties as related to producing and/or modifying AAV particles, insofar as they do not conflict with the present disclosure).

AAV particles may be modified to enhance the efficiency of delivery. Such modified AAV particles can be packaged efficiently and be used to successfully infect the target cells at high frequency and with minimal toxicity. In certain embodiments the capsids of the AAV particles are engineered according to the methods described in US Publication Number US 20130195801, the content of which is incorporated herein by reference in its entirety as related to modifying AAV particles to enhance the efficiency of delivery, insofar as it does not conflict with the present disclosure.

In certain embodiments, the AAV particles comprise a payload construct and/or region encoding a polypeptide or protein of the present disclosure, and may be introduced into mammalian cells. In certain embodiments, the AAV particles comprise a payload construct and/or region encoding a polypeptide or protein of the present disclosure, and may be introduced into insect cells.

In certain embodiments, the AAV particles of the present disclosure comprise a viral genome with at least one ITR region and a payload region. In certain embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes of the present disclosure may be comprised of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome comprising two ITRs. In certain embodiments, the ITRs are of the same serotype as one another. In another embodiment, the ITRs are of different serotypes. Non-limiting examples comprise zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length.

In certain embodiments, each ITR may be 141 nucleotides in length. In certain embodiments, each ITR may be 130 nucleotides in length. In certain embodiments, each ITR may be 119 nucleotides in length.

### Viral Genome Size

In certain embodiments, the AAV particle which includes a payload described herein may be single stranded or double stranded viral genome. The size of the viral genome may be small, medium, large or the maximum size. Additionally, the viral genome may include a promoter and a polyA tail.

In certain embodiments, the viral genome which includes a payload described herein may be a small single stranded viral genome. A small single stranded viral genome may be 2.1 to 3.5 kb in size such as about 2.1, 2.2, 2.3, 2.4, 2.5, 2.6, 2.7, 2.8, 2.9, 3.0, 3.1, 3.2, 3.3, 3.4, and 3.5 kb in size. As a non-limiting example, the small single stranded viral genome may be 3.2 kb in size. As another non-limiting example, the small single stranded viral genome may be 2.2 kb in size. Additionally, the viral genome may include a promoter and a polyA tail.

In certain embodiments, the viral genome which includes a payload described herein may be a small double stranded viral genome. A small double stranded viral genome may be 1.3 to 1.7 kb in size such as about 1.3, 1.4, 1.5, 1.6, and 1.7 kb in size. As a non-limiting example, the small double stranded viral genome may be 1.6 kb in size. Additionally, the viral genome may include a promoter and a polyA tail.

In certain embodiments, the viral genome which includes a payload described herein e.g., polynucleotide, siRNA or dsRNA, may be a medium single stranded viral genome. A medium single stranded viral genome may be 3.6 to 4.3 kb in size such as about 3.6, 3.7, 3.8, 3.9, 4.0, 4.1, 4.2 and 4.3 kb in size. As a non-limiting example, the medium single stranded viral genome may be 4.0 kb in size. Additionally, the viral genome may include a promoter and a polyA tail.

In certain embodiments, the viral genome which includes a payload described herein may be a medium double stranded viral genome. A medium double stranded viral genome may be 1.8 to 2.1 kb in size such as about 1.8, 1.9, 2.0, and 2.1 kb in size. As a non-limiting example, the medium double stranded viral genome may be 2.0 kb in size. Additionally, the viral genome may include a promoter and a polyA tail.

In certain embodiments, the viral genome which includes a payload described herein may be a large single stranded viral genome. A large single stranded viral genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded viral genome may be 4.7 kb in size. As another non-limiting example, the large single stranded viral genome may be 4.8 kb in size. As yet another non-limiting example, the large single stranded viral genome may be 6.0 kb in size. Additionally, the viral genome may include a promoter and a polyA tail.

In certain embodiments, the viral genome which includes a payload described herein may be a large double stranded viral genome. A large double stranded viral genome may be 2.2 to 3.0 kb in size such as about 2.2, 2.3, 2.4, 2.5, 2.6, 2.7, 2.8, 2.9 and 3.0 kb in size. As a non-limiting example, the large double stranded viral genome may be 2.4 kb in size. Additionally, the viral genome may include a promoter and a polyA tail.

In certain embodiments, an viral genome of the present disclosure can include at least one filler region. In certain embodiments, an viral genome of the present disclosure can include at least one multiple cloning site (MCS) region. In certain embodiments, an viral genome of the present disclosure can include at least one promoter region. In certain embodiments, an viral genome of the present disclosure can include at least one exon region. In certain embodiments, an viral genome of the present disclosure can include at least one intron region.

### Viral Genome Regions: Inverted Terminal Repeats (ITRs)

The AAV particles of the present disclosure include a viral genome with at least one Inverted Terminal Repeat (ITR) region and a payload region. In certain embodiments, the viral genome has two ITRs. These two ITRs flank the payload region at the 5′ and 3′ ends. The ITRs function as origins of replication including recognition sites for replication. ITRs include sequence regions which can be complementary and symmetrically arranged. ITRs incorporated into viral genomes of the present disclosure may be included of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome including two ITRs. In certain embodiments, the ITRs are of the same serotype as one another. In another embodiment, the ITRs are of different serotypes. Non-limiting examples include zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length, and those having at least 95% identity thereto.

In certain embodiments, each ITR may be 141 nucleotides in length. In certain embodiments, each ITR may be 130 nucleotides in length. In certain embodiments, each ITR may be 119 nucleotides in length.

In certain embodiments, the AAV particles include two ITRs and one ITR is 141 nucleotides in length and the other ITR is 130 nucleotides in length. In certain embodiments, the AAV particles include two ITRs and both ITR are 141 nucleotides in length.

Independently, each ITR may be about 75 to about 175 nucleotides in length. The ITR may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length. As a non-limiting example, the viral genome comprises an ITR that is about 141 nucleotides in length. As a non-limiting example, the viral genome comprises an ITR that is about 130 nucleotides in length. As a non-limiting example, the viral genome comprises an ITR that is about 105 nucleotides in length and 141 nucleotides in length. As a non-limiting example, the viral genome comprises an ITR that is about 105 nucleotides in length and 130 nucleotides in length. As a non-limiting example, the viral genome comprises an ITR that is about 130 nucleotides in length and 141 nucleotides in length.

### AAV Serotypes

AAV particles of the present disclosure may include or be derived from any natural or recombinant AAV serotype. According to the present disclosure, the AAV particles may utilize or be based on a serotype or include a peptide selected from any of the following: VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8 h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAVS, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAVrh20, AAVrh32/33, AAVrh39, AAVrh46, AAVrh73, AAVrh74, AAVhu.26, or variants or derivatives thereof.

The AAV-DJ sequence may include two mutations: (1) R587Q where arginine (R; Arg) at amino acid 587 is changed to glutamine (Q; GIn) and (2) R590T where arginine (R; Arg) at amino acid 590 is changed to threonine (T; Thr). As another non-limiting example, may include three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In certain embodiments, the AAV may be a serotype generated by the AAV9 capsid library with mutations in amino acids 390-627 (VP1 numbering) The serotype and corresponding nucleotide and amino acid substitutions may be, but is not limited to, AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

In certain embodiments, the AAV serotype may be, or may include a sequence, insert, modification or mutation as described in Patent Publications WO2015038958, WO2017100671, WO2016134375, WO2017083722, WO2017015102, WO2017058892, WO2017066764, U.S. Pat. Nos. 9,624,274, 9,475,845, US20160369298, US20170145405, the contents of which are herein incorporated by reference in their entirety.

In certain embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In certain embodiments, the AAV serotype may be as described in Jackson et al (Frontiers in Molecular Neuroscience 9:154 (2016)), the contents of which are herein incorporated by reference in their entirety.

In certain embodiments, the AAV serotype is selected for use due to its tropism for cells of the central nervous system. In certain embodiments, the cells of the central nervous system are neurons. In another embodiment, the cells of the central nervous system are astrocytes.

In certain embodiments, the AAV serotype is selected for use due to its tropism for cells of the muscle(s).

### Payloads

AAV particles of the present disclosure can comprise, or be produced using, at least one payload construct which comprises at least one payload region. In certain embodiments, the payload region may be located within a viral genome, such as the viral genome of a payload construct. At the 5′ and/or the 3′ end of the payload region there may be at least one inverted terminal repeat (ITR). Within the payload region, there may be a promoter region, an intron region and a coding region.

In certain embodiments, the payload region of the AAV particle comprises one or more nucleic acid sequences encoding one or more payload, such as a payload polypeptide or polynucleotide. In certain embodiments, the payload region of the AAV particle comprises one or more nucleic acid sequences encoding one or more polypeptides or proteins of interest. In certain embodiments, the payload region of the AAV particle comprises one or more nucleic acid sequences encoding one or more modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the payload region can be included in a payload construct used for producing AAV particles. In certain embodiments, a payload construct of the present disclosure can be a bacmid, also known as a baculovirus plasmid or recombinant baculovirus genome. In certain embodiments, a payload construct of the present disclosure can be a baculovirus expression vector (BEV). In certain embodiments, a payload construct of the present disclosure can be a BIIC which includes a BEV. As used herein, the term “payloadBac” refers to a bacmid (such as a BEV) comprising a payload construct and/or payload region. Viral production cells (e.g., Sf9 cells) may be transfected with payloadBacs and/or with BIICs comprising payloadBacs.

In certain embodiments, the AAV particles of the present disclosure comprise one or more nucleic acid sequences encoding one or more payload, such as a payload polypeptide or polynucleotide, which are useful in the field of medicine for the treatment, prophylaxis, palliation, or amelioration of diseases and/or disorders, including neurological diseases and/or disorders. In certain embodiments, the AAV particles of the present disclosure are useful in the field of medicine for the treatment, prophylaxis, palliation or amelioration of Friedreich's ataxia, or any disease stemming from a loss or partial loss of frataxin protein. In certain embodiments, the AAV particles of the present disclosure are useful in the field of medicine for the treatment, prophylaxis, palliation, or amelioration of Parkinson's Disease. In certain embodiments, the AAV particles of the present disclosure are useful in the field of medicine for the treatment, prophylaxis, palliation, or amelioration of Amyotrophic lateral sclerosis. In certain embodiments, the AAV particles of the present disclosure are useful in the field of medicine for the treatment, prophylaxis, palliation, or amelioration of Huntington's Disease. In certain embodiments, the AAV particles of the present disclosure are useful in the field of medicine for the treatment, prophylaxis, palliation, or amelioration of Alzheimer's Disease.

### Payloads: Polypeptides and Variants

In certain embodiments, the payload region of the AAV particle comprises one or more nucleic acid sequences encoding a polypeptide or protein of interest. In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the viral genome may express each of the one or more polypeptides in the single target cell.

Where the AAV particle payload region encodes a polypeptide, the polypeptide may be a peptide, polypeptide, or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the AAV particle comprises a viral genome with a payload region comprising a nucleic acid sequence encoding a protein of interest (i.e., a payload protein, therapeutic protein).

Amino acid sequences encoded by payload regions of the viral genomes of the disclosure may be translated as a whole polypeptide, a plurality of polypeptides or fragments of polypeptides, which independently may be encoded by one or more nucleic acids, fragments of nucleic acids or variants of any of the aforementioned. In certain embodiments, polypeptides can include proteins, polypeptides, and peptides of any size, structure, or function. In some instances, the polypeptide encoded is smaller than about 50 amino acids (i.e., peptide). If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer, or tetramer. They may also include single chain or multichain polypeptides and may be associated or linked. The term polypeptide may also apply to amino acid polymers in which one or more amino acid residues are an artificial chemical analogue of a corresponding naturally occurring amino acid.

In certain embodiments, the polypeptide can be a polypeptide variant which differs in amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and in certain embodiments, they will be at least about 80%, or at least about 90% identical (homologous) to a native or reference sequence.

In certain embodiments, the payload region of the AAV particle comprises one or more nucleic acid sequences encoding a polypeptide or protein of interest.

In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the viral genome may express each of the one or more polypeptides in the single target cell.

Where the AAV particle payload region encodes a polypeptide, the polypeptide may be a peptide, polypeptide or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the formulated AAV particles of the present disclosure may be used to reduce the decline of functional capacity and activities of daily living as measured by a standard evaluation system such as, but not limited to, the total functional capacity (TFC) scale.

In certain embodiments, the AAV particle comprises a viral genome with a payload region comprising a nucleic acid sequence encoding a protein of interest (i.e., a payload protein, therapeutic protein).

In certain embodiments, the payload region comprises a nucleic acid sequence encoding a protein including but not limited to an antibody, Aromatic L-Amino Acid Decarboxylase (AADC), ApoE2, Frataxin, survival motor neuron (SMN) protein, glucocerebrosidase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLB1) and/or gigaxonin (GAN).

In certain embodiments, the AAV particle includes a viral genome with a payload region comprising a nucleic acid sequence encoding AADC or any other payload known in the art for treating Parkinson's disease. As a non-limiting example, the payload may include a sequence such as NM_001082971.1 (GI: 132814447), NM_000790.3 (GI: 132814459), NM_001242886.1 (GI: 338968913), NM_001242887.1 (GI: 338968916), NM_001242888.1 (GI: 338968918), NM_001242889.1 (GI: 338968920), NM_001242890.1 (GI: 338968922) and fragment or variants thereof.

In certain embodiments, the AAV particle comprises a viral genome with a payload region comprising a nucleic acid sequence encoding frataxin or any other payload known in the art for treating Friedreich's Ataxia. As a non-limiting example, the payload may comprise a sequence such as NM_000144.4 (GI: 239787167), NM_181425.2 (GI: 239787185), NM_001161706.1 (GI: 239787197) and fragment or variants thereof.

In certain embodiments, the AAV particle comprises a viral genome with a payload region comprising a nucleic acid sequence encoding SMN or any other payload known in the art for treating spinal muscular atrophy (SMA). As a non-limiting example, the payload may comprise a sequence such as NM_001297715.1 (GI: 663070993), NM_000344.3 (GI: 196115055), NM_022874.2 (GI: 196115040) and fragment or variants thereof.

In certain embodiments, the AAV particle comprises a viral genome with a payload region comprising a nucleic acid sequence encoding any of the disease-associated proteins (and fragment or variants thereof) described in U. S. Patent publication No. 20180258424; the content of which is herein incorporated by reference in its entirety.

In certain embodiments, the AAV particle includes a viral genome with a payload region comprising a nucleic acid sequence encoding any of the disease-associated proteins (and fragment or variants thereof) described in any one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety

In certain embodiments, the formulated AAV particles of the present disclosure may be used to improve performance on any assessment used to measure symptoms of a neurodegenerative disorder/disease. Such assessments comprise, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B Ther Adv Neurol Disord 5(6):349-358 (2012), the contents of which are herein incorporated by reference in their entirety.

In certain embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

In certain embodiments an “amino acid sequence variant” is provided. The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and in certain embodiments, they will be at least about 80% or at least about 90% homologous to a native sequence. “Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to comprise polypeptide variants which differ by one or more amino acid alterations, e.g., substitutions, additions or deletions of amino acid residues that still maintain the properties of the parent polypeptide.

Sequence tags or amino acids, such as one or more lysines, can be added to the peptide sequences of the disclosure (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support.

In certain embodiments a “substitutional variant” is provided. “Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions comprise the substitution of a non-polar (hydrophobic) residue such as isoleucine, valine and leucine for another non-polar residue. Likewise, examples of conservative substitutions comprise the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions comprise the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In certain embodiments an “insertional variant” is provided. “Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

In certain embodiments a “deletional variant” is provided. “Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In certain embodiments, derivatives comprise native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present disclosure.

Other post-translational modifications comprise hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

“Features” when referring to proteins are defined as distinct amino acid sequence-based components of a molecule. Features of the proteins of the present disclosure comprise surface manifestations, local conformational shape, folds, loops, half-loops, domains, half-domains, sites, termini or any combination thereof.

As used herein when referring to polynucleotides the term “loop” refers to a structural feature which may serve to reverse the direction of the backbone of a polynucleotide such that two regions at a distance of the polynucleotide are brought together spatially. Loops may be open or closed. Closed loops or “cyclic” loops may include 2, 3, 4, 5, 6, 7, 8, 9, 10 or more nucleotides.

As used herein the term “domain” refers to a motif of a polynucleotide having one or more identifiable structural or functional characteristics or properties (e.g., binding capacity, serving as a site for interactions).

As used herein the terms “site” as it pertains to polynucleotides is used synonymously with “nucleic acid residue” and/or “nucleotide.” A site represents a position within a polynucleotide that may be modified, manipulated, altered, derivatized or varied.

As used herein the terms “termini” or “terminus” refers to an extremity of a polynucleotide. Such extremity is not limited only to the first or final site of the polynucleotide but may include additional nucleotides in the terminal regions. The polynucleotides of the present disclosure may be characterized as having both a 5′ and a 3′ terminus.

Once any of the features have been identified or defined as a component of a molecule of the disclosure, any of several manipulations and/or modifications of these features may be performed by moving, swapping, inverting, deleting, randomizing or duplicating. Furthermore, it is understood that manipulation of features may result in the same outcome as a modification to the molecules of the disclosure. For example, a manipulation which involves deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein, or any other suitable screening assay known in the art.

### Viral Genome Component: Promoters

In certain embodiments, the payload region of the viral genome comprises at least one element to enhance the payload target specificity and expression (See e.g., Powell et al. Viral Expression Cassette Elements to Enhance Transgene Target Specificity and Expression in Gene Therapy, 2015; the contents of which are herein incorporated by reference in their entirety). Non-limiting examples of elements to enhance payload target specificity and expression include promoters, endogenous miRNAs, post-transcriptional regulatory elements (PREs), polyadenylation (PolyA) signal sequences and upstream enhancers (USEs), CMV enhancers and introns.

In some embodiments, an AAV particle comprising an AAV capsid protein comprises a viral genome comprising a nucleic acid comprising a transgene encoding a payload, wherein the transgene is operably linked to a promoter. In some embodiments, the promoter is a species specific promoter, an inducible promoter, tissue-specific, or cell cycle-specific (Parr et al., Nat. Med. 3:1145-9 (1997); the contents of which are herein incorporated by reference in their entirety).

In some embodiments the promoter may be naturally occurring or non-naturally occurring. Non-limiting examples of promoters include those from viruses, plants, mammals, or humans. In some embodiments, the promoters may be those from human cells or systems. In some embodiments, the promoter may be truncated or mutated, e.g., a promoter variant.

In some embodiments, the promoter is a ubiquitous promoter, e.g., capable of expression in multiple tissues. In some embodiments the promoter is an human elongation factor 1α-subunit (EF1α) promoter, the cytomegalovirus (CMV) immediate-early enhancer and/or promoter, the chicken β-actin (CBA) promoter and its derivative CAG, β glucuronidase (GUSB) promoter, or ubiquitin C (UBC) promoter. In some embodiments, the promoter is a cell or tissue specific promoter, e.g., capable of expression in tissues or cells of the central or peripheral nervous systems, regions within (e.g., frontal cortex), and/or sub-sets of cells therein (e.g., excitatory neurons). In some embodiments, the promoter is a cell-type specific promoter capable of expression a payload in excitatory neurons (e.g., glutamatergic), inhibitory neurons (e.g., GABA-ergic), neurons of the sympathetic or parasympathetic nervous system, sensory neurons, neurons of the dorsal root ganglia, motor neurons, or supportive cells of the nervous systems such as microglia, astrocytes, oligodendrocytes, and/or Schwann cells.

In some embodiments, the promoter is a liver promoter (e.g., hAAT, TBG), skeletal muscle specific promoter (e.g., desmin, MCK, C512), B cell promoter, monocyte promoter, leukocyte promoter, macrophage promoter, pancreatic acinar cell promoter, endothelial cell promoter, lung tissue promoter, and/or cardiac or cardiovascular promoter (e.g., αMHC, cTnT, and CMV-MLC2k).

In some embodiments, the promoter is a tissue-specific promoter for payload expression in a cell or tissue of the central nervous system. In some embodiments, the promoter is synapsin (Syn) promoter, glutamate vesicular transporter (VGLUT) promoter, vesicular GABA transporter (VGAT) promoter, parvalbumin (PV) promoter, sodium channel Naᵥ 1.8 promoter, tyrosine hydroxylase (TH) promoter, choline acetyltransferase (ChaT) promoter, methyl-CpG binding protein 2 (MeCP2) promoter, Ca²⁺/calmodulin-dependent protein kinase II (CaMKII) promoter, metabotropic glutamate receptor 2 (mGluR2) promoter, neurofilament light (NFL) or heavy (NFH) promoter, neuron-specific enolase (NSE) promoter, β-globin minigene nβ2 promoter, preproenkephalin (PPE) promoter, enkephalin (Enk) promoter, and excitatory amino acid transporter 2 (EAAT2) promoter. In some embodiments, the promoter is a cell-type specific promoter capable of expression in an astrocyte, e.g., a glial fibrillary acidic protein (GFAP) promoter and a EAAT2 promoter. In some embodiments, the promoter is a cell-type specific promoter capable of expression in an oligodendrocyte, e.g., a myelin basic protein (MBP) promoter.

In some embodiments, the promoter is a GFAP promoter. In some embodiments, the promoter is a synapsin (syn or syn1) promoter, or a fragment thereof.

In some embodiments, the promoter comprises an insulin promoter or a fragment thereof.

In some embodiments, the promoter of the viral genome described herein (e.g., comprised within an AAV particle comprising an AAV capsid variant described herein) comprises an EF-1α promoter or variant thereof.

### Viral Genome Component: Untranslated Regions (UTRs)

In some embodiments, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs (e.g., CNS tissue, muscle, or DRG) may be engineered into UTRs to enhance stability and protein production. As a non-limiting example, a 5′ UTR from mRNA normally expressed in the brain (e.g., huntingtin) may be used in the viral genomes of the AAV particles described herein to enhance expression in neuronal cells or other cells of the central nervous system.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In one embodiment, the 5′UTR in the viral genome includes a Kozak sequence.

In one embodiment, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of a polynucleotide. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In one embodiment, the 3′ UTR of the viral genome may include an oligo(dT) sequence for templated addition of a poly-A tail.

In one embodiment, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, a microRNA sequence comprises a seed region, e.g., a sequence in the region of positions 2-8 of the mature microRNA, which has Watson-Crick sequence fully or partially complementarity to the miRNA target sequence of the nucleic acid.

In one embodiment, the viral genome may be engineered to include, alter or remove at least one miRNA binding site, full sequence or seed region.

Any UTR from any gene known in the art may be incorporated into the viral genome of the AAV particle. These UTRs, or portions thereof, may be placed in the same orientation as in the gene from which they were selected or they may be altered in orientation or location. In one embodiment, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In one embodiment, the viral genome of the AAV particle comprises at least one artificial UTR which is not a variant of a wild type UTR.

In one embodiment, the viral genome of the AAV particle comprises UTRs which have been selected from a family of transcripts whose proteins share a common function, structure, feature or property.

### Viral Genome Component: Introns

In some embodiments, the viral genome of the AAV particle as described herein (e.g., an AAV particle comprising an AAV capsid polypeptide) comprises an element to enhance the payload target specificity and expression (See e.g., Powell et al. Viral Expression Cassette Elements to Enhance Transgene Target Specificity and Expression in Gene Therapy, Discov. Med, 2015, 19(102): 49-57; the contents of which are herein incorporated by reference in their entirety) such as an intron. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

Viral Genome Component: Stuffer sequences

In some embodiments, the viral genome of an AAV particle described herein (e.g., an AAV particle comprising an AAV capsid polypeptide), comprises an element to improve packaging efficiency and expression, such as a stuffer or filler sequence. Non-limiting examples of stuffer sequences include albumin and/or alpha-1 antitrypsin. Any known viral, mammalian, or plant sequence may be manipulated for use as a stuffer sequence.

In one embodiment, the stuffer or filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000 nucleotides.

Viral Genome Component: miRNA

In some embodiments, the viral genome of an AAV particle described herein (e.g., an AAV particle comprising an AAV capsid polypeptide) comprises a sequence encoding a miRNA to reduce the expression of the payload in a tissue or cell, e.g., the DRG (dorsal root ganglion), or neurons of other ganglia, such as those of the sympathetic or parasympathetic nervous system. In some embodiments, a miRNA, e.g., a miR183, a miR182, and/or miR96, may be encoded in the viral genome to modulate, e.g., reduce the expression, of the viral genome in a DRG neuron. As another non-limiting example, a miR-122 miRNA may be encoded in the viral genome to modulate, e.g., reduce, the expression of the viral genome in the liver. In some embodiments, a miRNA, e.g., a miR-142-3p, may be encoded in the viral genome to modulate, e.g., reduce, the expression, of the viral genome in a cell or tissue of the hematopoietic lineage, including for example immune cells (e.g., antigen presenting cells or APC, including dendritic cells (DCs), macrophages, and B-lymphocytes). In some embodiments, a miRNA, e.g., a miR-1, may be encoded in the viral genome to modulate, e.g., reduce, the expression, of the viral genome in a cell or tissue of the heart.

### Viral Genome Component: miR Binding Site

Tissue- or cell-specific expression of the AAV viral particles disclosed herein can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome disclosed herein, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleotide sequence encoding a payload), e.g., before the polyA sequence, 5′-UTR region of the viral genome (e.g., 5′ relative to the nucleotide sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91).

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, in length. In some embodiments, the spacer comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91).

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% complimentary), to the miR in the host cell. In some embodiments, to complementary sequence of the nucleotide sequence encoding the miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complimentary to the miR in the host cell.

In some embodiments, an encoded miR binding site or sequence region is at least about 10 to about 125 nucleotides in length, e.g., at least about 10 to 50 nucleotides, 10 to 100 nucleotides, 50 to 100 nucleotides, 50 to 125 nucleotides, or 100 to 125 nucleotides in length. In some embodiments, an encoded miR binding site or sequence region is at least about 7 to about 28 nucleotides in length, e.g., at least about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., fully or partially complementary) to the seed sequence of a miRNA (e.g., a miR122, a miR142, a miR183, or a miR1).

In some embodiments, the encoded miR binding site is complementary (e.g., fully or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 92), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 92, e.g., wherein the modification can result in a mismatch between the encoded miR binding site and the corresponding miRNA. In some embodiments, the viral genome comprises at least 2, 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGTCACACTCCACACAAACACCATTGT CACACTCCA (SEQ ID NO: 93), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 93, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8, in length. In some embodiments, the spacer coding sequence or reverse complement thereof comprises one or more of (i) GGAT; (ii) CACGTG; (iii) GCATGC, or a repeat of one or more of (i)-(iii). In some embodiments, an encoded miR binding site series comprises at least 3-5 copies (e.g., 4 copies) of a miR122 binding site, with or without a spacer, wherein the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91).

In some embodiments, the encoded miR binding site is complementary (e.g., fully or partially complementary) to a miR expressed in the heart. In embodiments, the encoded miR binding site or encoded miR binding site series comprises a miR-1 binding site. In some embodiments, the encoded miR-1 binding site comprises the nucleotide sequence of ATACATACTTCTTTACATTCCA (SEQ ID NO: 94), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 94, e.g., wherein the modification can result in a mismatch between the encoded miR binding site and the corresponding miRNA. In some embodiments, the viral genome comprises at least 2, 3, 4, or 5 copies of the encoded miR-1 binding site, e.g., an encoded miR-1 binding site series. In some embodiments, the at least 2, 3, 4, or 5 copies (e.g., 2 or 3 copies) of the encoded miR-1 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91).

In some embodiments, the encoded miR binding site is complementary (e.g., fully or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site complementary to a miR expressed in hematopoietic lineage comprises a nucleotide sequence disclosed, e.g., in US 2018/0066279, the contents of which are incorporated by reference herein in its entirety.

In some embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 95), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 95, e.g., wherein the modification can result in a mismatch between the encoded miR binding site and the corresponding miRNA. In some embodiments, the viral genome comprises at least 2, 3, 4, or 5 copies of the encoded miR-142-3p binding site, e.g., an encoded miR-142-3p binding site series. In some embodiments, the at least 2, 3, 4, or 5 copies (e.g., 2 or 3 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91).

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary to a miR expressed in expressed in a DRG neuron comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGTGCCATA (SEQ ID NO: 96), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 96, e.g., wherein the modification can result in a mismatch between the encoded miR binding site and the corresponding miRNA. In some embodiments, the sequence complementary to the seed sequence corresponds to the double underlined of the encoded miR-183 binding site sequence. In some embodiments, the viral genome comprises at least comprises at least 2, 3, 4, or 5 copies (e.g., at least 2 or 3 copies) of the encoded miR183 binding site, e.g., an encoded miR183 binding site. In some embodiments, the at least 2, 3, 4, or 5 copies (e.g., 2 or 3 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91). In some embodiments, the spacer sequence comprises one or more of (i) GGAT; (ii) CACGTG; (iii) GCATGC, or a repeat of one or more of (i)-(iii).

In some embodiments, the encoded miR binding site or the encoded miR binding site series comprises a miR182 binding site sequence. In some embodiments, the encoded miR182 binding site comprises, the nucleotide sequence of AGTGTGAGTTCTACCATTGCCAAA (SEQ ID NO: 97), a sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 97, e.g., wherein the modification can result in a mismatch between the encoded miR binding site and the corresponding miRNA. In some embodiments, the viral genome comprises at least 2, 3, 4, or 5 copies of the encoded miR182 binding site, e.g., an encoded miR182 binding site series. In some embodiments, the at least 2, 3, 4, or 5 copies (e.g., 2 or 3 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91). In some embodiments, the spacer sequence comprises one or more of (i) GGAT; (ii) CACGTG; (iii) GCATGC, or a repeat of one or more of (i)-(iii).

In some embodiments, the encoded miR binding site or the encoded miR binding site series comprises a miR96 binding site sequence. In some embodiments, the encoded miR96 binding site comprises the nucleotide sequence of AGCAAAAATGTGCTAGTGCCAAA (SEQ ID NO: 98), a sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 98, e.g., wherein the modification can result in a mismatch between the encoded miR binding site and the corresponding miRNA. In some embodiments, the viral genome comprises at least 2, 3, 4, or 5 copies of the encoded miR96 binding site, e.g., an encoded miR96 binding site series. In some embodiments, the at least 2, 3, 4, or 5 copies (e.g., 2 or 3 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91). In some embodiments, the spacer sequence comprises one or more of (i) GGAT; (ii) CACGTG; (iii) GCATGC, or a repeat of one or more of (i)-(iii).

In some embodiments, the encoded miR binding site series comprises a miR122 binding site, a miR-1, a miR142 binding site, a miR183 binding site, a miR182 binding site, a miR 96 binding site, or a combination thereof. In some embodiments, the encoded miR binding site series comprises at least 2, 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91).

In some embodiments, an encoded miR binding site series comprises at least 2-5 copies (e.g., 2 or 3 copies) of a combination of at least two, three, four, five, or all of a miR-1, miR122 binding site, a miR142 binding site, a miR183 binding site, a miR182 binding site, a miR96 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91).

In some embodiments, an encoded miR binding site series comprises at least 2-5 copies (e.g., 2 or 3 copies) of a combination of a miR-122 binding site and a miR-1 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 91), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 91).

### Payloads: Modulatory Polynucleotides Targeting a Gene of Interest

The present disclosure comprises the use of formulated AAV particles whose viral genomes encode modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle comprises a viral genome with a payload region comprising a nucleic acid sequence encoding or comprising one or more modulatory polynucleotides. In certain embodiments, the AAV particle comprises a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features comprise (i) the ability to infect both dividing and non-dividing cells; (ii) a broad host range for infectivity, comprising human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, 1 or 2 nucleotide overhangs at the 3′ end of each strand.

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g., antisense strand RNA or antisense oligonucleotides). In many cases it requires higher concentration of the ss-siRNA to achieve the effective gene silencing potency of the corresponding duplex.

In certain embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In certain embodiments, the modulatory polynucleotide which comprises the payload (e.g., siRNA, miRNA or other RNAi agent described herein) comprises molecular scaffold which comprises a leading 5′ flanking sequence which may be of any length and may be derived in whole or in part from wild type microRNA sequence or be completely artificial. A 3′ flanking sequence may mirror the 5′ flanking sequence in size and origin. In certain embodiments, one or both of the 5′ and 3′ flanking sequences are absent.

In certain embodiments, the molecular scaffold may comprise one or more linkers known in the art. The linkers may separate regions or one molecular scaffold from another. As a non-limiting example, the molecular scaffold may be polycistronic.

In certain embodiments, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

In certain embodiments, the present disclosure presents the use of formulated AAV particles whose viral genomes encode modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle comprises a viral genome with a payload region comprising a nucleic acid sequence encoding or comprising one or more modulatory polynucleotides. In certain embodiments, the AAV particle comprises a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, the payload region comprises a nucleic acid sequence encoding a modulatory polynucleotide which interferes with a target gene expression and/or a target protein production. In certain embodiments, the gene expression or protein production to be inhibited/modified may comprise but are not limited to superoxide dismutase 1 (SOD1), chromosome 9 open reading frame 72 (C90RF72), TAR DNA binding protein (TARDBP), ataxin-3 (ATXN3), huntingtin (HTT), amyloid precursor protein (APP), apolipoprotein E (ApoE), microtubule-associated protein tau (MAPT), alpha-synuclein (SNCA), voltage-gated sodium channel alpha subunit 9 (SCN9A), and/or voltage-gated sodium channel alpha subunit 10 (SCN10A).

The present disclosure provides small interfering RNA (siRNA) duplexes (and modulatory polynucleotides encoding them) that target SOD1 mRNA to interfere with the gene expression and/or protein production of SOD1. The present disclosure also provides methods of their use for inhibiting gene expression and protein production of an allele of SOD1, for treating amyotrophic lateral sclerosis (ALS). In certain embodiments, the siRNA duplexes of the present disclosure may target SOD1 along any segment of the respective nucleotide sequence. In certain embodiments, the siRNA duplexes of the present disclosure may target SOD1 at the location of a SNP or variant within the nucleotide sequence.

The present disclosure provides small interfering RNA (siRNA) duplexes (and modulatory polynucleotides encoding them) that target HTT mRNA to interfere with the gene expression and/or protein production of HTT. The present disclosure also provides methods of their use for inhibiting gene expression and protein production of an allele of HTT, for treating Huntington's disease (HD). In certain embodiments, the siRNA duplexes of the present disclosure may target HTT along any segment of the respective nucleotide sequence. In certain embodiments, the siRNA duplexes of the present disclosure may target HTT at the location of a SNP or variant within the nucleotide sequence.

In certain embodiments, the AAV particle comprises a viral genome with a payload region comprising a nucleic acid sequence encoding any of the modulatory polynucleotides, RNAi molecules, siRNA molecules, dsRNA molecules, and/or RNA duplexes described in any one of the following International Publications: WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features comprise (i) the ability to infect both dividing and non-dividing cells; (ii) a broad host range for infectivity, comprising human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, 1 or 2 nucleotide overhangs at the 3′ end of each strand.

According to the present disclosure, each strand of the siRNA duplex targeting the gene of interest can be about 19 to 25, 19 to 24 or 19 to 21 nucleotides in length, such as about 19 nucleotides, 20 nucleotides, 21 nucleotides, 22 nucleotides, 23 nucleotides, 24 nucleotides, or 25 nucleotides in length.

In certain embodiments, an siRNA or dsRNA comprises at least two sequences that are complementary to each other. The dsRNA comprises a sense strand having a first sequence and an antisense strand having a second sequence. The antisense strand comprises a nucleotide sequence that is substantially complementary to at least part of an mRNA encoding a gene of interest, and the region of complementarity is 30 nucleotides or less, and at least 15 nucleotides in length. Generally, the dsRNA is 19 to 25, 19 to 24 or 19 to 21 nucleotides in length. In certain embodiments, the dsRNA is from about 15 to about 25 nucleotides in length, and in certain embodiments the dsRNA is from about 25 to about 30 nucleotides in length.

The dsRNA encoded in an expression vector upon contacting with a cell expressing protein encoded by the gene of interest, inhibits the expression of protein encoded by the gene of interest by at least 10%, at least 20%, at least 25%, at least 30%, at least 35%, at least 40%, at least 50%, at least 60%, at least 70%, at least 80%, or more, when assayed by methods known in the art or a method as described herein.

According to the present disclosure, the siRNA molecules are designed and tested for their ability in reducing mRNA levels in cultured cells.

In certain embodiments, the siRNA molecules are designed and tested for their ability in reducing levels of the gene of interest in cultured cells.

The present disclosure also provides pharmaceutical compositions comprising at least one siRNA duplex targeting the gene of interest and a pharmaceutically acceptable carrier. In certain embodiments, the siRNA duplex is encoded by a viral genome in an AAV particle.

In certain embodiments, the present disclosure provides methods for inhibiting/silencing gene expression in a cell. In some aspects, the inhibition of gene expression refers to an inhibition by at least about 20%, such as by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In certain embodiments, the encoded siRNA duplexes may be used to reduce the expression of protein or mRNA encoded by the gene of interest by at least about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of protein or mRNA may be reduced 50-90%. As a non-limiting example, the expression of protein or mRNA may be reduced 30-70%. As a non-limiting example, the expression of protein or mRNA may be reduced 40-70%.

In certain embodiments, the encoded siRNA duplexes may be used to reduce the expression of protein encoded by the gene of interest and/or transcribed mRNA in at least one region of the CNS. As a non-limiting example, the region is the neurons (e.g., cortical neurons).

In certain embodiments, the formulated AAV particles comprising such encoded siRNA molecules may be introduced directly into the central nervous system of the subject, for example, by infusion into the putamen.

In certain embodiments, the formulated AAV particles comprising such encoded siRNA molecules may be introduced directly into the central nervous system of the subject, for example, by infusion into the thalamus of a subject.

In certain embodiments, the formulated AAV particles comprising such encoded siRNA molecules may be introduced directly into the central nervous system of the subject, for example, by infusion into the white matter of a subject.

In certain embodiments, the formulated AAV particles comprising such encoded siRNA molecules may be introduced to the central nervous system of the subject, for example, by intravenous administration to a subject.

In certain embodiments, the pharmaceutical composition of the present disclosure is used as a solo therapy. In certain embodiments, the pharmaceutical composition of the present disclosure is used in combination therapy. The combination therapy may be in combination with one or more neuroprotective agents such as small molecule compounds, growth factors and hormones which have been tested for their neuroprotective effect on motor neuron degeneration.

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory, or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

In some embodiments, the modulatory polynucleotides of the viral genome may comprise at least one nucleic acid sequence encoding at least one siRNA molecule. The nucleic acid sequence may, independently if there is more than one, encode 1, 2, 3, 4, 5, 6, 7, 8, 9, or more than 9 siRNA molecules.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g., antisense strand RNA or antisense oligonucleotides). In many cases it requires higher concentration of the ss-siRNA to achieve the effective gene silencing potency of the corresponding duplex.

Any of the foregoing molecules may be encoded by an AAV particle or viral genome.

The encoded payload of the present disclosure may be introduced into cells by being encoded by the viral genome of an AAV particle. These AAV particles can be engineered and optimized to facilitate the entry into cells that are not readily amendable to transfection/transduction. Also, some synthetic viral vectors possess an ability to integrate the payload into the cell genome, thereby leading to stable payload expression and long-term therapeutic effect. In this manner, viral vectors are engineered as vehicles for specific delivery while lacking the deleterious replication and/or integration features found in wild-type virus.

In certain embodiments, the encoded payload is introduced into a cell by transfecting, infecting or transducing the cell with an AAV particle comprising nucleic acid sequences capable of producing the payload when processed in the cell. In certain embodiments, the payload is introduced into a cell by injecting into the cell or tissue an AAV particle comprising a nucleic acid sequence capable of producing the payload when processed in the cell.

Other methods for introducing AAV particles comprising the nucleic acid sequence for the payloads described herein may comprise photochemical internalization as described in U. S. Patent publication No. 20120264807, the content of which is incorporated herein by reference in its entirety as related to photochemical internalizations, insofar as it does not conflict with the present disclosure.

In certain embodiments, the formulations described herein may contain at least one AAV particle comprising the nucleic acid sequence encoding the payloads described herein. In certain embodiments, the payloads may target the gene of interest at one target site. In another embodiment, the formulation comprises a plurality of AAV particles, each AAV particle comprising a nucleic acid sequence encoding a payload targeting a gene of interest at a different target site. The gene of interest may be targeted at 2, 3, 4, 5 or more than 5 sites.

In certain embodiments, the AAV particles from any relevant species, such as, but not limited to, human, pig, dog, mouse, rat, or monkey may be introduced into cells.

In certain embodiments, the formulated AAV particles may be introduced into cells or tissues which are relevant to the disease to be treated. In certain embodiments, the formulated AAV particles may be introduced into cells which have a high level of endogenous expression of the target gene. In another embodiment, the formulated AAV particles may be introduced into cells which have a low level of endogenous expression of the target gene. In certain embodiments, the cells may be those which have a high efficiency of AAV transduction.

In certain embodiments, formulated AAV particles comprising a nucleic acid sequence encoding a payload of the present disclosure may be used to deliver the payload to the central nervous system (e.g., U.S. Pat. No. 6,180,613; the content of which is incorporated herein by reference in its entirety as related to the delivery and therapeutic use of siRNA molecules and AAV particles, insofar as it does not conflict with the present disclosure).

In certain embodiments, the formulated AAV particles comprising a nucleic acid sequence encoding a payload of the present disclosure may further comprise a modified capsid comprising peptides from non-viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

In certain embodiments, AAV particle comprising the nucleic acid sequence for the siRNA molecules of the present disclosure may be formulated for CNS delivery. Agents that cross the brain blood barrier may be used. For example, some cell penetrating peptides that can target siRNA molecules to the brain blood barrier endothelium may be used to formulate the siRNA duplexes targeting the gene of interest.

In certain embodiments, the formulated AAV particle comprising a nucleic acid sequence encoding a payload of the present disclosure may be administered directly to the CNS. As a non-limiting example, the vector comprises a nucleic acid sequence encoding an siRNA molecule targeting the gene of interest. As a non-limiting example, the vector comprises a nucleic acid sequence encoding an polypeptide targeting a gene of interest.

In certain embodiments, the formulated AAV particle may be administered to a subject (e.g., to the CNS of a subject) in a therapeutically effective amount.

### IV. AAV Particle Production

### General Viral Production Process

Mammalian cells and/or insect cells are often used as viral production cells for the production of rAAV particles. In various embodiments, the methods and systems disclosed herein employ insect cells, e.g., Sf9 cells.

AAV production systems using mammalian or insect cells present a range of complications. There is continued need for methods and systems which allow for effective and efficient large scale (commercial) production of rAAV particles in mammalian and insect cells.

The details of one or more embodiments of the present disclosure are set forth in the accompanying description below. Other features, objects, and advantages of the present disclosure will be apparent from the description, drawings, and the claims. In the description, the singular forms also include the plural unless the context clearly dictates otherwise. Unless defined otherwise, all technical and scientific terms used herein have the same meaning as commonly understood by one of ordinary skill in the art to which this present disclosure belongs. In the case of conflict with disclosures incorporated by reference, the present express description will control.

In certain embodiments, the constructs, polynucleotides, polypeptides, vectors, serotypes, capsids formulations, or particles of the present disclosure may be, may comprise, may be modified by, may be used by, may be used for, may be used with, or may be produced with any sequence, element, construct, system, target or process described in one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO2016137949, WO2017075335; the contents of which are each incorporated herein by reference in their entireties, insofar as they do not conflict with the present disclosure.

AAV production of the present disclosure comprises processes and methods for producing AAV particles and viral vectors which can contact a target cell to deliver a payload construct, e.g., a recombinant viral construct, which comprises a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

The present disclosure provides methods of producing AAV particles or viral vectors by (a) contacting a viral production cell with one or more viral expression constructs encoding at least one AAV capsid protein and/or at least one AAV replication protein, and one or more payload construct vectors, wherein said payload construct vector comprises a payload construct encoding a payload molecule selected from the group consisting of a transgene, a polynucleotide encoding protein, and a modulatory nucleic acid; (b) culturing said viral production cell under conditions such that at least one AAV particle or viral vector is produced, and (c) isolating said at least one AAV particle or viral vector.

In these methods a viral expression construct may encode at least one structural protein and/or at least one non-structural protein. The structural protein may comprise any of the native or wild type capsid proteins VP1, VP2, and/or VP3 or a chimeric protein. The non-structural protein may comprise any of the native or wild type Rep78, Rep68, Rep52 and/or Rep40 proteins or a chimeric protein.

In certain embodiments, an rAAV production method as disclosed herein comprises transient transfection, viral transduction and/or electroporation.

In certain embodiments, the viral production cell is selected from the group consisting of a mammalian cell and an insect cell. In certain embodiments, the insect cell comprises a Spodoptera frugiperda insect cell. In certain embodiments, the insect cell comprises an Sf9 insect cell. In certain embodiments, the insect cell comprises an Sf21 insect cell.

The payload construct vector of the present disclosure may comprise at least one inverted terminal repeat (ITR) and may comprise mammalian DNA.

Also provided are AAV particles and viral vectors produced according to the methods described herein.

The AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell) with at least one viral expression construct encoding at least one capsid protein and at least one AAV replication protein, and at least one payload construct vector. In certain embodiments, separate viral expression constructs encoding the at least one capsid protein and the at least one AAV replication protein may be used. The viral production cell may be contacted by transient transfection, viral transduction and/or electroporation. The payload construct vector may comprise a payload construct encoding a payload molecule such as, but not limited to, a transgene, a polynucleotide encoding protein, and a modulatory nucleic acid. The viral production cell can be cultured under conditions such that at least one AAV particle or viral vector is produced, isolated (e.g., using temperature-induced lysis, mechanical lysis and/or chemical lysis) and/or purified (e.g., using filtration, chromatography and/or immunoaffinity purification). As a non-limiting example, the payload construct vector may comprise mammalian DNA.

In certain embodiments, the AAV particles are produced in an insect cell (e.g., Spodoptera frugiperda (Sf9) cell) using the method described herein. As a non-limiting example, the insect cell is contacted using viral transduction which may comprise baculoviral transduction.

In another embodiment, the AAV particles are produced in a mammalian cell using the method described herein. As a non-limiting example, the mammalian cell is contacted using transient transfection.

In certain embodiments, the viral expression construct may encode at least one structural protein and at least one non-structural protein. As a non-limiting example, the structural protein may comprise VP1, VP2, and/or VP3 capsid proteins. As another non-limiting example, the non-structural protein may comprise Rep78, Rep68, Rep52, and/or Rep40 replication proteins.

In certain embodiments, the AAV particle production method described herein produces greater than 10¹, greater than 10², greater than 10³, greater than 10⁴, or greater than 10⁵ AAV particles in a viral production cell.

In certain embodiments, a process of the present disclosure comprises production of viral particles in a viral production cell using a viral production system which comprises at least one viral expression construct and at least one payload construct. The at least one viral expression construct and at least one payload construct can be co-transfected (e.g., dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, comprising Rep proteins which replicate the payload construct and Cap proteins which assemble to form a capsid that encloses the replicated payload constructs. The resulting AAV particle is extracted from the viral production cells and processed into a pharmaceutical preparation for administration.

In certain embodiments, the process for production of viral particles utilizes seed cultures of viral production cells that comprise one or more baculoviruses (e.g., a Baculoviral Expression Vector (BEV) or baculovirus infected insect cells (BIICs) that have been transfected with a viral expression construct (e.g., comprised in an expressionBac) and a payload construct (e.g., comprised in a payloadBac)). In certain embodiments, the seed cultures are harvested, divided into aliquots and frozen, and may be used at a later time point to initiate an infection of a naïve population of production cells.

Large scale production of AAV particles may utilize a bioreactor. The use of a bioreactor allows for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In another embodiment, the bioreactor is used for continuous production in which a portion of the culture is harvested at an experimentally determined time point for purification of AAV particles, and the remaining culture in the bioreactor is refreshed with additional growth media components.

In certain embodiments, AAV viral particles can be extracted from viral production cells in a process which comprises cell lysis, clarification, sterilization and purification. Cell lysis comprises any process that disrupts the structure of the viral production cell, thereby releasing AAV particles. In certain embodiments cell lysis may comprise thermal shock, chemical, or mechanical lysis methods. In some embodiments, cell lysis is done chemically. Clarification of the lysed cells can comprise the gross purification of the mixture of lysed cells, media components, and AAV particles. In certain embodiments, clarification comprises centrifugation and/or filtration, comprising but not limited to depth end, tangential flow, and/or hollow fiber filtration.

The end result of viral production is a purified collection of AAV particles which comprise two components: (1) a payload construct (e.g., a recombinant viral genome construct) and (2) a viral capsid.

In certain embodiments, a viral production system or process of the present disclosure comprises steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can comprise a single CP Purification step or can comprise multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

In certain embodiments, a viral production system or process of the present disclosure comprises steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. This expansion can include one or more small-volume expansion steps up to a working volume of 2000-5000 mL, followed by one or more large-volume expansion steps in large-scale bioreactors (e.g., Wave and/or N−1 bioreactors) up to a working volume of 25-500 L. The working volume of Viral Production Cells is seeded into a Production Bioreactor and can be further expanded to a working volume of 200-2500 L with a target VPC concentration for BIIC infection.

The working volume of VPCs in the Production Bioreactor is then co-infected with Rep/Cap BIICs and Payload BIICs, e.g., with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

In certain embodiments, a viral production system or process of the present disclosure comprises steps for producing a Drug Substance by processing, clarifying, and purifying a bulk harvest of AAV particles and Viral Production Cells. A bulk harvest of AAV particles and VPCs (within a Production Bioreactor) are processed through cellular disruption and lysis (e.g., chemical lysis and/or mechanical lysis), followed by nuclease treatment of the lysis pool, thereby producing a crude lysate pool. The crude lysate pool is processed through one or more filtration and clarification steps, comprising depth filtration and/or microfiltration to provide a clarified lysate pool. The clarified lysate pool is processed through one or more chromatography and purification steps, comprising one or more affinity chromatography (AFC) steps and one or more ion-exchange chromatography (AEX or CEX) steps, either in series or alternating, to provide a purified product pool. The purified product pool is then optionally processed through nanofiltration, and then through tangential flow filtration (TFF). The TFF process comprises one or more diafiltration (DF) steps and one or more ultrafiltration (UF) steps, either in series or alternating. The product pool is further processed through viral retention filtration (VRF) and another filtration step to provide a drug substance pool. The drug substance pool can be further filtered, then aliquoted into vials for storage and treatment.

### Large Scale Viral Production Systems

In certain embodiments, AAV particle production may be modified to increase the scale of production. Large scale viral production methods according to the present disclosure may comprise any of the processes or processing steps taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety.

Methods of increasing AAV particle production scale typically comprise increasing the number of viral production cells. In certain embodiments, viral production cells comprise adherent cells. To increase the scale of AAV particle production by adherent viral production cells, larger cell culture surfaces are required. In certain embodiments, large-scale production methods comprise the use of roller bottles to increase cell culture surfaces. Other cell culture substrates with increased surface areas are known in the art. Examples of additional adherent cell culture products with increased surface areas comprise, but are not limited to iCELLis (Pall Corp, Port Washington, NY), CELLSTACK®, CELLCUBE® (Corning Corp., Corning, NY) and NUNC™ CELL FACTORY™ (Thermo Scientific, Waltham, MA.) In certain embodiments, large-scale adherent cell surfaces may comprise from about 1,000 cm² to about 100,000 cm².

In certain embodiments, large-scale viral production methods of the present disclosure may comprise the use of suspension cell cultures. Suspension cell culture can allow for significantly increased numbers of cells. Typically, the number of adherent cells that can be grown on about 10-50 cm² of surface area can be grown in about 1 cm³ volume in suspension.

In certain embodiments, large-scale cell cultures may comprise from about 1.0×10⁷ to about 9.9×10⁹ cells, from about 1.0×10⁸ to about 9.9×10¹⁰ cells, from about 1.0×10⁹ to about 9.9×10¹¹ cells, from about 1.0×10¹⁰ to about 9.9×10¹² cells, from about 1.0×10¹¹ to about 9.9×10¹³ cells, from about 1.0×10¹² to about 9.9×10¹⁴ cells, from about 1.0×10¹³ to about 9.9×10¹⁵ cells, from about 1.0×10¹⁴ to about 9.9×10¹⁶ cells, from about 1.0×10¹⁵ to about 9.9×10¹⁷ cells, or from about 1.0×10¹⁶ to about 9.9×10¹⁸ cells. In certain embodiments, large-scale cell cultures may comprise at least 1.0×10¹² AAV particles. In certain embodiments, large-scale cell cultures may comprise at least 1.0×10¹³ AAV particles. In certain embodiments, large-scale cell cultures may comprise at least 1.0×10¹⁴ AAV particles. In certain embodiments, large-scale cell cultures may comprise at least 1.0×10¹⁵ AAV particles. In certain embodiments, large-scale cell cultures may comprise at least 1.0×10¹⁶ AAV particles. In certain embodiments, large-scale cell cultures may comprise at least 1.0×10¹⁷ AAV particles. In certain embodiments, large-scale cell cultures may comprise at least 1.0×10¹⁸ AAV particles.

Transfection of replication cells in large-scale culture formats may be carried out according to any methods known in the art. For large-scale adherent cell cultures, transfection methods may comprise, but are not limited to the use of inorganic compounds (e.g., calcium phosphate) organic compounds (e.g., polyethyleneimine (PEI)) or the use of non-chemical methods (e.g., electroporation). With cells grown in suspension, transfection methods may comprise, but are not limited to the use of inorganic compounds (e.g., calcium phosphate,) organic compounds (e.g., polyethyleneimine (PEI)) or the use of non-chemical methods (e.g., electroporation). In certain embodiments, transfection of large-scale suspension cultures may be carried out according to the section entitled “Transfection Procedure” described in Feng, L. et al., 2008. Biotechnol Appl Biochem. 50:121-32, the contents of which are herein incorporated by reference in their entirety. According to such embodiments, PEI-DNA complexes may be formed for introduction of plasmids to be transfected. In certain embodiments, cells being transfected with PEI-DNA complexes may be ‘shocked’ prior to transfection. This comprises lowering cell culture temperatures to 4° C. for a period of about 1 hour. In certain embodiments, cell cultures may be shocked for a period of from about 10 minutes to about 5 hours. In certain embodiments, cell cultures may be shocked at a temperature of from about 0° C. to about 20° C.

In certain embodiments, transfections may comprise one or more vectors for expression of an RNA effector molecule to reduce expression of nucleic acids from one or more payload construct. Such methods may enhance the production of AAV particles by reducing cellular resources wasted on expressing payload constructs. In certain embodiments, such methods may be carried according to those taught in US Publication No. US2014/0099666, the contents of which are herein incorporated by reference in their entirety.

### Bioreactors

In certain embodiments, suspension cell culture bioreactors may be used for large scale production of AAV particles. In certain embodiments, bioreactors comprise stirred tank reactors. Such reactors generally comprise a vessel, typically cylindrical in shape, with a stirrer (e.g., impeller.) In certain embodiments, such bioreactor vessels may be placed within a water jacket to control vessel temperature and/or to minimize effects from ambient temperature changes.

Bioreactor vessel volume may range in size from about 500 ml to about 2 L, from about 2 L to about 5 L, from about 5 L to about 20 L, from about 20 L to about 50 L, from about 50 L to about 100 L, from about 100 L to about 500 L, from about 500 L to about 2,000 L, from about 2,000 L to about 10,000 L, from about 10,000 L to about 20,000 L, from about 20,000 L to about 50,000 L, or more than 50,000 L. Vessel bottoms may be rounded or flat. In certain embodiments, animal cell cultures may be maintained in bioreactors with rounded vessel bottoms.

In certain embodiments, bioreactor vessels may be warmed through the use of a thermocirculator. Thermocirculators pump heated water around water jackets. In certain embodiments, heated water may be pumped through pipes (e.g., coiled pipes) that are present within bioreactor vessels. In certain embodiments, warm air may be circulated around bioreactors, comprising, but not limited to air space directly above culture medium. Additionally, pH and CO₂ levels may be maintained to optimize cell viability.

In certain embodiments, bioreactors may comprise hollow-fiber reactors. Hollow-fiber bioreactors may support the culture of both anchorage dependent and anchorage independent cells. Further bioreactors may comprise, but are not limited to, packed-bed or fixed-bed bioreactors. Such bioreactors may comprise vessels with glass beads for adherent cell attachment. Further packed-bed reactors may comprise ceramic beads.

In certain embodiments, viral particles are produced through the use of a disposable bioreactor. In certain embodiments, bioreactors may comprise GE WAVE bioreactor, a GE Xcellerax Bioreactor, a Sartorius Biostat Bioreactor, a ThermoFisher Hyclone Bioreactor, or a Pall Allegro Bioreactor.

In certain embodiments, AAV particle production in cell bioreactor cultures may be carried out according to the methods or systems taught in U.S. Pat. Nos. 5,064,764, 6,194,191, 6,566,118, 8,137,948 or US Patent Application No. US2011/0229971, the contents of each of which are herein incorporated by reference in their entirety.

### Expansion of Viral Production Cell (VPC) Mixtures

In certain embodiments, an AAV particle or viral vector of the present disclosure may be produced in a viral production cell (VPC), such as an insect cell. Production cells can be sourced from a Cell Bank (CB) and are often stored in frozen cell banks.

In certain embodiments, a viral production cell from a Cell Bank is provided in frozen form. The vial of frozen cells is thawed, typically until ice crystal dissipate. In certain embodiments, the frozen cells are thawed at a temperature between 10-50° C., 15-40° C., 20-30° C., 25-50° C., 30-45° C., 35-40° C., or 37-39° C. In certain embodiments, the frozen viral production cells are thawed using a heated water bath.

In certain embodiments, a thawed CB cell mixture will have a cell density of 1.0×10⁴-1.0×10⁹ cells/mL. In certain embodiments, the thawed CB cell mixture has a cell density of 1.0×10⁴-2.5×10⁴ cells/mL, 2.5×10⁴-5.0×10⁴ cells/mL, 5.0×10⁴-7.5×10⁴ cells/mL, 7.5×10⁴-1.0×10⁵ cells/mL, 1.0×10⁵-2.5×10⁵ cells/mL, 2.5×10⁵-5.0×10⁵ cells/mL, 5.0×10⁵-7.5×10⁵ cells/mL, 7.5×10⁵-1.0×10⁶ cells/mL, 1.0×10⁶-2.5×10⁶ cells/mL, 2.5×10⁶-5.0×10⁶ cells/mL, 5.0×10⁶-7.5×10⁶ cells/mL, 7.5×10⁶-1.0×10⁷ cells/mL, 1.0×10⁷-2.5×10⁷ cells/mL, 2.5×10⁷-5.0×10⁷ cells/mL, 5.0×10⁷-7.5×10⁷ cells/mL, 7.5×10⁷-1.0×10⁸ cells/mL, 1.0×10⁸-2.5×10⁸ cells/mL, 2.5×10⁸-5.0×10⁸ cells/mL, 5.0×10⁸-7.5×10⁸ cells/mL, or 7.5×10⁸-1.0×10⁹ cells/mL.

In certain embodiments, the volume of the CB cell mixture is expanded. This process is commonly referred to as a Seed Train, Seed Expansion, or CB Cellular Expansion. Cellular/Seed expansion can comprise successive steps of seeding and expanding a cell mixture through multiple expansion steps using successively larger working volumes. In certain embodiments, cellular expansion can comprise one, two, three, four, five, six, seven, or more than seven expansion steps. In certain embodiments, the working volume in the cellular expansion can comprise one or more of the following working volumes or working volume ranges: 5 mL, 10 mL, 20 mL, 5-20 mL, 25 mL, 30 mL, 40 mL, 50 mL, 20-50 mL, 75 mL, 100 mL, 125 mL, 150 mL, 175 mL, 200 mL, 50-200 mL, 250 mL, 300 mL, 400 mL, 500 mL, 750 mL, 1000 mL, 250-1000 mL, 1250 mL, 1500 mL, 1750 mL, 2000 mL, 1000-2000 mL, 2250 mL, 2500 mL, 2750 mL, 3000 mL, 2000-3000 mL, 3500 mL, 4000 mL, 4500 mL, 5000 mL, 3000-5000 mL, 5.5 L, 6.0 L, 7.0 L, 8.0 L, 9.0 L, 10.0 L, and 5.0-10.0 L.

In certain embodiments, a volume of cells from a first expanded cell mixture can be used to seed a second, separate Seed Train/Seed Expansion (instead of using thawed CB cell mixture). This process is commonly referred to as rolling inoculum. In certain embodiments, rolling inoculum is used in a series of two or more (e.g., two, three, four or five) separate Seed Trains/Seed Expansions.

In certain embodiments, large-volume cellular expansion can comprise the use of a bioreactor, such as a GE WAVE bioreactor, a GE Xcellerex Bioreactor, a Sartorius Biostat Bioreactor, a ThermoFisher Hyclone Bioreactor, or a Pall Allegro Bioreactor.

In certain embodiments, the cell density within a working volume is expanded to a target output cell density. In certain embodiments, the output cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the output cell density of a working volume provides a seeding cell density for a larger, successive working volume. In certain embodiments, the seeding cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, cellular expansion can last for 1-50 days. Each cellular expansion step or the total cellular expansion can last for 1-10 days, 1-5 days, 1-3 days, 2-3 days, 2-4 days, 2-5 days, 2-6 days, 3-4 days, 3-5 days, 3-6 days, 3-8 days, 4-5 days, 4-6 days, 4-8 days, 5-6 days, or 5-8 days. In certain embodiments, each cellular expansion step or the total cellular expansion can last for 1-100 generations, 1-1000 generations, 100-1000 generation, 100 generations or more, or 1000 generation or more.

In certain embodiments, infected or transfected production cells can be expanded in the same manner as CB cell mixtures, as set forth in the present disclosure.

### Infection of Viral Production Cells

In certain embodiments, AAV particles of the present disclosure are produced in a viral production cell (VPC), such as an insect cell, by infecting the VPC with a viral vector which comprises an AAV expression construct and/or a viral vector which comprises an AAV payload construct. In certain embodiments, the VPC is infected with an Expression BEV, which comprises an AAV expression construct and a Payload BEV which comprises an AAV payload construct.

In certain embodiments, AAV particles are produced by infecting a VPC with a viral vector which comprises both an AAV expression construct and an AAV payload construct. In certain embodiments, the VPC is infected with a single BEV which comprises both an AAV expression construct and an AAV payload construct.

In certain embodiments, VPCs (such as insect cells) are infected using Infection BIICs in an infection process which comprises the following steps: (i) A collection of VPCs are seeded into a Production Bioreactor; (ii) The seeded VPCs can optionally be expanded to a target working volume and cell density; (iii) Infection BIICs which comprise Expression BEVs and Infection BIICs which comprise Payload BEVs are injected into the Production Bioreactor, resulting in infected viral production cells; and (iv) incubation of the infected viral production cells to produce AAV particles within the viral production cells.

In certain embodiments, the VPC density at infection is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-3.4×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.1×10⁶, 3.2×10⁶, 3.3×10⁶, 3.4×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL. In certain embodiments, the VPC density at infection is 2.0-3.5×10⁶ cells/mL. In certain embodiments, the VPC density at infection is 3.5-5.0×10⁶ cells/mL. In certain embodiments, the VPC density at infection is 5.0-7.5×10⁶ cells/mL. In certain embodiments, the VPC density at infection is 5.0-10.0×10⁶ cells/mL.

In certain embodiments, the VPC density at infection is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-3.4×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.1×10⁶, 3.2×10⁶, 3.3×10⁶, 3.4×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL. In certain embodiments, the VPC density at infection is 2.0-3.5×10⁶ cells/mL. In certain embodiments, the VPC density at infection is 3.5-5.0×10⁶ cells/mL. In certain embodiments, the VPC density at infection is 5.0-7.5×10⁶ cells/mL. In certain embodiments, the VPC density at infection is 5.0-10.0×10⁶ cells/mL.

In certain embodiments, Infection BIICs are combined with the VPCs in target ratios of VPC-to-BIIC. In certain embodiments, the VPC-to-BIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to BIIC volume). In certain embodiments, the VPC-to-BIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to BIIC volume). In certain embodiments, the VPC-to-BIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to BIIC cells). In certain embodiments, the VPC-to-BIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to BIIC cells).

In certain embodiments, Infection BIICs which comprise Expression BEVs are combined with the VPCs in target ratios of VPC-to-expressionBIIC. In certain embodiments, the VPC-to-expressionBIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to expressionBIIC volume). In certain embodiments, the VPC-to-expressionBIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to expressionBIIC volume). In certain embodiments, the VPC-to-expressionBIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to expressionBIIC cells). In certain embodiments, the VPC-to-expressionBIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to expressionBIIC cells).

In certain embodiments, Infection BIICs which comprise Payload BEVs are combined with the VPCs in target ratios of VPC-to-payloadBIIC. In certain embodiments, the VPC-to-payloadBIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to payloadBIIC volume). In certain embodiments, the VPC-to-payloadBIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to payloadBIIC volume). In certain embodiments, the VPC-to-payloadBIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to payloadBIIC cells). In certain embodiments, the VPC-to-payloadBIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to payloadBIIC cells).

In certain embodiments, Infection BIICs which comprise Expression BEVs and Infection BIICs which comprise Payload BEVs are combined with the VPCs in target payloadBIIC-to-expressionBIIC ratios. In certain embodiments, the ratio of payloadBIIC-to-expressionBIIC is 10:1, 9:1, 8:1, 7:1, 6:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:9, 1:10, 1:11, 1:12, 1:13, 1:14, 1:15, 1:16, 1:17, 1:18, 1:19, or 1:20. In certain embodiments, the ratio of payloadBIIC-to-expressionBIIC is between 6.5-7.5:1, 6-7:1, 5.5-6.5:1, 5-6:1, 4.5-5.5:1, 4-5:1, 3.5-4.5:1, 3-4:1, 2.5-3.5:1, 2-3:1, 1.5-2.5:1, 1-2:1, 1-1.5:1, 1:1-1.5, 1:1-2, 1:1.5-2.5, 1:2-3, 1:2.5-3.5, 1:3-4, 1:3.5-4.5, 1:4-5, 1:4.5-5.5, 1:5-6, 1:5.5-6.5, 1:6-7, 1:6.5-7.5, 1:7-8, 1:7.5-8.5, 1:8-9, 1:8.5-9.5, 1:9-10, 1:9.5-10.5, 1:10-11, 1:10.5-11.5, 1:11-12, 1:11.5-12.5, 1:12-13, 1:13.5-14.5, or 1: 14-15. In certain embodiments, the ratio of payloadBIIC-to-expressionBIIC is greater than 1:1. In certain embodiments, the ratio of payloadBIIC-to-expressionBIIC is between 1:1 and 1:12. In certain embodiments, the ratio of payloadBIIC-to-expressionBIIC is between 1:1 and 1:6. In certain embodiments, the ratio of payloadBIIC-to-expressionBIIC is between 1:3 and 1:6. In certain embodiments, the ratio of payloadBIIC-to-expressionBIIC is about 1:1. In certain embodiments, the ratio of payloadBIIC-to-expressionBIIC is about 1:2. In certain embodiments, the ratio of payloadBIIC-to-expressionBIIC is about 1:3. In certain embodiments, the ratio of payloadBIIC-to-expressionBIIC is about 1:4. In certain embodiments, the ratio of payloadBIIC-to-expressionBIIC is about 1:5. In certain embodiments, the ratio of payloadBIIC-to-expressionBIIC is about 1:6. In certain embodiments, the ratio of payloadBIIC-to-expressionBIIC is about 1:7. In certain embodiments, the ratio of payloadBIIC-to-expressionBIIC is about 1:8. In certain embodiments, the ratio of payloadBIIC-to-expressionBIIC is about 1:9.

In certain embodiments, infected Viral Production Cells are incubated under a certain Dissolved Oxygen (DO) Content (DO %). In certain embodiments, infected Viral Production Cells are incubated under a DO % between 10%-50%, 20%-40%, 10%-20%, 15%-25%, 20%-30%, 25%-35%, 30%-40%, 35%-45%, 40%-50%, 10%-15%, 15%-20%, 20%-25%, 25%-30%, 30%-35%, 35%-40%, 40%-45%, or 45%-50%. In certain embodiments, infected Viral Production Cells are incubated under a DO % of about 10%, about 15%, about 20%, about 25%, about 30%, about 35%, about 40%, about 45%, about 50%. In certain embodiments, infected Viral Production Cells are incubated under a DO % between 20%-30% or about 25%. In certain embodiments, infected Viral Production Cells are incubated under a DO % between 25%-35% or about 30%. In certain embodiments, infected Viral Production Cells are incubated under a DO % between 30%-40% or about 35%. In certain embodiments, infected Viral Production Cells are incubated under a DO % between 35%-45% or about 40%.

### Cell Lysis

Cells of the present disclosure, comprising, but not limited to viral production cells, may be subjected to cell lysis according to any methods known in the art. Cell lysis may be carried out to obtain one or more agents (e.g., viral particles) present within any cells of the disclosure. In certain embodiments, a bulk harvest of AAV particles and viral production cells is subjected to cell lysis according to the present disclosure.

In certain embodiments, cell lysis may be carried out according to any of the methods or systems presented in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Cell lysis methods and systems may be chemical or mechanical. Chemical cell lysis typically comprises contacting one or more cells with one or more chemical lysis agent under chemical lysis conditions. Mechanical lysis typically comprises subjecting one or more cells to cell lysis carried out by mechanical force. Lysis can also be completed by allowing the cells to degrade after reaching ˜0% viability.

In certain embodiments, chemical lysis may be used to lyse cells. As used herein, the term “chemical lysis agent” refers to any agent that may aid in the disruption of a cell. In certain embodiments, lysis agents are introduced in solutions, termed lysis solutions or lysis buffers. As used herein, the term “chemical lysis solution” refers to a solution (typically aqueous) comprising one or more lysis agent. In addition to lysis agents, lysis solutions may comprise one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may comprise one or more solubilizing agent. As used herein, the term “solubilizing agent” refers to a compound that enhances the solubility of one or more components of a solution and/or the solubility of one or more entities to which solutions are applied. In certain embodiments, solubilizing agents enhance protein solubility. In certain embodiments, solubilizing agents are selected based on their ability to enhance protein solubility while maintaining protein conformation and/or activity.

Exemplary lysis agents may comprise any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In certain embodiments, lysis agents may be selected from lysis salts, amphoteric agents, cationic agents, ionic detergents, and non-ionic detergents. Lysis salts may comprise, but are not limited to, sodium chloride (NaCl) and potassium chloride (KCl.) Further lysis salts may comprise any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety.

In certain embodiments, cell lysates agents include amino acids such as arginine, or acidified amino acid mixtures such as arginine HCl.

In certain embodiments, the cell lysate solution comprises a stabilizing additive. In certain embodiments, the stabilizing additive can comprise trehalose, glycine betaine, mannitol, potassium citrate, CuCl₂, proline, xylitol, NDSB 201, CTAB and K₂PO₄. In certain embodiments, the stabilizing additive can comprise amino acids such as arginine, or acidified amino acid mixtures such as arginine HCl. In certain embodiments, the stabilizing additive can comprise 0.1 M arginine or arginine HCl. In certain embodiments, the stabilizing additive can comprise 0.2 M arginine or arginine HCl. In certain embodiments, the stabilizing additive can comprise 0.25 M arginine or arginine HCl. In certain embodiments, the stabilizing additive can comprise 0.3 M arginine or arginine HCl. In certain embodiments, the stabilizing additive can comprise 0.4 M arginine or arginine HCl. In certain embodiments, the stabilizing additive can comprise 0.5 M arginine or arginine HCl. In certain embodiments, the stabilizing additive can comprise 0.6 M arginine or arginine HCl. In certain embodiments, the stabilizing additive can comprise 0.7 M arginine or arginine HCl. In certain embodiments, the stabilizing additive can comprise 0.8 M arginine or arginine HCl. In certain embodiments, the stabilizing additive can comprise 0.9 M arginine or arginine HCl. In certain embodiments, the stabilizing additive can comprise 1.0M arginine or arginine HCl.

Concentrations of salts may be increased or decreased to obtain an effective concentration for the rupture of cell membranes. Amphoteric agents, as referred to herein, are compounds capable of reacting as an acid or a base. Amphoteric agents may comprise, but are not limited to lysophosphatidylcholine, 3-((3-Cholamidopropyl) dimethylammonium)-1-propanesulfonate (CHAPS), ZWITTERGENT® and the like. Cationic agents may comprise, but are not limited to, cetyltrimethylammonium bromide (C (16) TAB) and Benzalkonium chloride. Lysis agents comprising detergents may comprise ionic detergents or non-ionic detergents.

Detergents may function to break apart or dissolve cell structures comprising, but not limited to cell membranes, cell walls, lipids, carbohydrates, lipoproteins, and glycoproteins. Exemplary ionic detergents comprise any of those taught in U.S. Pat. Nos. 7,625,570 and 6,593,123 or US Publication No. US2014/0087361, the contents of each of which are herein incorporated by reference in their entirety. In certain embodiments, the lysis solution comprises one or more ionic detergents. Example of ionic detergents for use in a lysis solution comprise, but are not limited to, sodium dodecyl sulfate (SDS), cholate and deoxycholate. In certain embodiments, ionic detergents may be comprised in lysis solutions as a solubilizing agent. In certain embodiments, the lysis solution comprises one or more nonionic detergents. Non-ionic detergents for use in a lysis solution may comprise, but are not limited to, octylglucoside, digitonin, lubrol, C12E8, TWEEN®-20, TWEEN®-80, Triton X-100, Triton X-114, Brij-35, Brij-58, and Noniodet P-40. Non-ionic detergents are typically weaker lysis agents but may be comprised as solubilizing agents for solubilizing cellular and/or viral proteins. In certain embodiments, the lysis solution comprises one or more zwitterionic detergents. Zwitterionic detergents for use in a lysis solution may comprise, but are not limited to: Lauryl dimethylamine N-oxide (LDAO); N,N-Dimethyl-N-dodecylglycine betaine (Empigen® BB); 3-(N,N-Dimethylmyristylammonio) propanesulfonate (Zwittergent® 3-10); n-Dodecyl-N,N-dimethyl-3-ammonio-1-propanesulfonate (Zwittergent® 3-12); n-Tetradecyl-N,N-dimethyl-3-ammonio-1-propanesulfonate (Zwittergent® 3-14); 3-(N,N-Dimethyl palmitylammonio) propanesulfonate (Zwittergent® 3-16); 3-((3-cholamidopropyl) dimethylammonio)-1-propanesulfonate (CHAPS); and 3-([3-Cholamidopropyl] dimethylammonio)-2-hydroxy-1-propanesulfonate (CHAPSO).

In certain embodiments, the lysis solution comprises Triton X-100 (octyl phenol ethoxylate), such as 0.5% w/v of Triton X-100. In certain embodiments, the lysis solution comprises Lauryldimethylamine N-oxide (LDAO), such as 0.184% w/v (4×CMC) of LDAO. In certain embodiments, the lysis solution comprises a seed oil surfactant such as Ecosurf™ SA-9. In certain embodiments, the lysis solution comprises N,N-Dimethyl-N-dodecylglycine betaine (Empigen® BB). In certain embodiments, the lysis solution comprises a Zwittergent® detergent, such as Zwittergent® 3-12 (n-Dodecyl-N,N-dimethyl-3-ammonio-1-propanesulfonate), Zwittergent® 3-14 (n-Tetradecyl-N,N-dimethyl-3-ammonio-1-propanesulfonate), or Zwittergent® 3-16 (3-(N,N-Dimethyl palmitylammonio)propanesulfonate).

Further lysis agents may comprise enzymes and urea. In certain embodiments, one or more lysis agents may be combined in a lysis solution in order to enhance one or more of cell lysis and protein solubility. In certain embodiments, enzyme inhibitors may be comprised in lysis solutions in order to prevent proteolysis that may be triggered by cell membrane disruption.

In certain embodiments, the lysis solution comprises between 0.1-1.0% w/v, between 0.2-0.8% w/v, between 0.3-0.7% w/v, between 0.4-0.6% w/v, or about 0.5% w/v of a cell lysis agent (e.g., detergent). In certain embodiments, the lysis solution comprises between 0.3-0.35% w/v, between 0.35-0.4% w/v, between 0.4-0.45% w/v, between 0.45-0.5% w/v, between 0.5-0.55% w/v, between 0.55-0.6% w/v, between 0.6-0.65% w/v, or between 0.65-0.7% w/v of a cell lysis agent (e.g., detergent).

In certain embodiments, cell lysates generated from adherent cell cultures may be treated with one more nuclease, such as Benzonase nuclease (Grade I, 99% pure) or c-LEcta Denarase nuclease (formerly Sartorius Denarase). In certain embodiments, nuclease is added to lower the viscosity of the lysates caused by liberated DNA.

In certain embodiments, chemical lysis uses a single chemical lysis mixture. In certain embodiments, chemical lysis uses several lysis agents added in series to provide a final chemical lysis mixture.

In certain embodiments, a chemical lysis mixture comprises an acidified amino acid mixture (such as arginine HCl), a non-ionic detergent (such as Triton X-100), and a nuclease (such as Benzonase nuclease). In certain embodiments, the chemical lysis mixture can comprise an acid or base to provide a target lysis pH.

In certain embodiments, the lysis solution comprises 0.5% w/v Triton X-100 (octyl phenol ethoxylate) and 200 mM arginine hydrochloride. In certain embodiments, the lysis solution comprises 0.5% w/v Triton X-100 (octyl phenol ethoxylate) and 200 mM arginine hydrochloride, and lacks detectable nuclease. In certain embodiments, the lysis solution consists of 0.5% w/v Triton X-100 (octyl phenol ethoxylate) and 200 mM arginine hydrochloride.

In certain embodiments, chemical lysis is conducted under chemical lysis conditions. As used herein, the term “chemical lysis conditions” refers to any combination of environmental conditions (e.g., temperature, pressure, pH, etc.) in which targets cells can be lysed by a chemical lysis agent.

In certain embodiments, the lysis pH is between 3.0-3.5, 3.5-4.0, 4.0-4.5, 4.5-5.0, 5.0-5.5, 5.5-6.0, 6.0-6.5, 6.5-7.0, 7.0-7.5, or 7.5-8.0. In certain embodiments, the lysis pH is between 6.0-7.0, 6.5-7.0, 6.5-7.5, or 7.0-7.5.

In certain embodiments, the lysis temperature is between 15-35° C., between 20-30° C., between 25-39° C., between 20-21° C., between 20-22° C., between 21-22° C., between 21-23° C., between 22-23° C., between 22-24° C., between 23-24° C., between 23-25° C., between 24-25° C., between 24-26° C., between 25-26° C., between 25-27° C., between 26-27° C., between 26-28° C., between 27-28° C., between 27-29° C., between 28-29° C., between 28-30° C., between 29-30° C., between 29-31° C., between 30-31° C., between 30-32° C., between 31-32° C., or between 31-33° C.

In certain embodiments, the lysis solution comprises 0.5% w/v Triton X-100 (octyl phenol ethoxylate) and 200 mM arginine hydrochloride, and lysis conditions comprise a duration of at least 4 hours (e.g., 4-6 hours, e.g., 4 hours) at 26° C.-28° C. (e.g., 27° C.). In certain embodiments, the lysis solution comprises 0.5% w/v Triton X-100 (octyl phenol ethoxylate) and 200 mM arginine hydrochloride, and lacks detectable nuclease, and lysis conditions comprise a duration of at least 4 hours (e.g., 4-6 hours, e.g., 4 hours) at 26° C.-28° C. (e.g., 27° C.). In certain embodiments, the lysis solution consists of 0.5% w/v Triton X-100 (octyl phenol ethoxylate) and 200 mM arginine hydrochloride, and lysis conditions comprise a duration of at least 4 hours (e.g., 4-6 hours, e.g., 4 hours) at 26° C.-28° C. (e.g., 27° C.).

In certain embodiments, mechanical cell lysis is carried out. Mechanical cell lysis methods may comprise the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In certain embodiments, lysis conditions comprise increased or decreased temperatures. According to certain embodiments, lysis conditions comprise changes in temperature to promote cellular disruption. Cell lysis carried out according to such embodiments may comprise freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In certain embodiments, one or more cryoprotectants are comprised in cell solutions undergoing freeze-thaw lysis. As used herein, the term “cryoprotectant” refers to an agent used to protect one or more substance from damage due to freezing. Cryoprotectants may comprise any of those taught in US Publication No. US2013/0323302 or U.S. Pat. Nos. 6,503,888, 6,180,613, 7,888,096, 7,091,030, the contents of each of which are herein incorporated by reference in their entirety. In certain embodiments, cryoprotectants may comprise, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose, and urea. In certain embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may comprise, but are not limited to mechanical forces, sonic forces, gravitational forces, optical forces, electrical forces, and the like. Cell lysis carried out by mechanical force is referred to herein as “mechanical lysis.” Mechanical forces that may be used according to mechanical lysis may comprise high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g., high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g., viral particles.) Other mechanical lysis methods may comprise physical disruption of cells by scraping.

Cell lysis methods may be selected based on the cell culture format of cells to be lysed. For example, with adherent cell cultures, some chemical and mechanical lysis methods may be used. Such mechanical lysis methods may comprise freeze-thaw lysis or scraping. In another example, chemical lysis of adherent cell cultures may be carried out through incubation with lysis solutions comprising surfactant, such as Triton-X-100.

In certain embodiments, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification and Purification: General

Cell lysates comprising viral particles may be subjected to clarification and purification. Clarification generally refers to the initial steps taken in the purification of viral particles from cell lysates and serves to prepare lysates for further purification by removing larger, insoluble debris from a bulk lysis harvest. Viral production can comprise clarification steps at any point in the viral production process. Clarification steps may comprise, but are not limited to, centrifugation and filtration. During clarification, centrifugation may be carried out at low speeds to remove larger debris only. Similarly, filtration may be carried out using filters with larger pore sizes so that only larger debris is removed.

Purification generally refers to the final steps taken in the purification and concentration of viral particles from cell lysates by removing smaller debris from a clarified lysis harvest in preparing a final Pooled Drug Substance. Viral production can comprise purification steps at any point in the viral production process. Purification steps may comprise, but are not limited to, filtration and chromatography. Filtration may be carried out using filters with smaller pore sizes to remove smaller debris from the product or with larger pore sizes to retain larger debris from the product. Filtration may be used to alter the concentration and/or contents of a viral production pool or stream. Chromatography may be carried out to selectively separate target particles from a pool of impurities.

Large-scale production of high-concentration AAV formulations is complicated by the tendency for high concentrations of AAV particles to aggregate or agglomerate. Small scale clarification and concentration systems, such as dialysis cassettes or spin centrifugation, are generally not sufficiently scalable for large-scale production. The present disclosure provides embodiments of a clarification, purification, and concentration system for processing large volumes of high-concentration AAV production formulations. In certain embodiments, the large-volume clarification system comprises one or more of the following processing steps: Depth Filtration, Microfiltration (e.g., 0.2 μm Filtration), Affinity Chromatography, Ion Exchange Chromatography such as anion exchange chromatography (AEX) or cation exchange chromatography (CEX), a tangential flow filtration system (TFF), Nanofiltration (e.g., Virus Retentive Filtration (VRF)), Final Filtration (FF), and Fill Filtration.

Objectives of viral clarification and purification comprise high throughput processing of cell lysates and to optimize ultimate viral recovery. Advantages of comprising clarification and purification steps of the present disclosure comprise scalability for processing of larger volumes of lysate. In certain embodiments, clarification and purification may be carried out according to any of the methods or systems presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

In certain embodiments, the compositions comprising at least one AAV particle may be isolated or purified using the methods or systems described in U.S. Pat. Nos. 6,146,874, 6,660,514, 8,283,151 or U.S. Pat. No. 8,524,446, the contents of which are herein incorporated by reference in their entirety.

### Clarification and Purification: Centrifugation

According to certain embodiments, cell lysates may be clarified by one or more centrifugation steps. Centrifugation may be used to pellet insoluble particles in the lysate. During clarification, centrifugation strength (which can be expressed in terms of gravitational units (g), which represents multiples of standard gravitational force) may be lower than in subsequent purification steps. In certain embodiments, centrifugation may be carried out on cell lysates at a gravitation force from about 200 g to about 800 g, from about 500 g to about 1500 g, from about 1000 g to about 5000 g, from about 1200 g to about 10000 g or from about 8000 g to about 15000 g. In certain embodiments, cell lysate centrifugation is carried out at 8000 g for 15 minutes. In certain embodiments, density gradient centrifugation may be carried out in order to partition particulates in the cell lysate by sedimentation rate. Gradients used according to methods or systems of the present disclosure may comprise, but are not limited to, cesium chloride gradients and iodixanol step gradients. In certain embodiments, centrifugation uses a decanter centrifuge system. In certain embodiments, centrifugation uses a disc-stack centrifuge system. In certain embodiments, centrifugation comprises ultracentrifugation, such two-cycle CsCl gradient ultracentrifugation or iodixanol discontinuous density gradient ultracentrifugation.

### Clarification and Purification: Filtration

In certain embodiments, one or more microfiltration, nanofiltration and/or ultrafiltration steps may be used during clarification, purification and/or sterilization. The one or more microfiltration, nanofiltration or ultrafiltration steps can comprise the use of a filtration system such as EMD Millipore Express SHC XL10 0.5/0.2 μm filter, EMD Millipore Express SHCXL6000 0.5/0.2 μm filter, EMD Millipore Express SHCXL150 filter, EMD Millipore Millipak Gamma Gold 0.22 μm filter (dual-in-line sterilizing grade filters), a Pall Supor EKV, 0.2 μm sterilizing-grade filter, Asahi Planova 35N, Asahi Planova 20N, Asahi Planova 75N, Asahi Planova BioEx, Millipore Viresolve NFR or a Sartorius Sartopore 2XLG, 0.8/0.2 μm.

In certain embodiments, one or more microfiltration steps may be used during clarification, purification and/or sterilization. Microfiltration utilizes microfiltration membranes with pore sizes typically between 0.1 μm and 10 μm. Microfiltration is generally used for general clarification, sterilization, and removal of microparticulates. In certain embodiments, microfiltration is used to remove aggregated clumps of viral particles. In certain embodiments, a production process or system of the present disclosure comprises at least one microfiltration step. The one or more microfiltration steps can comprise a Depth Filtration step with a Depth Filtration system, such as EMD Millipore Millistak⁺ POD filter (D0HC media series), Millipore MC0SP23CL3 filter (COSP media series), or Sartorius Sartopore filter series. Microfiltration systems of the present disclosure can be pre-rinsed, packed, equilibrated, flushed, processed, eluted, washed or cleaned with formulations known to those in the art, comprising AAV pharmaceutical, processing and storage formulations of the present disclosure. In certain embodiments, clarification comprises use of a COSP media series filter. In some embodiments, the COSP media series filter is effective to reduce or prevent 0.2-micron filter clogging.

In certain embodiments, one or more ultrafiltration steps may be used during clarification and purification. The ultrafiltration steps can be used for concentrating, formulating, desalting or dehydrating either processing and/or formulation solutions of the present disclosure. Ultrafiltration utilizes ultrafiltration membranes, with pore sizes typically between 0.001 and 0.1 μm. Ultrafiltration membranes can also be defined by their molecular weight cutoff (MWCO) and can have a range from 1 kD to 500 kD. Ultrafiltration is generally used for concentrating and formulating dissolved biomolecules such as proteins, peptides, plasmids, viral particles, nucleic acids, and carbohydrates. Ultrafiltration systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more nanofiltration steps may be used during clarification and purification. Nanofiltration utilizes nanofiltration membranes, with pore sizes typically less than 100 nm. Nanofiltration is generally used for removal of unwanted endogenous viral impurities (e.g., baculovirus). In certain embodiments, nanofiltration can comprise viral removal filtration (VRF). VRF filters can have a filtration size typically between 15 nm and 100 nm. Examples of VRF filters comprise (but are not limited to): Planova 15N, Planova 20N, and Planova 35N (Asahi-Kasei Corp, Tokyo, Japan); and Viresolve NFP and Viresolve NFR (Millipore Corp, Billerica, MA, USA). Nanofiltration systems of the present disclosure can be pre-rinsed, packed, equilibrated, flushed, processed, eluted, washed or cleaned with formulations known to those in the art, comprising AAV pharmaceutical, processing and storage formulations of the present disclosure. In certain embodiments, nanofiltration is used to remove aggregated clumps of viral particles.

In certain embodiments, one or more tangential flow filtration (TFF) (also known as cross-flow filtration) steps may be used during clarification and purification. Tangential flow filtration is a form of membrane filtration in which a feed stream (which comprises the target agent/particle to be clarified and concentrated) flows from a feed tank into a filtration module or cartridge. Within the TFF filtration module, the feed stream passes parallel to a membrane surface, such that one portion of the stream passes through the membrane (permeate/filtrate) while the remainder of the stream (retentate) is recirculated back through the filtration system and into the feed tank.

In certain embodiments, the TFF filtration module can be a flat plate module (stacked planar cassette), a spiral wound module (spiral-wound membrane layers), or a hollow fiber module (bundle of membrane tubes). Examples of TFF systems for use in the present disclosure comprise, but are not limited to: Spectrum mPES Hollow Fiber TFF system (0.5 mm fiber ID, 100 kDA MWCO) or Millipore Ultracel PLCTK system with Pellicon-3 cassette (0.57 m², 30 kDA MWCO).

New buffer materials can be added to the TFF feed tank as the feed stream is circulated through the TFF filtration system. In certain embodiments, buffer materials can be fully replenished as the flow stream circulates through the TFF filtration system. In this embodiment, buffer material is added to the stream in equal amounts to the buffer material lost in the permeate, resulting in a constant concentration. In certain embodiments, buffer materials can be reduced as the flow stream circulates through the filtration system. In this embodiment, a reduced amount of buffer material is added to the stream relative to the buffer material lost in the permeate, resulting in an increased concentration. In certain embodiments, buffer materials can be replaced as the flow stream circulates through the filtration system. In this embodiment, the buffer added to stream is different from buffer materials lost in the permeate, resulting in an eventual replacement of buffer material in the stream. TFF systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, a TFF load pool can be spiked with an excipient or diluent prior to filtration. In certain embodiments, a TFF load pool is spiked with a high-salt mixture (such as sodium chloride or potassium chloride) prior to filtration. In certain embodiments, a TFF load pool is spiked with a high-sugar mixture (such as 50% w/v sucrose) prior to filtration.

The effectiveness of TFF processing can depend on several factors, comprising (but not limited to): shear stress from flow design, cross-flow rate, filtrate flow control, transmembrane pressure (TMP), membrane conditioning, membrane composition (e.g., hollow fiber construction) and design (e.g., surface area), system flow design, reservoir design, and mixing strategy. In certain embodiment, the filtration membrane can be exposed to pre-TFF membrane conditioning.

In certain embodiments, TFF processing can comprise one or more microfiltration stages. In certain embodiments, TFF processing can comprise one or more ultrafiltration stages. In certain embodiments, TFF processing can comprise one or more nanofiltration stages.

In certain embodiments, TFF processing can comprise one or more concentration stages, such as an ultrafiltration (UF) or microfiltration (MF) concentration stage. In the concentration stage, a reduced amount of buffer material is replaced as the stream circulates through the filtration system (relative to the amount of buffer material lost as permeate). The failure to completely replace all of the buffer material lost in the permeate results in an increased concentration of viral particles within the filtration stream. In certain embodiments, an increased amount of buffer material is replaced as the stream circulates through the filtration system. The incorporation of excess buffer material relative to the amount of buffer material lost in the permeate results in a decreased concentration of viral particles within the filtration stream.

In certain embodiments, TFF processing can comprise one or more diafiltration (DF) stages. The diafiltration stage comprises replacement of a first buffer material (such as a high salt material) within a second buffer material (such a low-salt or zero-salt material). In this embodiment, a second buffer is added to flow stream which is different from a first buffer material lost in the permeate, resulting in an eventual replacement of buffer material in the stream.

In certain embodiments, TFF processing can comprise multiple stages in series. In certain embodiments, a TFF processing process can comprise an ultrafiltration (UF) concentration stage followed by a diafiltration stage (DF). In certain embodiments, TFF comprising UF followed by DF results in increased rAAV recovery relative to TFF comprising DF followed by UF. In some embodiments, TFF comprising UF followed by DF results in about 70-80% recovery of rAAV.

In certain embodiments, a TFF processing can comprise a diafiltration stage followed by an ultrafiltration concentration stage. In certain embodiments, a TFF processing can comprise a first diafiltration stage, followed by an ultrafiltration concentration stage, followed by a second diafiltration stage. In certain embodiments, a TFF processing can comprise a first diafiltration stage which incorporates a high-salt-low-sugar buffer material into the flow stream, followed by an ultrafiltration/concentration stage which results in a high concentration of the viral material in the flow stream, followed by a second diafiltration stage which incorporates a low-salt-high-sugar or zero-salt-high-sugar buffer material into the flow stream. In certain embodiments, the salt can be sodium chloride, sodium phosphate, potassium chloride, potassium phosphate, or a combination thereof. In certain embodiments, the sugar can be sucrose, such as a 5% w/v sucrose mixture or a 7% w/v sucrose mixture.

In certain embodiments, the one or more TFF steps can comprise a formulation diafiltration step in which at least a portion of the liquid media of the viral production pool is replaced with a high-sucrose formulation buffer. In certain embodiments, the high-sucrose formulation buffer comprises between 6-8% w/v of a sugar or sugar substitute and between 90-100 mM of an alkali chloride salt. In certain embodiments, the high-sucrose formulation buffer comprises 7% w/v of sucrose and between 90-100 mM sodium chloride. In certain embodiments, the high-sucrose formulation buffer comprises 7% w/v of sucrose, 10 mM Sodium Phosphate, between 95-100 mM sodium chloride, and 0.001% (w/v) Poloxamer 188. In certain embodiments, the formulation diafiltration step is the final diafiltration step in the one or more TFF steps. In certain embodiments, the formulation diafiltration step is the only diafiltration step in the one or more TFF steps.

In certain embodiments, TFF processing can comprise multiple stages which occur contemporaneously. As a non-limiting example, a TFF clarification process can comprise an ultrafiltration stage which occurs contemporaneously with a concentration stage.

Methods of cell lysate clarification and purification by filtration are well understood in the art and may be carried out according to a variety of available methods comprising, but not limited to passive filtration and flow filtration. Filters used may comprise a variety of materials and pore sizes. For example, cell lysate filters may comprise pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may comprise, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In certain embodiments, clarification may comprise filtration through a filter with 2.0 μM pore size to remove large debris, followed by passage through a filter with 0.45 μM pore size to remove intact cells.

Filter materials may be composed of a variety of materials. Such materials may comprise, but are not limited to, polymeric materials and metal materials (e.g., sintered metal and pored aluminum.) Exemplary materials may comprise, but are not limited to nylon, cellulose materials (e.g., cellulose acetate), polyvinylidene fluoride (PVDF), polyethersulfone, polyamide, polysulfone, polypropylene, and polyethylene terephthalate. In certain embodiments, filters useful for clarification of cell lysates may comprise, but are not limited to ULTIPLEAT PROFILE™ filters (Pall Corporation, Port Washington, NY), SUPOR™ membrane filters (Pall Corporation, Port Washington, NY).

In certain embodiments, flow filtration may be carried out to increase filtration speed and/or effectiveness. In certain embodiments, flow filtration may comprise vacuum filtration. According to such methods, a vacuum is created on the side of the filter opposite that of cell lysate to be filtered. In certain embodiments, cell lysates may be passed through filters by centrifugal forces. In certain embodiments, a pump is used to force cell lysate through clarification filters. Flow rate of cell lysate through one or more filters may be modulated by adjusting one of channel size and/or fluid pressure.

### Clarification and Purification: Chromatography

In certain embodiments, AAV particles in a formulation may be clarified and purified from cell lysates through one or more chromatography steps using one or more different methods of chromatography. Chromatography refers to any number of methods known in the art for selectively separating out one or more elements from a mixture. Such methods may comprise, but are not limited to, ion exchange chromatography (e.g., cation exchange chromatography and anion exchange chromatography), affinity chromatography (e.g., immunoaffinity chromatography, metal affinity chromatography, pseudo affinity chromatography such as Blue Sepharose resins), hydrophobic interaction chromatography (HIC), size-exclusion chromatography, and multimodal chromatography (MMC) (chromatographic methods that utilize more than one form of interaction between the stationary phase and analytes). In certain embodiments, methods or systems of viral chromatography may comprise any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Chromatography systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more ion exchange (IEX) chromatography steps may be used to isolate viral particles. The ion exchange step can comprise anion exchange (AEX) chromatography, cation exchange (CEX) chromatography, or a combination thereof. In certain embodiments, ion exchange chromatography is used in a bind/elute mode. Bind/elute IEX can be used by binding viral particles to a stationary phase based on charge-charge interactions between capsid proteins (or other charged components) of the viral particles and charged sites present on the stationary phase. This process can comprise the use of a column through which viral preparations (e.g., clarified lysates) are passed. After application of viral preparations to the charged stationary phase (e.g., column), bound viral particles may then be eluted from the stationary phase by applying an elution solution to disrupt the charge-charge interactions. Elution solutions may be optimized by adjusting salt concentration and/or pH to enhance recovery of bound viral particles. In certain embodiments, the elution solution can comprise a nuclease such as Benzonase nuclease. Depending on the charge of viral capsids being isolated, cation or anion exchange chromatography methods may be selected. In certain embodiments, ion exchange chromatography is used in a flow-through mode. Flow-through IEX can be used by binding non-viral impurities or unwanted viral particles to a stationary phase (based on charge-charge interactions) and allowing the target viral particles in the viral preparation to “flow through” the IEX system into a collection pool.

Methods or systems of ion exchange chromatography may comprise, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In certain embodiments, the IEX process uses an AEX chromatography system such as a Sartorius Sartobind Q membrane, a Sartorius Sartobind STIC membrane, a Millipore Fractogel TMAE HiCap(m) Flow-Through membrane, a GE Q Sepharose HP membrane, Poros XQ or Poros HQ. In certain embodiments, the IEX process uses a CEX system such as a Poros XS membrane. In certain embodiments, the AEX system comprises a stationary phase which comprises a trimethylammoniumethyl (TMAE) functional group. In certain embodiments, the IEX process uses a Multimodal Chromatography (MMC) system such as a Nuvia aPrime 4A membrane.

In certain embodiments, one or more affinity chromatography steps, such as immunoaffinity chromatography, may be used to isolate viral particles. Immunoaffinity chromatography is a form of chromatography that utilizes one or more immune compounds (e.g., antibodies or antibody-related structures) to retain viral particles. Immune compounds may bind specifically to one or more structures on viral particle surfaces, comprising, but not limited to one or more viral coat protein. In certain embodiments, immune compounds may be specific for a particular viral variant. In certain embodiments, immune compounds may bind to multiple viral variants. In certain embodiments, immune compounds may comprise recombinant single-chain antibodies. Such recombinant single chain antibodies may comprise those described in Smith, R. H. et al., 2009. Mol. Ther. 17(11):1888-96, the contents of which are herein incorporated by reference in their entirety. Such immune compounds (e.g., recombinant protein ligands) are capable of binding to several AAV capsid variants, comprising, but not limited to AAV1, AAV2, AAV3, AAV5, AAV6 and/or AAV8 or any of those taught herein. In some embodiments, such immune compounds (e.g., recombinant protein ligands) are capable of binding to at least AAV2. In certain embodiments, the AFC process uses a GE AVB Sepharose HP column resin, Poros CaptureSelect AAV8 resins (ThermoFisher), Poros CaptureSelect AAV9 resins (ThermoFisher) and Poros CaptureSelect AAVX resins (ThermoFisher).

In certain embodiments, one or more affinity chromatography steps precedes one or more anion exchange chromatography steps. In certain embodiments, one or more anion exchange chromatography steps precedes one or more affinity chromatography steps.

In certain embodiments, one or more size-exclusion chromatography (SEC) steps may be used to isolate viral particles. SEC may comprise the use of a gel to separate particles according to size. In viral particle purification, SEC filtration is sometimes referred to as “polishing.” In certain embodiments, SEC may be carried out to generate a final product that is near-homogenous. Such final products may in certain embodiments be used in pre-clinical studies and/or clinical studies (Kotin, R. M. 2011. Human Molecular Genetics. 20(1):R2-R6, the contents of which are herein incorporated by reference in their entirety.) In certain embodiments, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In some embodiments, purification of recombinant AAV produces a total rAAV process yield of 30-50%.

### IV. Therapeutic Applications and Methods of Treatment

The present disclosure provides a method for treating a disease, disorder and/or condition in a subject, including a human subject, comprising administering to the subject an AAV particle described herein, e.g., an AAV particle comprising an AAV capsid polypeptide, or administering to the subject any of the described compositions, including a pharmaceutical composition, described herein.

Provided in the present disclosure are methods for introducing (e.g., delivering) an AAV particle described herein (e.g., an AAV particle comprising an AAV capsid polypeptide) into cells. In some embodiments, the method comprises introducing into said cells an AAV particle or vector described herein in an amount sufficient to modulate, e.g., increase, the production of a target gene, mRNA, and/or protein. In some embodiments, the method comprises introducing into said cells an AAV particle or vector described herein in an amount sufficient to modulate, e.g., decrease, expression of a target gene, mRNA, and/or protein. In some aspects, the cells may be neurons such as but not limited to, motor, hippocampal, entorhinal, thalamic, cortical, sensory, sympathetic, or parasympathetic neurons, and glial cells such as astrocytes, microglia, and/or oligodendrocytes. In other aspects, the cells may be a muscle cell, e.g., a cell of a diaphragm, a quadriceps, or a heart (e.g., a heart atrium or a heart ventricle).

Disclosed in the present disclosure are methods for treating a neurological disease/disorder or a neurodegenerative disorder, a muscular or neuromuscular disorder, or a neurooncological disorder associated with aberrant, e.g., insufficient or increased, function/presence of a protein, e.g., a target protein in a subject in need of treatment.

In some embodiments, the method comprises administering to the subject a therapeutically effective amount of a composition comprising AAV particles described herein. As a non-limiting example, the AAV particles can increase target gene expression, increase target protein production, and thus reduce one or more symptoms of neurological disease in the subject such that the subject is therapeutically treated.

In other embodiments, the method comprises administering to the subject a therapeutically effective amount of a composition comprising AAV particles (e.g., an AAV particle comprising an AAV capsid polypeptide) comprising a viral genome with a nucleic acid sequence encoding one or more siRNA molecules. As a non-limiting example, the siRNA molecules can silence target gene expression, inhibit target protein production, and reduce one or more symptoms of neurological disease in the subject such that the subject is therapeutically treated.

In some embodiments, the composition comprising the AAV particles described is administered to the central nervous system of the subject via systemic administration. In some embodiments, the systemic administration is intravenous (IV) injection. In some embodiments, the AAV particle described herein or a pharmaceutical composition comprising an AAV particle described herein is administered by focused ultrasound (FUS), e.g., coupled with the intravenous administration of microbubbles (FUS-MB) or MRI-guided FUS coupled with intravenous administration.

In some embodiments, the composition comprising the AAV particle described herein is administered to the central nervous system of the subject via intraventricular administration. In some embodiments, the composition comprising the AAV particle described herein is administered via intra-cisterna magna injection (ICM).

In some embodiments, the composition comprising an AAV particle described herein is administered to the central nervous system of the subject via intraventricular injection and intravenous injection.

In some embodiments, the composition comprising the AAV particle described herein is administered to the central nervous system of the subject via ICM injection and intravenous injection at a specific dose per subject. As a non-limiting example, the AAV particles are administered via ICM injection at a dose of 1×104 VG per subject. As a non-limiting example, the AAV particles are administered via IV injection at a dose of 2×1013 VG per subject.

In some embodiments, the composition comprising the AAV particle described herein is administered to the central nervous system of the subject. In other embodiments, the composition comprising the AAV particle described herein is administered to a CNS tissue of a subject (e.g., putamen, hippocampus, thalamus, or cortex of the subject).

In some embodiments, the composition comprising the AAV particle described herein is administered to the central nervous system of the subject via intraparenchymal injection. Non-limiting examples of intraparenchymal injections include intraputamenal, intracortical, intrathalamic, intrastriatal, intrahippocampal or into the entorhinal cortex.

In some embodiments, the composition comprising the AAV particle described herein is administered to the central nervous system of the subject via intraparenchymal injection and intravenous injection.

In some embodiments, the composition comprising the AAV particle described herein is administered to the central nervous system of the subject via intraventricular injection, intraparenchymal injection and intravenous injection.

In some embodiments, the composition comprising an AAV particle described herein or a plurality of particles of the present disclosure is administered to a muscle of the subject via intravenous injection. In some embodiments, the composition comprising an AAV particle of a plurality of particles of the present disclosure is administered to a muscle of the subject via intramuscular injection.

In some embodiments, an AAV particle described herein may be delivered into specific types of cells, including, but not limited to, thalamic, hippocampal, entorhinal, cortical, motor, sensory, excitatory, inhibitory, sympathetic, or parasympathetic neurons; glial cells including oligodendrocytes, astrocytes and microglia; and/or other cells surrounding neurons such as T cells. In some embodiments, an AAV particle described herein may be delivered into a muscle cell, e.g., a cell of the quadriceps, diaphragm, liver, and/or heart (e.g., heart atrium or heart ventricle).

In some embodiments, an AAV particle, e.g., a plurality of particles, described herein may be delivered to a cell or region of the midbrain. In some embodiments, an AAV particle, e.g., a plurality of particles, described herein may be delivered to a cell or region of the brains stem.

In some embodiments, an AAV particle, e.g., a plurality of particles, described herein may be delivered to neurons in the putamen, hippocampus, thalamus and/or cortex.

In some embodiments, an AAV particle, e.g., a plurality of particles, described herein may be used as a therapy for a genetic disorder, e.g., an autosomal dominant genetic disorder, an autosomal recessive disorder, X-linked dominant genetic disorder, an X-linked recessive genetic disorder, or a Y-linked genetic disorder. In some embodiments, the genetic disorder is a monogenetic disorder or a polygenic disorder. In some embodiments, treatment of a genetic disorder, e.g., a monogenic disorder, comprises the use of an AAV particle described herein for a gene replacement therapy.

In some embodiments, an AAV particle, e.g., a plurality of particles, described herein may be used as a therapy for a neurological disease.

In some embodiments, an AAV particle, e.g., a plurality of particles, described herein may be used as a therapy for tauopathies.

In some embodiments, an AAV particle, e.g., a plurality of particles, described herein may be used as a therapy for Alzheimer's Disease.

In some embodiments, an AAV particle, e.g., a plurality of particles, of the present disclosure may be used as a therapy for Amyotrophic Lateral Sclerosis.

In some embodiments, an AAV particle, e.g., a plurality of particles, described herein may be used as a therapy for Huntington's Disease.

In some embodiments, an AAV particle, e.g., a plurality of particles, described herein may be used as a therapy for Parkinson's Disease.

In some embodiments, an AAV particle, e.g., a plurality of particles, described herein may be used as a therapy for Gaucher disease (GD) (e.g., Type 1 GD, Type 2 GD, or Type 3 GD). In some embodiments, an AAV particle, e.g., a plurality of particles, described herein may be used as a therapy for Parkinson's disease associated with a GBA mutation. In some embodiments, an AAV particle, e.g., a plurality of particles, described herein may be used as a therapy for dementia with Lewy Bodies (DLB).

In some embodiments, an AAV particle, e.g., a plurality of particles, described herein may be used as a therapy for spinal muscular atrophy.

In some embodiments, an AAV particle, e.g., a plurality of particles, described herein may be used as a therapy for a leukodystrophy, e.g., Alexander disease, autosomal dominant leukodystrophy with autonomic diseases (ADLD), Canavan disease, cerebrotendinous xanthomatosis (CTX), metachromatic leukodystrophy (MLD), Pelizaeus-Merzbacher disease, or Refsum disease.

In some embodiments, an AAV particle, e.g., a plurality of particles, described herein may be used as a therapy for Friedreich's Ataxia.

In some embodiments, an AAV particle, e.g., a plurality of particles, described herein may be used as a therapy for chronic or neuropathic pain.

In some embodiments, an AAV particle, e.g., a plurality of particles, described herein may be used as a therapy for a disease associated with expression of HER2, e.g., a disease associated with overexpression of HER2. In some embodiments, the AAV particle described herein is useful for the treatment, prophylaxis, palliation or amelioration of a HER2-positive cancer. In some embodiments, the HER2-positive cancer is a HER2-positive solid tumor. Additionally, or alternatively, the HER2-positive cancer may be a locally advanced or metastatic HER2-positive cancer. In some instances, the HER2-positive cancer is a HER2-positive breast cancer or a HER2-positive gastric cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer. In some instances, the HER2-positive cancer is prostate cancer. In some embodiments, the HER2-positive cancer has metastasized to the central nervous system (CNS). In some instances, the metastasized HER2-cancer has formed CNS neoplasms.

In some embodiments, an AAV particle, e.g., a plurality of particles, described herein may be used as a therapy for a neuro-oncological disorder. In some embodiments, the neuro-oncological disorder is a cancer of primary CNS origin (e.g., a cancer of a CNS cell and/or CNS tissue). In some embodiments, the neuro-oncological disorder is metastatic cancer in a CNS cell, CNS region, and/or a CNS tissue. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

In some embodiments, an AAV particle, e.g., a plurality of particles, described herein may be used as a therapy for a muscular disorder or a neuromuscular disorder.

In some embodiments, an AAV particle, e.g., a plurality of particles, described herein may be used as a therapy for a cardiac disease or heart disease and/or method of improving (e.g., enhancing) cardiac function in a subject. In some embodiments, the cardiac disease is a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction.

In some embodiments, administration of the AAV particle described herein to a subject may increase target gene, mRNA, and/or protein levels in a subject, relative to a control, e.g., the gene, mRNA, and/or mRNA levels in the subject prior to receiving AAV particle. The target gene, mRNA, and/or protein levels may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS, or a muscle, a region of a muscle, or a cell of a muscle, of a subject. In some embodiments, cell of the CNS comprises an astrocyte, microglia, cortical neuron, hippocampal neuron, DRG and/or sympathetic neuron, sensory neuron, oligodendrocyte, motor neuron, or combination thereof. As a non-limiting example, the AAV particles may increase the gene, mRNA, and/or protein levels of a target protein by fold increases over baseline. In some embodiments, AAV particles lead to 5-6 times higher levels of a target gene, mRNA, or protein.

In some embodiments, administration of the AAV particle described herein (e.g., an AAV particle comprising an AAV capsid polypeptide), e.g., an AAV particle comprising a nucleic acid encoding a siRNA molecule or an antibody or antibody fragment, to a subject may decrease target gene, mRNA, and/or protein levels in a subject, relative to a control, e.g., the gene, mRNA, and/or mRNA levels in the subject prior to receiving AAV particle. The target gene, mRNA, and/or protein levels may be decreased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS, or a muscle, a region of a muscle, or a cell of a muscle, of a subject. In some embodiments, cell of the CNS comprises an astrocyte, microglia, cortical neuron, hippocampal neuron, DRG and/or sympathetic neuron, sensory neuron, oligodendrocyte, motor neuron, or combination thereof. As a non-limiting example, the AAV particles may decrease the gene, mRNA, and/or protein levels of a target protein by fold decreases over baseline.

In some embodiments, the AAV particles described herein may be used to increase target protein and reduce symptoms of neurological disease in a subject. In some embodiments, the AAV particles described herein may be used to decrease target protein and reduce symptoms of neurological disease in a subject.

In some embodiments, the AAV particles described herein may be used to reduce the decline of functional capacity and activities of daily living as measured by a standard evaluation system such as, but not limited to, the total functional capacity (TFC) scale.

In some embodiments, the AAV particles described herein may be used to improve performance on any assessment used to measure symptoms of neurological disease. Such assessments include, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B (Ther Adv Neurol Disord. 5(6):349-358 (2012)), the contents of which are herein incorporated by reference in their entirety.

In some embodiments, the present composition is administered as a solo therapeutic or as combination therapeutic for the treatment of a neurological disease/disorder or a neurodegenerative disorder, a muscular disorder or neuromuscular disorder, and/or a neuro-oncological disorder.

The AAV particles encoding the target protein may be used in combination with one or more other therapeutic agents. In some embodiments, compositions can be administered concurrently with, prior to, or subsequent to, additional therapeutic or medical procedures. In general, each agent will be administered at a dose and/or on a time schedule determined for that agent.

Therapeutic agents that may be used in combination with the AAV particles described herein can be small molecule compounds which are antioxidants, anti-inflammatory agents, anti-apoptosis agents, calcium regulators, anti-glutamatergic agents, structural protein inhibitors, compounds involved in muscle function, and compounds involved in metal ion regulation. As a non-limiting example, the combination therapy may be in combination with one or more neuroprotective agents such as small molecule compounds, growth factors and hormones which have been tested for their neuroprotective effect on motor neuron degeneration.

Compounds tested for treating neurological disease which may be used in combination with the AAV particles described herein include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK30 (lithium) or PP2A, immunization with Aβ peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetylcholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the AAV particles described herein for treating neurological disease. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the AAV particle described herein may be co-administered with AAV particles expressing neurotrophic factors such as AAV-IGF-I (See e.g., Vincent et al., Neuromolecular medicine, 2004, 6, 79-85; the contents of which are incorporated herein by reference in their entirety) and AAV-GDNF (See e.g., Wang et al., J Neurosci., 2002, 22, 6920-6928; the contents of which are incorporated herein by reference in their entirety).

In some embodiments, administration of the AAV particles described herein to a subject will modulate, e.g., increase or decrease, the expression of a target protein in a subject and the modulation, e.g., increase or decrease of the presence, level, activity, and/or expression of the target protein will reduce the effects and/or symptoms of a neurological disease/disorder or a neurodegenerative disorder, a muscular disorder or neuromuscular disorder, and/or a neuro-oncological disorder in a subject.

In some embodiments, the AAV particle described herein is administered to a subject prophylactically, to prevent onset of disease. In another embodiment, the AAV particle (e.g., an AAV described herein is administered to treat (e.g., lessen the effects of) a disease or symptoms thereof. In yet another embodiment, the AAV particle described herein is administered to cure (eliminate) a disease. In another embodiment, the AAV particle described herein is administered to prevent or slow progression of disease. In yet another embodiment, the AAV particle described herein is used to reverse the deleterious effects of a disease. Disease status and/or progression may be determined or monitored by standard methods known in the art.

In some embodiments, the AAV particle described herein is useful for treatment, prophylaxis, palliation or amelioration of a genetic disorder, e.g., an autosomal dominant genetic disorder, an autosomal recessive disorder, X-linked dominant genetic disorder, an X-linked recessive genetic disorder, or a Y-linked genetic disorder. In some embodiments, the genetic disorder is a monogenetic disorder or a polygenic disorder. In some embodiments, treatment of a genetic disorder, e.g., a monogenic disorder, comprises the use of an AAV particle described herein for a gene replacement therapy.

In some embodiments, provided herein is method for treating a neurological disorder and/or neurodegenerative disorder in a subject, comprising administering to the subject an effective amount of a pharmaceutical composition described herein or an AAV particle, e.g., a plurality of particles, comprising an AAV capsid polypeptide described herein. In some embodiments, treatment of a neurological disorder and/or neurodegenerative disorder comprises prevention of said neurological disorder and/or neurological disorder.

In some embodiments, the AAV particle described herein is useful for the treatment, prophylaxis, palliation or amelioration of neurological diseases and/or disorders. In some embodiments, the AAV particle described herein is useful for the treatment, prophylaxis, palliation or amelioration of tauopathy.

In some embodiments, the AAV particle described herein is for the treatment, prophylaxis, palliation or amelioration of Alzheimer's Disease. In some embodiments, treatment of Alzheimer's Disease comprises the use of an AAV particle described herein for a gene replacement therapy. In some embodiments, the payload encoded by an AAV particle comprising a capsid polypeptide described herein comprises an ApoE2 protein, ApoE4 protein, an ApoE3 protein, BDNF protein, CYP46A1 protein, Klotho protein, fractalkine (FKN) protein, neprilysin protein (NEP), CD74 protein, caveolin-1, or a combination or variant thereof. In some embodiments, treatment of Alzheimer's Disease comprises the use of an AAV particle described for a reduction in the expression of a tau gene and/or protein, a synuclein gene and/or protein, or a combination or variant thereof. In some embodiments, the payload encoded by an AAV particle comprising a capsid polypeptide described herein comprises an antibody that binds to tau or synuclein, an RNAi agent for inhibiting tau or synuclein, a gene editing system (e.g., a CRISPR-Cas system) for altering tau or synuclein expression, or a combination thereof.

In some embodiments, the AAV particle described herein is for the treatment, prophylaxis, palliation or amelioration of frontal temporal dementia. In some embodiments, treatment of frontal temporal dementia comprises the use of an AAV particle described herein for a gene replacement therapy. In some embodiments, the payload encoded by an AAV particle comprising a capsid polypeptide described herein comprises a progranulin protein or variant thereof.

In some embodiments, the AAV particle described herein is useful the treatment, prophylaxis, palliation or amelioration of Friedreich's ataxia, or any disease stemming from a loss or partial loss of frataxin protein. In some embodiments, treatment of Friedreich's ataxia comprises the use of an AAV particle described herein for a gene replacement therapy.

In some embodiments, the AAV particle described herein is useful for the treatment, prophylaxis, palliation or amelioration of Parkinson's Disease. In some embodiments, treatment of Parkinson's disease comprises the use of an AAV particle described herein for a gene replacement therapy. In some embodiments, the payload encoded by an AAV particle comprising a capsid variant described herein comprises an AADC protein, GAD protein, GDNF protein, TH-GCH1 protein, GBA protein, AIMP2-DX2 protein, or a combination or variant thereof. In some embodiments, treatment of Parkinson's disease comprises the use of an AAV particle described herein for a gene knock-down therapy or a gene editing therapy (e.g., knock-out, repression, or correction). In some embodiments, the payload encoded by an AAV particle comprising a capsid variant described herein comprises a modulator, e.g., an RNAi agent or a CRISPR-Cas system, for altering expression of an alpha-synuclein gene, mRNA, and/or protein, or variant thereof.

In some embodiments, the AAV particle described herein is useful for the treatment, prophylaxis, palliation or amelioration of an AADC deficiency. In some embodiments, treatment of AADC deficiency comprises the use of an AAV particle described herein for a gene replacement therapy. In some embodiments, the payload encoded by an AAV particle comprising a capsid variant described herein comprises an AADC protein or variant thereof.

In some embodiments, the AAV particle described herein is useful for the treatment, prophylaxis, palliation or amelioration of Amyotrophic lateral sclerosis (ALS). In some embodiments, treatment of ALS comprises the use of an AAV particle described herein for a gene replacement therapy. In some embodiments, the payload encoded by an AAV particle comprising a capsid variant described herein comprises an TDP-43 protein, UPF1 protein, C9orf72 protein, CCNF protein, HSF1 protein, Factor H protein, NGF protein, ADAR2 protein, GDNF protein, VEGF protein, HGF protein, NRTN protein, AIMP2-DX2 protein, or a combination or variant thereof. In some embodiments, treatment of ALS comprises the use of an AAV particle described herein for a gene knock-down therapy or a gene editing therapy (e.g., knock-out, repression, or correction). In some embodiments, the payload encoded by an AAV particle comprising a capsid polypeptide described herein comprises a modulator, e.g., an RNAi agent or a CRISPR-Cas system, for altering expression of a SOD1 or C90RF72 gene, mRNA, and/or protein, or a combination or variant thereof.

In some embodiments, the AAV particle described herein is useful for the treatment, prophylaxis, palliation or amelioration of Huntington's Disease. In some embodiments, treatment of ALS comprises the use of an AAV particle described herein for a gene knock-down (e.g., knock-out) therapy or a gene editing therapy (e.g., knock-out, repression, or correction). In some embodiments, the payload encoded by an AAV particle comprising a capsid variant described herein comprises a modulator, e.g., an RNAi agent or a CRISPR-Cas system, for altering expression of an HTT gene, mRNA, and/or protein, or a variant thereof.

In some embodiments, the AAV particle described herein is useful for the treatment, prophylaxis, palliation or amelioration of spinal muscular atrophy. In some embodiments, treatment of spinal muscular atrophy comprises the use of an AAV particle described herein for a gene replacement therapy. In some embodiments, the payload encoded by an AAV particle comprising a capsid variant described herein comprises an SMN1 protein, an SMN2 protein, or a combination or variant thereof.

In some embodiments, the AAV particle described herein is useful for the treatment, prophylaxis, palliation or amelioration of multiple system atrophy. In some embodiments, treatment of multiple system atrophy comprises the use of an AAV particle described herein for a gene replacement therapy.

In some embodiments, the AAV particle described herein is useful for the treatment, prophylaxis, palliation or amelioration of Gaucher disease (GD) (e.g., Type 1 GD, Type 2 GD, or Type 3 GD). In some embodiments, the AAV particle described herein is useful for the treatment, prophylaxis, palliation or amelioration of Parkinson's disease associated with a GBA mutation. In some embodiments, the AAV particle described herein is useful for the treatment, prophylaxis, palliation or amelioration of dementia with Lewy Bodies (DLB).

In some embodiments, the AAV particle described herein is useful for treatment, prophylaxis, palliation or amelioration of a leukodystrophy, e.g., Alexander disease, autosomal dominant leukodystrophy with autonomic diseases (ADLD), adrenoleukodystrophy (ALD), Canavan disease, cerebrotendinous xanthomatosis (CTX), metachromatic leukodystrophy (MLD), Pelizaeus-Merzbacher disease, or Refsum disease. In some embodiments, treatment of MLD comprises the use of an AAV particle described herein for a gene replacement therapy. In some embodiments, the payload encoded by an AAV particle comprising a capsid variant described herein comprises an ARSA protein or variant thereof. In some embodiments, treatment of ALD comprises the use of an AAV particle described herein for a gene replacement therapy. In some embodiments, the payload encoded by an AAV particle comprising a capsid polypeptide described herein comprises an ABCD-1 protein or variant thereof.

In some embodiments, the AAV particle described herein is useful for the treatment, prophylaxis, palliation, or amelioration of megalencephalic leukoencephalopathy (MLC). In some embodiments, treatment of MLC comprises the use of an AAV particle described herein for a gene replacement therapy. In some embodiments, the payload encoded by an AAV particle comprising a capsid polypeptide described herein comprises an MLC1 protein or variant thereof.

In some embodiments, the AAV particle described herein is useful for the treatment, prophylaxis, palliation, or amelioration of Krabbe disease. In some embodiments, treatment of Krabbe disease comprises the use of an AAV particle described herein for a gene replacement therapy. In some embodiments, the payload encoded by an AAV particle comprising a capsid polypeptide described herein comprises a GALC protein or variant thereof.

In some embodiments, the AAV particle described herein is useful for the treatment, prophylaxis, palliation, or amelioration of Mucopolysaccharidosis, e.g., a Type I (MPS I), Type II (MPS II), Type IIIA (MPS IIIA), Type IIIB (MPS IIIB), or Type IIIC (MPS IIIC). In some embodiments, treatment of Mucopolysaccharidosis comprises the use of an AAV particle described herein for a gene replacement therapy or a gene editing therapy (e.g., enhancement or correction). In some embodiments, the payload encoded or corrected by an AAV particle comprising a capsid polypeptide described herein comprises an IDUA protein, IDS protein, SGSH protein, NAGLU protein, HGSNAT protein, or a combination or variant thereof.

In some embodiments, the AAV particle described herein is useful for the treatment, prophylaxis, palliation, or amelioration of Batten/NCL. In some embodiments, treatment of Batten/NCL comprises the use of an AAV particle described herein for a gene replacement therapy. In some embodiments, the payload encoded by an AAV particle described herein comprises a CLN1 protein, CLN2 protein, CLN3 protein, CLN5 protein, CLN6 protein, CLN7 protein, CLN8 protein, or a combination or variant thereof.

In some embodiments, the AAV particle described herein is useful for the treatment, prophylaxis, palliation or amelioration of Rett Syndrome. In some embodiments, treatment of Rett Syndrome comprises the use of an AAV particle described herein (e.g., an AAV particle comprising an AAV capsid polypeptide described herein) for a gene replacement therapy. In some embodiments, the payload encoded by an AAV particle comprising a capsid polypeptide described herein comprises an MeCP2 protein or variant thereof.

In some embodiments, the AAV particle described herein is useful for the treatment, prophylaxis, palliation, or amelioration of Angelman Syndrome. In some embodiments, treatment of Angelman Syndrome comprises the use of an AAV particle described herein for a gene replacement therapy. In some embodiments, the payload encoded by an AAV particle comprising a capsid polypeptide described herein comprises a UBE3A protein or variant thereof.

In some embodiments, the AAV particle described herein is useful for the treatment, prophylaxis, palliation, or amelioration of Fragile X Syndrome. In some embodiments, treatment of Fragile X Syndrome comprises the use of an AAV particle described herein for a gene replacement therapy. In some embodiments, the payload encoded by an AAV particle comprising a capsid polypeptide described herein comprises a Reelin protein, a DgkK protein, a FMR1 protein, or a combination or variant thereof.

In some embodiments, the AAV particle described herein is useful for the treatment, prophylaxis, palliation, or amelioration of Canavan Disease. In some embodiments, treatment of Canavan Disease comprises the use of an AAV particle described herein for a gene replacement therapy. In some embodiments, the payload encoded by an AAV particle comprising a capsid polypeptide described herein comprises an ASPA protein or variant thereof.

In some embodiments, the AAV particle described herein is useful for the treatment, prophylaxis, palliation, or amelioration of a Gangliosidosis, e.g., a GM1 Gangliosidosis or a GM2 Gangliosidosis (e.g., Tay Sachs Sandhoff). In some embodiments, treatment of a Gangliosidosis, e.g., a GM1 Gangliosidosis or a GM2 Gangliosidosis (e.g., Tay Sachs Sandhoff), comprises the use of an AAV particle described herein for a gene replacement therapy. In some embodiments, the payload encoded by an AAV particle comprising a capsid polypeptide described herein comprises a GLB1 protein, a HEXA protein, a HEXB protein, a GM2A protein, or a combination or variant thereof.

In some embodiments, the AAV particle described herein is useful for the treatment, prophylaxis, palliation, or amelioration of GM3 Synthase Deficiency. In some embodiments, treatment of GM3 Synthase Deficiency comprises the use of an AAV particle described herein for a gene replacement therapy. In some embodiments, the payload encoded by an AAV particle comprising a capsid polypeptide described herein comprises an ST3GAL5 protein or variant thereof.

In some embodiments, the AAV particle described herein is useful for the treatment, prophylaxis, palliation, or amelioration of a Niemann-Pick disorder, e.g., a Niemann-Pick A or a Niemann-Pick C1 (NPC-1). In some embodiments, treatment of a Niemann-Pick disorder, e.g., a Niemann-Pick A or a Niemann-Pick C1 (NPC-1) comprises the use of an AAV particle described herein for a gene replacement therapy. In some embodiments, the payload encoded by an AAV particle comprising a capsid polypeptide described herein comprises an ASM protein, an NPC1 protein, or variant thereof.

In some embodiments, the AAV particle described herein is useful for the treatment, prophylaxis, palliation, or amelioration of Schwannoma (e.g., Neuroma). In some embodiments, treatment of Schwannoma (e.g., Neuroma) comprises the use of an AAV particle described herein for a gene replacement therapy. In some embodiments, the payload encoded by an AAV particle comprising a capsid polypeptide described herein comprises a Caspase-1 protein or variant thereof.

In some embodiments, the AAV particle described herein is useful for the treatment, prophylaxis, palliation, or amelioration of a Tuberous Sclerosis, e.g., Tuberous Sclerosis Type 1 or Tuberous Sclerosis Type 2. In some embodiments, treatment of Tuberous Sclerosis, e.g., Tuberous Sclerosis Type 1 or Tuberous Sclerosis Type 2 comprises the use of an AAV particle described herein for a gene replacement therapy. In some embodiments, the payload encoded by an AAV particle comprising a capsid polypeptide described herein comprises a TSC1 protein, a TSC2 protein, or variant thereof.

In some embodiments, the AAV particle described herein is useful for the treatment, prophylaxis, palliation, or amelioration of a CDKL5 Deficiency. In some embodiments, treatment of a CDKL5 Deficiency comprises the use of an AAV particle described herein for a gene replacement therapy. In some embodiments, the payload encoded by an AAV particle comprising a capsid polypeptide described herein comprises a CDKL5 protein or variant thereof.

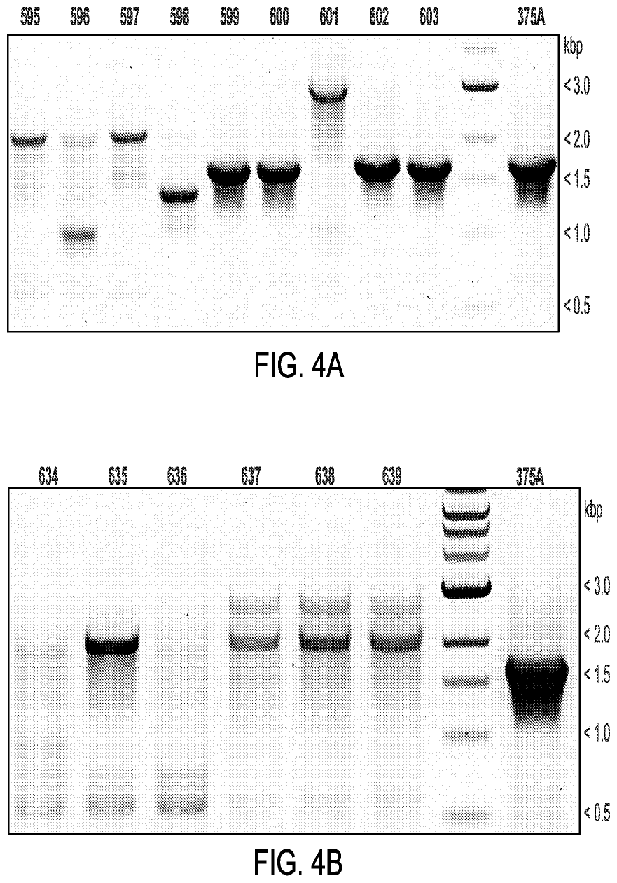

In some embodiments, the AAV particle described herein is useful for the treatment, prophylaxis, palliation, or amelioration of a Charcot-Marie-Tooth disorder, e.g., a Charcot-Marie-Tooth Type 1X (CMT1X) disorder, a Charcot-Marie-Tooth Type 2A (CMT2A) disorder, or a Charcot-Marie-Tooth Type 4J (CMT4J) disorder. In some embodiments, treatment of a Charcot-Marie-Tooth disorder, e.g., a Charcot-Marie-Tooth Type 1X (CMT1X) disorder, a Charcot-Marie-Tooth Type 2A (CMT2A) disorder, or a Charcot-Marie-Tooth Type 4J (CMT4J) disorder, comprises the use of an AAV particle described herein for a gene replacement therapy. In some embodiments, the payload encoded by an AAV particle comprising a capsid polypeptide described herein comprises a GJB1 protein, a MFN2 protein, a FIG. 4 protein, or variant thereof.

In some embodiments, the AAV particle described herein is useful for the treatment, prophylaxis, palliation, or amelioration of an Aspartylglucosaminuria (AGU). In some embodiments, treatment of an AGU comprises the use of an AAV particle described herein for a gene replacement therapy. In some embodiments, the payload encoded by an AAV particle comprising a capsid polypeptide described herein comprises an AGA protein or variant thereof.

In some embodiments, the AAV particle described herein is useful for the treatment, prophylaxis, palliation, or amelioration of a Leigh Syndrome. In some embodiments, treatment of a Leigh Syndrome comprises the use of an AAV particle described herein for a gene replacement therapy. In some embodiments, the payload encoded by an AAV particle comprising a capsid polypeptide described herein comprises a SURF1 protein or variant thereof.

In some embodiments, the AAV particle described herein is useful for the treatment, prophylaxis, palliation, or amelioration of epilepsy. In some embodiments, treatment of epilepsy comprises the use of an AAV particle described herein for a gene replacement therapy. In some embodiments, the payload encoded by an AAV particle comprising a capsid polypeptide described herein comprises an NPY/Y2 protein, a Galanin protein, a Dynorphin protein, an AIMP2-DX2 protein, an SLC6A1 protein, an SLC13A5 protein, a KCNQ2 protein, or variant thereof.

In some embodiments, the AAV particle described herein is useful for the treatment, prophylaxis, palliation, or amelioration of a Dravet Syndrome. In some embodiments, treatment of Dravet Syndrome comprises the use of an AAV particle described herein for a gene replacement therapy. In some embodiments, the payload encoded by an AAV particle comprising a capsid polypeptide described herein comprises an SCN1a protein, or variant thereof.

In some embodiments, the AAV particle described herein is useful for the treatment, prophylaxis, palliation, or amelioration of a Duchenne muscular dystrophy (DMD). In some embodiments, treatment of DMD comprises the use of an AAV particle described herein for a gene replacement therapy or enhancement (e.g., correction of exon-skipping), or a gene editing therapy (e.g., enhancement or correction). In some embodiments, the payload encoded or corrected by an AAV particle comprising a capsid polypeptide described herein comprises a Dystrophin gene and/or protein, a Utrophin gene and/or protein, or a GALGT2 gene and/or protein, or a Follistatin gene and/or protein, or a combination or variant thereof.

In some embodiments, the AAV particle described herein is useful for the treatment, prophylaxis, palliation, or amelioration of Pompe Disease. In some embodiments, treatment of Pompe Disease comprises the use of an AAV particle described herein for a gene replacement therapy. In some embodiments, the payload encoded by an AAV particle comprising a capsid polypeptide described herein comprises a GAA protein, or variant thereof.

In some embodiments, the AAV particle described herein is useful for the treatment, prophylaxis, palliation, or amelioration of Limb-Girdle Muscular Dystrophy (LGMD2A). In some embodiments, treatment of LGMD2A comprises the use of an AAV particle described herein for a gene replacement therapy. In some embodiments, the payload encoded by an AAV particle comprising a capsid polypeptide described herein comprises a CAPN-3 protein, DYSF protein, a SGCG protein, a SGCA protein, a SGCB protein, a FKRP protein, a ANO5 protein, or a combination or variant thereof.

In some embodiments, the AAV particle described herein is useful for the treatment, prophylaxis, palliation, or amelioration of chronic or neuropathic pain.

In some embodiments, the AAV particle described herein is useful for treatment, prophylaxis, palliation, or amelioration of a disease associated with the central nervous system.

In some embodiments, the AAV particle described herein is useful for treatment, prophylaxis, palliation, or amelioration of a disease associated with the peripheral nervous system.

In some embodiments, the AAV particle described herein is useful for treatment, prophylaxis, palliation, or amelioration of a neuro-oncological disorder in a subject. In some embodiments, treatment of a neuro-oncological disorder comprises prevention of said neuro-oncological disorder. In some embodiments, a neuro-oncological disorder comprises a cancer of a primary CNS origin (e.g., a CNS cell, a tissue, or a region), or a metastatic cancer in a CNS cell, tissue, or region. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

In some embodiments, the AAV particle described herein is useful for the treatment, prophylaxis, palliation, or amelioration of a disease associated with expression of HER2, e.g., a disease associated with overexpression of HER2. In some embodiments, the AAV particle described herein is useful for the treatment, prophylaxis, palliation, or amelioration of a HER2-positive cancer. In some embodiments, the HER2-positive cancer is a HER2-positive solid tumor. Additionally, or alternatively, the HER2-positive cancer may be a locally advanced or metastatic HER2-positive cancer. In some instances, the HER2-positive cancer is a HER2-positive breast cancer or a HER2-positive gastric cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer. In some instances, the HER2-positive cancer is prostate cancer. In some embodiments, the HER2-positive cancer has metastasized to the central nervous system (CNS). In some instances, the metastasized HER2-cancer has formed CNS neoplasms.

In some embodiments, the AAV particle described herein is useful for treatment, prophylaxis, palliation, or amelioration of a muscular disorder and/or neuromuscular disorder in a subject. In some embodiments, treatment of a muscular disorder and/or neuromuscular disorder comprises prevention of said muscular disorder and/or neuromuscular disorder.

In some embodiments, the AAV particle described herein is useful for treatment, prophylaxis, palliation or amelioration of a cardiac disease or heart disease and/or method of improving (e.g., enhancing) cardiac function in a subject. In some embodiments, the cardiac disease is a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction. In some embodiments, the cardiac disease is a disease associated with expression, e.g., aberrant expression, of LAMP2B, MYBPC3, TNNI3, LMNA, BAG3, DWORF, PKP2, Cx43, TAZ, CASQ2, SERCA2a, I-1c, S100A1 and/or ARC, S100A1, ASCL1, miR133, Mydelta3, Sav, or a combination or variant thereof. In some embodiments, treatment of a cardiac disorder described herein comprises the use of an AAV particle described herein for a gene replacement therapy.

In some embodiments, the cardiac disease is a genetic disorder, e.g., an autosomal dominant genetic disorder, an autosomal recessive disorder, or an X-linked recessive genetic disorder. In some embodiments, the cardiomyopathy is a genetic disorder, e.g., a genetic disorder associated with an abnormality (e.g., mutation, insertion, rearrangement and/or deletion) in a gene chosen from TTN, LMNA, MYH7, MYH6, SCN5A, TNNT2, RBM20, TNNI3, MYL2, MYL3, PKP2, DSP, DSG2, DSC2, JUP, or a combination thereof. In some embodiments, the cardiac disorder is a dilated cardiomyopathy, e.g., a dilated cardiomyopathy associated with an abnormality (e.g., mutation, insertion, rearrangement and/or deletion) in a gene chosen from TTN, LMNA, MIH7, BAG3, MIPN, TNNT2, SCN5A, RBN20, TNPO, LAMA4, VCL, LDB3, TCAP, PSEN1/2, ACTN2, CRYAB, TPM1, ABCC9, ACTC1, PDLIM3, ILK, TNNC1, TNNI3, PLN, DES, SGCD, CSRP3, MIH6, EYA4, ANKRD1, DMD, GATAD1, TAZ/G4.5, or combination thereof. In some embodiments, the cardiac disorder is a hypertrophic cardiomyopathy, e.g., a hypertrophic cardiomyopathy associated with an abnormality (e.g., mutation, insertion, rearrangement and/or deletion) in a gene chosen from MYH7, TNNT2, TNNI3, TPM1, MYL2, MYL3, ACTC1, CSRP3, TTN, ACTN2, MYH6, TCAP, TNNC1, or a combination thereof. In some embodiments, the cardiac disorder is an arrhythmogenic ventricular cardiomyopathy, e.g., an arrhythmogenic ventricular cardiomyopathy associated with an abnormality (e.g., mutation, insertion, rearrangement and/or deletion) in a gene chosen from PKP2, DSG2, DSP, RYR2, DSC2, TGFB3, TMEM43, DES, TTN, LMNA, or a combination thereof.

In some embodiments, the AAV particle described herein is administered to a subject having at least one of the diseases or symptoms described herein. In some embodiments, an AAV particle of the present disclosure is administered to a subject having or diagnosed with having a disease or disorder described herein.

Any neurological disease or disorder, neurodegenerative disorder, muscular disorder, neuromuscular disorder, and/or neuro-oncological disorder may be treated with the AAV particles of the disclosure, or pharmaceutical compositions thereof, including but not limited to, Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Baló's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia—Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoffs Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus—Neurological Sequelae, Lyme Disease—Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy-Congenital, Myopathy-Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy—Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

### V. Pharmaceutical Compositions and Formulations

Provided herein are pharmaceutical compositions comprising an AAV particle described herein. In some embodiments, the pharmaceutical composition comprises at least one active ingredients. In some embodiments, the pharmaceutical composition comprises a pharmaceutically acceptable excipient.

In some embodiments, an AAV particle described herein is formulated using an excipient to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the payload. Formulations of the present disclosure can include, without limitation, saline, liposomes, lipid nanoparticles, polymers, peptides, proteins, cells transfected with viral vectors (e.g., for transfer or transplantation into a subject) and combinations thereof.

In some embodiments, the relative amount of the active ingredient (e.g. an AAV particle described herein), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the pharmaceutical composition comprising an AAV particle described herein may comprise an AAV capsid polypeptide and a viral genome encoding a payload, e.g., a payload described herein, with or without a pharmaceutically acceptable excipient.

The present disclosure also provides in some embodiments, a pharmaceutical composition suitable for administration to a subject, e.g., a human. In some embodiments, the pharmaceutical composition is administered to a subject, e.g., a human.

### VI. Kits

Also provided herein are kits comprising an AAV particle described herein, e.g., an AAV particle produced using a baculovirus (or baculoviruses) produced using a variant baculovirus genome, a baculovirus expression construct, a bacmid, and/or a BAC described herein, and instructions for use.

### VII. Administration

In some embodiments, an AAV particle disclosed herein may be administered by a to a subject by a delivery route, e.g., a localized delivery route or a systemic delivery route.

In some embodiments, an AAV particle described herein may be administered via such a route that it is able to cross the blood-brain barrier, vascular barrier, or other epithelial barrier. In some embodiments, an AAV particle described herein may be administered in any suitable form, either as a liquid solution or suspension, as a solid form suitable for liquid solution or suspension in a liquid solution. In some embodiments, an AAV particle may be formulated with any appropriate and pharmaceutically acceptable excipient.

In some embodiments, the AAV particle described herein is administered intramuscularly, intravenously, intracerebrally, intrathecally, intracerebroventricularly, via intraparenchymal administration, or via intra-cisterna magna injection (ICM).

In some embodiments, an AAV particle described herein may be delivered to a subject via a single route administration. In some embodiments, an AAV particle described herein may be delivered to a subject via a multi-site route of administration. In some embodiments, a subject may be administered at 2, 3, 4, 5, or more than 5 sites.

In some embodiments, an AAV particle described herein is administered via a bolus infusion. In some embodiments, an AAV particle of the present disclosure is administered via sustained delivery over a period of minutes, hours, or days. In some embodiments, the infusion rate may be changed depending on the subject, distribution, formulation, and/or another delivery parameter. In some embodiments, an AAV particle described herein is administered using a controlled release. In some embodiments, an AAV particle described herein is administered using a sustained release, e.g., a release profile that conforms to a release rate over a specific period of time.

In some embodiments, an AAV particle described herein may be delivered by more than one route of administration. As non-limiting examples of combination administrations, an AAV particle may be delivered by intrathecal and intracerebroventricular, or by intravenous and intraparenchymal administration.

### Intravenous Administration

In some embodiments, an AAV particle described herein may be administered to a subject by systemic administration. In some embodiments, the systemic administration is intravenous administration. In another embodiment, the systemic administration is intraarterial administration. In some embodiments, an AAV particle described herein may be administered to a subject by intravenous administration. In some embodiments, the intravenous administration may be achieved by subcutaneous delivery. In some embodiments, the AAV particle is administered to the subject via focused ultrasound (FUS), e.g., coupled with the intravenous administration of microbubbles (FUS-MB) or MRI-guided FUS coupled with intravenous administration, e.g., as described in Terstappen et al. (Nat Rev Drug Discovery, https://doi.org/10.1038/s41573-021-00139-y (2021)), the contents of which are incorporated herein by reference in its entirety. In some embodiments, the AAV particle is administered to the subject intravenously. In some embodiments, the subject is a human.

### Administration to the CNS

In some embodiments, an AAV particle described herein may be delivered by direct injection into the brain. As a non-limiting example, the brain delivery may be by intrahippocampal administration. In some embodiments, an AAV particle described herein may be administered to a subject by intraparenchymal administration. In some embodiments, the intraparenchymal administration is to tissue of the central nervous system. In some embodiments, an AAV particle described herein may be administered to a subject by intracranial delivery (See, e.g., U.S. Pat. No. 8,119,611; the content of which is incorporated herein by reference in its entirety). In some embodiments, an AAV particle described herein may be delivered by injection into the CSF pathway. Non-limiting examples of delivery to the CSF pathway include intrathecal and intracerebroventricular administration. In some embodiments, an AAV particle described herein may be administered via intracisternal magna (ICM) injection.

In some embodiments, an AAV particle described herein may be delivered to the brain by systemic delivery. As a non-limiting example, the systemic delivery may be by intravascular administration. As a non-limiting example, the systemic or intravascular administration may be intravenous.

In some embodiments, an AAV particle described herein may be delivered by an intraocular delivery route. A non-limiting example of an intraocular administration includes an intravitreal injection.

### Intramuscular Administration

In some embodiments, an AAV particle described herein may be delivered by intramuscular administration. Without wishing to be bound by theory, it is believed in some embodiments, that the multi-nucleated nature of muscle cells provides an advantage to gene transduction subsequent to AAV delivery. In some embodiments, cells of the muscle are capable of expressing recombinant proteins with the appropriate post-translational modifications. Without wishing to be bound by theory, it is believed in some embodiments, the enrichment of muscle tissue with vascular structures allows for transfer to the blood stream and whole-body delivery. Examples of intramuscular administration include systemic (e.g., intravenous), subcutaneous or directly into the muscle. In some embodiments, more than one injection is administered. In some embodiments, an AAV particle described herein may be delivered by an intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In some embodiments, an AAV particle described herein is administered to a subject and transduces the muscle of a subject. As a non-limiting example, an AAV particle is administered by intramuscular administration. In some embodiments, an AAV particle described herein may be administered to a subject by subcutaneous administration. In some embodiments, the intramuscular administration is via systemic delivery. In some embodiments, the intramuscular administration is via intravenous delivery. In some embodiments, the intramuscular administration is via direct injection to the muscle.

In some embodiments, the muscle is transduced by administration, e.g., intramuscular administration. In some embodiments, an intramuscular delivery comprises administration at one site. In some embodiments, an intramuscular delivery comprises administration at more than one site. In some embodiments, an intramuscular delivery comprises administration at two, three, four, or more sites. In some embodiments, intramuscular delivery is combined with at least one other method of administration.

In some embodiments, an AAV particle described herein may be administered to a subject by peripheral injections. Non-limiting examples of peripheral injections include intraperitoneal, intramuscular, intravenous, conjunctival, or joint injection. It was disclosed in the art that the peripheral administration of AAV vectors can be transported to the central nervous system, for example, to the motor neurons (e.g., U.S. Patent Publication Nos. US20100240739 and US20100130594; the content of each of which is incorporated herein by reference in their entirety).

In some embodiments, an AAV particle described herein may be administered to a subject by intraparenchymal administration. In some embodiments, the intraparenchymal administration is to muscle tissue. In some embodiments, an AAV particle described herein is delivered as described in Bright et al 2015 (Neurobiol Aging. 36(2):693-709), the contents of which are herein incorporated by reference in their entirety. In some embodiments, an AAV particle described herein is administered to the gastrocnemius muscle of a subject. In some embodiments, an AAV particle described herein is administered to the bicep femorii of the subject. In some embodiments, an AAV particles described herein is administered to the tibialis anterior muscles. In some embodiments, an AAV particle described herein is administered to the soleus muscle.

### Depot Administration

In some embodiments, a pharmaceutical composition and/or an AAV particle described herein (e.g., an AAV particle comprising an AAV capsid polypeptide) are formulated in depots for extended release. Generally, specific organs or tissues are targeted for administration.

In some embodiments, a pharmaceutical composition and/or an AAV particle described herein (e.g., an AAV particle comprising an AAV capsid polypeptide) are spatially retained within or proximal to target tissues. Provided are methods of providing a pharmaceutical composition, an AAV particle, to target tissues of mammalian subjects by contacting target tissues (which comprise one or more target cells) with the pharmaceutical composition and/or the AAV particle, under conditions such that they are substantially retained in target tissues, e.g., such that at least 10, 20, 30, 40, 50, 60, 70, 80, 85, 90, 95, 96, 97, 98, 99, 99.9, 99.99 or greater than 99.99% of the composition is retained in the target tissues. In some embodiments, retention is determined by measuring the amount of pharmaceutical composition and/or AAV particle, that enter a target cell or a plurality of target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of a pharmaceutical composition and/or an AAV particle, administered to a subject are present intracellularly at a period of time following administration. For example, intramuscular injection to a subject may be performed using aqueous compositions comprising a pharmaceutical composition and/or an AAV particle described herein and a transfection reagent, and retention is determined by measuring the amount of the pharmaceutical composition and/or the AAV particle, present in the muscle cell or plurality of muscle cells.

In some embodiments, provided are methods of providing a pharmaceutical composition and/or an AAV particle described herein (e.g., an AAV particle comprising an AAV capsid polypeptide) to a tissue of a subject, by contacting the tissue (comprising a cell, e.g., a plurality of cells) with the pharmaceutical composition and/or the AAV particle under conditions such that they are substantially retained in the tissue. In some embodiments, a pharmaceutical composition and/or AAV particle described herein comprise a sufficient amount of an active ingredient such that the effect of interest is produced in at least one cell. In some embodiments, a pharmaceutical composition and/or an AAV particle generally comprise one or more cell penetration agents. In some embodiments, the disclosure provides a naked formulations (such as without cell penetration agents or other agents), with or without pharmaceutically acceptable carriers.

### VIII. Definitions

At various places in the present disclosure, substituents or properties of compounds of the present disclosure are disclosed in groups or in ranges. It is specifically intended that the present disclosure comprise each and every individual or sub-combination of the members of such groups and ranges.

Unless stated otherwise, the following terms and phrases have the meanings described below. The definitions are not meant to be limiting in nature and serve to provide a clearer understanding of certain aspects of the present disclosure.

Adeno-associated virus: The term “adeno-associated virus” or “AAV” as used herein refers to members of the dependovirus genus comprising any particle, sequence, gene, protein, or component derived therefrom.

AAV expression construct: As used herein, “AAV expression construct” refers to a polynucleotide comprising nucleotide sequences encoding at least an AAV capsid protein (e.g., a VP1 protein, a VP2 protein, and/or a VP3 protein), and/or an AAV rep protein (e.g., a Rep52, Rep40, Rep68, or Rep78 protein, or a combination thereof). In some embodiments, the AAV expression construct further comprises a nucleotide sequence encoding a payload (e.g., a payload encoding region). In some embodiments, the AAV expression construct comprises at least a portion of a baculovirus genome (e.g. a variant baculovirus genome).

AAV Particle: As used herein, an “AAV particle” refers to a particle or a virion comprising an AAV capsid, e.g., an AAV capsid variant, and a polynucleotide, e.g., a viral genome or a vector genome. In some embodiments, the viral genome of the AAV particle comprises at least one payload region and at least one ITR. In some embodiments, an AAV particle of the disclosure is an AAV particle comprising an AAV variant. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. It is to be understood that reference to the AAV particle of the disclosure also includes pharmaceutical compositions thereof, even if not explicitly recited.

Administering: As used herein, the term “administering” refers to providing a pharmaceutical agent or composition to a subject.

Amelioration: As used herein, the term “amelioration” or “ameliorating” refers to a lessening of severity of at least one indicator of a condition or disease. For example, in the context of neurodegeneration disorder, amelioration comprises the reduction of neuron loss.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals comprise, but are not limited to, mammals, birds, reptiles, amphibians, fish, and worms. In certain embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. As used herein, the term “about” means+/−10% of the recited value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Baculoviral expression vector (BEV): As used herein a BEV is a baculoviral expression vector, e.g., a polynucleotide vector of baculoviral origin. A baculovirus expression vector (BEV) is a recombinant baculovirus that has been genetically modified to lead the expression of a foreign gene. Systems using BEVs are known as baculoviral expression vector systems (BEVSs).

mBEV or modified BEV: As used herein, a modified BEV is an expression vector of baculoviral origin which has been altered from a starting BEV (whether wild type or artificial), e.g., by the disruption, addition and/or deletion and/or duplication and/or inversion of one or more: genes; gene fragments; cleavage sites; restriction sites; sequence regions; sequence(s) encoding a payload or gene of interest; or combinations of the foregoing.

Baculovirus genome: As used herein a “baculovirus genome” comprises a wild-type or altered baculovirus or portion thereof. In some embodiments, the wild-type or altered baculovirus comprises a Autographa californica multiple nucleopolyhedrovirus (AcMNPV) (e.g., an AcMNPV strain E2, C6, or HR3), Bombyx mori nucleopolyhedrovirus (BmNPV), Anticarsia gemmatalis nucleopolyhedrovirus (AgMNPV), Orgyia pseudotsugata nucleopolyhedrovirus (OpMNPV), or Thysanoplusia orichalcea nucleopolyhedrovirus (ThorMNPV). In some embodiments, a variant baculovirus genome is an altered baculovirus genome or a portion thereof.

BIIC: As used herein a BIIC is a baculoviral infected insect cell.

Capsid: As used herein, the term “capsid” refers to the exterior, e.g., a protein shell, of a virus particle, e.g., an AAV particle, that is substantially (e.g., >50%, >60%, >70%, >80%, >90%, >95%, >99%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). As a non-limiting example, a nucleic acid sequence which comprises an AAV capsid protein can be codon optimized for expression in insect cells or in a particular insect cell such Spodoptera frugiperda cells.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Delivery: As used herein, “delivery” refers to the act or manner of delivering an AAV particle, a compound, substance, entity, moiety, cargo or payload.

Encapsulate: As used herein, the term “encapsulate” means to enclose, surround or encase.

Engineered: As used herein, embodiments of the present disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

ExpressionBac: As used herein, “expressionBac” or “rep/cap bac” refers to a an AAV expression construct and/or region comprising a baculovirus genome. In some embodiments, the AAV expression construct comprising the expressionBac comprises one or more polynucleotides encoding capsid and/or replication genes for an AAV, such as but not limited to AAV2. For example, the one or more polynucleotides encoding capsid and/or replication genes for an AAV may encode VP1, VP2, VP3, Rep52, and/or Rep78, and these polynucleotides may be present in the construct in one or more open reading frames, e.g., in two open reading frames.

Expression BIIC: As used herein, “expression BIIC” or “rep/cap BIIC” refers to an insect cell comprising an AAV expression construct comprising a baculovirus genome (e.g., expressionBac). In some embodiments, the insect cell is an Sf9 cell.

Formulation: As used herein, a “formulation” comprises at least one AAV particle and a delivery agent or excipient.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g., between polynucleotide molecules (e.g., DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In certain embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of which are each incorporated herein by reference in their entireties, insofar as they do not conflict with the present disclosure. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences comprise, but are not limited to those disclosed in Carillo, H., and Lipman, D., SIAM J Applied Math., 48:1073 (1988); the contents of which are each incorporated herein by reference in their entireties, insofar as they does not conflict with the present disclosure. Techniques for determining identity are codified in publicly available computer programs. Exemplary computer software to determine homology between two sequences comprise, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the present disclosure. Molecules may be modified in many ways comprising chemically, structurally, and functionally. As used herein, embodiments of the disclosure are “modified” when they have or possess a feature or property, whether structural or chemical, that varies from a starting point, wild type or native molecule.

Mutation: As used herein, the term “mutation” refers to any changing of the structure of a gene, resulting in a variant (also called “mutant”) form. In some embodiments, mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Non-human vertebrate: As used herein, a “non-human vertebrate” comprises all vertebrates except Homo sapiens, comprising wild and domesticated species. Examples of non-human vertebrates comprise, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic Acid: As used herein, the term “nucleic acid”, “polynucleotide” and “oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms comprise double- and single-stranded DNA, as well as double- and single stranded RNA.

Open reading frame: As used herein, “open reading frame” or “ORF” refers to a sequence which does not contain a stop codon within the given reading frame, other than at the end of the reading frame.

Operably linked: As used herein, the phrase “operably linked” refers to a functional connection between two or more molecules, constructs, transcripts, entities, moieties, or the like. As a non-limiting example, a promoter is “operably linked” to a nucleotide sequence when the promoter sequence controls and/or regulates the transcription of the nucleotide sequence.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, or a modulatory nucleic acid or regulatory nucleic acid.

PayloadBac: As used herein, “payloadBac” refers to a baculovirus comprising a payload construct and/or region. In some embodiments, the payload construct and/or region of the payloadBac comprises a polynucleotide encoding the payload.

Payload BIIC: As used herein, “payloadBIIC” refers to an insect cell comprising one or more baculovirus (e.g., payloadBac) comprising a payload construct and/or region. In some embodiments, the payload construct and/or region comprises a polynucleotide encoding the payload. In some embodiments, the insect cell is an Sf9 cell.

Payload construct: As used herein, “payload construct” is one or more vector construct which comprises a polynucleotide region encoding or comprising a payload that is flanked on one or both sides by an inverted terminal repeat (ITR) sequence. The payload construct may present a template that is replicated in a viral production cell to produce a therapeutic viral genome.

Payload construct vector: As used herein, “payload construct vector” is a vector encoding or comprising a payload construct, and regulatory regions for replication and expression of the payload construct in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient.

Pharmaceutically acceptable salts: The present disclosure also comprises pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid).

Pharmaceutically acceptable solvate: The term “pharmaceutically acceptable solvate,” as used herein, means a compound of the present disclosure wherein molecules of a suitable solvent are incorporated in the crystal lattice.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In certain embodiments, when referring to a protein or protein module, a region may comprise a linear sequence of amino acids along the protein or protein module or may comprise a three-dimensional area, an epitope and/or a cluster of epitopes. In certain embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In certain embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In certain embodiments, N-terminal regions may comprise any length of amino acids that comprises the N-terminus but does not comprise the C-terminus. In certain embodiments, C-terminal regions may comprise any length of amino acids, which comprise the C-terminus, but do not comprise the N-terminus.

In certain embodiments, when referring to a polynucleotide, a region may comprise a linear sequence of nucleic acids along the polynucleotide or may comprise a three-dimensional area, secondary structure, or tertiary structure. In certain embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In certain embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In certain embodiments, 5′ regions may comprise any length of nucleic acids that comprises the 5′ terminus but does not comprise the 3′ terminus. In certain embodiments, 3′ regions may comprise any length of nucleic acids, which comprise the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, comprising plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

Self-complementary viral particle: As used herein, a “self-complementary viral particle” is a particle comprised of at least two components, a protein capsid and a polynucleotide sequence encoding a self-complementary genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” comprises a siRNA strand having sufficient complementarity to a section of about 10-50 nucleotides of the mRNA of the gene targeted for silencing and a siRNA strand having sufficient complementarity to form a duplex with the other siRNA strand.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. In certain embodiments, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, such as 21 nucleotides (or nucleotide analogs), for example, 19, 20, 21 or 22 nucleotides. The term “long” siRNA refers to a siRNA comprising 24-60 nucleotides, such as about 24-25 nucleotides, for example, 23, 24, 25 or 26 nucleotides. Short siRNAs may, in some instances, comprise fewer than 19 nucleotides, e.g., 16, 17 or 18 nucleotides, or as few as 5 nucleotides, provided that the shorter siRNA retains the ability to mediate RNAi. Likewise, long siRNAs may, in some instances, comprise more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Signal Sequences: As used herein, the phrase “signal sequences” refers to a sequence which can direct the transport or localization of a protein.

Similarity: As used herein, the term “similarity” refers to the overall relatedness between polymeric molecules, e.g., between polynucleotide molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Subject: As used herein, the term “subject” or “patient” refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, will receive treatment, or is under care by a trained professional for a particular disease or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Synthetic: As used herein, the term “synthetic” or “chemically synthesized” in the context of a nucleic acid sequence refers to a nucleic acid molecule that is, at least in part, formed through a chemical process, as opposed to molecules of natural origin, or molecules derived via template-based amplification of molecules of natural origin. In some embodiments, chemically-synthesized DNA is non-templated (e.g., the sequence is arbitrarily decided and does not physically depend on a parental sequence), unlike natural DNA replication or in vitro polymerase reactions like PCR.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, such as a mammal, a human, or a human patient.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In certain embodiments, a therapeutically effective amount is provided in a single dose. In certain embodiments, a therapeutically effective amount is administered in a dosage regimen comprising a plurality of doses. Those skilled in the art will appreciate that in certain embodiments, a unit dosage form may be considered to comprise a therapeutically effective amount of a particular agent or entity if it comprises an amount that is effective when administered as part of such a dosage regimen.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Variant: As used herein, the term “variant” refers to a polypeptide or polynucleotide that has an amino acid or a nucleotide sequence that is substantially identical, e.g., having at least 70%, 75%, 80%, 85%, 90%, 95% or 99% sequence identity to a reference sequence. In some embodiments, the variant is a functional variant.

Functional Variant: As used herein, the term “functional variant” refers to a polypeptide variant or a polynucleotide variant that has at least one activity of the reference sequence.

Vector: As used herein, the term “vector” refers to any molecule or moiety which transports, transduces or otherwise acts as a carrier of a heterologous molecule. In some embodiments, vectors may be plasmids. In some embodiments, vectors may be viruses. An AAV particle is an example of a vector. Vectors of the present disclosure may be produced recombinantly and may be based on and/or may comprise adeno-associated virus (AAV) parent or reference sequences. The heterologous molecule may be a polynucleotide and/or a polypeptide.

Viral genome: As used herein, the terms “viral genome” refer to the nucleic acid sequence(s) encapsulated in an AAV particle. A viral genome comprises a nucleic acid sequence with at least one payload region encoding a payload and at least one ITR.

Those skilled in the art will recognize or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

### EQUIVALENTS AND SCOPE

The disclosures of each and every patent, patent application, publication, and sequences cited herein are hereby incorporated herein by reference in their entirety. Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

The present disclosure is further illustrated by the following non-limiting examples.

### EXAMPLES

### Example 1. Producing and Testing Bacmid 1260

### Materials and Methods

### Cells and Antisera

Spodoptera frugiperda cells were a Voyager Therapeutics clonal isolate of the Sf9 cell line (Summers and Smith, Tex. Agric. Exp. Stn. Bull. 1555 (1987), which is hereby incorporated by reference in its entirety). These cells were grown in suspension in ESF AF culture medium (Expression Systems, 99-300-01, Davis CA). Human embryonic kidney cells were from the HEK 293T cell line (ATCC CRL-1573). These cells were maintained in Dulbecco's Modified Eagle Medium (DMEM) (ThermoFisher Scientific, Waltham, MA). Anti-Lac Repressor antibody was mouse monoclonal antibody Anti-LacI [9A5] (ab33832) (Abcam, Cambridge MA). Anti-capsid antibody was the mouse monoclonal antibody, anti-AAV VP1/VP2/VP3 antibody (B1) (03-61058) (American Research Products, Waltham MA). The secondary antibody used for all Enhanced Chemiluminescence (ECL) Western blots was a horse radish peroxidase (HRP) conjugated goat anti-mouse IgG H&L antibody (Abcam, Goat Anti-Mouse IgG H&L (ab6789) (Abcam, Waltham MA).

### Bacmid Cloning Vector

The bacmid (Bacmid 375) was derived from bacmid bMON14272 (Luckow et al., J Virol., 67(8):4566-79 (1993), which is hereby incorporated by reference in its entirety) which contains a mini-F replicon and the genome for the baculovirus AcMNPV E2 (GenBank KM667940.1). Bac375 differed from the bMON14272 bacmid by being modified to lack the v-cath gene (Slack et al., J Gen Virol., 76 (Pt 5):1091-8 (1995), which is hereby incorporated by reference in its entirety). The V-CATH protease degrades AAV capsid proteins (Galibert et al., PLoS One., 13(11):e0207414 2018), which is hereby incorporated by reference in its entirety) and the PHPN capsid used in this study was very susceptible to V-CATH protease cleavage at the tissue tropism loop in this AAV9 variant. The bMON14272 sequence was deduced from GenBank sequences referenced by Luckow and the AcMNPV E2 genome sequence (Luckow et al., J Virol., 67(8):4566-79 (1993), which is hereby incorporated by reference in its entirety). To create the Bacmid 375, the v-cath gene was deleted from bMON14272 by homologous recombination in Sf9 cells like as described in Slack (Slack et al., J Gen Virol., 76 (Pt 5):1091-8 (1995), which is hereby incorporated by reference in its entirety). In the present example, the bMON14272 bacmid was linearized by partial digest with EcoNI restriction endonuclease (REN) (New England Biolabs, R0521S) and co-transfected into Sf9 cells with a synthetically made (GenScript) homologous recombination shuttle plasmid encoding for AcMNPV C6 (GenBank L22858.1) regions 104,916-106,115 and 107,938-109,137. Homologous recombination with this shuttle plasmid deleted a region corresponding to 106,187 to 108,010 of the AcMNPV E2 genome. This removed 98% the 5′ end of the v-cath ORF (GenBank AIU57069.1) and 50% of the 5′ end of the ChiA gene ORF (GenBank AIU56979.1). This region is referred to as the v-cath (VC) locus although most of VC is deleted. The deleted VC region was replaced by the sequence GGTACCICGCTACCITAGGACCGTTATAGTTAGGTACCGAATTC (SEQ ID NO: 99) which contains an I-CeuI REN recognition site (underlined). This modification enabled T4 DNA ligase cloning of foreign gene cassettes into the baculovirus genome following methods described by Lihoradova (Lihoradova et al., J Virol Methods., 140(1-2):59-65 (2006), which is hereby incorporated by reference in its entirety). The donor baculovirus was clonally isolated after homologous recombination in Sf9 cells by transforming the baculovirus DNA into NEB 10-beta E. coli (New England Biolabs, Ipswich MA, C3020K) and selecting colonies on 50 ug/ml Kanamycin LB Agar plates.

### Tn7 Locus Cloning

Insertion of foreign genes into the attTn7 site (Tn7 locus) of Bacmid 375 was done by transforming NEB 10-beta E. coli with Bacmid 375, the T7 transposase helper plasmid pMON7124 and a foreign gene containing Tn7L/Tn7R Bac 375 (Luckow et al., J Virol., 67(8):4566-79 (1993), which is hereby incorporated by reference in its entirety). White colony clones were isolated on LB Agar plates with 7 μg/mL Gentamycin, 50 μg/mL Kanamycin, 10 μg/mL Tetracycline, 100 μg/mL Bluo-gal, and 40 ug/mL IPTG with X-gal (Teknova, Hollister CA, L1924). Bacmid clones were cultured in LB media, purified of total DNA, retransformed into NEB 10-Beta E. coli, colony isolated on 50 ug/ml Kanamycin LB Agar plates and screened for colonies lacking helper plasmid and Bac 375. Recombinant Tn7 locus bacmids were confirmed using PCR and primers JS175-Tn7locus-LP TTCACACAGGAAACAGCTATGACCATG (SEQ ID NO: 100) and JS174-Tn7locus-RP CGCGCGTAATACGACTCACTATAGG (SEQ ID NO: 101).

### Bac375-RepCap

The Bac375-RepCap bacmid (FIG. 1A) was similar in design to Bac-RepCap (Smith et al., Mol Ther., 17(11):1888-96 (2009), which is hereby incorporated by reference in its entirety) with Rep and Cap genes in opposite orientation in the Tn7 locus of the bacmid. A RepCap Bac 375 called BACRC015 was made synthetically (Aldeveron, Madison WI). The BACRC015 Bac 375 followed the design of plasmid, pSR657 (Smith et al., Mol Ther., 17(11):1888-96 (2009), which is hereby incorporated by reference in its entirety) (Addgene plasmid #65214; http://n2t.net/addgene:65214; RRID:Addgene_65214). BACRC015 had a RepCap gene cassette flanked by Tn7L, Tn7R sequences for Tn7 transposition into the bacmid. The polh promoter and modified AAV2 Rep gene was identical to pSR657. The Cap gene had an AcMNPV p10 promoter with 39 bp AAV2 leader sequence driving Cap gene expression and non-canonical CTG translational start codon for the VP1 ORF of the Cap gene as in pSR657. The serotype of the Cap gene ORF in BACRC015 was an AAV9 neurotropic variant called AAV-PHPN (Chan et al., Nat Neurosci., 20(8):1172-1179 (2017); Kumar et al., Nature Methods, 17:541-550, 2020), which are hereby incorporated by reference in their entirety) (GenBank MF187357.1). Relative to that sequence, AAV-PHPN used here had mutations T1344A, A1347G and T1803C. A1347G resulted in a K449R mutation in the capsid ORF that was found not to reduce AAV potency on HEK 293T cells. The other mutations were silent. The BACRC015 Bac 375 was used to introduce the RepCap cassette into Bacmid 375 to create bacmid Bac375-RepCap.

### Bac375-LacRepCap

Four synthesized gene cassettes were cloned into Bacmid 375 to create the bacmid Bac375-LacRepCap (FIG. 1B). Synthetic portions of gene cassettes were made in pUC57 Bac 375s by GenScript (Piscataway, NJ) and were cloned into the Bac375 bacmid by either by Tn7 transposition (Luckow et al., J Virol., 67(8):4566-79 (1993), which is hereby incorporated by reference in its entirety) or by T4 ligase cloning (Lihoradova et al., J Virol Methods., 140(1-2):59-65 (2006), which is hereby incorporated by reference in its entirety). Gene cassettes that were cloned by T4 ligase ligation were cut from Bac 375s using either I-CeuI. AvrII or FseI enzymes (New England Biolabs) and agarose gel purified using the QIAEX II Gel Extraction Kit (Qiagen, Germantown MD). Bacmid DNA for T4 ligase ligations was purified by Qiagen Large-Construct Kit, linearized using I-CeuI, AvrII or FseI and then added to T4 ligase reactions with gel purified synthetic gene cassette fragments. T4 ligase ligations were done in 50 ul volumes and included 600 ng of cut bacmid DNA and 100 ng to 1285 ng of synthetic gene cassette (1:9 to 1:50 molar ratio). Ligations were transformed into NEB 10-Beta E. coli. and bacmid clones were selected on kanamycin-agar plates and screened by PCR.

The Bac375-LacRepCap baculovirus was made in the following steps. An AvrII-polh-LacR-AvrII cassette was T4 ligase ligated into the unique AvrII site in the egt locus of Bac375 to create Bac375-LacR. The second step was to T4 ligase clone an ICeuI-lacO-p10-lacO-VP1-ICeuI cassette into the I-CeuI site in the VC locus to create Bac375-lacOVP1-LacR (bacmid 1095). This is the LacVP1 baculovirus that was used to see if LacR could regulate a p10 promoter. The next step was T4 ligase clone an FseI-lacO-p10-lacO-VP2-gta-FseI cassette into the FseI site in the gta locus of Bac375-lacOVP1-LacR to create Bac375-lacOVP1-lacOVP2-LacR (bacmid 1186). The final step was to Tn7 transposition insert a Tn7L-gp64-polh-LacR-polh-Rep2-p10-VP3-Tn7R cassette in into the attTn7 site to product the final bacmid construct Bac375-LacR-Rep-VP3-lacOVP1-lacOVP2-LacR (bacmid 1260) call Bac375-LacRepCap.

### sf9 Cell Culture & Baculovirus Infections

Sf9 cells were maintained in ESF AF media (Expression Systems, Davis CA). Small scale 50 ml mini bioreactor (Corning, 431720) cultures were shaken on an orbital shaker at 265 rpm at 28° C. while large shaker flasks (250 ml to 1 L) were agitated at 150 rpm. Baculovirus inoculums were baculovirus infected insect cells (BIICs) stored at −80° C. BIIC titers were determined by TCID50 assay in 96-well plates using a proprietary GFP reporter Sf9 cell line. TCID50 units were calculated using the Reed & Muench 1938 method. Sf9 cells were infected at a cell density of 3.0×10⁶ cells/ml Infections and were monitored using a Cellometer™ Auto T4 (Nexcelom Bioscience, Lawrence MA). Uninfected cell diameters were 15-16 uM and infected cell diameters were 18-20 uM.

Sucrose Cushion rAAV Purification

Twenty-five ml volumes of BEV infected Sf9 cells (6.0×10⁶ cells/ml) were lysed in culture media by adding 1.25 ml of 10% w/v Triton X-100 Spike (Cytiva, RR17714.03), 1.25 ml of 2 M Arginine Spike (Cytiva, RR17714.03) and 1.2 ul of 250 U/ul benzonase nuclease (Millipore, E1014). The 50 ml mini bioreactor tubes were placed back at 28° C. and shaken for 24 h at 260 rpm in the orbital shaker. A 300 ul aliquot was set aside for crude lysate rAAV titer determination and the remaining was centrifuged for 7 min at 7,000×g. Soluble lysate supernatants were transferred to open top, 38.5 ml, 25×89 mm, thin wall Utra-Clear centrifugation tubes (Beckman, 344058). Using capillary tube and syringe, lysates were under laid with 6 ml of 20% w/w sucrose in PBS-F68 (Teknova, 2P9568). Tubes were placed in an SW32 Ti rotor and centrifuged at 30,000 rpm (67,214×g min to 153,445×g max) for 1 h at room temperature in a Beckman Optima LE80 Ultracentrifuge. The supernatant lysate upper layer and sucrose solution were aspirated away leaving only the sucrose cushion pellet at the bottom of the tube. 500 ul of PBS-F68 was added to the pellets and tubes were left for overnight at 4° C. Pellets were suspended in the PBS-F68, transferred to 1.5 ml cryotubes and vortexed. After vortexing, cryotubes were centrifuged for 7 min at 20,000×g in a micro centrifuge. The supernatants were collected and transferred to fresh 2.0 ml cryotubes. These supernatants were stored at 4° C. while experiments were conducted and at −20° C. when longer term storage was needed. This one-step sucrose cushion centrifugation method rapidly purifies rAAV capsids for potency assays and does not require affinity purification or iodixanol ultracentrifugation step gradient purification.

### Affinity Purification, CE-SDS Analysis, and Empty/Full Ratios

Eight hundred ml cultures of BEV infected Sf9 cells were lysed in culture media supplemented with 0.5% v/v Triton X-100, 200 uM arginine and benzonase as described earlier. Cell lysate rAAV capsids were affinity captured using POROS™ Capture Select™ AAV9 affinity resin (Thermofisher, A27354). Resin captured rAAV were washed with 20 mM Tris, 1M NaCl (pH 8.0), bridge washed with 50 mM Na₂PO₄, 350 mM NaCl (pH 5.5), and then eluted with 200 mM glycine, 50 mM NaCl (pH 3.0). Eluted rAAV were neutralized by adding Tris-base to 150 mM final concentration. At all steps in purification contained 0.001% v/v pluronic F-68 (Thermofisher, 24040032). Capillary electrophoresis sodium dodecyl sulfate (CE-SDS) was done using a Sciex PA-800 capillary electrophoresis system. Affinity chromatography purified rAAV capsid samples were measured for total protein concentrations using a BCA kit (Pierce Cat. 23227). Samples were diluted in water to a total protein concentration of 100 ug/ml. Quantification of separated proteins was done by measuring peak area absorbances at 220 nm and was normalized to the molecular weight to accurately calculate ratios of VP1, VP2 and VP3. Empty-full ratios of capsids were determined using size exclusion chromatography multiangle light scattering (SEC-MALS) as described by McIntosh et al., Sci Rep., 11(1):3012 (2021), which is hereby incorporated by reference in its entirety.

### Western Blots

For total cell lysate (CL) Western blots, Sf9 cells were collected by centrifugation for 2 min, 20,000×g and then cell pellets were resuspended in PBS, pH 7.4 (Gibco 10010072) at a concentration 2.0×10³ cells/ul. 4×NuPAGE™ LDS Sample Buffer (Invitrogen NP0007) and 10×NuPAGE™ Sample Reducing Agent (Invitrogen NP0007) were combined and diluted to 2× concentrations in PBS, pH 7.4. The resulting 2×LDS Sample Buffer/Reducing Agent was combined with equal volumes of PBS diluted cells. For sucrose cushion (SC) Western blots, 40 ul of PBS, pH 7.4 solubilized SC pellets were diluted with 15 ul of 4×LDS Sample Buffer and 6 ul of 10× Sample Reducing agent. The final SC sample represented material generated from 2.0×10⁵ Sf9 cells/ul. Samples were vortexed and then heat denatured for 10 min at 80° C. Volumes of 10 ul were loaded onto NuPAGE™ 4-12% Bis-Tris 1.0 mm×17 well gels (Invitrogen NP0329BOX). Proteins were fractionated by electrophoresis in 1×MOPS (Boston BioProducts BP-178) for 80 min at 130 V and then transferred to nitrocellulose (Trans-Blot Turbo Transfer Pack, Midi format, 0.2 um nitrocellulose, Bio-Rad 1704159) using a BioRad Trans-Blot Turbo Transfer System, (25V, 1 Amp, 30 min). Blots were blocked for 1 h with 5% w/v Blotting-Grade Blocker (Bio-Rad, 1706404) diluted in 1×TBS-T (Boston BioProducts IBB-180×). Primary mouse monoclonal antibodies were diluted to 1:2000 in TBS-T and incubated with blocked blots for 90 min. Secondary anti-mouse HRP conjugated antibodies were diluted 1:10,000 in TBS-T and incubated 90 min with primary antibody probed blots after they had been washed with 2×TBS-T three times. Western blot signals were detected using Clarity Western ECL Substrate (Bio-Rad, 170-5060) and an Azure Imager c300 (Azure Biosystems, Dublin CA). All images were collected as Tiff files and analyzed using ImageJ software (Schneider et al., Nat Methods., 9(7):671-675, 2012), which is hereby incorporated by reference in its entirety).

rAAV Transduction Assays

HEK 293T cells were seeded into 96-well plates at 1.0×10⁴ cells/well in 80 ul of Opti-MEM™ I Reduced Serum Medium, GlutaMAX™ (Gibco, 51985091) supplemented with 5% v/v fetal calf serum. After 2 days of incubation at 37° C. in 5% v/v CO₂, cells were transduced with rAAV containing sucrose cushion samples. In a U-bottom 96-well plate, 20 ul volumes of 1/10, 1/20, 1/40 and 1/80 diluted sucrose cushion samples in PBS-F68 were combined with 200 ul of Opti-MEM media and 8 ul of 100× Antibiotic-Antimycotic (Gibco, 15240112). Thirty-five ul volumes of these rAAV dilutions were transferred to the wells of the HEK 293T cell plates. This was done in triplicate for each rAAV sample.

Determination of rAAV Titer by Q-PCR

Before initiating quantitative PCR (Q-PCR), rAAV samples were predigested with DNaseI to eliminate non capsid associated DNA. DNaseI (2 U/ul, Teknova 3D1401) was diluted 30-fold in 1×DNAseI buffer (10 mM Tris-HCl, 2.5 mM MgCl₂, 0.5 mM CaCl₂, pH 7.6) and 95 ul volumes of the resulting DNAseI reaction buffer were combined with 5 ul volumes of rAAV samples. Samples were incubated for 1 h at 37° C. and then DNAseI digestions were stopped by adding 125 ul volumes of proteinase K/EDTA reaction buffer which contained 1 mg/ml proteinase K (Teknova P2050), 111 mM NaCl, 0.11% w/v sarkosyl, and 4 mM EDTA pH 8.0. After adding proteinase K/EDTA reaction buffer, samples were heated at 55° C. for 1 h followed by heat inactivation of proteinase K at 95° C. for 10 min. DNAseI/Proteinase K treated rAAV samples were then diluted 40-fold into 200 ul of 10 mM Tris pH 7.5. Four ul volumes of resulting tris diluted rAAV samples were added to 16 ul volumes of Q-PCR reaction mixes in 200-ul capacity, 96-well PCR plates. Ten-fold serially diluted DNA standards were included on the same PCR plates to enable quantification. All Q-PCR samples were run in triplicate. Q-PCR reaction mixes included TaqMan Fast Advanced Mastermix (ThermoFisher, 44445556) diluted to 1.2× and 6 uM primers specific for the CMV promoter; Forward Primer 5′-TACGGTAAACIGCCCACIT-3′ (SEQ ID NO: 102), Reverse Primer 5′-GTCCCATAAGGTCATGTACTGG-3′ (SEQ ID NO: 103), and Probe Primer 5′-FAM-GTCCCATAAGGTCATGTACIUG-ZEN-3′ (SEQ ID NO: 104). Q-PCR reactions were done on a Lightcycler 480 (Roche), beginning with a 10 min, 95° C. heat denaturation, then 45 cycles of 95° C., 10 sec, 60° C. 10 sec and finishing step of, 72° C. 10 sec. Resulting data were analyzed on MS-Excel.

### SEAP Activity Assay Assays

At 4 days post transduction with rAAV, the media from HEK 293T cells was transferred from 96-well tissue culture plates to 96-well, PCR plates, that were sealed with aluminum covers and heated at 65° C. for 15 min to inactivate cellular alkaline phosphatases. Fifty ul volumes of heat-treated media were transferred into 96-well microtiter plates and combined with 50 ul volumes of p-nitrophenylphosphate (pNPP) solution (Surmodics Inc., BioFX AP-Yellow One, Fisher Scientific, NC9444916). Samples were incubated for 24 h at room temperature in the dark to pNPP reaction endpoint. The optical density at 405 nm (OD405) was read using a BioTek Synergy HTX microplate reader. Every assay included a recombinant shrimp alkaline phosphatase (NEB, M0371S) standard which had been serially diluted in Opti-MEM media and not heat treated. Shrimp alkaline phosphatase was confirmed by the same assay to have equal unit phosphatase unit activity as calf intestinal alkaline phosphatase (Invitrogen, 18009027).

### Statical Calculations

Standard deviations for Q-PCR derived rAAV genome titers or SEAP activity assays were calculated from the average of three repeated samples. Standard deviations for potency (SEAP activity/rAAV genome) were calculated from the square route of the sum of the squared coefficients of variation of Q-PCR titer and SEAP activity.

### Production of Bacmid 639

An AvrII-polh-NLS-LacR-AvrII cassette was synthesized for cloning into the unique AvrII site of the Bacmid 375. The 5′ end began with the AvrII site region CCTAGGGCTAGCGTATAC (SEQ ID NO: 105) followed by a 92 bp polh promoter corresponding to 4,429 to 4,520 AcMNPV E2 (GenBank KM667940.1). After the polh promoter, was the sequence ATGACGCAACCTAAGAAGAAGAGGAAGGTTCCCGGGCAAGTGACT (SEQ ID NO: 106) which encodes for the peptide MTQPKKKRKVPGQVT (SEQ ID NO: 107). The “PKKKRKV” (SEQ ID NO: 108) region in the peptide was an SV40 large T antigen nuclear localization signal (NLS) (Kalderon et al., Cell, 39:499-509 (1984), which is hereby incorporated by reference in its entirety) and the remaining linker peptide designed to be on the N-terminus of the LacR ORF. The LacR ORF had an in frame ATG start codon followed and corresponded to the E. coli Lac gene 82 to 1161 (GenBank J01636.1). Following the NLS-LacR region was a 135 bp SV40 Large T antigen polyadenylation signal corresponding to 2668-2534 of the SV40 genome (GenBank NC_001669.1). The cassette 3′ end had a AvrII site region ACTAGTCCTAGG (SEQ ID NO: 109).

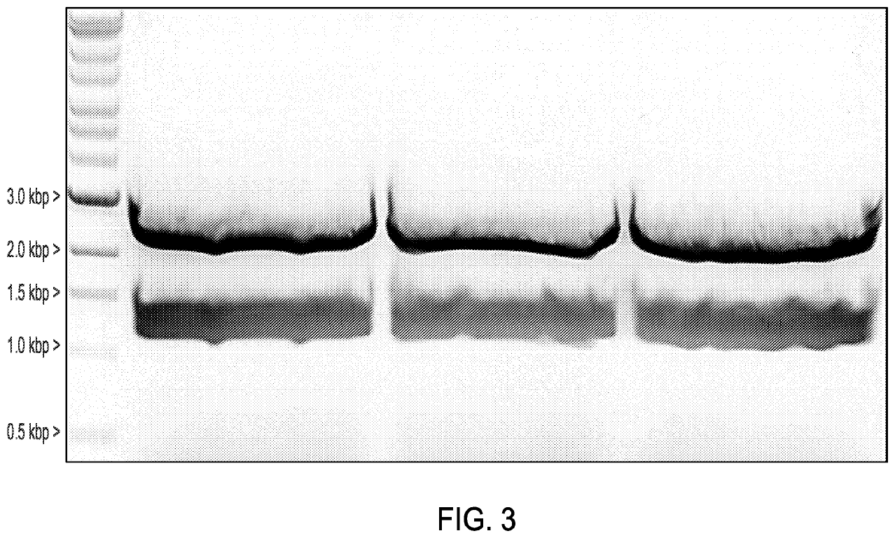

The plasmid containing the AvrII-polh-NLS-LacR-AvrII polynucleotide (SEQ ID NO: 15) was produced using pUC57 production plasmid vector (GenScript Biotech Corp). The polh-NLS-LacR-pUC57 (2,716 bp) was then digested overnight using 10× Cut Smart Buffer (New England Biolabs, Inc.) and AvrII enzyme (5 U/μL) in water (20 ng/μL final concentration). The resulting polh-NLS-LacR insert (1,379 bp) was purified using gel purification. The process was repeated to collect additional polh-NLS-LacR insert as needed. FIG. 3 shows clear separation of the polh-NLS-LacR insert (1,379 bp) from the polh-NLS-LacR-pUC57 plasmid (2,716 bp).

A donor baculovirus plasmid (i.e., bacmid) was provided which included an AvrII egt region, such as the AcMNPV bacmid bMON14272 (Invitrogen Life Technologies) or a variant thereof. The bacmid was then digested using 10× Cut Smart Buffer (New England Biolabs, Inc.) and AvrII enzyme (5 U/μL) in water (50 ng/μL final concentration) at 37° C. for 2 hours, resulting in a single-cut bacmid at the AvrII egt locus. The process was repeated to collect additional AvrII-cut bacmid as needed.

The AvrII-cut bacmid was ligated with the polh-NLS-LacR insert by combining 10 μL AvrII-cut bacmid (500 ng), 30 μL polh-NLS-LacR insert (600 ng), 5 μL 10×T4 ligase buffer and 2 μL T4 ligase enzyme (400 U/μL), and then incubating at 37° C. for 4 hours. In one alternative, the AvrII-cut bacmid was ligated with the polh-NLS-LacR insert by combining 10 μL AvrII-cut bacmid (500 ng), 10 μL polh-NLS-LacR insert (200 ng), 25 μL H₂O, 5 μL 10×T4 ligase buffer and 2 μL T4 ligase enzyme (400 U/μL), and then incubating at 37° C. for 4 hours. In one alternative, the AvrII-cut bacmid was ligated with the polh-NLS-LacR insert by combining 20 μL AvrII-cut bacmid (1000 ng), 10 μL polh-NLS-LacR insert (200 ng), 20 μL H₂O, 5.5 μL 10×T4 ligase buffer, 2.5 μL T4 ligase enzyme (400 U/μL) and 2.0 μL of 10 mM ATP, and then incubating at 37° C. for 4 hours.

The resulting aqueous phase was then combined with 2 μL sodium acetate and 100-120 μL of ice-cold ethanol. Precipitated DNA pellets were collected and resuspended in 40 μL Tris-EDTA buffer. The resulting ligated plasmid DNA was then transformed into electroporated NEB 10-Beta E. coli (New England Biolabs, Inc.).

Bacterial colonies were grown and screened by colony-pick PCR to test for LacR insertion into the AvrII-cut bacmid.

Colony PCR screening was completed using a combination of two primers: Primer 101-JS101-LP-EGT upstream (SEQ ID NO: 16) and Primer 102-JS102-RP-EGT downstream (SEQ ID NO: 17). Positive PCR results had a target of about 3111 bp based on the primers used. It was noted that the use of primer 102 resulted in an artifact amplicon fragment of about ˜2 kb which appeared in several of the PCR screening columns.

PCR screening showed that Colony 601 (FIG. 4A) and Colonies 637-639 (FIG. 4B) had strong bands around 3111 bp, indicating possible LacR insertion into the AvrII-cut bacmids of those bacterial colonies.

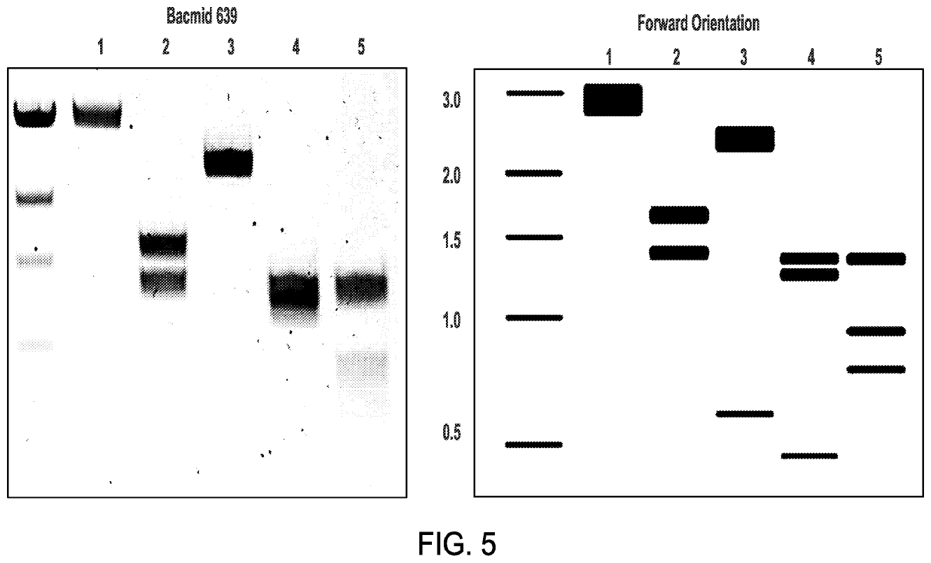

REN digestion analysis of Colony 639 (FIG. 5) showed that Colony 639 corresponded with the forward orientation of LacR insertion and the same orientation as the Ac-egt ORF.

Cloned cassette co-linearity with the disrupted egt ORF was determined by SmaI REN digest of the purified PCR amplicon and observed 600 bp and 2511 bp fragments.

### Production of Bacmid 1095

An ICeuI-lacO-p10-lacO-VP1-ICeuI cassette was synthesized for cloning into the unique I-CeuI site in the VC locus of Bacmid 375. The 5′ end of the cassette contained an I-CeuI site sequence

(SEQ ID NO: 110)
TCGCTACCTTAGGACCGTTATAGTTATGACTAACTAAACTAGTGTATACT.

This was followed by a lacO-p10-lacO region. This region had two lacO sequences GATTGTGAGCGCTCACAATT (SEQ ID NO: 14) flanking a 186 bp AcMNPV p10 promoter corresponding 118,726 to 118,906 of AcMNPV E2 genome (GenBank KM667940.1). The p10 promoter was modified to have 3 internal ATG start codons changed to TTG. The lacO-p10-lacO promoter was followed by the VP1 ORF (nt 1 to 2232) of the AAV-PHPN capsid. It had the same capsid sequence as BACRC015 except this ORF had an ATG start codon. The VP1 ORF was followed by the sequence GCTAGCACGCGTAGCTGATGCATAGCATGCGGTA (SEQ ID NO: 111) and a 115 bp UL23 gene thymidine kinase polyadenylation (TK-PolyA) region corresponding to HSV-1 (GenBank MN159379.1) (46,696 to 46582). The TK poly A sequence was followed by the I-CeuI containing sequence

(SEQ ID NO: 112)
TCTAGATTAGTTAGTCATCGCTACCTTAGGACCGTTATAGTTA.

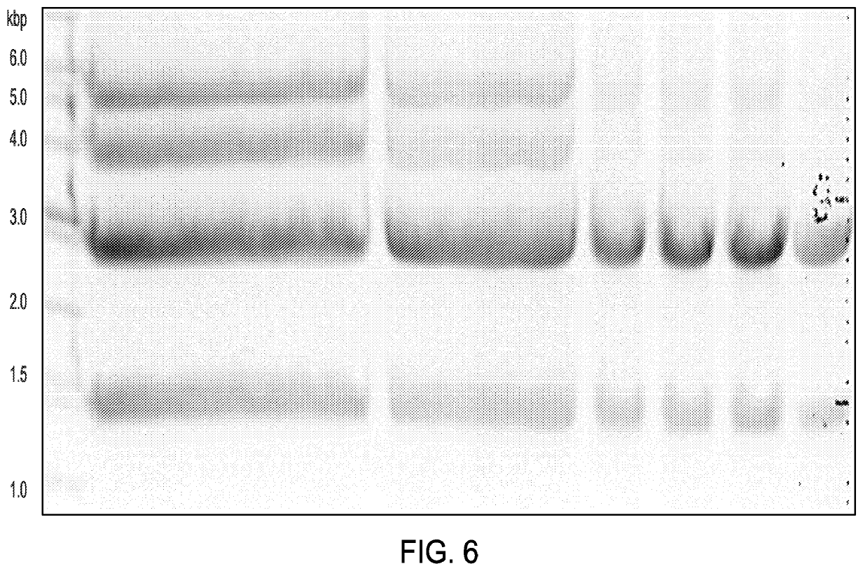

The plasmid containing the LacO-p10-LacO-VP1 polynucleotide (SEQ ID NO: 18) was produced using pUC57 production plasmid vector (Thermo Fisher Scientific Inc). 50 g of LacO-p10-LacO-VP1-pUC57 was then digested using 10× Cut Smart Buffer (New England Biolabs, Inc.), I-CeuI enzyme (5 U/μL) and BsaI enzyme (20 U/μL) in water (167 ng/μL final concentration) at 37° C. for 2 hours, followed by exposure to 75° C. for 10 minutes to inactivate the enzymes. The resulting LacO-p10-LacO-VP1 insert (2,679 bp) was purified using gel purification (electrophoresis in a 0.8% w/v agarose, 1×TAE gel, 80 min, 120V), with 7800 ng of recovered product. The process was repeated to collect additional LacO-p10-LacO-VP1 insert as needed. The gel presented in FIG. 6 shows clear separation of the LacO-p10-LacO-VP1 insert (2,679 bp) from remaining LacO-p10-LacO-VP1-pUC57 plasmids (5,365 bp) and pUC57 fragments (˜1300 bp after being cut in half by BsaI enzyme).

Bacmids from Colony 639 were provided, with each having an I-CeuI region. 6 μg of the 639 Bacmid was digested using 10× Cut Smart Buffer (New England Biolabs, Inc.) and I-CeuI enzyme (5 U/μL) in water (76 ng/μL final concentration) at 37° C. for 2 hours, followed by exposure to 75° C. for 10 minutes to inactivate the enzymes, resulting in a 639 Bacmid single-cut at the I-CeuI locus. The process was repeated to collect additional I-CeuI-cut 639 Bacmid as needed.

The I-CeuI-cut 639 Bacmid was ligated with the LacO-p10-LacO-VP1 insert by combining 25 μL I-CeuI-cut 639 Bacmid (600 ng), 25 μL LacO-p10-LacO-VP1 insert (1275 ng), 1 μL 100 mM ATP, 0 μL 1× Cut Smart buffer and 3 μL T4 ligase enzyme (400 U/μL), and then incubating at 37° C. In one alternative, the I-CeuI-cut 639 Bacmid was ligated with the LacO-p10-LacO-VP1 insert by combining 25 μL I-CeuI-cut 639 Bacmid (600 ng), 10 μL LacO-p10-LacO-VP1 insert (500 ng), 1 μL 100 mM ATP, 15 μL 1× Cut Smart buffer and 3 μL T4 ligase enzyme (400 U/μL), and then incubating at 37° C. In one alternative, the I-CeuI-cut 639 Bacmid was ligated with the LacO-p10-LacO-VP1 insert by combining 25 μL I-CeuI-cut 639 Bacmid (600 ng), 5 μL LacO-p10-LacO-VP1 insert (250 ng), 1 μL 100 mM ATP, 20 μL 1× Cut Smart buffer and 3 μL T4 ligase enzyme (400 U/μL), and then incubating at 37° C. In one alternative, the I-CeuI-cut 601 Bacmid was ligated with the LacO-p10-LacO-VP1 insert by combining 25 μL I-CeuI-cut 639 Bacmid (600 ng), 2 μL LacO-p10-LacO-VP1 insert (100 ng), 1 μL 100 mM ATP, 20 μL 1×Cut Smart buffer and 3 μL T4 ligase enzyme (400 U/μL), and then incubating at 37° C.

The resulting aqueous phases were then mixed with 2 μL 3M sodium acetate and 2 volumes of ice-cold ethanol, then chilled at −20° C. for 20 minutes. Precipitated DNA pellets were collected by centrifuge and resuspended in 80 μL Tris-EDTA buffer. The resulting ligated plasmid DNA was then transformed into electroporated NEB 10-Beta E. coli (New England Biolabs, Inc.).

Bacterial colonies were grown and screened by colony-pick PCR to test for LacO-p10-LacO-VP1 insertion into the I-CeuI-cut 639 Bacmid.

Colony PCR screening was completed using a combination of four primers: Primer JS16-Lef7-LP1 (SEQ ID NO: 19), Primer JS17-gp64UTR-RP (SEQ ID NO: 20), Primer JS61-VP3-primer2 (SEQ ID NO: 21) and Primer JS92-AAP-RP1 (SEQ ID NO: 22). Positive PCR results had a target of about 3838 bp (JS16-JS17), 1398 bp (JS16-JS61) or 1092 bp (JS92-JS17) based on the primers used.

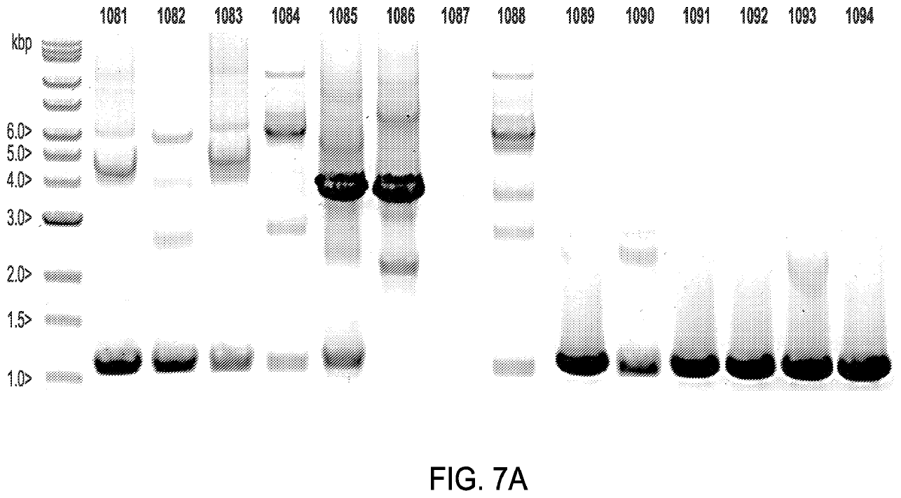

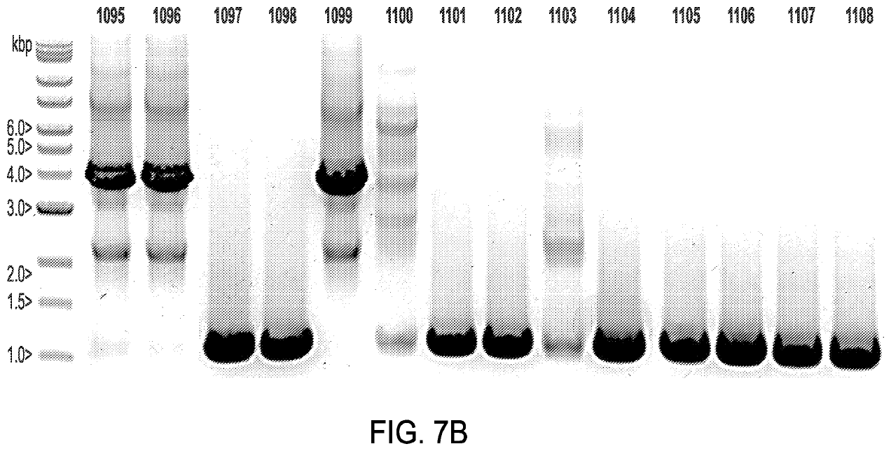

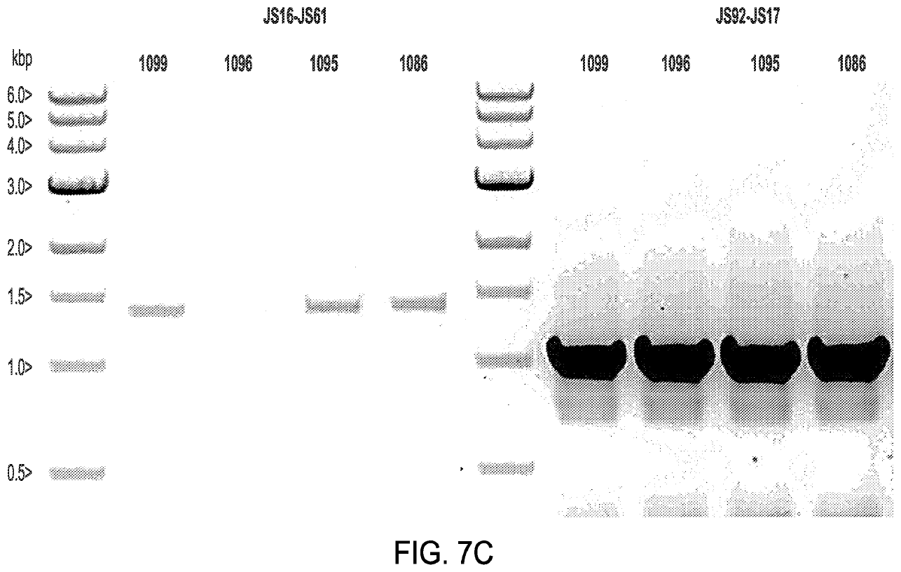

Results of JS16-JS17 PCR screening of the bacterial colonies for LacO-p10-LacO-VP1 insertion into the I-CeuI-cut 639 Bacmid are shown in FIGS. 7A-7C. PCR screening showed that Colonies 1085-1086 (FIG. 7A), Colonies 1095-1096 (FIG. 7B) and Colony 1099 (FIG. 7B) had strong bands around 3838 bp, indicating likely insertion of LacO-p10-LacO-VP1 insertion into the I-CeuI-cut 639 Bacmid of those bacterial colonies. Strong bands for the remaining colonies around 1159 bp correlated with empty I-CeuI sites in the I-CeuI-cut 639 Bacmid.

Results of JS16-JS61 and JS92-JS17 PCR screening of Colonies 1086, 1095, 1096 and 1099 for LacO-p10-LacO-VP1 insertion into the I-CeuI-cut 639 Bacmid are shown in FIG. 7C. PCR screening showed that Colonies 1086, 1095 and 1099 had strong bands around 1398 bp and 1092 bp, indicating likely insertion of LacO-p10-LacO-VP1 insertion into the I-CeuI-cut 639 Bacmids. Colony 1096 had a strong band around 1398 bp but no discernable band around 1092 bp. VP1 ORF insertion was confirmed to be in the same direction as the upstream baculovirus gp64 gene using primers JS17-gp64UTR-RP (SEQ ID NO: 20) and JS92-AAP-RP1 (SEQ ID NO: 22).

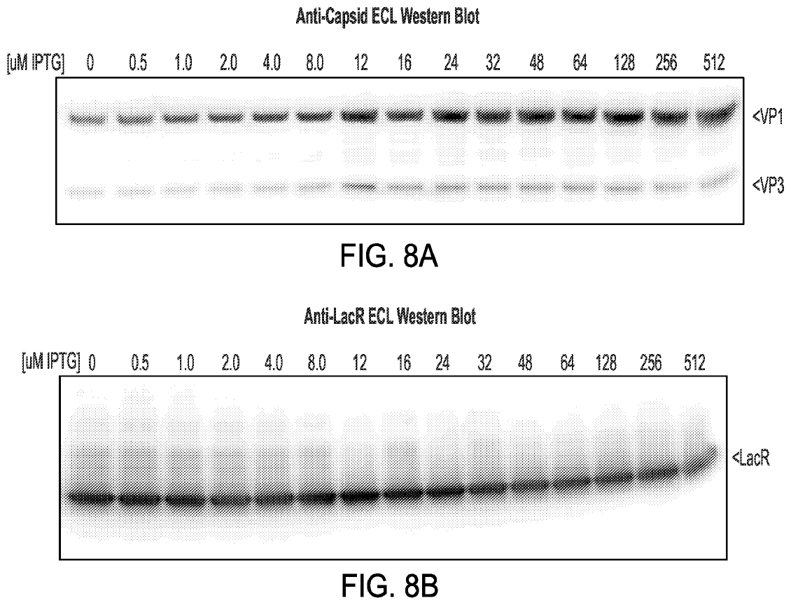

Colony 1095 was tested using Anti-AAV Capsid ECL Western Blot and Anti-LacR ECL Western Blot, with isopropyl-β-D-thiogalactose (IPTG) being used as the inducer element. Bacmids from Colony 1095 were infected into Sf9 cells under different IPTG concentrations, and the total cell lysates at 3 days post infection were analyzed using Western Blot. Results are shown in FIG. 8A and FIG. 8B.

Results in FIG. 8A show that VP1 production was being regulated by the LacR, with a lower concentration of IPTG resulting in lower VP1 production and a higher concentration of IPTG resulting in higher VP1 production.

### Production of Bacmid 1186

An FseI-lacO-p10-lacO-VP2-gta-FseI cassette was created for cloning into the unique FseI REN site in the Bac375 bacmid. In short, the bacmids from Colony 1095 were provided and digested using Cut Smart Buffer and FseI enzyme in water at 37° C. The product was gel purified to provide FseI-cut 1095 Bacmid. The process was repeated to collect additional FseI-cut 1095 Bacmid as needed. The FseI-cut 1095 Bacmid was ligated with the FseI-LacO-p10-LacO-VP2-AClef12-ACgta-FseI polynucleotide inserts (SEQ ID NO: 23) by combining 25 μL FseI-cut 1095 Bacmid, 25 μL of gel purified FseI-LacO-p10-LacO-VP2-AClef12-ACgta-FseI polynucleotide insert, 5.5 μL 10× T4 ligase buffer and 2 μL T4 ligase enzyme (400 U/μL), and then incubating at 19° C. for 3 hours. The resulting bacmids were then gel purified.

Two PCR products (Left and Right) were amplified, cut with MluI, ligated together, PCR amplified again and ligated as a single FseI-lacO-p10-VP2-gta-FseI cassette into the Bacmid 375 genome. The 3,106 bp left PCR product was amplified from a synthetic pUC57-FseI-lacO-P10-VP2-FseI construct and the 576 bp right PCR product was amplified from the AcMNPV E2 genome. The right PCR product restored the baculovirus gta gene promoter and ORF which were disrupted by cassette insertion into the FseI site in the bacmid. The left PCR product was amplified from the pUC57-FseI-lacO-P10-VP2-FseI synthetic construct using primers JS134-pUC57-LP1 (TATAAGGCCGGCCGTATCACGAGGCCCTTTCGT) (SEQ ID NO: 113) and JS135-pUC57-RP1 GCTTTGGCCGGCCTACCGCCTTTGAGTGAGCTG (SEQ ID NO: 114). The 5′ end of this left PCR product was the sequence TATAAGGCCGGC (SEQ ID NO: 115), followed by 465 bp pUC57 (GeneBank Y14837.1) region 2689 to 431 and the FseI site containing sequence CCTAGGGGCCGGCCTGACTAACTAAACTAGTGTATACT (SEQ ID NO: 116). This sequence was followed by the same lacO-p10-lacO promoter region described in the ICeuI-lacO-p10-lacO-VP1-ICeuI cassette. The lacO-p10-lacO promoter was followed by AAV-PHPN capsid VP2 ORF (nt 412 to 2232). This ORF was based on the same BACRC015 sequence used to make Bac375-RepCap but had a C412A nt base mutation such that VP2 had an ATG start codon instead of CTG. The VP2 ORF was followed was followed by the sequence, GCTAGCACGCGTAGCTGATGCATAGCATGCGGTA (SEQ ID NO: 117) and a 115 bp UL23 gene thymidine kinase polyadenylation (TK-PolyA) region corresponding to HSV-1 (GenBank MN159379.1) (46,696 to 46582). The TK poly A sequence was followed by the sequence TCTAGATTAGTTAGTCAGGCCGGCCCCTAGG (SEQ ID NO: 118) and the pUC57 (GeneBank Y14837.1) region 432-789. The 3′ end of the left PCR sequence was completed by the sequence GGCCGGCCAAAGC (SEQ ID NO: 119). The left PCR product was an FseI-lacO-p10-lacO-VP2-FseI cassette intended for the FseI site of Bacmid 375. This disrupted the gta gene ORF that was reported non-essential for baculovirus replication in insect cell culture (Katsuma et al., J Gen Virol., 89(Pt 12):3039-3046 (2008), which is hereby incorporated by reference in its entirety). The gta gene was found to be essential to AcMNPV replication in Sf9 cells. The left PCR product was cut at an MluI site 7 bp downstream of the VP2 ORF to permit ligation a MluI-gta-FseI right PCR product. This enabled restoration of the disrupted gta gene promoter and 5′ end but omitted the HSV-1 poly-A region downstream of VP2. The MluI left PCR fragment was gel purified and T4 ligase ligated to the MluI cut, right PCR product of the gta gene 5′ end. The right PCR product was amplified from the AcMNPV E2 (region 33,799 to 34,279) using primers JS142-gta-UTR-LP-MluI GATCACGCGTCGCGTTACACGTACATGAATTAC (SEQ ID NO: 120) and JS145-gta-RP-SpeI GATCACTAGTGCGATTAACATTAGCACAGAGA (SEQ ID NO: 121). The ligation produced a 3,153 bp FseI-lacO-p10-lacO-VP2-gta-FseI gene product. The ligation product was amplified by a second round of PCR using primers JS134-pUC57-LP1 and JS145-gta-RP-SpeI.

Bacterial colonies were grown and screened by colony-pick PCR to test for FseI-LacO-p10-LacO-VP2-AClef12-ACgta-FseI insertion into the FseI-cut 1095 Bacmid. Colonies 1180-1194 were subjected to Colony PCR screening using three combinations of two primers: (i) Primer JS61-VP3-primer2 (SEQ ID NO: 21) and Primer JS91-gta-RP1 (SEQ ID NO: 24), with positive PCR results having a target of about 715 bp based on the primers used; (ii) Primer JS124-gta-LP10 (SEQ ID NO: 25) and Primer JS92-AAP-RP1 (SEQ ID NO: 22), with positive PCR results having a target of about 1199 bp based on the primers used; and (iii) JS124-gta-LP10 (SEQ ID NO: 25) and Primer JS91-gta-RP1 (SEQ ID NO: 24), with positive PCR results having a target of about 3262 bp based on the primers used.

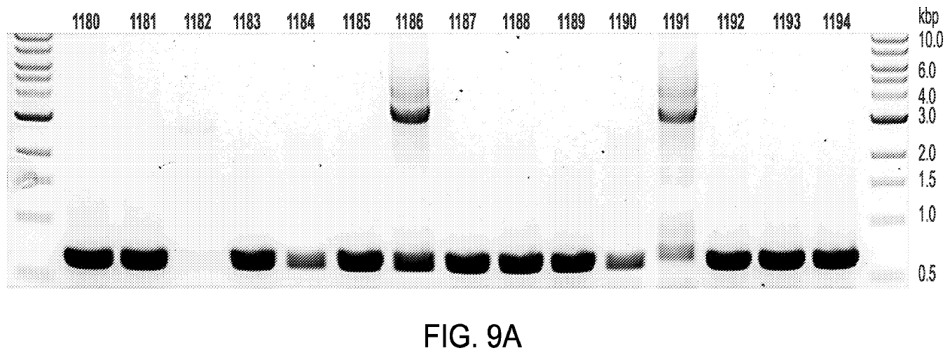

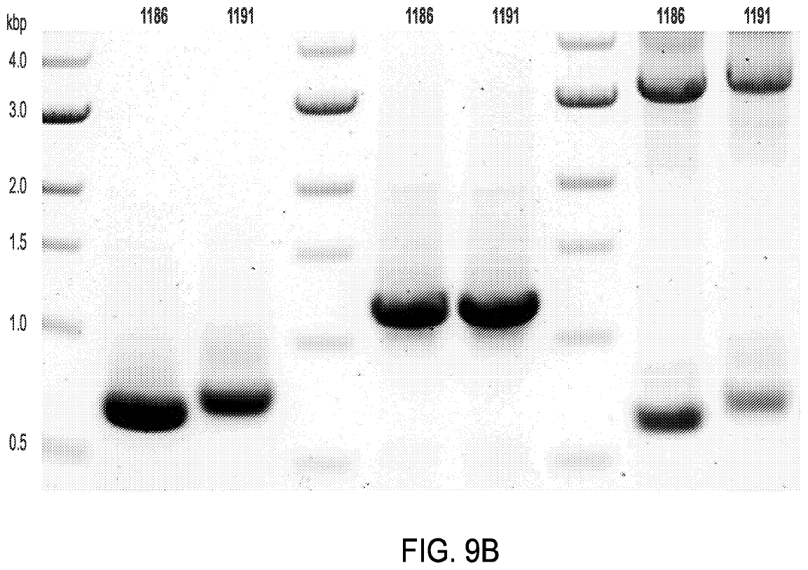

Results of JS124-JS91 PCR screening of Colonies 1180-1194 are shown in FIG. 9A. PCR screening showed that Colonies 1186 and 1191 (FIG. 9A) had strong bands around 3262 bp, indicating likely insertion of FseI-LacO-p10-LacO-VP2-AClef12-ACgta-FseI into the I-FseI-cut 1095 Bacmid of those bacterial colonies. Orientation in the same direction as the gta ORF was confirmed using primers JS61-VP3-primer2 (SEQ ID NO: 21) and JS91-gta-RP1 (SEQ ID NO: 24).

Results of JS61-JS91 (715 bp), JS124-JS92 (1199 bp) and JS124-JS91 (3262 bp) PCR screening of Colonies 1186 and 1191 are shown in FIG. 9B. PCR screening showed that both Colonies 1186 and 1191 (FIG. 9B) had strong bands around 715 bp, 1199 bp and 3262 bp, strongly indicating likely insertion of FseI-LacO-p10-LacO-VP2-AClef12-ACgta-FseI into Bacmids 1186 and 1191 of those bacterial colonies. PCR screening bands for Colony 1186 were slightly stronger than bands for Colony 1191.

The resulting bacmids which provided positive PCR results (including Bacmid 1186) thus included an FseI-LacO-p10-LacO-VP2-AClef12-ACgta-FseI polynucleotide insert (SEQ ID NO: 23).

### Production of Plasmid 1259

A Tn7L-gp64-polh-LacR-polh-Rep2-p10-VP3-Tn7R cassette was engineered as the Bac 375 for transposition into the attTn7 site of the bacmid. Plasmid 1259 was prepared to include an AAV Rep sequence (encoding Rep78 and Rep52 proteins) under a polh promoter, an AAVPHPN Cap sequence encoding VP3-only under a p10 promoter (SEQ ID NO: 26), and an opgp64-polh-NLS-LacR region (SEQ ID NO: 27).

Bac 375 1249 containing VP3-only AAV viral expression constructs were first prepared to include an AAV Rep sequence (encoding Rep78 and Rep52 proteins) under a polh promoter, and an AAVPHPN Cap sequence encoding VP3-only under a p10 promoter. The VP3-only sequence was produced by providing a shuttle plasmid which included an AAVPHPN VP1 sequence and then digesting it with SmaI and Bsu36I enzymes to excise VP1 and VP2 ORF 5′ ends from the VP construct in the plasmid (i.e., to remove AAV2 leader sequence GGGGGATCCTGTTAAA (SEQ ID NO: 122) and VP1 ORF gene sequence region (1 to 585)). The SmaI is a blunt cutting restriction enzyme and the Bsu36I site was blunt filled by incubating with Q5 polymerase. The resulting cut plasmid was phosphorylated with T4 kinase, gel purified and ligated back together with T4 ligase. The resulting Plasmid 1249 included an AAVPHPN Cap sequence encoding VP3-only under a p10 promoter (SEQ ID NO: 26). The AAV2 leader sequence was a CTCGACGAAGACTTGATCACCC (SEQ ID NO: 123) and the remaining unique VP1 gene region was the sequence TCAGGTGTGGGATCTCTTACA (SEQ ID NO: 124). Next to this sequence was the start ATG for VP3. A second copy of LacR was then cloned into the Bac 375. The second copy was included to ensure abundant and early LacR expression. The second inserted LacR was enhanced to have a hybrid early/late/very late promoter instead of just a very late promoter.

Plasmid 1259 was then prepared by providing Plasmid 1249 and then digesting the plasmid with AvrII, just inside the Tn7L sequence after the Rep78 sequence in Plasmid 1249. Primers JS146-AvrIIOpgp64promLP ACATCCTAGGTACAATCAAATTATCGCAAG (SEQ ID NO: 125) and JS147-NheIOpgp64promRP GTGAGCTAGCCITGTAGGTCITGTAGTGT (SEQ ID NO: 126) were used to amplify a synthetically made promoter based on region 154-319 of the OpMNPV gp64 gene (GenBank M22446.1). A G243C mutation was made in the OpMPNV gp64 promoter to remove a minicistron ATG (Chang and Blissard, J Virol., 71(10):7448-60 (1997), which is hereby incorporated by reference in its entirety). The polh-LacR gene was amplified from the AvrII-polh-LacR-AvrII cassette using primers JS155-polh-Mut1-NheI AGGGCTAGCGTATACATCTTGGAGATAATTAAATTGATAAC (SEQ ID NO: 127) and JS156-NLSLacR-RP ACACAGGAAACAGCTATGACCATGAT (SEQ ID NO: 128). Primer JS155 introduced point A to T mutations into the 92 bp polh promoter at positions 4 and 19 to eliminate internal ATG start codons. The AvrII-cut 1249 Plasmid was ligated with the AvrII-opgp64-polh-NLS-LacR-AvrII polynucleotide insert (SEQ ID NO: 28) by combining 22.5 μL AvrII-cut 1249 Plasmid, 22.5 μL of gel purified AvrII-opgp64-polh-NLS-LacR-AvrII polynucleotide insert, 5.0 μL 10× T4 ligase buffer and 2 μL T4 ligase enzyme (400 U/μL), and then incubating at 19° C. for 2 hours. The resulting 1,693 bp gp64-polh-LacR ligation product was gel purified and PCR amplified using primers JS146 and JS156.

The resulting plasmid phase was combined with 1 μL 3M sodium acetate and 100 μL of ethanol. Precipitated DNA pellets were collected by centrifuge and resuspended in 100 μL Tris-EDTA buffer. The resulting ligated plasmid DNA was then transformed into electroporated NEB 10-Beta E. coli (New England Biolabs, Inc.).

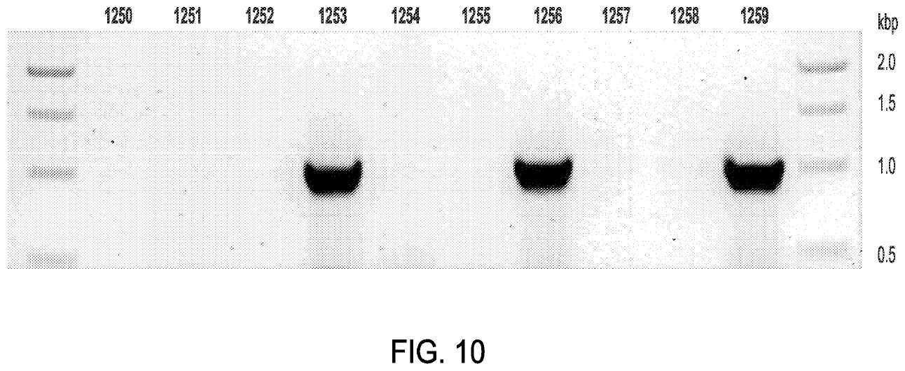

Bacterial colonies 1250-1259 were grown and screened by colony-pick PCR to test for AvrII-opgp64-polh-NLS-LacR-AvrII insertion into the AvrII-cut 1249 Plasmid. Colony PCR screening was completed using a combination of two primers: Primer JS95-LacR-RP1 (SEQ ID NO: 29) and Primer JS42-Rep78-RP_backwards (SEQ ID NO: 30), with positive PCR results having a target of about 1037 bp based on the primers used. Results of JS95-JS42 PCR screening of Colonies 1250-1259 are shown in FIG. 10. PCR screening showed that Colonies 1253, 1256 and 1259 (FIG. 10) had strong bands around 1037 bp, indicating likely insertion of opgp64-polh-NLS-LacR into the AvrII-cut 1249 Plasmid of those bacterial colonies. The AvrII site was between the Tn7L and Rep gene.

Plasmid 1259 thus included the opgp64-polh-NLS-LacR-Rep-VP3 sequence of SEQ ID NO: 31.

### Production of Bacmid AA654

A pUC57 based Bac 375 (SEAP-GFP) was synthesized (GenScript) for Tn7 transposition of an ITR-SEAP-AcV5-GFP-ITR cassette into the attTn7 site of the bacmid to create Bac375-ITR-SEAP-AcV5-GFP. The SEAP-GFP design was based on Teng & Wu, where the SEAP and GFP protein were translated in frame as a chimera with both enzymatic and fluorescent reporter activity (Teng et al., Biotechnol Lett., 29(7):1019-24 (2007), which is hereby incorporated by reference in its entirety). A sequence encoding for the baculovirus GP64 AcV5 epitope (Monsma et al., J. Virol., 69:2583-2595 (1995), which is hereby incorporated by reference in its entirety) was also included to enable monoclonal antibody detection. The SEAP alkaline phosphatase enzymatic activity was measured.

The full size of this ITR-SEAP-AcV5-GFP-ITR cassette to be packaged in rAAV particles was 3,723 bp. The Bac 375 included pUC57 (GeneBank Y14837.1) region 473-495, the sequence CGGATCC, pUC57 regions 496-2710 and 1-396, and the sequence CCCCGC. The following Tn7L-ITR-SEAP-GFP-ITR-Tn7 cassette contained Tn7L region 2-166 from the Tn7 transposon (GenBank MN628641.1). This was followed by the sequence CGGATCTCGACCAATTGAC (SEQ ID NO: 129), region 4,171-4,203 of plasmid pBR322 (GenBank J01749.1) and the sequence GACCTGCAGGCAG (SEQ ID NO: 130). The next sequence was a 5′ ITR region corresponding to region 4664 to 4489 of AAV2 (AF043303.1) and then the sequence CGTCGACATAACGCGTC (SEQ ID NO: 131). After this 5′ ITR containing region was a CMV early enhancer/chicken R actin (CAG) promoter comprised of human cytomegalovirus IE1 gene promoter (GenBank X09322.1) region 544 to 923 and chicken beta actin promoter (GenBank X00182.1) regions 280-373 and 376-547. After the CAG promoter was the sequence CAAGCTT and an SV40 intron containing sequence identical to region 4837 to 5032 of the vector pTR-CB-GFP (GenBank MK225672.1). Downstream of the intron region was the SEAP ORF for Homo sapiens alkaline phosphatase, placental (ALPP) (GenBank NM_001632.5) region 53-1568. There were 5 codon optimization changes; C94G, C328A, C649G, C715T, and C1066T. The ALPP ORF did not have a stop codon and was in frame with the short linker ORF sequence, ACGCTAGC, and then a sequence encoding for the AcV5 epitope that which was mammalian codon optimized into the sequence AGTTGGAAAGACGCCTCAGGTTGGTCC (SEQ ID NO: 132). The epitope region was followed by the linker ORF sequence TTCGCTAGCGGTACCGGT (SEQ ID NO: 133). This linker continued in frame with the free use GFP (fuGFP) ORF from designed plasmid pUS252 (Addgene_127674). The start codon ATG of fuGFP was not include in this 723 bp ORF sequence. The fuGFP ORF was also codon optimized for mammalian cell expression and had the following sequence; GTATCAAGTGGGGAGGACATATTCAGCGGTTTUGTGCCAATCCTTATCGAGTTGGAGGGAGATGTCAACGG TCATCGGTTTTCCGTAAGAGGGGAGGGATACGGCGATGCTCTAATGGGAAACTGGAAATCAAATTCATCT GTACTACAGGGCGCCTGCCGGTGCCGTGGCCCACGCTGGTCACAACACTTTCCTACGGGGTCCAGTGCTTTG CAAAGTATCCGGAGCACATGAGACAGAACGATTTTTAAAAGTGCCATGCCCGACGGCTACGTTCAAGAG AGAACTATAAGTTTCAAAGAAGATGGAACATATAAAACAAGAGCGGAGGTCAAATTTGAAGGAGAAGCGC TCGTCAACAGAATTGATCTGAAAGGCCTGGAGTTAAAGAAGACGGTAATATTCTCGGACACAAACTGGAA TACTCCTTTAATTCCCACTATGTATATATAACTGCAGACAAAAACCGCAACGGTCTTGAGGCCCAATTCCGC ATCAGACATAATGTAGACGATGGTAGCGTCCAACTGGCGGACCACTATCAACAGAACACGCCCATTGGGGA GGGTCCAGTCCTCTTGCCCGAACAACACTACTTGACAACCAACAGTGTCCTCTCAAAGGATCCCCAGGAAC GCCGGGACCACATGGTCCTGGTAGAGTTCGTAACTGCTGCGGGCCTGAGCCTTGGGATGGATGAACTTTAT AAATCTTAA (SEQ ID NO: 134). The fuGFP ORF was followed by the sequence GGTACCTAGTAGTCCGGACTCAGATAGTCTCGAGGACGGGGTGAACTACGCCTGAGGATCC(SEQ ID NO: 135) and then the polyadenylation site 3′ UTR of rabbit beta-globin (GenBank V00878.1) region 1272 to 1399. This polyadenylation site region was followed by the sequence TTAGGTAGATAAGTAGCATGGCGGGTTAATCATTAACTACAAGGAACCCCTAGTGATGGAGCTTGGC(SEQ ID NO: 136) and then the 3′ ITR containing sequence corresponding to region 4535 to 4664 of AAV2 (AF043303.1). This AAV sequence was followed by the sequence, CTGCCTGCAGGCCITAATTAAGCAAGCTGTA (SEQ ID NO: 137). Much of the remaining part of the synthetic sequence was identical to pSR657 region 3978 to 2604 with exception of a T3851A mutation. This pSR657 region included a gentamycin resistance gene and a 255 bp Tn7R sequence corresponding to 13,844 to 14,068 of the Tn7 transposon (GenBank MN628641.1) The final part of the synthetic sequence in pUC57 was GGCGTAATCATGGTCATAGCGGATC (SEQ ID NO: 138).

### Production of Bacmid 1260 (AA656)

LacR-Rep-VP3-LacOVP1C^{ICeu}-LacOVP2^{FseI}-LacR^{AvrII} Bacmids were produced by incorporating the opgp64-polh-NLS-LacR-Rep-VP3 construct (SEQ ID NO: 31) from Plasmid 1259 into the Tn7L region of Bacmid 1186 using standard Tn7 helper plasmids and cloning procedures known in the art. The resulting bacmids were then gel purified and bacterial colonies were grown and tested for opgp64-polh-NLS-LacR-Rep-VP3 insertion into the 1186 Bacmid. Bacterial colony 1260 was selected for further validation and testing.

Colony 1260 was subjected to Colony PCR screening using three combinations of two primers: (i) Primer JS95-LacR-RP1 (SEQ ID NO: 29) and Primer JS42-Rep78-RP_backwards (SEQ ID NO: 30), with positive PCR results having a target of about 1037 bp based on the primers used; (ii) Primer JS124-gta-LP10 (SEQ ID NO: 25) and Primer JS92-AAP-RP1 (SEQ ID NO: 22), with positive PCR results having a target of about 1199 bp based on the primers used; and (iii) Primer JS17-gp64UTR-RP (SEQ ID NO: 20) and Primer JS92-AAP-RP1 (SEQ ID NO: 22), with positive PCR results having a target of about 1092 bp based on the primers used.

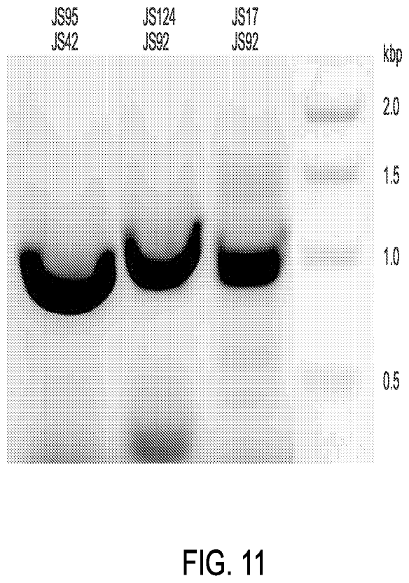

Results of JS95-JS42, JS124-JS92 and JS17-JS92 PCR screening of Colony 1260 are shown in FIG. 11. PCR screening showed that Colony 1260 (FIG. 11) had strong bands around 1037 bp (first column), 1199 bp (second column), and 1092 bp (third column) indicating likely insertion of opgp64-polh-NLS-LacR-Rep-VP3 into the 1186 Bacmid of those bacterial colonies.

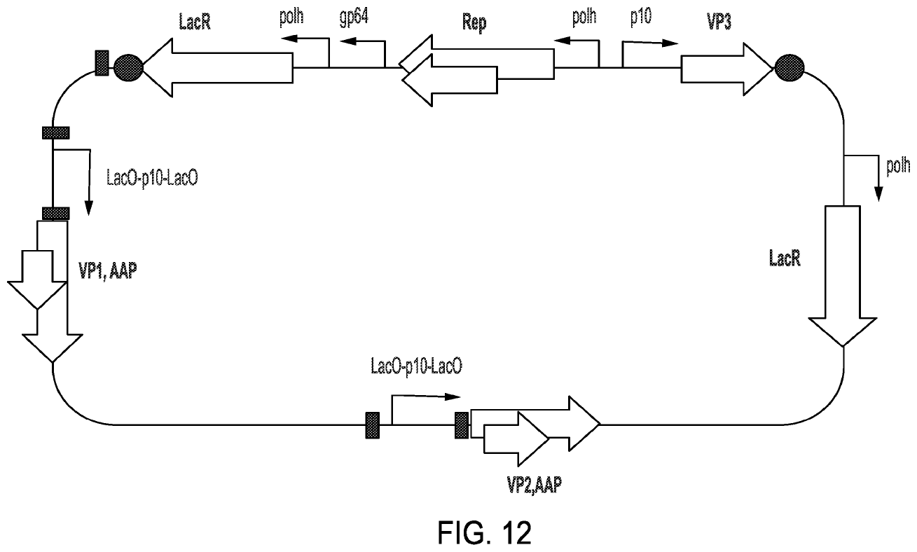

A graphical representation of certain components and coding regions in Bacmid 1260 is presented in FIG. 12, including (i) opgp64-polh-NLS-LacR region, (ii) Rep coding region under a polh promoter; (iii) VP3-only coding region under a p10 promoter, (iv) LacOVP^{ICeu} region (v) LacOVP2^{FseI} region, and (vi) LacR^{AvrII} region under a polh promoter.

### Testing AA656 (Bacmid1260)

### BIIC Coinfection Ratio Study

Samples of Bacmid1260 (AA656) were grown for further study and testing. sf9 cells were expanded in large culture up to a VPC density of about 3×10⁶ vc/mL, and then seeded into eighteen separate flasks at 10 mL working volume. AA654_ITR-SEAP-GFP payloadBIIC material and AA656 expressionBIIC material were prepared according to Table 1.

TABLE 1
BIIC preparations for Bacmid AA656 testing

| | | BIIC | ESF Media | Total |
|---|---|---|---|---|
| | | Volume | Volume | Volume |
| Material | TCID50/mL | (μl) | (μL) | (μL) |
| AA654 | 3.0 × 10⁷ | 58 | 4942 | 5000 |
| AA656 | 3.0 × 10⁷ | 96 | 4904 | 5000 |

Sf9 cells were then co-infected with payloadBIIC material (AA654) and expressionBac material (AA656) at different co-infection ratios in the presence of 0 uM or 200 uM IPTG, according to the conditions in Table 2.

TABLE 2
BIIC Ratio characterization of Bacmid AA656

| | payloadBIIC/ | AA654 | AA656 | |
|---|---|---|---|---|
| | expressionBIIC | (μL) | (μL) | IPTG |
| Culture ID | Ratio | [TCID50/cell] | [TCID50/cell] | (μM) |
| 1 | 9:1 | 90 [0.09] | 10 [0.01] | 200 |
| 2 | 6:1 | 60 [0.06] | 10 [0.01] | 200 |
| 3 | 3:1 | 30 [0.03] | 10 [0.01] | 200 |
| 4 | 1:1 | 10 [0.01] | 10 [0.01] | 200 |
| 5 | 1:3 | 10 [0.01] | 30 [0.03] | 200 |
| 6 | 1:6 | 10 [0.01] | 60 [0.06] | 200 |
| 7 | 1:9 | 10 [0.01] | 90 [0.09] | 200 |
| 8 | 1:12 | 10 [0.01] | 120 [0.12] | 200 |
| 9 | 9:1 | 90 [0.09] | 10 [0.01] | 0 |
| 10 | 6:1 | 60 [0.06] | 10 [0.01] | 0 |
| 11 | 3:1 | 30 [0.03] | 10 [0.01] | 0 |
| 12 | 1:1 | 10 [0.01] | 10 [0.01] | 0 |
| 13 | 1:3 | 10 [0.01] | 30 [0.03] | 0 |
| 14 | 1:6 | 10 [0.01] | 60 [0.06] | 0 |
| 15 | 1:9 | 10 [0.01] | 90 [0.09] | 0 |
| 16 | 1:12 | 10 [0.01] | 120 [0.12] | 0 |
| 17 | 0:1 | — | 10 [0.01] | 0 |
| 18 | 1:0 | 10 [0.01] | — | 0 |

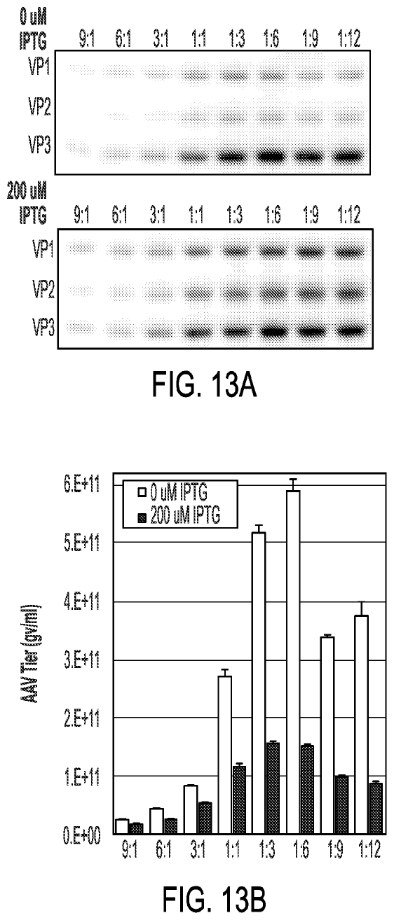

Cell lysate protein samples were collected from each culture sample, fractionated by SDS-PAGE, and then Western blot probed with anti-capsid antibody (FIG. 13A). Crude cell lysate titers for ITR-SEAP-GFP-ITR were also determined by Q-PCR (FIG. 13B). Results showed unexpectedly that payloadBIIC/expressionBIIC ratios between 1:1 to 1:12 provided increased VP protein production and increased AAV titer (vg/mL) compared to payloadBIIC/expressionBIIC ratios between 1:1 to 9:1.

### IPTG Concentration Study—Titer and Potency

Samples of Bacmid1260 (AA656) were grown for further study and testing. sf9 cells expanded in large culture up to a VPC density of about 3×10⁶ vc/mL, and then seeded into thirty separate flasks at 25 mL working volume (about 7.5×10⁷ vc/flask).

ITR-SEAP-GFP payloadBIIC material (AA654), AA656 expressionBIIC material, and Bac420 expressionBIIC material were prepared according to Table 3 (PHPN.Bac420 was used as Control comparison for AA656).

TABLE 3
BIIC preparations for Bacmid AA656 testing

| | | BIIC | ESF Media | Total |
|---|---|---|---|---|
| | | Volume | Volume | Volume |
| Material | TCID50/mL | (μl) | (μL) | (μL) |
| AA654 | 2.58 × 10⁹ | 12 | 225 | 237 |
| AA656 | 1.57 × 10⁹ | 46 | 871 | 917 |
| Bac420 | 3.29 × 10⁸ | 30 | 572 | 602 |

Sf9 cells were then co-infected with payloadBIIC material (AA654) and expressionBac material (AA656 or Bac420) according to the conditions in Table 4.

TABLE 4
IPTG characterization of Bacmid AA656

| | payloadBIIC/ | AA654 | AA656 | Bac420 | | |
|---|---|---|---|---|---|---|
| | expressionBIIC | (μL) | (μL) | (μL) | ESF | IPTG |
| Culture ID | Ratio | [MOI] | [MOI] | [MOI] | (μL) | (μM) |
| 1 | 1:0 | 6 [0.01] | — | — | 494 | 0 |
| 2 | 3:1 | 17 [0.03] | — | 46 [0.01] | 437 | 200 |
| 3 | 3:1 | 17 [0.03] | — | 46 [0.01] | 437 | 0 |
| 4 | 1:1 | 6 [0.01] | — | 46 [0.01] | 449 | 0 |
| 5 | 1:3 | 6 [0.01] | — | 137 [0.03] | 357 | 0 |
| 6 | 1:6 | 6 [0.01] | — | 247 [0.06] | 221 | 0 |
| 7 | 1:1 | 6 [0.01] | 10 [0.01] | — | 485 | 0 |
| 8 | 1:1 | 6 [0.01] | 10 [0.01] | — | 485 | 1 |
| 9 | 1:1 | 6 [0.01] | 10 [0.01] | — | 485 | 2 |
| 10 | 1:1 | 6 [0.01] | 10 [0.01] | — | 485 | 5 |
| 11 | 1:1 | 6 [0.01] | 10 [0.01] | — | 485 | 10 |
| 12 | 1:1 | 6 [0.01] | 10 [0.01] | — | 485 | 20 |
| 13 | 1:1 | 6 [0.01] | 10 [0.01] | — | 485 | 50 |
| 14 | 1:1 | 6 [0.01] | 10 [0.01] | — | 485 | 200 |
| 15 | 1:3 | 6 [0.01] | 29 [0.03] | — | 466 | 0 |
| 16 | 1:3 | 6 [0.01] | 29 [0.03] | — | 466 | 1 |
| 17 | 1:3 | 6 [0.01] | 29 [0.03] | — | 466 | 2 |
| 18 | 1:3 | 6 [0.01] | 29 [0.03] | — | 466 | 5 |
| 19 | 1:3 | 6 [0.01] | 29 [0.03] | — | 466 | 10 |
| 20 | 1:3 | 6 [0.01] | 29 [0.03] | — | 466 | 20 |
| 21 | 1:3 | 6 [0.01] | 29 [0.03] | — | 466 | 50 |
| 22 | 1:3 | 6 [0.01] | 29 [0.03] | — | 466 | 200 |
| 23 | 1:6 | 6 [0.01] | 57 [0.06] | — | 437 | 0 |
| 24 | 1:6 | 6 [0.01] | 57 [0.06] | — | 437 | 1 |
| 25 | 1:6 | 6 [0.01] | 57 [0.06] | — | 437 | 2 |
| 26 | 1:6 | 6 [0.01] | 57 [0.06] | — | 437 | 5 |
| 27 | 1:6 | 6 [0.01] | 57 [0.06] | — | 437 | 10 |
| 28 | 1:6 | 6 [0.01] | 57 [0.06] | — | 437 | 20 |
| 29 | 1:6 | 6 [0.01] | 57 [0.06] | — | 437 | 50 |
| 30 | 1:6 | 6 [0.01] | 57 [0.06] | — | 437 | 200 |

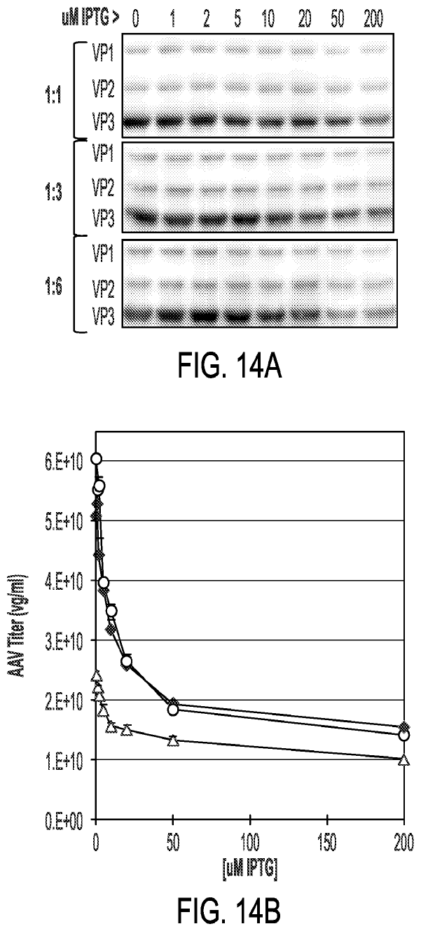

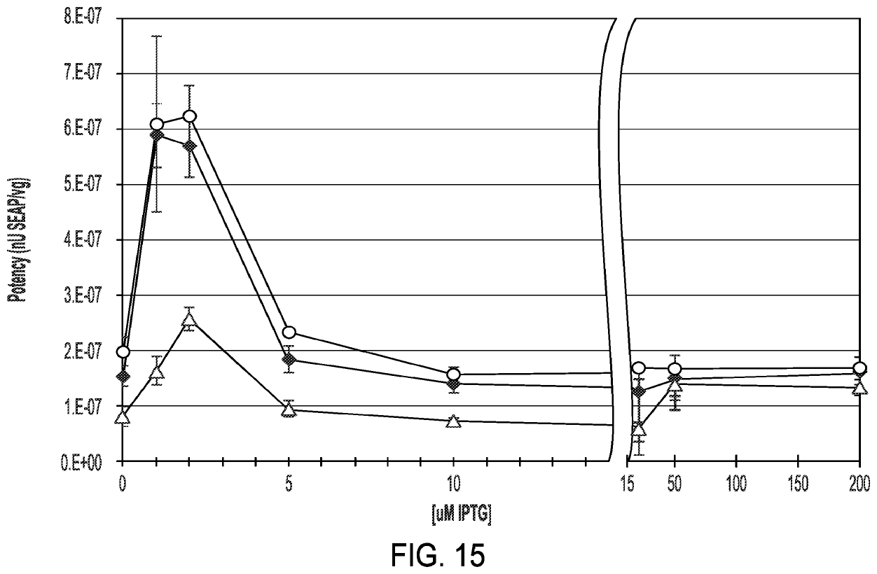

Cell lysate samples were collected from each culture sample, processed and pelleted through sucrose cushion ultracentrifugation, fractionated by SDS-PAGE, and then Western blot probed with anti-capsid antibody (FIG. 14A). Clarified cell lysate titers for ITR-SEAP-GFP-ITR were also determined by Q-PCR (FIG. 14B) [1:1 (open triangle), 1:3 (black diamond) and 1:6 (open circle)]. Purified AAV samples from the clarified cell lysate were collected, transduced onto 293 HEK cells, and alkaline phosphatase activity (i.e., potency) was measured relative to AAV sample genome titer (FIG. 15) [1:1 (open triangle), 1:3 (black diamond) and 1:6 (open circle)].

Results showed that payloadBIIC/expressionBIIC ratios between 1:3 to 1:6 provided increased AAV titer (vg/mL) and SEAP payload potency (nU SEAP/vg) compared to a payloadBIIC/expressionBIIC ratio of 1:1.

Results also showed that expressionBIIC ratios between 1:1 to 1:6 provided highest AAV titer results (vg/mL) at IPTG concentrations between 0.0 to 2.0 μM, and highest SEAP payload potency (nU SEAP/vg) at IPTG concentrations between 1.0 to 2.0 μM.

### Results

### Separately Expressing VP1, VP2 and VP3 ORFs was an Alternative Approach to Addressing the Complexity of Modulating Translation Context of Three Overlapping VP ORFs

Potent rAAVs were previously produced based on AAV1 and AAV2 serotypes in insect cells using baculovirus constructs based on the Smith design (Smith et al., Mol Ther., 17(11):1888-96 (2009), which is hereby incorporated by reference in its entirety). When this same design was used to express AAV9 serotype based AAV PHPN, the resulting product was less potent than AAV1 and AAV2 serotypes. The problem was identified to be deficient abundances of VP1 and VP2 relative to VP3. VP1:VP2:VP3 ratios were typically 1:1:15 instead of the desired 1:1:10 ratio. Initial approaches to remedy this VP1 and VP2 deficiency included inserting 5′ UTR stem loop structures upstream of VP1, changing the VP1 translational initiation context (Kondratov et al., Mol Ther., 25(12):2661-2675 (2017), which is hereby incorporated by reference in its entirety) and changing the translational context downstream of the translational initiation site of VP1 as described by Urabe (Urabe et al., Hum Gene Ther., 13(16):1935-43 (2002), which is hereby incorporated by reference in its entirety). These approaches were able to change abundances of VP1 and VP2 relative to VP3, but they were not readily controllable. The BEV system is not limited for foreign gene capacity, and this led to the idea of taking advantage of the BEV system's versatility and separating expression of VP1, VP2 and VP3 ORFs thus escaping the complexity of regulating translation of the overlapping capsid VP ORFs.

Nonconventional Recombinant BEV Cloning Methods Enabled Construction of a Single Baculovirus with Separately Cloned Rep, VP1, VP2, VP3 Gene Cassettes

Recombinant BEV based rAAV production platforms have only been reported using commercial BEV cloning kits using either homologous recombination (Kitts et al., Nucleic Acids Res., 18(19):5667-72 (1990), which is hereby incorporated by reference in its entirety) or Tn7 bacmid transposition (Luckow et al., J Virol., 67(8):4566-79 (1993), which is hereby incorporated by reference in its entirety). Foreign gene insertion is limited to a single location in the polyhedrin locus of the baculovirus genome. The VP1, VP2 and VP3 ORFs have common sequences that would be prone to recombination with each other if cloned into the same location. Also, large foreign multigene cassettes are not stable over passage in the baculovirus (Wu et al., Mol Ther Methods Clin Dev., 10:38-47 (2018), which is hereby incorporated by reference in its entirety). One option would have been to make separate recombinant baculoviruses for VP1, VP2 and VP3 and then optimize VP ratios by co-infection as was done by Ruffing (Ruffing et al., J Virol., 66(12):6922-30 (1992), which is hereby incorporated by reference in its entirety). This process was found to be impractical as it required optimization of the co-infection of five BEVs if one includes separate Rep BEV and an ITR BEV. Even co-infecting three recombinant BEVs leads to a product with low in potency (Urabe et al., Hum Gene Ther., 13(16):1935-43 (2002), which is hereby incorporated by reference in its entirety). Another option was to make insect cell lines that produced components of the RepCap baculovirus when infected by a BEV as was done by Mietzsch et al., Hum Gene Ther., 25(3):212-22 (2014), which is hereby incorporated by reference in its entirety. However, the selection and amplification of stable insect cell line clones is too long a process and not versatile for the production of multiple capsid serotypes. BEV cloning technologies have been developed which allow foreign gene insertions into other locations in the baculovirus genome (Lihoradova et al., J Virol Methods., 140(1-2):59-65 (2006); Sari et al., Advanced Technologies for Protein Complex Production and Characterization, 896: 199-215 (2016), which are hereby incorporated by reference in their entirety). The Bac375 baculovirus cloning vector was developed for rAAV production and used it as a unique backbone to clone foreign genes into four distinct locations in the baculovirus genome either by Tn7 transposition or by restriction endonuclease T4 ligase cloning (FIG. 1B).

LacR Regulatable Very Late p10 Promoters were Designed with Two lacO Elements to Enable Maximal Repression of VP1 and VP2

Baculoviruses encode for 156 genes that are expressed in a temporal cascade of early, late, and very late phases (Chen et al., J Virol., 87(11):6391-405 (2013), which is hereby incorporated by reference in its entirety). Only the genes, polyhedrin (polh) and p10 are expressed in the very late phase and their hyperexpression represents 24% and 7.5% of total cell mRNA transcripts respectively (Chen et al., J Virol., 87(11):6391-405 (2013), which is hereby incorporated by reference in its entirety). To ensure abundant rAAV capsid production, recombinant BEVs are engineered with polh and p10 promoters driving Rep and Cap gene expression, respectively. The lac inducible vaccinia virus expression system placed a single lacO sequence between promoter and translation start site to regulate gene expression (Fuerst et al., Proc Natl Acad Sci USA., 86(8):2549-53 (1989); Wyatt et al., mBio., 8(3):e00790-17 (2017), which are hereby incorporated by reference in their entirety). Transcription from very late baculovirus promoters is much greater than transcription from the vaccina expression system and a more robust lacO promoter design was needed. The LacR protein is a tetramer that binds to two lacO sequences simultaneously (Oehler et al., EMBO J., 9(4):973-9 (1990), which is hereby incorporated by reference in its entirety). In the E. coli lac operon, there are lacO upstream and downstream of inducible promoters and this double lacO configuration was found to be better for transcription repression than a single lacO (Oehler et al., EMBO J., 9(4):973-9 (1990), which is hereby incorporated by reference in its entirety). For lacOVP1 and lacOVP2 constructs, lacO was placed on either side of p10 promoters spaced 188 bp apart to create lacO-p10-lacO promoters (FIG. 1D).

LacR was Confirmed to be Able to Regulate Expression of a lacOVP1 Construct in the Context of Baculovirus Infection

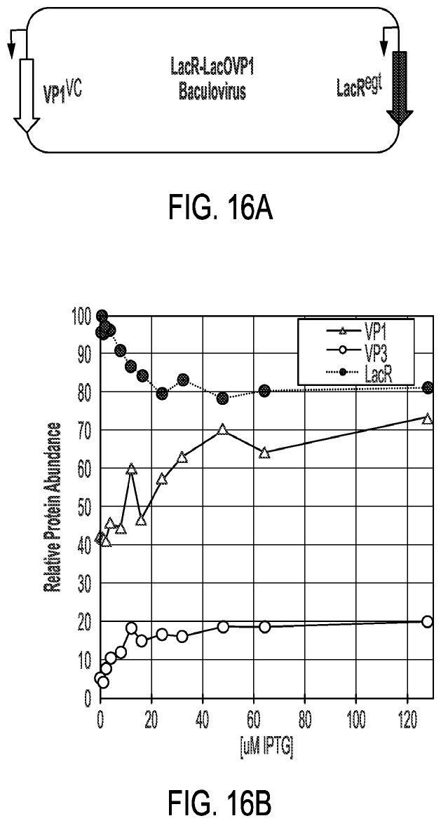

No published examples were found that apply LacR to regulate very late baculovirus promoters in the context of baculovirus infection. The Slack & Blissard study described only LacR regulation of plasmids transiently transfected into Sf9 cell (Slack et al., J Virol., 71(12):9579-87 (1997), which is hereby incorporated by reference in its entirety). It was thus important to make an initial recombinant BEV construct which confirmed the ability of LacR to regulate a very late baculovirus promoter. Very late promoter hyper expression occurs after baculovirus DNA replication when there are many very late promoters on multiple genomes available for baculovirus transcription factors to access. The LacR protein needed to be made abundantly enough to saturate the many very late promoters containing lacO regulation sites. The first inducible construct made was LacR-lacOVP1 (FIG. 16A), a baculovirus designed to test LacR's ability to repress VP1 under control of a lacO-p10-lacO promoter. The LacR gene in the egt locus was expressed from a polh promoter (FIG. 1F) and VP1 gene was in the v-cath locus under a lacO-p10-lacO promoter (FIG. 1D). The LacR-lacOVP1 baculovirus was evaluated for regulation of VP1 expression under different IPTG concentrations (FIG. 16). A gradient of VP1 expression proportional to IPTG concentration was observed. VP1 expression was maximal at 50 uM IPTG and minimal at 0 uM IPTG and the range repression on this baculovirus construct was about 50% based on ECL Western signal. This shallow repression by LacR was attributed to the strength of baculovirus very late promoter hyper expression. The goal of this regulation system was to modulate the expression of VP1 and VP2 relative to VP3. Inability to attain complete repression of lacOVP1 was not of concern because VP1 expression is essential for functional rAAV capsids to be made. In Western blots, it was noticed that there was a 60.6 kDa protein in addition to the expected 82.1 kDa VP1 protein. It was concluded this 60.6 kDa protein was from leaking translational scanning and resulting translation of VP3 ORF inside the VP1 ORF. The 60.6 kDa protein abundance had the same response to IPTG as did VP1. No translation products for the 67.0 kDa VP2 protein were seen. It was unexpected to see LacR protein abundance correlating inversely to IPTG concentration. This observation could be explained by competition for very late baculovirus transcription factors as IPTG induction released LacR repression of the lacO-p10-lacO promoter controlling VP1 expression.

### Time Course of Baculovirus Infection in Sf9 Cells Reveals Shifting VP Ratios in Inducible System

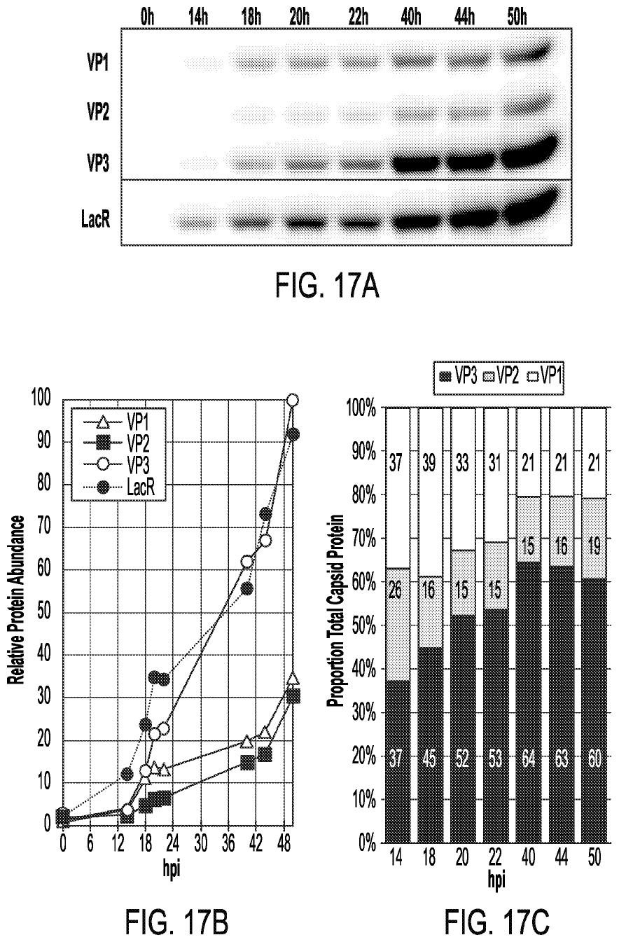

After verifying that LacR could regulate the expression from a lacO-p10-lacO promoter driving expression of VP1, the complete RepLacCap baculovirus was constructed with VP1 and VP2 separated under lacO-p10-lacO promoters, VP3 under a p10 promoter and Rep under a polh promoter (FIG. 1B). Baculovirus very late gene transcription increases 70-fold between 12 h and 24 h post infection as baculovirus DNA replication subsides (Chen et al., J Virol., 87(11):6391-405 (2013), which is hereby incorporated by reference in its entirety). During the rapid onset of very late gene transcription, it would be difficult for LacR to be initially present in sufficient abundance to repress lacO-p10-lacO promoters. To enhance LacR regulation, a second copy of the LacR was included in this baculovirus. The second LacR gene was designed with a hybrid early/late/very late baculovirus promoter to increase LacR abundance prior to the burst of very late gene promoter transcription (FIG. 1G). The hybrid promoter early/late region was a 166 base pair promoter of the OpMNPV gp64 gene (Blissard et al., J Virol. 65(11):5820-7 (1991), which is hereby incorporated by reference in its entirety) and the very late part of the promoter was a modified polh promoter lacking internal ATG start codons. A time course experiment was done to evaluate LacR and capsid VP protein production (FIG. 17). LacR expression at 14 hpi preceded very late promoter driven expression of VP1, VP2 and VP3 capsid proteins as expected. However, between 14 hpi and 24 hpi VP1 was more abundant than VP2 and similar in abundance to VP3 (FIG. 17C). The lacO-p10-lacO very late promoter of VP1 should have had the same temporal expression profile as VP2. This VP1 translation was attributed to leaky scanning translation of early and late baculovirus promoter mRNA transcripts which would not be affected by LacR. The lacOVP1 cassette was oriented downstream of the baculovirus early/late gp64 gene. Similar to the observed downstream leaky scanning translation of VP3 from lacOVP1 transcripts, there may be leaky scanning and downstream translation of VP1 from gp64 gene transcripts. The consequence of this is the loss of LacR regulation when this is occurring as the gp64 promoter does not have lacO sequences. The lacOVP2 gene was cloned downstream of the baculovirus gta gene which has a promoter that is 50-fold less transcribed than the gp64 promoter (Chen et al., J Virol., 87(11):6391-405 (2013), which is hereby incorporated by reference in its entirety). This may explain why VP2 less abundant a times prior to the onset of very late transcription. At least 75% of the VP1 and VP2 capsid proteins were made after 40 hpi when only very late gene transcription would be expected to be happening. It was decided to continue working with the current construct design with the acknowledgment that the cumulative capsid ratios seen in Western blots had imperfect homogeneity over the life cycle of baculovirus infection.

There is Competition Among Very Late Promoters as IPTG Induces LacR Repression of lacO Regulated Very Late Promoters

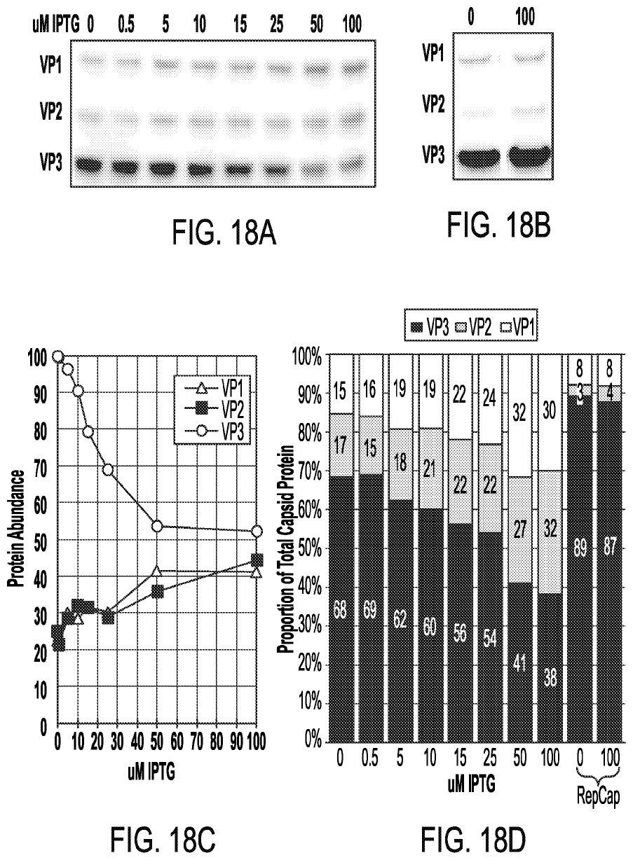

After the time course expression experiment, the RepLacCap baculovirus was titrated for IPTG induction of lacOVP1 and lacOVP2 expression (FIG. 18). As with the LacR-lacOVP1 baculovirus, the responsive concentrations of IPTG were between 0 uM and 50 uM. As expected, VP1 and VP2 abundances increased proportionally in response to IPTG induction of LacR repression of lacO-p10-lacO promoters. Estimated VP1:VP2:VP3 capsid ratios in infected Sf9 cells ranged of ranged from 15:17:68 at 0 uM IPTG to 30:32:38 at 50 uM and 100 uM IPTG. Capsid VP ratios produced in RepCap baculovirus infected Sf9 cells were unaffected by IPTG and VP1:VP2:VP3 ratios were 8:3:89 and 8:4:87 at 0 uM and 100 uM IPTG respectively. VP1 and VP2 expression was not able to be repressed enough to obtain a 1:1:10 ratio. When VP1 and VP2 became more abundant, there was an unexpected reciprocal drop in VP3 abundance. This can be explained by the lacO-p10-lacO promoters for VP1 and VP2 drawing away very late transcription factors from the p10 promoter driving expression of VP3.

### Increasing Abundances of VP1 and VP2 LED to Reduced Capsid Titers at Three ITR:LacRepCap Baculovirus Co-Infection Ratios

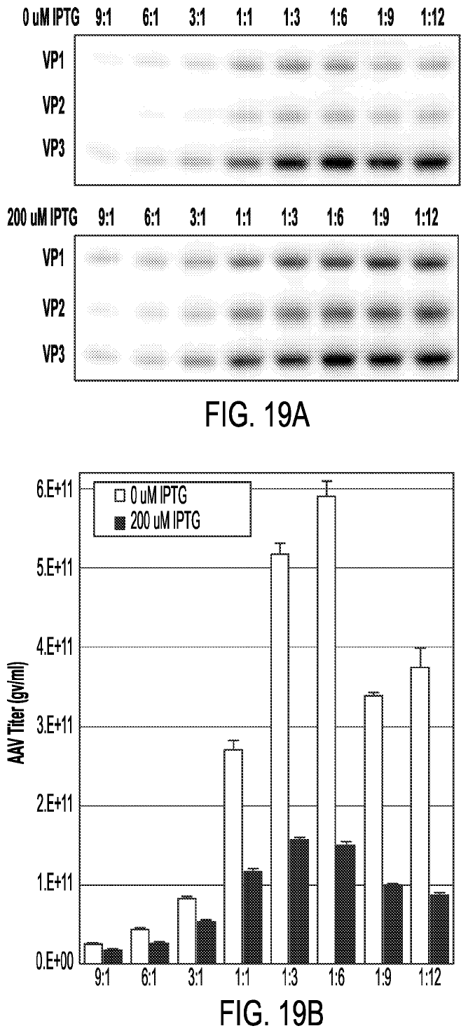

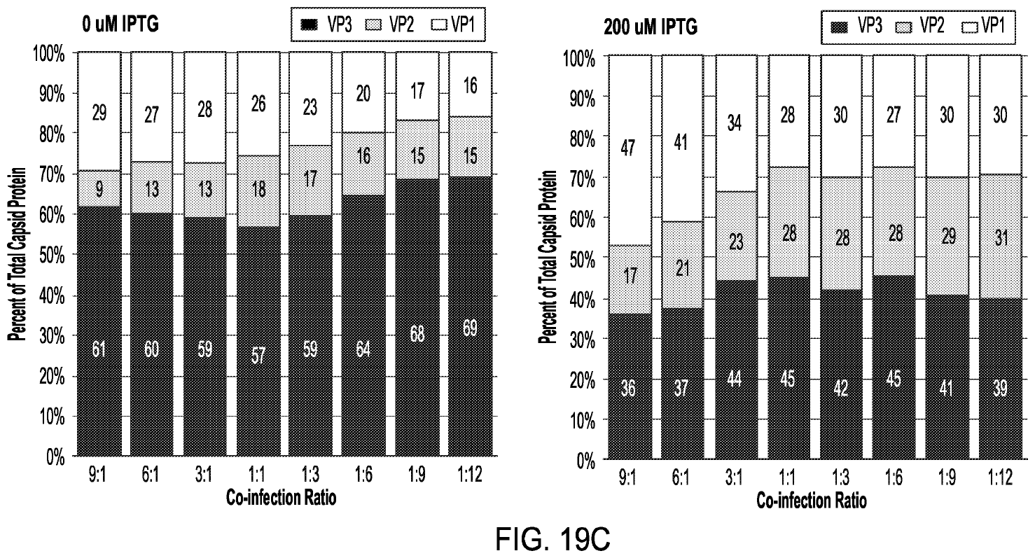

After determining IPTG concentrations for the regulation of capsid ratios, co-infection ratios for the LacRepCap baculovirus with a transgene carrying ITR-SEAP-GFP baculovirus were then optimized. Sf9 cells were co-infected with ITR-GFP-SEAP and LacRepCap baculoviruses at co-infection ratios ranging from 9:1 to 1:12. In these 25 ml scale shake flask experiments, initial infection MOIs ranged from 0.01 to 0.12 TCID50 units per cell with for example the 9:1 ratio co-infection receiving 0.09 TCID50 units ITR baculovirus and 0.01 TCID50 units of LacRepCap baculovirus per cell. Low MOI infections were done to better emulate larger scale production of rAAV in the BEV Sf9 system. For the non-Lac regulated ITR:RepCap baculovirus the optimal co-infection ratio is often 3:1. It was surprising to find that the 3:1 co-infection ratio of ITR:LacRepCap produced a low titer rAAV compared to ratios as high as 1:12 (FIG. 19B). It is possible that the LacRepCap baculovirus was replicating more slowly but it had a similar BV growth curve as the RepCap baculovirus. Another explanation for needing more of the LacRepCap baculovirus could be a reduced abundance of Rep protein in the LacRepCap baculovirus due LacR and VP expression drawing away very late baculovirus transcription factors from the polh promoter driving Rep expression. Western blot analysis for Rep expression did not indicate deficiency of Rep expression in the LacRepCap baculovirus compared to Rep expression from the RepCap baculovirus. Higher ITR:RepCap co-infection ratios of 9:1, 6:1 and 3:1 also produced more abundant VP1 expression relative to VP2 expression. This resembled the overabundance of VP1 that was observed in the time course experiment of LacRepCap at 18 hpi and 22 hpi and could also be attributed to VP1 translation from gp64 gene transcripts (FIG. 17). In this case, the excessive ITR baculoviruses co-infecting Sf9 cells with LacRepCap baculoviruses were providing more early and late transcription factors to express the gp64 gene upstream of lacOVP1. Despite the imbalance in VP1 and VP2 at high co-infection ratios, there was still Lac regulated control of VP ratios. Under all co-infection conditions, rAAV titers were higher when VP1 and VP2 expression was repressed by LacR.

A High Throughput Method was Developed to Purify rAAV from BEV Infected Sf9 Cells

This inducible system required optimizing both IPTG concentrations and ITR:LacRepCap baculovirus co-infection ratios for rAAV potency on HEK 293T cells. Screening of IPTG concentrations and co-infection conditions often required 25 to 30 samples per experiment. Affinity purification and iodixanol discontinuous gradient ultracentrifugation purification (Buclez et al., Mol Ther Methods Clin Dev., (3)16035:1-10 (2016), which is hereby incorporated by reference in its entirety) of AAV capsid was also found to be too labor intensive and time consuming. A simple, fast and reproducible method of rAAV purification was developed based on a single step sucrose cushion ultracentrifugation (Chen et al., J Virol. Methods 281:113863 (2020), which is hereby incorporated by reference in its entirety). A rapid and consistent recovery of pelleted rAAV particles was achieved through 20% sucrose after only 1 h of ultracentrifugation. No difference in yields of empty and full capsids was observed using this method. The soluble fractions of PBS resuspended sucrose cushion pellets were found to be suitable for HEK 293T cell transduction assays and did not require buffer exchange. This method allowed 30 samples to be processed in one day on one centrifuge. Control empty capsids were found to purify through 20% sucrose cushions at similar abundance to DNA containing capsids produced in presence of ITR transgenes. rAAV Q-PCR titers suggest that only about 1% of total capsids in cell lysates were being recovered after 1 h of ultracentrifugation but this was sufficient to carry out several 96-well plate HEK 293T transduction assays. When rAAV capsids were purified by affinity purification, the yields were much higher and subjecting those samples to sucrose cushion purification reduced the recoverable yield. Affinity and affinity/sucrose cushion purified capsids had the same VP ratios and relative potencies on HEK 293T cells.

The only problem observed with purifying rAAV capsids using only the sucrose cushion was that baculovirus capsids co-purified. When these samples were used to transduce HEK 293T cells, there was a SEAP background from the ITR-SEAP BEV control group which was not detected in the RepCapLac BEV alone control group. Initially, it was thought the background SEAP activity was coming from baculovirus virion transduction of HEK 239T cells. However, the baculovirus envelope fusion protein GP64 could not detect by Western blot and the background SEAP activity did not diminish after lysate treatments with higher concentrations of detergents. Later it was noticed that the SEAP background arose from the sucrose cushion samples used for transduction assays. It was discovered that there was SEAP expression by the baculovirus in Sf9 cells from the mammalian CMV promoter in the ITR-SEAP BEV construct. Resulting SEAP enzyme may be associated with baculovirus capsids. The baculovirus capsid protein ORF1629 has an affinity for phosphatase enzymes (Katsuma et al., PLoS Pathog., 8(4):e1002644 (2012), which is hereby incorporated by reference in its entirety) and the SEAP expressed in Sf9 cells likely co-purified with baculovirus capsids. The ITR-SEAP alone control SEAP activities were subtracted as background from all ITR:RepCapLac co-infection groups. The background SEAP activity in purified rAAV samples also disappeared after affinity purification.

Co-Infection of ITR:LacRepCap Baculoviruses at Three Ratios Showed Decline in Assembled Capsids as VP1 and VP2 Abundances were Increased Relative to VP3

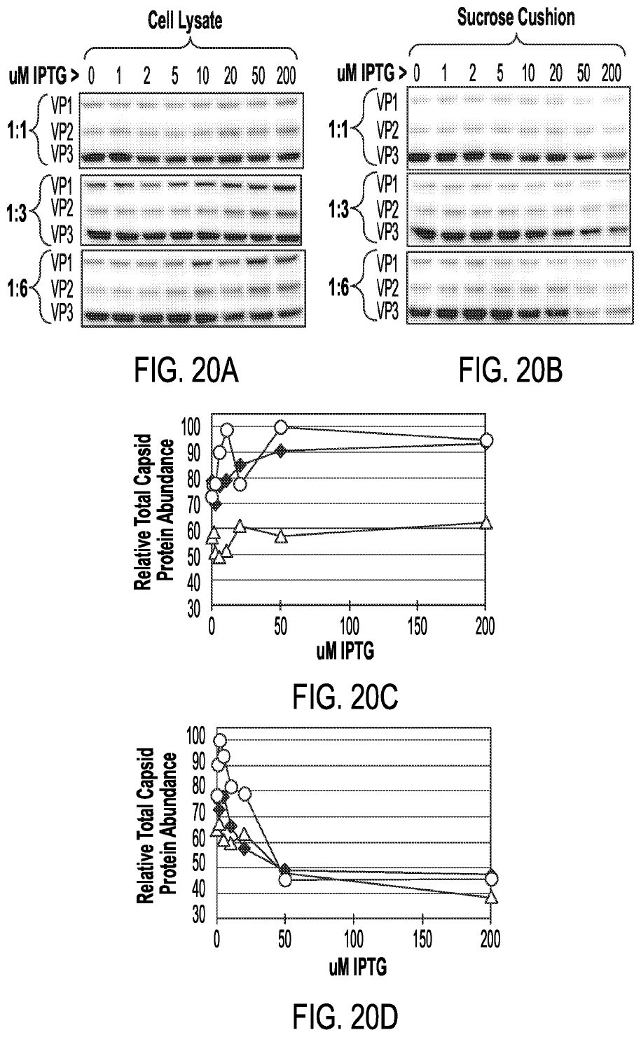

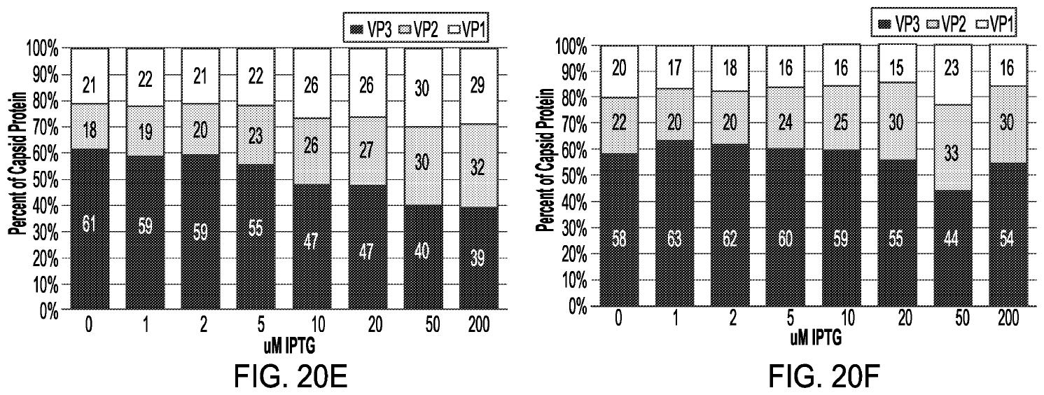

The ITR:LacRepCap co-infection ratios of 1:1, 1:3 and 1:6 were selected for further optimization with IPTG concentration. Twenty-five ml scale ITR:LacRepCap co-infections of Sf9 cells were done at eight different IPTG concentrations ranging from 0 uM to 200 uM and the rAAV capsids were purified by 20% sucrose cushion from the cell lysates of baculovirus infected insect cells. Western blots were used to detected capsid proteins in cell lysates and in the corresponding sucrose cushion pellets (FIG. 20). Total capsid proteins present in cell lysates was only changed marginally as IPTG concentrations went up. The 1:1 co-infection ratio cell lysate had lower total capsid protein yield relative to the 1:3 and 1:6 co-infection ratios. Total capsid protein abundance in sucrose cushion purified samples was more affected over varying IPTG concentrations. Increasing the expression of VP1 and VP2 relative to VP3 reduced the yield of assembled capsids passing through the sucrose cushion. For all three co-infection groups there was a drop in the abundance of total capsid proteins as the IPTG concentration was increased. The capsid ratios in cell lysates differed from the capsid ratios in corresponding sucrose cushion samples. (FIGS. 20E and 20F). Interestingly, the abundance of VP1 in sucrose cushion samples relative to VP2 and VP3 was more constant over the 0 to 200 uM IPTG compared to the cell lysate samples. There appears to be a limit to the amount of VP1 that can be incorporated into assembled rAAV capsids and less of a strict limit for VP2 incorporation into capsids.

The Potency of rAAV Capsids was Tunable Using the Lac Inducible System in Small Scale Productions

Potency was determined by transducing HEK 293T cells with rAAV capsids containing the ITR-SEAP-GFP reporter gene. Measured SEAP reporter activity from transduced HEK 293T cells and expressing that activity relative to rAAV titer (FIG. 8). There was a drop in the rAAV titer of sucrose cushion purified capsids with at increased IPTG concentrations (FIG. 8A). This was expected given the lower abundance of capsid proteins in sucrose cushion samples as IPTG concentrations increased (FIG. 20D). Potency did not follow capsid abundance and capsid titer which both peaked at 0 uM IPTG. Instead, potency was highest between 1 uM and 2 uM IPTG for all three co-infection groups (FIG. 8B). Potency dropped by six-fold at 0 uM IPTG and at 5 uM IPTG. Western blots did show significant differences in VP ratios between 0 uM and 5 uM IPTG. It is possible that there were different ratios over the baculovirus infection cycle which cannot be observed at the cumulative final harvest time point.

Larger Scale Production of rAAV in Sf9 Cells Showed that there was Preferential Lac Regulation of VP2 Expression

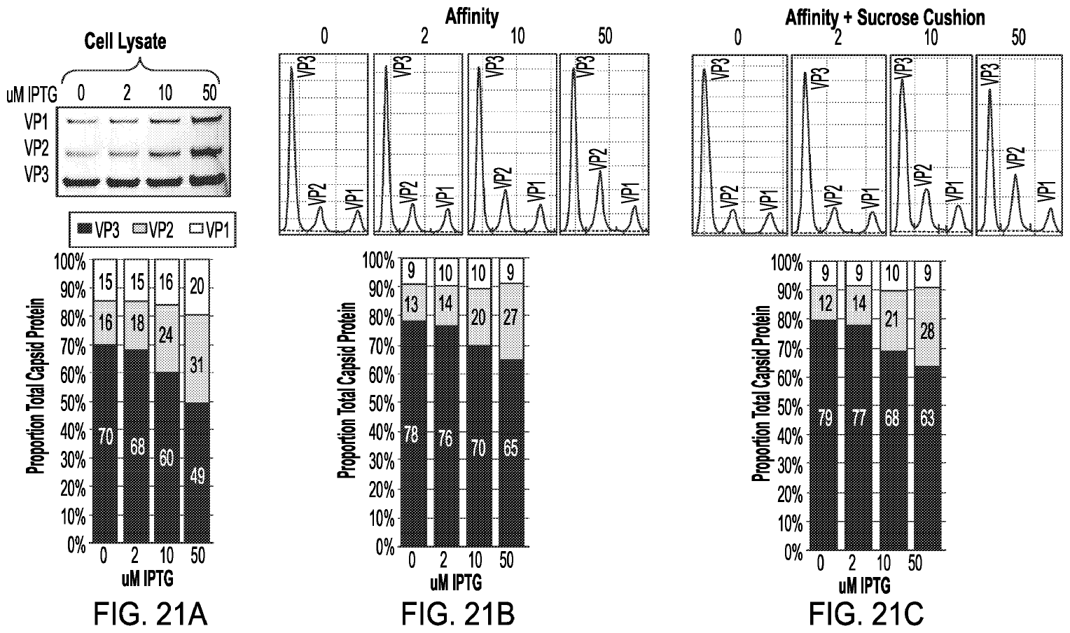

Data presented thus far was generated from sucrose cushion purified rAAV samples generated at small 25 ml scale. A common method to produce rAAV for drug therapeutics involves larger scale Sf9 cultures and affinity chromatography-based purification (Mietzsch et al., Mol Ther Methods Clin Dev., 19:362-373 (2020), which is hereby incorporated by reference in its entirety). This was scaled up to 800 ml Sf9 cultures and co-infected these cells with ITR-SEAP-GFP and LacRepCap baculoviruses at 1:6 ratio (0.1:0.6 moi). These co-infections were done at four IPTG concentrations; 0 uM, 2 uM, 10 uM and 50 uM IPTG. Sf9 cell lysates made at this time were subjected to affinity chromatography to purify rAAV capsids. A portion of those affinity purified capsids were further purified by sucrose cushion ultracentrifugation. Expressed capsid VP ratios in cell lysates were determined by Western blot (FIG. 21A) and by CE-SDS for both affinity purified capsids (FIG. 21B) and affinity/sucrose cushion purified capsids (FIG. 21C). The most noticeable trend was the nearly constant proportion of VP1 in both affinity and affinity/sucrose cushion purified rAAV capsids regardless of the IPTG concentration. In contrast, the relative abundance of VP2 in purified capsids increased with IPTG concentration just as it did in cell lysates. These data show that capsid assembly is not stochastic with there being a limit to the amount of VP1 that can be incorporated into capsids.

### Larger Scale Production Did not Show Expected Optimal Potency at 2 uM IPTG

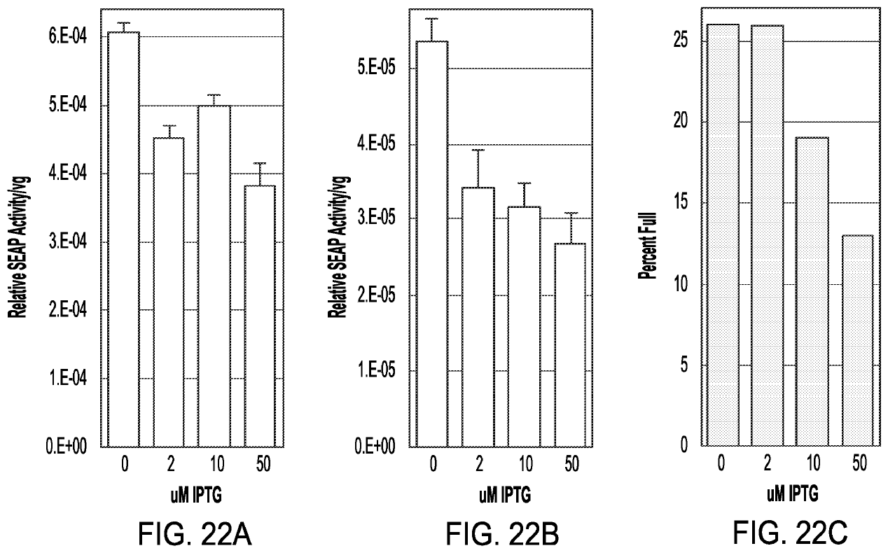

An increase in potency was not observed when capsids were produced in presence of 2 uM IPTG as was observed in smaller scale experiments (FIG. 8B). Instead, rAAV potency on HEK 293T cells was highest at 0 uM IPTG (FIGS. 22A and 22B). The percent of capsids containing ITR-SEAP-GFP transgenes in affinity purified samples was measured using SEC-MALS and was found highest at 0 uM IPTG and lowest at 50 uM IPTG (FIG. 22C).

### Discussion

The E. coli Lac repressor (LacR) was selected as a regulator of VP expression due to its success in other eukaryotic virus platforms such as Vaccinia (Fuerst et al., Proc Natl Acad Sci USA., 86(8):2549-53 (1989); Zhao et al., J Virol Methods., 160(1-2):101-10 (2009); Wyatt et al., mBio., 8(3):e00790-17 (2017), which are hereby incorporated by reference in their entirety) and adenoviruses (Matthews et al., J Gen Virol., 80 (Pt 2):345-353 (1999), which is hereby incorporated by reference in its entirety). LacR is a component of the Lac Operon, that was the first described inducible regulatory system (Jacob and Monod, J Mol Biol., 3:318-56 (1961), which is hereby incorporated by reference in its entirety) and which has been well characterized (For review see Lewis, M., C R Biol., 328(6):521-48 (2005), which is hereby incorporated by reference in its entirety) including being solved for crystal structure (Friedman et al., Science, 268(5218):1721-7 (1995), which is hereby incorporated by reference in its entirety). LacR was also shown to be functional in insect cells and was allosterically regulated (induced) by Isopropyl β-D-1-thiogalactopyranoside (IPTG) (Slack and Blissard, J Virol., 71(12):9579-87 (1997); Slack and Blissard., J Gen Virol., 82(Pt 10):2519-2529 (2001), which are hereby incorporated by reference in their entirety).

The present application demonstrates a novel method for expressing rAAV capsid VPs at desired ratios, including for example, neurospecific capsid AAV9-PHPN (Chan et al., Nat Neurosci., 20(8):1172-1179 (2017), which is hereby incorporated by reference in its entirety), in the BEV system. Instead of expressing VP1, VP2 and VP3 from a common ORF, VP1, VP2 and VP3 ORFs were separated to be expressed independently in a single BEV construct. The VP1 and VP2 ORFs were placed under the E. coli lac repressor (LacR) inducible regulation in the context of the BEV expression system. Controlling the abundances of VP1 and VP2 relative to VP3 affects the potency of rAAV capsids. A rapid method of purification of rAAV capsids suitable for mammalian cells transduction assays was also developed that enabled the screening of many conditions.

Described herein is a Lac repressor inducible system to empirically regulate the separated expression of VP1 and VP2 proteins relative to VP3 in the context of an expression vector (e.g., BEV). This example demonstrates the use of this system to tune the abundance, titer and potency of a rAAV9 serotype derivative called rAAV-PHPN. VP1:VP2:VP3 ratios of 1:1:8 gave optimal potency for this rAAV. It was discovered that ratios of capsid proteins expressed were different than ratios that ultimately were in purified capsids. Over expressed VP1 did not become incorporated into capsids and overabundance of VP2 correlated with reduced rAAV titers. This work demonstrates a novel technology for controlling production of rAAV in the BEV system and shows a new perspective on the biology of rAAV capsid assembly.

The present application discloses a method to regulate AAV capsid VP ratios and to improve the potency of capsids (e.g., rAAV9 serotype capsids) produced in the BEV system. The VP1, VP2 and VP3 ORFs were separately cloned and stably expressed from three different loci in the BEV genome. The E. coli LacR gene was also cloned and expressed into another unique loci into the BEV genome. This modular recombinant baculovirus design was stable and allowed for independent modifications of the various elements. Engineering the baculovirus p10 promoters of VP1 and VP2 genes with double LacO's enabled IPTG inducible LacR regulation. This is the first report of a very late “hyperexpressed” baculovirus promoter being placed under inducible regulation. Not surprisingly due to the strength of p10 transcription, the repression of VP1 and VP2 expression was not complete. Repression was sufficient for the intended purpose of regulating the expression of VP1 and VP2 relative to VP3 for successful AAV capsid assembly.

IPTG induction of LacR repression of VP1 and VP2 expression relative to VP3 was tunable such that the potency of resulting capsids could be optimized in small 25-ml scale Sf9 cultures. This did not translate to larger scale production with the maximum amount of repression have producing the most potent rAAV. A possible reason for this was that the co-infection ratios at larger scale needed to be further optimized. As shown in FIG. 8, having too little LacRepCap baculovirus relative to ITR baculovirus reduced the ability to tune potency with IPTG. At very least, this LacR based system without IPTG throttles VP1 and VP2 expression relative to VP3 expression in temporal synchrony with the very late baculovirus promoter.

There was consistent decline in total assembled capsids and rAAV titers as expressed VP1 and VP2 abundances is increased relative to VP3. The data agree with the findings of Gao that showed VP1 and VP2 abundances influenced capsid yield (Gao et al., Mol Ther Methods Clin Dev., 1(9):20139 (2014), which is hereby incorporated by reference in its entirety). The trend of expressed VP1 and VP2 abundance affecting yield was observed to be consistent with repeated experiments and with other rAAV9 capsid variants. The results contradict the findings of Bosma and show a decline in the percent full at IPTG induction as the resulting increased expression of VP1 and VP2 (FIG. 22C) (Bosma et al., Gene Ther., 25(6):415-424 (2018), which is hereby incorporated by reference in its entirety). A major difference in that study was that it used a rAAV5 serotype which does not require expression of the frame shifted AAP protein found nestled in the common VP1/VP2/VP3 ORFs.

The impact of AAP expression was not addressed as VP1 and VP2 expression was modulated by IPTG induction. Both VP1 and VP2 ORFs have complete copies of the AAP ORF. The canonical ATG start codons for VP1 and VP2 would likely lead to less translational scanning and thus less translation from the downstream non-canonical CTG start codon for AAP. However, as VP1 and VP2 transcription increased with IPTG induction, there would be more AAP produced as are result of leaking translational scanning.

Capsid ratio analysis methods like CE-SDS or Western blot used here show only the mean distribution of VP1, VP2 and VP3 in purified capsids. The VP ratios found in individual capsids is a random Poisson distribution of VP1, VP2 and VP3 dependent on starting abundances of VP's during assembly (Worner et al., Nature Methods, (4):395-398 (2020), which is hereby incorporated by reference in its entirety). To complicate this further, the baculovirus expression system does not offer a steady state expression of VP1, VP2 and VP3. Data from Smith et al., 2009 show VP ratios varying at 24 h, 48 h, 72 h and 96 h post infection. The decoupled VP1, VP2, VP3 design evaluated in this study also had different temporal expression of VP1, VP2 and VP3 over the time course of infection.

There was a difference between expressed VP ratios in Sf9 cells and the VP ratios that appeared in assembled capsids purified through sucrose cushion or by affinity purification. The data show that an upper limit to the amount of VP1 that which can be included in assembled capsids. This is best illustrated in FIG. 21 where crude cell lysate abundance of VP1 was 20% at 50 uM IPTG and was only 9% in the affinity/sucrose cushion purified capsids. At the same time, VP2 abundance in the cell lysate was 31% and was 27% in the affinity/sucrose cushion purified capsids. It is concluded that assembled capsids tolerate a higher abundance of VP2 compared to VP1. Overabundance of VP1 leads to reduced levels of capsid assembly and reduced potency. Under abundance of VP1 in capsids does not affect capsid assembly but does reduce capsid potency.

The lac inducible system presented here is an alternative way to manufacture rAAV therapeutic drug products in the BEV platform that differs significantly from the more standard BEV design. With the system of the present application, affinity purified rAAV were produced with potencies that are higher than from affinity purified rAAV capsids generated from the standard BEV design and also from triple plasmid transfected HEK 293T cells. Commonly, comparisons between BEV and HEK 293T platforms are done with rAAV particles that are enriched for full capsids by CsCl ultracentrifugation (Rumachik et al., Mol Ther Methods Clin Dev., 18:98-118 (2020), which is hereby incorporated by reference in its entirety). This difficult rAAV purification technique is not applicable to large scale cGMP drug production. Thus, the lac inducible system of the present application provides a realistic alternative to the standard BEV design.

In the current study, it was found that IPTG was well tolerated by Sf9 cells and were able to culture Sf9 cells in IPTG concentrations as high as 18 mM IPTG with no effect on cell growth. This concentration of IPTG was 1000-fold more than the working IPTG concentration range needed as our inducible system. After rAAV virions are purified from baculovirus infected insect cell lysates by affinity chromatography, IPTG would also not be expected to be present in the final drug product. The cost of the ESF AF insect cell culture media used in this study was $62/L. Animal free high purity IPTG (mw 238.3) cost $205 for 5 g (CAS 367-93-1 Calbiochem). Even at the highest foreseeable concentration of 100 uM IPTG, 24 mg of IPTG/L costing $0.98/L would be needed, creating a 1.6% increase in media cost. The optimal IPTG concentration for rAAV potency in this study was 2 uM IPTG thus cutting IPTG cost to 0.03% of media cost. There is no basis for IPTG being too costly or being too toxic at the working concentrations for this system. Finally, even if it were an issue, allolactose could be substituted for IPTG.

This new method to produce rAAV in the BEV platform based on Lac inducible expression of VP1 and VP2 capsid proteins enables the production of potent capsids with better VP ratios and is applicable to any capsid serotype.

### Example 2. Rep78-Only/Rep52-Only Cassettes

### Polh-Rep78-Only

Polynucleotide cassettes were engineered to include a polh promoter (very late) and a Rep78-only coding sequence (i.e., very little translation of Rep52 protein from Rep78 ORF). The polynucleotide was engineered to be inserted/cloned into suitable baculovirus plasmids or vectors using Gibson Assembly methods. A polh-Rep78-only polynucleotide (with Gibson Assembly sequences) is presented in SEQ ID NO: 32.
polh_MC_atgRep78

Polynucleotide cassettes were engineered to include a polh promoter (very late), a minicistron insert (SEQ ID NO: 4), and an atgRep78 (i.e., Rep78-only) coding sequence (see Table 5). Each polh_MC_atgRep78 cassette was engineered to be inserted/cloned into suitable baculovirus plasmids or vectors using Gibson Assembly methods. Table 5 presents further details of the polh_MC_atgRep78 constructs (“Proximity” in Table 5 represents the separation of the minicistron insert from the ATG start codon, i.e., number of intermediate nucleotide base pairs).

TABLE 5
polh_MC_atgRep78 Designs

| Construct | minicistron insert | minicistron insert | polh_MC_atgRep78 |
|---|---|---|---|
| Number | (SEQ ID NO) | proximity (bp) | (SEQ ID NO) |
| MC1 | 4 | 3 bp | 33 |
| MC2 | 4 | 20 bp | 34 |
| MC3 | 4 | 37 bp | 35 |
| MC4 | 4 | 54 bp | 36 |
| MC5 | 4 | 71 bp | 37 |

polh-Rep52-Only

Polynucleotide cassettes were engineered to include a polh promoter (very late) and a Rep52-only coding sequence (i.e., includes the entire coding sequence of the Rep52 protein from wild-type AAV2, but does not include the normally upstream coding sequence of the Rep78 protein). The polh promoter was included to drive the expression of the Rep52 coding sequence. The polynucleotide was engineered to be inserted/cloned into suitable baculovirus plasmids or vectors using Gibson Assembly methods. A polh-Rep52 polynucleotide (with Gibson Assembly sequences) is presented in SEQ ID NO: 38.

### Example 3. Production of Bacmid AA753

### Production of Bacmid AA742

A baculovirus genome (i.e., bacmid) which was a v-cath-inactivated variant from bMON14272 (Invitrogen Life Technologies) was provided (Bacmid AA737). The AA737 bacmid included: (i) a VP-sequence insertion scaffold (SEQ ID NO: 39) in the gta locus of the baculovirus genome, which included a LacO-p10-LacO expression control region (SEQ ID NO: 40); and (ii) a VP-sequence insertion scaffold (SEQ ID NO: 41) in the chiA locus of the baculovirus genome, which included a LacO-p10-LacO expression control region (SEQ ID NO: 40). The inclusion of the gta VP-sequence insertion scaffold (SEQ ID NO: 39) was confirmed by PCR amplification using primer set Lef12seq_F (SEQ ID NO: 42) and GTAseq_R (SEQ ID NO: 43), followed by confirmational sequencing of the PCR products. The inclusion of the chia VP-sequence insertion scaffold (SEQ ID NO: 41) was confirmed by PCR amplification using primer set ChiAseq_F (SEQ ID NO: 44) and gp64seq_R (SEQ ID NO: 45), followed by confirmational sequencing of the PCR products.

Plasmids which included a polh_MC1_atgRep78 sequence (SEQ ID NO: 33) were provided. Rep78-containing fragments were obtained by PCR amplification using primer set VP3scaff.f1_F (SEQ ID NO: 46) and VP3scaff.f1_R (SEQ ID NO: 47), followed by DpnI digestion of the PCR products for 1 hour at 37° C. to remove residual plasmid template elements, thereby providing polh_MC1_atgRep78 insert fragments which included the sequence of SEQ ID NO: 47. AMP gBlock insert fragments (which included a VP-sequence insertion scaffold) were also provided (SEQ ID NO: 49).

8.0 μg of bacmid DNA AA737 was digested with AscI enzyme in a 20 μL reaction at 37° C. for 2 hours, followed by heat-inactivated by incubating the reaction at 80° C. for 20 minutes. The polh_MC1_atgRep78 insert fragments and AMP gBlock insert fragments were then incorporated into the AscI-digested AA737 bacmid genomes by Gibson assembly reaction: 8 μL of AscI-digested AA737+1 μL of polh_MC1_atgRep78 insert fragments (30 ng/μL)+1 μL of AMP gBlock insert fragments (20 ng/μl). The mixture was incubated 50° C. for 2 hours, transformed into electrocompetent NEB10b E. Coli samples, and plated on KAN/CARB plates for colony expansion and screening.

Incorporation of the polh_MC1_atgRep78 insert and AMP gBlock insert into the AscI-digested AA737 bacmid was confirmed by PCR amplification of Bacmid Colony AA742 using primer set KANseq_F (SEQ ID NO: 50) and MiniFseq_R (SEQ ID NO: 51), followed by confirmational sequencing of the PCR products.

### Production of Bacmid AA745

8 μg of Bacmid AA742 was provided and digested with AvrII to provide AvrII-digested AA742 bacmids.

Plasmids which included a polh-atgRep52 sequence with Gibson Assembly sequences (SEQ ID NO: 38) were provided. polh-atgRep52-containing fragments were obtained by PCR amplification using primer set Rep52_AvrIINheIgibs_F (SEQ ID NO: 52) and Rep52_AvrIINheIgibs_R (SEQ ID NO: 53), thereby providing polh-atgRep52 insert fragments. The polh-atgRep52 insert fragments were then incorporated into the AvrII-digested AA742 bacmid genomes by Gibson assembly reaction: 9 μL of AvrII-digested AA742+1 μL of polh-atgRep52 insert fragments (30 ng/μL)+10 μL of Hifi Assembly Mix. The mixture was incubated 50° C. for 1 hour, transformed into electrocompetent NEB10b E. Coli samples, and plated on KAN plates for colony expansion and screening.

Incorporation of the polh-atgRep52 insert into the AvrII-digested AA742 bacmid was confirmed by PCR amplification of Bacmid Colony AA745 using primer set EGT_seq2_F (SEQ ID NO: 54) and EGT_seq2_R (SEQ ID NO: 55), followed by confirmational sequencing of the PCR products.

### Production of Bacmid AA749

Plasmids which included an I-SceI-flanked CAM-resistance cassette (SEQ ID NO: 56) were provided. I-SceI-flanked CAM-resistance fragments with Gibson Assembly sequences were obtained by PCR amplification using primer set ISceICAM_p26gibs_F (SEQ ID NO: 57) and ISceICAM_p74gibs_R (SEQ ID NO: 58), thereby providing ISceICAM_gibs insert fragments.

5 μg of Bacmid AA745 DNA was then digested with Cas9 enzyme and sgRNAs for cutting the p26-to-p74 region of the AA745 baculovirus genome, using 10 μL of the following 30 μL mastermix (per 5 μg of DNA): 11.59 μL of nuclease-free water, 7.00 μL of 10× Cas9 Nuclease Rxn Buffer (NEBuffer3.1), 3.79 μL S.py Cas9 (1000 nM), 6.31 μL (300 nM) of sgP26_s1_t1 (SEQ ID NO: 59), and 6.31 μL (300 nM) of sgP74_s1_t1 (SEQ ID NO: 60).

The polh-atgRep52 insert fragments were then incorporated into the p26-to-p74-digested AA745 bacmid genomes by Gibson assembly reaction: 9 μL of p26-to-p74-digested AA745+1 μL of ISceICAM_gibs insert fragments (52 ng/μL)+10 μL of 2× Hifi Assembly Mastermix. The mixture was incubated 50° C. for 1.5 hours, transformed into electrocompetent NEB10b E. Coli samples, and plated on CAM plates for colony expansion and screening.

Incorporation of the ISceICAM_gibs insert into the p26-to-p74-digested AA745 bacmid (and corresponding removal of the p26-to-p74 region) was confirmed by digesting 5 μg of Bacmid Colony AA749 with I-SceI enzyme overnight at 37° C. Western Blot analysis confirmed that bacmid AA749 yielded a digest pattern which included a ˜900 bp band, consistent with the presence of an ISceI-flanked CAM cassette within the bacmid.

### Production of Bacmid AA753

Plasmids which included a gp64_polh_LacR sequence (SEQ ID NO: 13) with Gibson Assembly sequences were provided. gp64_polh_LacR insert fragments were obtained by PCR amplification using primer set gp64polhLacRp26gibs_F (SEQ ID NO: 61) and LacRp26gibs_R (SEQ ID NO: 62), thereby providing gp64_polh_LacR_gibs insert fragments.

5.0 μg of bacmid DNA AA749 was digested with I-SceI enzyme at 37° C. for 2 hours to provide I-SceI-digested AA749 bacmid. The gp64_polh_LacR insert fragments were then incorporated into the I-SceI-digested AA749 bacmid genomes by Gibson assembly reaction: 9 μL of I-SceI-digested AA749+1 μL of gp64_polh_LacR_gibs insert fragments (30 ng/μL)+10 μL of 2×Hifi Assembly Mastermix. The mixture was incubated 50° C. for 2 hours, desalted by ethanol precipitation, transformed into electrocompetent NEB10b E. Coli samples, and plated on KAN plates for colony expansion and screening.

Incorporation of the gp64_polh_LacR insert into the I-SceI-digested AA749 bacmid was confirmed by confirmational sequencing of Bacmid Colony AA753.

### Example 4. Testing of Bacmid AA753

### Insertion of PHPN VP-Coding Sequences

Plasmids which included a PHPN VP1/VP2/VP3 coding sequence (SEQ ID NO: 63) was provided. atgVP1 (PHPN) fragments with Gibson Assembly sequences were obtained by PCR amplification using primer set GGAKoz_VP1_2m_gibs_F (SEQ ID NO: 64) and VP1gibs_R (SEQ ID NO: 65), thereby providing atgVP1_gibs insert fragments (SEQ ID NO: 66). About 15.0 μg of bacmid AA753 DNA was digested with I-CeuI enzyme in a 20 μL batch at 37° C. for 3 hours to provide I-CeuI-digested AA735 bacmid. The atgVP1_gibs insert fragments were then incorporated into the I-SceI-digested AA749 bacmid genomes by Gibson assembly reaction: 8 μL of I-CeuI-digested AA735+2 μL of atgVP1_gibs insert fragments (30 ng/μL)+10 μL of 2×Hifi Assembly Mastermix. The mixture was incubated 50° C. for 1.5 hours, desalted by ethanol precipitation, transformed into electrocompetent NEB10b E. Coli samples, and plated on KAN plates for colony expansion and screening. Incorporation of the atgVP1 insert into the VP-sequence insertion scaffold (and flanking a LacO-p10-LacO expression control region of SEQ ID NO: 40) in the chiA locus of the I-CeuI-digested AA753 bacmid was confirmed by PCR amplification of Bacmid Colony AA825 using primer set LacBac_chiAseq3_F (SEQ ID NO: 67) and LacBac_chiAseq3_R (SEQ ID NO: 68), followed by confirmational sequencing of the PCR products, thus showing Bacmid AA825 to include the target LacO-p10-LacO_atgVP1 construct of SEQ ID NO: 69.

atgVP2 (PHPN) fragments with Gibson Assembly sequences were obtained by PCR amplification from the PHPN VP1/VP2/VP3 coding sequence (SEQ ID NO: 63) using primer set CGTKoz_VP2gibs_F (SEQ ID NO: 70) and VP2gibs_R (SEQ ID NO: 71), thereby providing atgVP2_gibs insert fragments (SEQ ID NO: 72). About 5.0 μg of bacmid AA825 DNA was digested with 1 μL SrfI enzyme in a 10 μL batch at 37° C. for 1-2 hours to provide SrfI-digested AA825 bacmid. The atgVP2_gibs insert fragments were then incorporated into the SrfI-digested AA825 bacmid genomes by Gibson assembly reaction: 4 μL of SrfI-digested AA825+1 μL of atgVP2_gibs insert fragments (30 ng/μL)+5 μL of 2×Hifi Assembly Mastermix. The mixture was incubated 50° C. for about 1 hour, transformed into electrocompetent NEB10b E. Coli samples, and plated on KAN plates for colony expansion and screening. Incorporation of the atgVP2 insert into the VP-sequence insertion scaffold (and flanking a LacO-p10-LacO expression control region of SEQ ID NO: 40) in the gta locus of the SrfI-digested AA825 bacmid was confirmed by PCR amplification of Bacmid Colony AA847 using primer set Lef12seq_F (SEQ ID NO: 42) and GTAseq_R (SEQ ID NO: 43), followed by confirmational sequencing of the PCR products, thus showing Bacmid AA847 to include the target LacO-p10-LacO_atgVP2 construct of SEQ ID NO: 73.

atgVP3 (PHPN) fragments with Gibson Assembly sequences were obtained by PCR amplification from the PHPN VP1/VP2/VP3 coding sequence (SEQ ID NO: 63) using primer set AAGKoz_VP3gibs_F (SEQ ID NO: 74) and VP3gibs_R (SEQ ID NO: 75), thereby providing atgVP3_gibs insert fragments (SEQ ID NO: 76). About 5.0 μg of bacmid AA847 DNA was digested with 1 μL AscI enzyme in a 10 μL batch at 37° C. for no more than 1 hour to provide AscI-digested AA847 bacmid. The atgVP3_gibs insert fragments were then incorporated into the AscI-digested AA847 bacmid genomes by Gibson assembly reaction: 4 μL of AscI-digested AA847+1 μL of atgVP3_gibs insert fragments (30 ng/μL)+5 μL of 2×Hifi Assembly Mastermix. The mixture was incubated 50° C. for about 2.5 hours, transformed into electrocompetent NEB10b E. Coli samples, and plated on KAN plates for colony expansion and screening. Incorporation of the atgVP3 insert into the polh locus of the AscI-digested AA847 bacmid was confirmed by PCR amplification of Bacmid Colonies AA879, AA886, and AA887 using primer set LacBacVP3_F (SEQ ID NO: 77) and LacBacVP3_R (SEQ ID NO: 78), followed by confirmational sequencing of the PCR products, thus showing Bacmids AA879, AA886, and AA887 to include the target p10_atgVP3 of SEQ ID NO: 79.

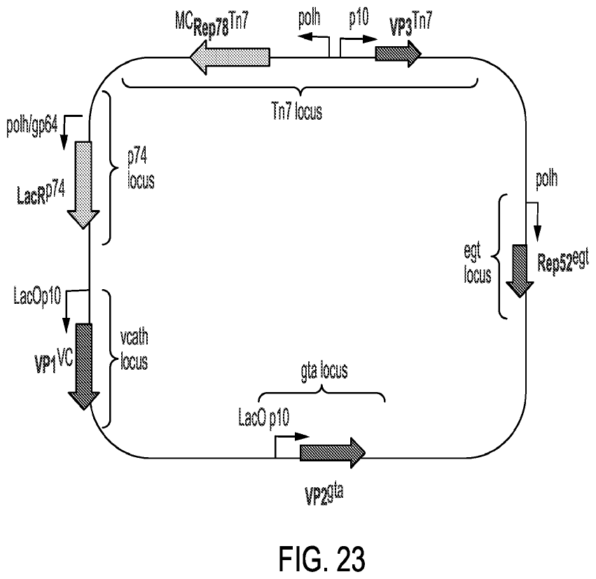

Bacmids AA879, AA886, and AA887 thus included: (i) a polh_MC1_atgRep78 sequence in the Tn7/polh locus; (ii) a polh-atgRep52 sequence in the egt locus; (iii) a gp64_polh_LacR sequence in the p74 locus; (iv) a LacO-p10-LacO_atgVP1 sequence in the chiA/v-cath locus; (v) a LacO-p10-LacO_atgVP2 sequence in the gta locus; and (vi) a p10_atgVP3 sequence in the Tn7/polh locus. A graphical representation of certain components and coding regions in Bacmids AA879, AA886, and AA887 is presented in FIG. 23.

### IPTG Regulation Testing of VP1/VP2/VP3 Sequences in AA879, AA886, and AA887

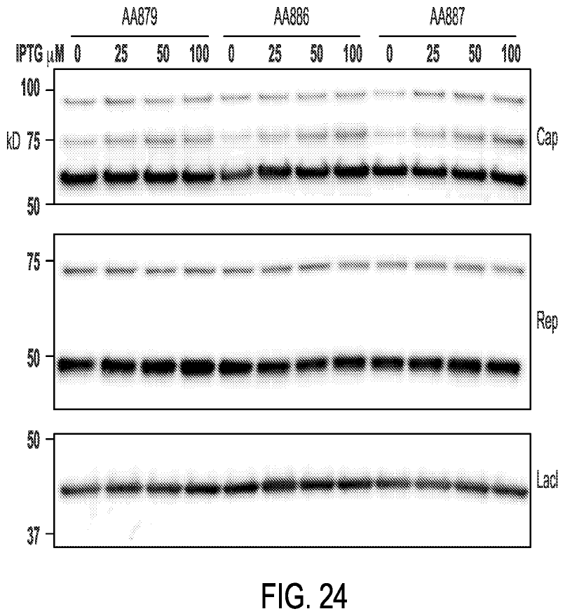

Samples of bacmids AA879, AA886, and AA887 were provided. 70 mL culture samples of sf9 cells were prepared at a VPC density of about 2×10⁶ vc/mL, with 15 mL of the sf9 culture sample then aliquoted into each of four 50 mL Corning mini-bioreactor tubes. Isopropyl β-d-1-thiogalactopyranoside (IPTG) stock (20 mM in ESF media, 0.2 μm-filtered) was then added to the four tubes at the following different volumes (20 mM): 0 μL, 19 μL, 38 μL, and 75 μL; thereby providing the following final working IPTG concentrations within the four culture samples: 0 μM, 25 μM, 50 μM, and 100 μM. 5 mL from each the four culture samples was then aliquoted into 3× replicate mini-bioreactors for each IPTG concentration sample (twelve mini-bioreactors total). Bacmids AA879, AA886, and AA887 were then added to the each mini-bioreactors at set to incubate at 27° C., such that each of the three bacmids was combined with each of the four IPTG concentrations in the culture samples. Samples from each of the twelve cultures were collected, lysed, and then analyzed by Western blot analysis for AAV Rep proteins (Rep78 and Rep52), AAV Cap proteins (VP1, VP2, and VP3), and LacI proteins. Results of the Western blot analysis are shown in FIG. 24. Western blot analysis confirmed Rep protein production, VP protein production, and LacI protein production from each test culture.

Bacmid AA887 was further analyzed with a full IPTG characterization. A 100 mL culture sample of sf9 cells was prepared at a VPC density of about 2×10⁶ vc/mL, and then transfect with 2 mL of Bacmid AA887 material. The culture was incubated for about 24 hours, and 5.3 mL of the Bacmid AA887 culture was then aliquoted into each of twelve 50 mL Corning mini-bioreactor tubes. Isopropyl β-d-1-thiogalactopyranoside (IPTG) stock and ESF media was then added to the twelve tubes according to the conditions listed in Table 6, and then incubated at 27° C. (shaking).

TABLE 6
IPTG characterization of Bacmid AA887

| | AA887 | IPTG Stock | IPTG | ESF | Final IPTG |
|---|---|---|---|---|---|
| Culture | Culture | Concentration | Stock | Media | Concentration |
| ID | (mL) | (μM) | (μL) | (μL) | (μM) |
| 1 | 5.3 mL | 1.00 × 10³ | 0.0 | 300.0 | 0.00 |
| 2 | | | 5.3 | 294.7 | 1.00 |
| 3 | | | 11.4 | 288.6 | 2.15 |
| 4 | | | 24.6 | 275.4 | 4.64 |
| 5 | | | 53.0 | 247.0 | 10.00 |
| 6 | | | 114.2 | 185.8 | 21.54 |
| 7 | | | 246.0 | 54.0 | 46.42 |
| 8 | | 1.00 × 10⁵ | 5.3 | 294.7 | 100.00 |
| 9 | | | 11.4 | 288.6 | 215.44 |
| 10 | | | 24.6 | 275.4 | 464.16 |
| 11 | | | 53.0 | 247.0 | 1000.00 |
| 12 | | | 114.2 | 185.8 | 2154.43 |

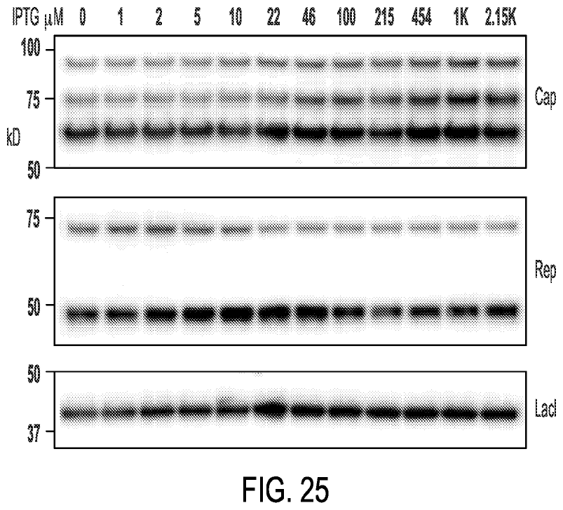

Samples from each of the twelve cultures were collected, lysed, and then analyzed by Western blot analysis for AAV Rep proteins (Rep78 and Rep52), AAV Cap proteins (VP1, VP2, and VP3), and LacI proteins. Results of the Western blot analysis are shown in FIG. 25. Western blot analysis confirmed Rep protein production, VP protein production, and LacI protein production from each test culture.

### Example 5. Production and Testing of Bacmids AA904 and AA935

### Production of Bacmid AA866

Bacmid AA735 (Example 4) was engineered to allow for more efficient cloning and screening of VP sequences into the v-cath locus by incorporating an AvrII-flanked chloramphenicol (CAM) expression cassette into the v-cath locus of the AA735 bacmid genome. Polynucleotide cassettes were engineered to include the CAM gBlock sequence of SEQ ID NO: 80, which includes an AvrII-flanked CAM expression sequence. About 5.0 μg of bacmid AA753 DNA was digested with I-CeuI enzyme at 37° C. for 3 hours to provide I-CeuI-digested AA735 bacmid. The CAM gBlock sequence was then incorporated into the I-CeuI-digested AA735 bacmid genomes by Gibson assembly reaction: 5 μL of I-CeuI-digested AA735+1 μL of CAM gBlock insert fragments (20 ng/μL)+6 μL of 2×Hifi Assembly Mastermix. The mixture was incubated 50° C. for 1 hour, transformed into electrocompetent NEB10b E. coli samples, and plated on KAN+CAM plates for colony expansion and screening. Bacmid Colony AA866 was selected for further development and screening.

### Insertion of VP-Coding Sequences for CG085 and CG088

Plasmids were provided which included VP1, VP2 and VP3 coding sequence for two different AAV candidate capsids, CG085 (SEQ ID NO: 81) and CG088 (SEQ ID NO: 83). AAV capsids CG085 (SEQ ID NO: 82) and CG088 (SEQ ID NO: 84) are AAV9 variants. atgVP1 fragments with Gibson Assembly sequences were obtained for both CG085 and CG088 by PCR amplification using primer set cgtVP1_LBGibs_F (SEQ ID NO: 85) and VP1_LBGibs_R (SEQ ID NO: 86), thereby providing atgVP1_gibs insert fragments for both CG085 and CG088. atgVP2 fragments with Gibson Assembly sequences were obtained for both CG085 and CG088 by PCR amplification using primer set cgtVP2_LBGibs_F (SEQ ID NO: 87) and VP2_LBGibs_R (SEQ ID NO: 88), thereby providing atgVP2_gibs insert fragments for both CG085 and CG088. atgVP3 fragments with Gibson Assembly sequences were obtained for both CG085 and CG088 by PCR amplification using primer set cgtVP3_LBGibs_F (SEQ ID NO: 89) and VP3_LBGibs_R (SEQ ID NO: 90), thereby providing atgVP3_gibs insert fragments for both CG085 and CG088.

PCR products for CG085atgVP1_gibs, CG085atgVP2_gibs, and CG085atgVP3_gibs were prepared in 30 ng/μL samples. About 20.0 μg of bacmid AA866 DNA was digested in a 30 μL Cutsmart reaction by adding 0.4 μL of SrfI enzyme mixture, 0.8 μL of AscI enzyme mixture, 1.7 μL of AvrII enzyme mixture, and 3 μL of 10× Cutsmart buffer. The following AA866 digestion fragments were collected by gel extraction: (i) AA866_AvrII_SrfI_AscI_1 (˜38 kbp in size), (ii) AA866_AvrII_SrfI_AscI_2 (˜72.4 kbp in size), and (iii) AA866_AvrII_SrfI_AscI_3 (˜33 kbp in size). CG085 VP sequences were then incorporated into the digested AA866 bacmid genome by Gibson assembly reaction according to Table 7.

TABLE 7
CG085/AA866 Gibson Assembly

| | Concentration | Volume Added |
|---|---|---|
| Fragment ID | (ng/μL) | (μL) |
| AA866_AvrII_SrfI_AscI_1 | 184.00 | 6.38 |
| AA866_AvrII_SrfI_AscI_2 | 350.00 | |
| AA866_AvrII_SrfI_AscI_3 | 160.00 | |
| CG085atgVP1_gib | 50.00 | 1.40 |
| CG085atgVP2_gib | 50.00 | 1.13 |
| CG085atgVP3_gib | 39.00 | 1.32 |
| H20 | — | — |
| 2X Hifi Mastermix | — | 10.23 |

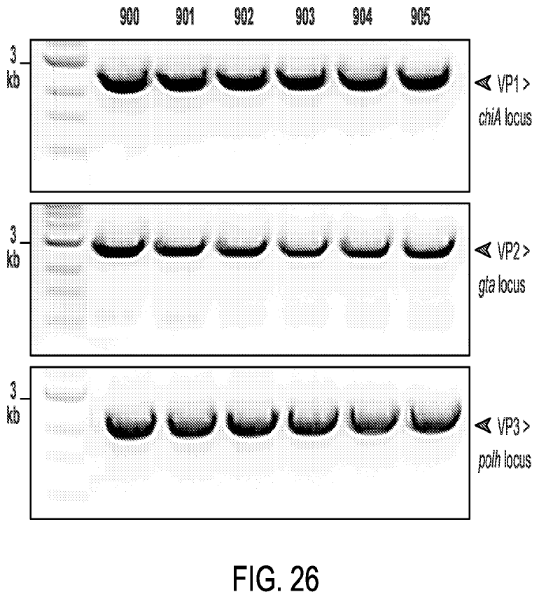

The mixture was incubated at 50° C., transformed into electrocompetent NEB10b E. Coli samples, and plated on KAN plates for colony expansion and replica-plated on KAN versus CAM versus CARB plates to screen for loss of CAM and CARB-resistance. Single-mixture incorporation of the CG085atgVP1, CG085atgVP2, and CG085atgVP3 inserts into the digested AA866 bacmid was confirmed by PCR amplification of Bacmid Colonies AA900 to AA905 using: (i) ChiAseq_F (SEQ ID NO: 44) and gp64seq_R (SEQ ID NO: 45), (ii) primer set Lef12seq_F (SEQ ID NO: 42) and GTAseq_R (SEQ ID NO: 43), and (iii) primer set LacBacVP3_F (SEQ ID NO: 77) and LacBacVP3_R (SEQ ID NO: 78). Gel analysis for AAV Cap proteins (VP1, VP2, and VP3) is shown in FIG. 26. Incorporation was further confirmed by confirmational sequencing of Bacmid AA904 PCR products, thus showing Bacmid AA904 to include the CG085atgVP1, CG085atgVP2, and CG085atgVP3 inserts.

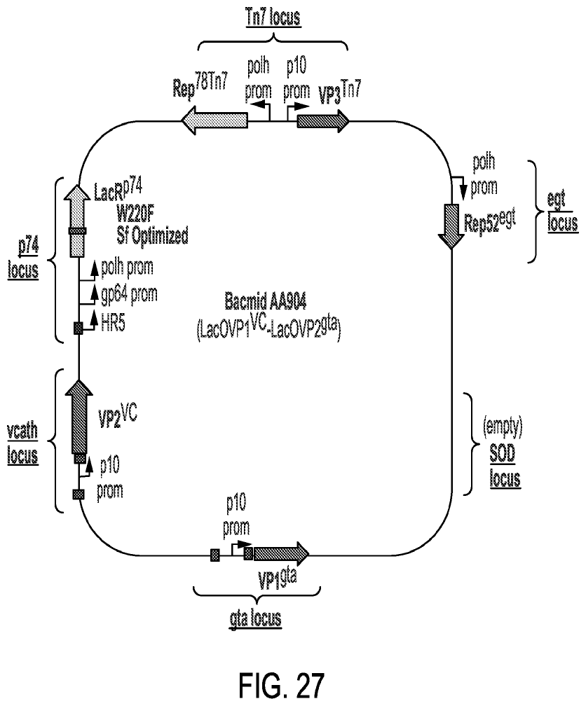

A graphical representation of certain components and coding regions in Bacmid AA904 is presented in FIG. 27. Bacmid AA904 has Rep78 and Rep52 ORFs separately expressed under the regulation of polh promoters. The Rep78-VP3 cassette was located in the Tn7 locus and the Rep52 cassette was in the egt locus. A single copy of LacR was cloned into the p74 locus downstream from a naturally occurring homologous repeat region called HR5. The LacR promoter was aligned with an HR5 enhancer element in the p74 locus. In order to compensate for the single LacR copy, the LacR was codon optimized for Sf9 and a W220F mutation was made into the LacR ORF to improve repression by LacR (Lafranconi et al., Microb Cell Fact 12:67 (2013), which is hereby incorporated by reference in its entirety). LacR also was codon optimized for translational initiation with a better Kozak context, which resulted in a T2A change of the NLS-LacR ORF.

PCR products for CG088atgVP1_gibs, CG088atgVP2_gibs, and CG088atgVP3_gibs were prepared in 30 ng/μL samples; and were then incorporated into bacmid AA866 using the same single-mixture procedures used for incorporation of CG085 VP fragments (see above). The resulting mixture was incubated at 50° C., transformed into electrocompetent NEB10b E. Coli samples, and plated on KAN plates for colony expansion and replica-plated on KAN versus CAM versus CARB plates to screen for loss of CAM and CARB-resistance. Single-mixture incorporation of the CG088atgVP1, CG088atgVP2, and CG088atgVP3 inserts into the digested AA866 bacmid was confirmed by PCR amplification of Bacmid Colonies AA935 to AA937 using the same primer sets as CG085 (see above), followed by confirmational sequencing of Bacmid AA935 PCR products, thus showing Bacmid AA935 to include the CG088atgVP1, CG088atgVP2, and CG088atgVP3 inserts.

Bacmids AA904 and AA935 were then each transfected into separate 35 mL Sf9 cell cultures (VCD of about 2.2×10⁶) to generate corresponding BacCG085 and BacCG088 pre-BIIC stocks.

### Testing AA904 and AA935

Samples of bacmids AA904 and AA935 were provided. sf9 cells were expanded in large culture up to a VPC density of about 3×10⁶ vc/mL, and then seeded into twenty-one separate flasks at 25 mL working volume (about 7.5×10⁷ vc/flask).

ITR-SEAP-GFP payloadBIIC material (AA890), AA904 expressionBIIC material, and AA935 expressionBIIC material were prepared according to Table 8.

TABLE 8
BIIC preparations for Bacmid AA904/AA935 testing

| | | BIIC | ESF Media | Total |
|---|---|---|---|---|
| | | Volume | Volume | Volume |
| Material | TCID50/mL | (μl) | (μL) | (μL) |
| AA890 | 1.41 × 10⁸ | 166 | 3162 | 3329 |
| AA904 | 1.80 × 10⁸ | 165 | 3136 | 3301 |
| AA935 | 3.43 × 10⁸ | 79 | 1510 | 1589 |

Sf9 cells were then co-infected with payloadBIIC material (AA890) and expressionBac material (AA904 or AA935) according to the conditions in Table 9.

TABLE 9
IPTG characterization of Bacmid AA656

| | payloadBIIC/ | AA890 | AA904 | AA935 | |
|---|---|---|---|---|---|
| Culture | expressionBIIC | (μL) | (μL) | (μL) | IPTG |
| ID | Ratio | [MOI] | [MOI] | [MOI] | (μM) |
| 1 | 0:0 (Control) | — | — | — | 0 |
| 2 | 3:1 | 107 [0.01] | — | — | 0 |
| 3 | 1:0 | 107 [0.01] | — | — | 1000 |
| 4 | 1:0 | — | 83 [0.01] | — | 0 |
| 5 | 0:1 | — | — | 44 [0.01] | 0 |
| 7 | 1:4 | 107 [0.01] | 333 [0.04] | — | 0 |
| 8 | 1:4 | 107 [0.01] | 333 [0.04] | — | 5 |
| 9 | 1:4 | 107 [0.01] | 333 [0.04] | — | 10 |
| 10 | 1:4 | 107 [0.01] | 333 [0.04] | — | 25 |
| 11 | 1:4 | 107 [0.01] | 333 [0.04] | — | 50 |
| 12 | 1:4 | 107 [0.01] | 333 [0.04] | — | 100 |
| 13 | 1:4 | 107 [0.01] | 333 [0.04] | — | 200 |
| 14 | 1:4 | 107 [0.01] | 333 [0.04] | — | 1000 |
| 15 | 1:4 | 107 [0.01] | — | 175 [0.04] | 0 |
| 16 | 1:4 | 107 [0.01] | — | 175 [0.04] | 5 |
| 17 | 1:4 | 107 [0.01] | — | 175 [0.04] | 10 |
| 18 | 1:4 | 107 [0.01] | — | 175 [0.04] | 25 |
| 19 | 1:4 | 107 [0.01] | — | 175 [0.04] | 50 |
| 20 | 1:4 | 107 [0.01] | — | 175 [0.04] | 100 |
| 21 | 1:4 | 107 [0.01] | — | 175 [0.04] | 200 |
| 22 | 1:4 | 107 [0.01] | — | 175 [0.04] | 1000 |

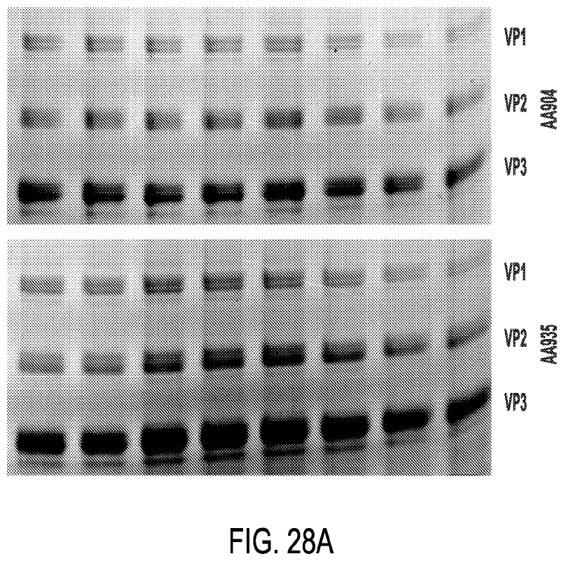

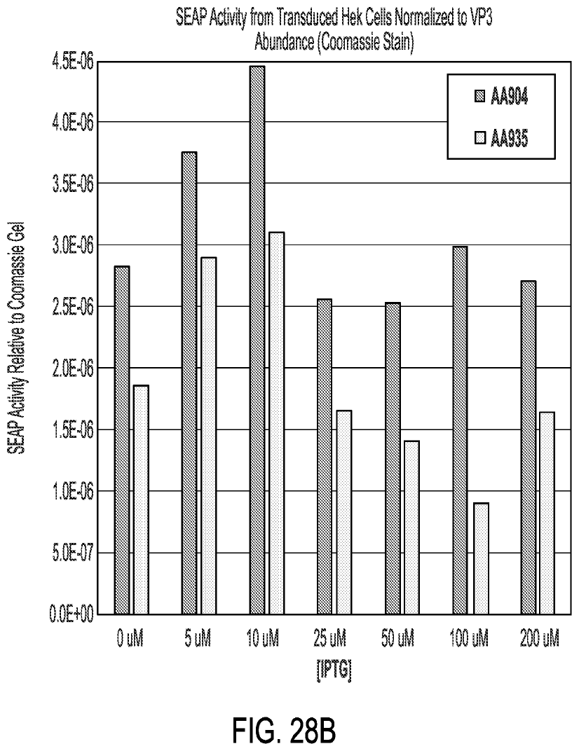

Cell lysate samples were collected from each culture sample, which were then processed and pelleted trough sucrose cushion ultracentrifugation. Purified AAV samples from the clarified cell lysate were collected, transduced onto 293 HEK cells, and alkaline phosphatase activity (i.e., SEAP potency) was measured. The measured SEAP activity was then normalized to AAV VP3 capsid abundances, which were determined from Coomassie stained SDS-PAGE gels (FIG. 28A). Results of SEAP Normalization analysis are shown in FIG. 28B.

Results also showed that Normalized SEAP payload potency (nU SEAP/vg) was highest for both AA904 and AA935 at IPTG concentrations between about 5.0 and about 10.0 μM. SEAP payload potency for AA904 was higher than AA935, though both candidate capsids provide for strong SEAP activity in AAV particles.

### Example 6. Bacmid Expression of Capsid Variants

The bacmid constructs disclosed in Example 6 were utilized in the expression of the capsid proteins of Table 10.

TABLE 10
Capsid Sequences

| Description | SEQ ID NO: | Sequence Information |
|---|---|---|
| CG085 | 82 | MAADGYLPDWLEDNLSEGIREWWALKPGAPQPKANQQHQDNARGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGAQ |
| | | PAKKRLNFGQTGDTESVPDPQPIGEPPAAPSGVGSLTMASGGGAPVADNNEGADG |
| | | VGSSSGNWHCDSQWLGDRVITTSTRIWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVEMIPQYGYLILNDG |
| | | SQAVGRSSFYCLEYFPSQMLRIGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKITNPVATESYGQVATNHQSPLNGAVHLYAQAQTGWVQN |
| | | QGILPGMVWQDRDVYLQGPIWAKIPHIDGNFHPSPLMGGFGMKHPPPQILIKNTP |
| | | VPADPPTAFNKDKLNSFITQYSTGQVSVEIEWELQKENSKRWNPEIQYTSNYYKS |
| | | NNVEFAVNTEGVYSEPRPIGTRYLTRNL |
| CG088 | 84 | MAADGYLPDWLEDNLSEGIREWWALKPGAPQPKANQQHQDNARGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGAQ |
| | | PAKKRLNFGQTGDTESVPDPQPIGEPPAAPSGVGSLTMASGGGAPVADNNEGADG |
| | | VGSSSGNWHCDSQWLGDRVITTSTRIWALPTYNNHLYKQISNSTSGGSSNDNAYF |
| | | GYSTPWGYFDFNRFHCHFSPRDWQRLINNNWGFRPKRLNFKLFNIQVKEVIDNNG |
| | | VKTIANNLISTVQVFTDSDYQLPYVLGSAHEGCLPPFPADVEMIPQYGYLILNDG |
| | | SQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSITVTQ |
| | | NNNSEFAWPGASSWALNGRNSLMNPGPAMASHKEGEDRFFPLSGSLIFGKQGTGR |
| | | DNVDADKVMITNEEEIKITNPVATESYGQVATNHQSAQIVMNSLKAQAQTGWVQN |
| | | QGILPGMVWQDRDVYLQGPIWAKIPHTDGNFHPSPLMGGFGMKHPPPQILIKNTP |
| | | VPADPPTAFNKDKLNSFITQYSTGQVSVEIEWELQKENSKRWNPEIQYTSNYYKS |
| | | NNVEFAVNTEGVYSEPRPIGTRYLTRNL |

### Example 7. Additional Bacmid Constructs

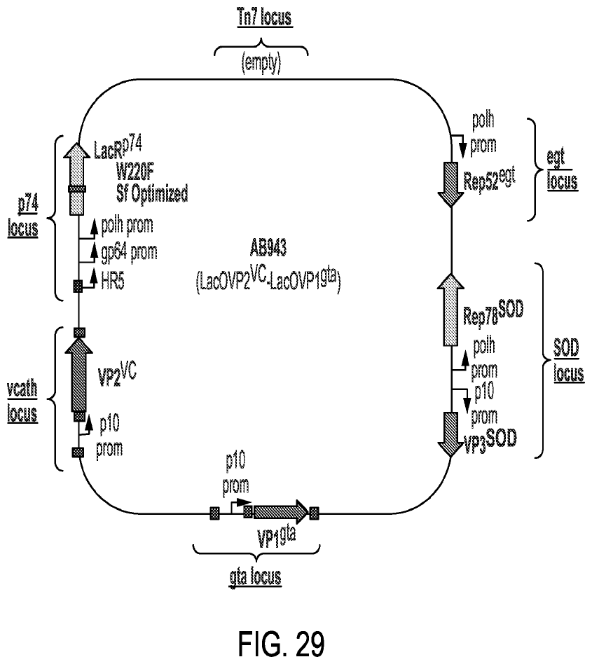

Further bacmids for AAV expression were designed based on the constructs described above. A graphical representation of certain components and coding regions in these bacmids is presented in FIG. 29. The Rep78-VP3 cassette was moved out of the Tn7 locus and cloned by Gibson assembly into the SOD locus at the AbsI REN site in the middle of the SOD ORF. The Rep78 and Rep52 ORFs were separately expressed under the regulation of polh promoters in egt and SOD loci, respectively. The VP1 and VP2 ORFs were swapped in location to improve the repression of VP1 with VP1 in gta locus and VP2 in vcath locus. The LacO-p10-LacO promoters driving expression of VP1 and VP2 were left unchanged. The LacOs were cloned onto the 3′ ends of VP1 and VP2 ORFs to improve repression by LacO as described by Oehler et al., EMBO J. 9(4):973-9 (1990), which is hereby incorporated by reference in its entirety.

A single copy of LacR was cloned into the p74 locus and was codon optimized for Sf9 and a W220F mutation was made into the LacR ORF. The NLS-LacR genes are 73.85% identical to the non-codon optimized, and the translated proteins differ by 2 amino acid changes (T2A) and (W235F), as shown in Table 11.

TABLE 11
LacR Codon Optimization for Sf9

| Description | SEQ ID NO: | Sequence Information |
|---|---|---|
| NLS-LacR | 139 | ATGACGCAACCTAAGAAGAAGAGGAAGGTTCCCGGGCAAGTGACTATGAAACCAGTAACGTTAT |
| (DNA) | | ACGATGTCGCAGAGTATGCCGGTGTCTCTTATCAGACCGTTTCCCGCGTGGTGAACCAGGCCAG |
| | | CCACGTTTCTGCGAAAACGCGGGAAAAAGTGGAAGCGGCGATGGCGGAGCTGAATTACATTCCC |
| | | AACCGCGTGGCACAACAACTGGCGGGCAAACAGTCGTTGCTGATTGGCGTTGCCACCTCCAGTC |
| | | TGGCCCTGCACGCGCCGTCGCAAATTGTCGCGGCGATTAAATCTCGCGCCGATCAACTGGGTGC |
| | | CAGCGTGGTGGTGTCGATGGTAGAACGAAGCGGCGTCGAAGCCTGTAAAGCGGCGGTGCACAAT |
| | | CTTCTCGCGCAACGCGTCAGTGGGCTGATCATTAACTATCCGCTGGATGACCAGGATGCCATTG |
| | | CTGTGGAAGCTGCCTGCACTAATGTTCCGGCGTTATTTCTTGATGTCTCTGACCAGACACCCAT |
| | | CAACAGTATTATTTTCTCCCATGAAGACGGTACGCGACTGGGCGTGGAGCATCTGGTCGCATTG |
| | | GGTCACCAGCAAATCGCGCTGTTAGCGGGCCCATTAAGTTCTGTCTCGGCGCGTCTGCGTCTGG |
| | | CTGGCTGGCATAAATATCTCACTCGCAATCAAATTCAGCCGATAGCGGAACGGGAAGGCGACTG |
| | | GAGTGCCATGTCCGGTTTTCAACAAACCATGCAAATGCTGAATGAGGGCATCGTTCCCACTGCG |
| | | ATGCTGGTTGCCAACGATCAGATGGCGCTGGGCGCAATGCGCGCCATTACCGAGTCCGGGCTGC |
| | | GCGTTGGTGCGGATATCTCGGTAGTGGGATACGACGATACCGAAGACAGCTCATGTTATATCCC |
| | | GCCGTCAACCACCATCAAACAGGATTTTCGCCTGCTGGGGCAAACCAGCGTGGACCGCTTGCTG |
| | | CAACTCTCTCAGGGCCAGGCGGTGAAGGGCAATCAGCTGTTGCCCGTCTCACTGGTGAAAAGAA |
| | | AAACCACCCTGGCGCCCAATACGCAAACCGCCTCTCCCCGCGCGTTGGCCGATTCATTAATGCA |
| | | GCTGGCACGACAGGTTTCCCGACTGGAAAGCGGGCAGTGA |
| NLS-LacR | 140 | MTQPKKKRKVPGQVTMKPVTLYDVAEYAGVSYQTVSRVVNQASHVSAKTREKVEAAMAELNYIP |
| (Protein) | | NRVAQQLAGKQSLLIGVATSSLALHAPSQIVAAIKSRADQLGASVVVSMVERSGVEACKAAVHN |
| | | LLAQRVSGLIINYPLDDQDAIAVEAACTNVPALFLDVSDQTPINSIIFSHEDGTRLGVEHLVAL |
| | | GHQQIALLAGPLSSVSARLRLAGWHKYLTRNQIQPIAEREGDWSAMSGFQQTMQMLNEGIVPTA |
| | | MLVANDQMALGAMRAITESGLRVGADISVVGYDDTEDSSCYIPPSTTIKQDFRLLGQTSVDRLL |
| | | QLSQGQAVKGNQLLPVSLVKRKTTLAPNTQTASPRALADSLMQLARQVSRLESGQ |
| Sf | 141 | ATGGCACAGCCCAAAAAGAAGCGTAAGGTGCCCGGCCAGGTCACGATGAAACCTGTCACCCTTT |
| Optimized | | ATGACGTTGCCGAATATGCGGGTGTTTCGTATCAGACAGTTTCACGCGTAGTGAACCAAGCTTC |
| NLS-LacR | | TCACGTCAGCGCCAAGACCAGGGAAAAGGTAGAGGCTGCCATGGCAGAACTGAATTATATACCC |
| (DNA) | | AACAGAGTCGCACAACAGCTTGCAGGAAAACAGAGTCTCTTGATCGGTGTTGCCACATCTTCGT |
| | | TGGCACTTCACGCTCCTAGTCAAATCGTTGCTGCTATCAAGAGTAGGGCAGATCAACTTGGTGC |
| | | CTCGGTAGTTGTGAGTATGGTAGAGCGCTCCGGCGTCGAAGCTTGTAAGGCCGCTGTGCATAAT |
| | | CTCCTCGCACAGCGCGTTAGCGGTCTTATAATTAATTACCCACTTGATGACCAGGACGCTATAG |
| | | CTGTAGAAGCGGCATGTACTAACGTGCCAGCGCTGTTTTTGGACGTATCCGACCAAACACCCAT |
| | | TAACTCCATTATTTTCTCACACGAGGATGGCACGAGGCTCGGCGTGGAGCATCTTGTAGCATTG |
| | | GGTCACCAACAAATCGCCCTTCTTGCTGGCCCATTGTCTTCAGTCTCTGCTCGCCTTCGTCTTG |
| | | CGGGCTGGCATAAGTATCTTACGCGCAACCAAATACAACCCATTGCAGAACGTGAGGGCGACTt |
| | | cTCGGCCATGTCGGGATTTCAACAAACTATGCAAATGCTGAACGAGGGAATCGTTCCTACCGCA |
| | | ATGCTTGTAGCAAACGATCAAATGGCCTTGGGAGCCATGCGCGCTATTACCGAAAGCGGATTGA |
| | | GAGTTGGTGCTGACATTAGCGTGGTAGGCTACGACGACACTGAGGACTCTAGTTGTTATATTCC |
| | | TCCCttgACTACGATAAAGCAGGACTTTAGGCTCCTTGGTCAGACATCTGTGGACAGACTGCTC |
| | | CAGTTGAGCCAGGGACAAGCGGTTAAGGGCAATCAGCTCCTCCCCGTTTCCCTTGTGAAAAGGA |
| | | AGACTACGCTGGCCCCCAATACGCAAACTGCCTCACCGCGTGCTCTCGCGGATTCTCTCATGCA |
| | | GCTGGCCAGGCAAGTAAGCAGGCTTGAATCAGGCCAGTAA |
| Sf | 142 | MAQPKKKRKVPGQVTMKPVTLYDVAEYAGVSYQTVSRVVNQASHVSAKTREKVEAAMAELNYIP |
| Optimized | | NRVAQQLAGKQSLLIGVATSSLALHAPSQIVAAIKSRADQLGASVVVSMVERSGVEACKAAVHN |
| NLS-LacR | | LLAQRVSGLIINYPLDDQDAIAVEAACTNVPALFLDVSDQTPINSIIFSHEDGTRLGVEHLVAL |
| (Protein) | | GHQQIALLAGPLSSVSARLRLAGWHKYLTRNQIQPIAEREGDFSAMSGFQQTMQMLNEGIVPTA |
| | | MLVANDQMALGAMRAITESGLRVGADISVVGYDDTEDSSCYIPPLTTIKQDFRLLGQTSVDRLL |
| | | QLSQGQAVKGNQLLPVSLVKRKTTLAPNTQTASPRALADSLMQLARQVSRLESGQ |

### Example 8. Evaluation of Expressed AAVs and Transfected Cells

AA904 and HEK derived capsids were measured for alkaline phosphatase activity (i.e., SEAP potency) after purification using two different methods. After cesium chloride purification (CsCl) the potency of HEK derived capsids increased, but no affect was found on the potency of the AA904 capsids. Affinity exchange chromatography (AEX) purification of the AA904 derived capsids showed the same potency as the cesium chloride purified capsids. The potency of the capsids is shown in Table 12.

TABLE 12
SEAP Activity

| | SEAP | | |
|---|---|---|---|
| Sample | Activity/vg | R² | STDEV |
| AA904-AEX | 9.01E−02 | 0.977 | 2.05E−03 |
| HEK-AEX | 7.32E−02 | 0.979 | 1.57E−03 |
| AA904-AEX/CsCl | 8.12E−02 | 0.979 | 1.67E−03 |
| HEK-AEX/CsCl | 4.75E−01 | 0.926 | 3.52E−02 |

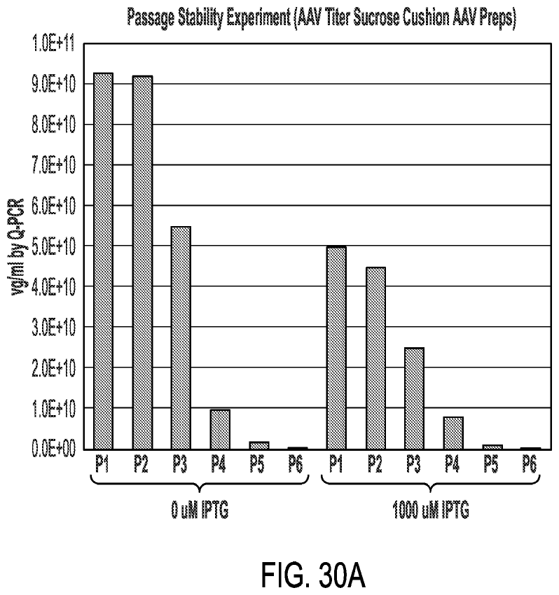

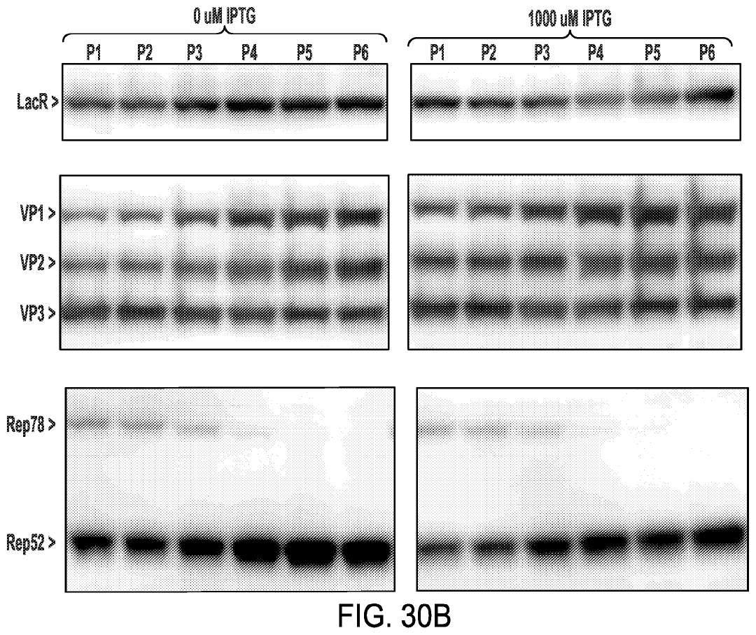

The passage stability of the transfected cells was analyzed by Q-PCR under two different concentrations of IPTG (0 uM and 1000 uM). After 6 passages, the cells displayed little to no AAV titer (FIG. 30A). Western blot analysis (FIG. 30B) indicated that upon serial passage a loss of Rep78 in the Tn7 locus occurred. This loss in Rep78 is what motivated the move of the Rep78-VP3 cassette out of the Tn7 locus and into the SOD locus as discussed in Example 7.

### Example 9. Additional Bacmid Constructs

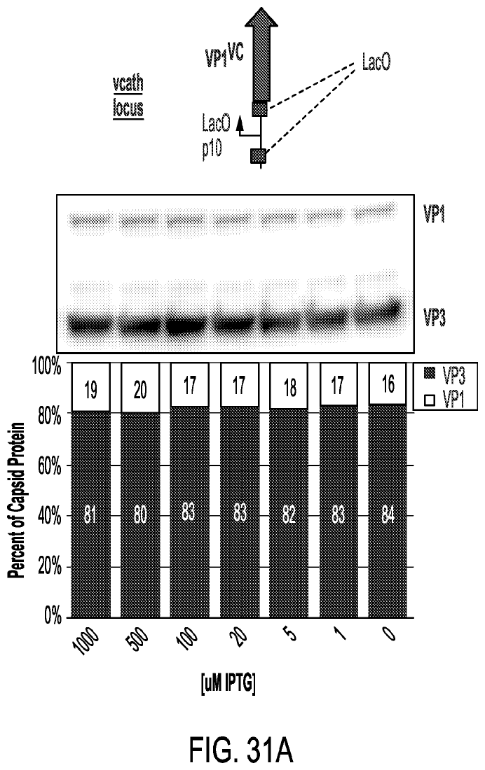

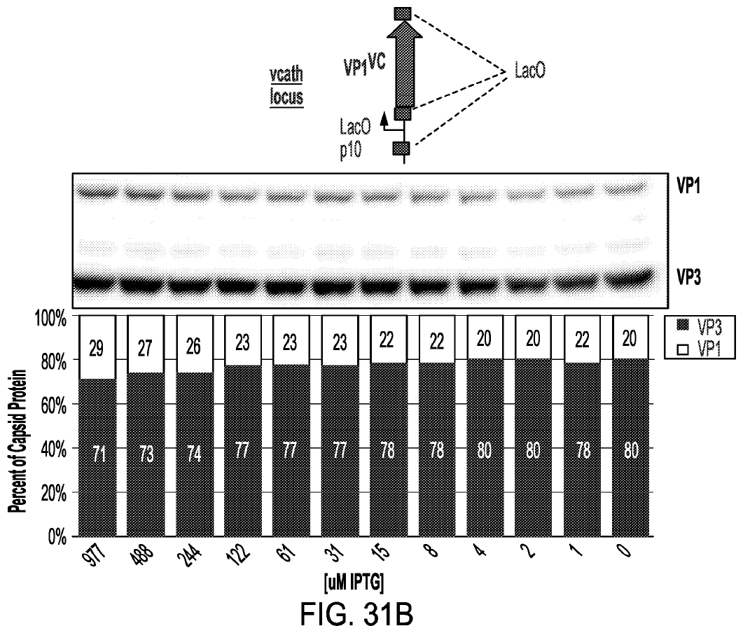

Bacmid constructs were designed to incorporate a third LacO into the 3′ end of the VP1 ORF. This was done to try and improve the repression achieved by LacO in a construct expressing VP1 and VP3, but not VP2. The data show the additional LacO improved the overall range of repression of VP1 expression by LacR, (25% for double LacO, 30% for triple LacO) (FIGS. 31A and 31B, respectively). However, moving the Rep78-VP3 cassette from Tn7 locus to the SOD locus in resulted in reduced VP3 expression, and thus the relative abundance of VP1 was higher regardless of IPTG induction. This change in locus put the VP1 abundance out of the useful inducible range for modulating VP ratio dependent AAV capsid potency.

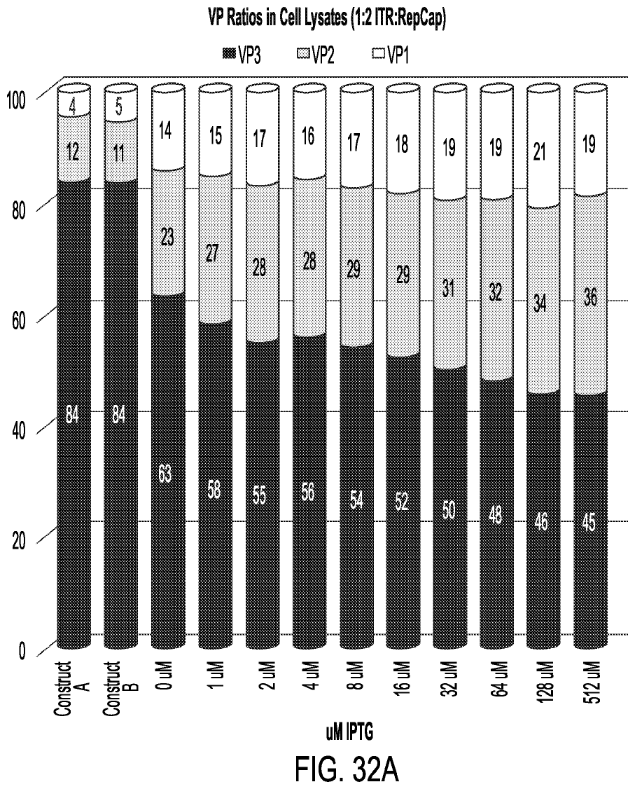

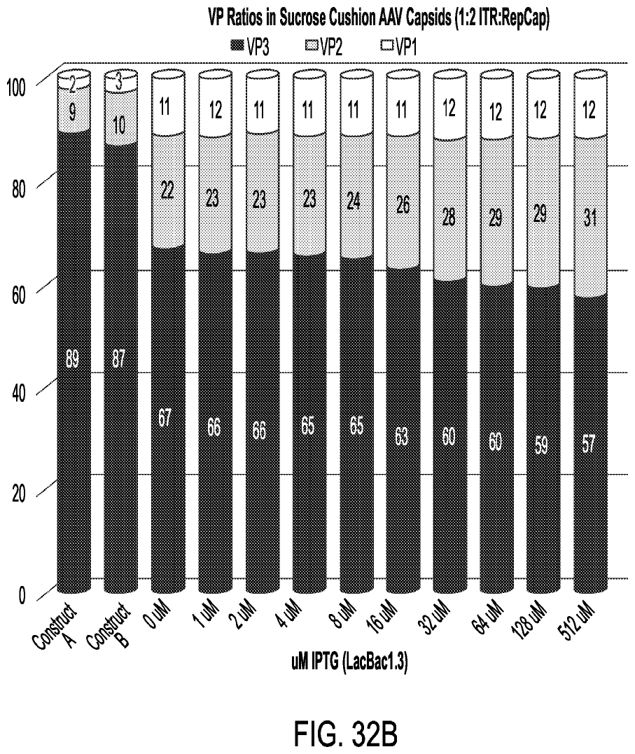

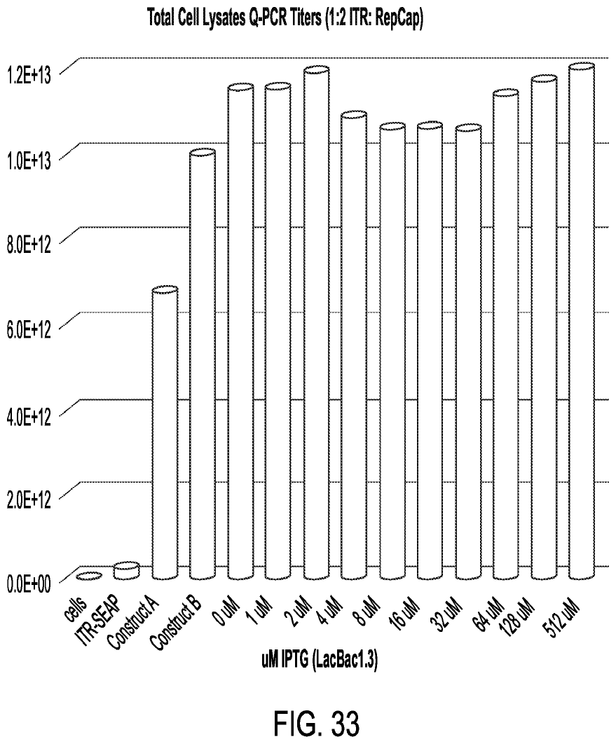

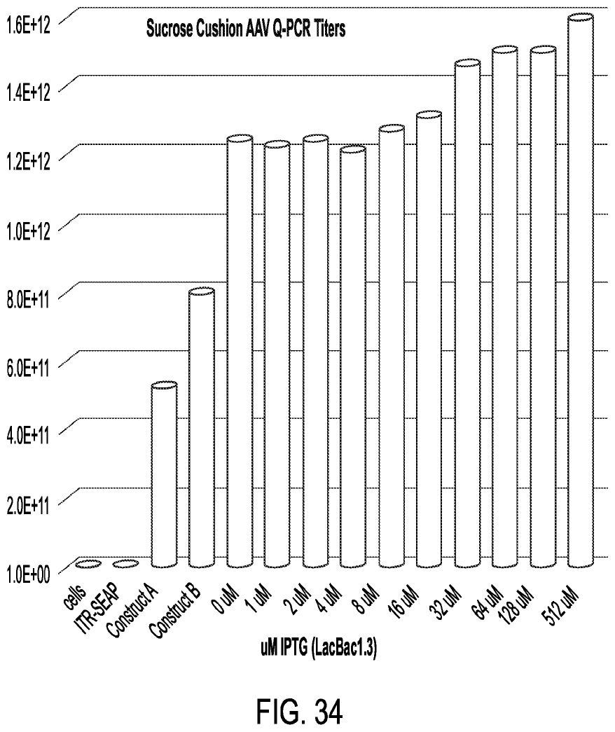

The bacmids described in Example 7, including LacO in the 3′ ends of VP1 and VP2 ORFs, produced a VP1 expression of 14 to 19% of the total capsid protein in comparison to the 20 to 29% obtained from the VP1 and VP3 expressing constructs. In the VP1/VP2/VP3 expressing constructs, VP2 expression drew away some very late transcription and contributed additional VP3 translation thus shifting ratios. The abundance of VP2 was too high relative to VP1 and VP3. This was not surprising because when VP1 and VP2 were in opposite positions, there was too much VP1 relative to VP2 and VP3. Plots of the VP ratios of the cell lysates and sucrose purified AAV capsids are shown in FIGS. 32A and 32B, respectively. The Q-PCR determined AAV titers in crude cell lysates did not drastically change regardless of the amount of IPTG added (FIG. 33). This differs from the previous constructs which showed AAV titer being affected by IPTG concentration (FIG. 14A-14B). It is suspected that the abundance of VP2 was negating the earlier observed IPTG tunable genome packaging into AAV particles. Additionally, the Q-PCR determined AAV titers in sucrose cushion purified samples became higher as IPTG concentrations were increased (FIG. 34). This indicates improved packaging into capsids was achieved as the VP1 abundance was increased. VP1 abundance became constant regardless of IPTG concentration. The AAV titers and yields were determined to be highest with an IPTG concentration of 50 uM, producing a fully induced system least repressed by LacR.

In further bacmids, the LacOVP1 cassette was moved from its original position in the v-cath locus to the SOD locus. This change resulted in a decrease in VP1 abundance. Additionally, the LacOVP2 cassette was moved from its original position in the gta locus to the v-cath locus, resulting in an increase in VP2 abundance. This reciprocal change in VP1 and VP2 expression due to changing cassette cloning loci in the bacmids reduced the ability tune AAV titer and potency with IPTG induction of LacR.

The constructs of the present application do not rely on screening large random libraries or trial and error. The non-canonical CTG and ACG translational start codons were removed from the constructs, capsid proteins VP1, VP2 and VP3 were expresses independently, and an E. coli Lac repressor is used to control VP1 and VP2 expression. The AAV titer and potency can be optimized using a single construct.

Future work will focus on improving the repression of Lac Repressor and simplifying the system. Future designs will also include moving the lacOVP1 cassette away from downstream of the highly transcribed gp64 gene to a location in the baculovirus genome which has less transcriptionally active at early times post infection. Incorporation of weak Kozak or non-canonical VP2/VP3 constructs may also be incorporated into the constructs.

We claim:

1. An AAV expression construct comprising:
(i) at least two Rep-coding regions, each comprising a nucleotide sequence encoding a Rep protein independently chosen from Rep52, Rep40, Rep68, or Rep78 protein,
(ii) at least two VP coding regions comprising a nucleotide sequence encoding a VP protein chosen independently chosen from a VP1 protein, a VP2 protein, a VP3 protein, or a combination thereof,
(iii) at least one transcriptional regulator element coding sequence (e.g., a lac repressor sequence); and
(iv) at least one regulator binding sequence (e.g., a lacO sequence), wherein the at least one regulator binding sequence is operably linked to the VP1 and/or VP2 sequence, and wherein AAV expression construct comprises a variant baculovirus genome,
wherein the at least two Rep-coding regions and/or the at least two VP coding regions each comprise a different nucleotide sequence and/or is present in different location;
wherein the AAV expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from gta, egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.

2. The AAV expression construct of claim 1, wherein the variant baculovirus genome comprises a nucleotide sequence or a portion thereof from a baculovirus genome selected from Autographa californica multiple nucleopolyhedrovirus (AcMNPV) (e.g., an AcMNPV strain E2, C6, or HR3), Bombyx mori nucleopolyhedrovirus (BmNPV), Anticarsia gemmatalis nucleopolyhedrovirus (AgMNPV), Orgyia pseudotsugata nucleopolyhedrovirus (OpMNPV), or Thysanoplusia orichalcea nucleopolyhedrovirus (ThorMNPV).

3. The AAV expression construct of claim 1 or claim 2, wherein the at least two Rep-coding regions each comprise a different nucleotide sequence and is present in different locations in the variant baculovirus genome.

4. The AAV expression construct of any one of claims 1-3, wherein the first Rep-coding region comprises a first a first open reading frame (ORF) comprising a start codon and a nucleotide sequence encoding a Rep78 protein and the second Rep-coding region comprises a second ORF comprising a start codon and a nucleotide sequence encoding a Rep52 protein, optionally, wherein:
(a)(i) the first Rep-coding region comprises the nucleotide sequence of SEQ ID NO: 143, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 143; or a nucleotide sequence having at least 10, 20, 50, 100, 150, 200, 250, 300, 350, 400, or 450 but no more than 500 modifications (e.g., substitutions) relative to SEQ ID NO: 143; or
(a)(ii) the first Rep-coding region encodes the amino acid sequence of SEQ ID NO: 144; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 144; or an amino acid sequence comprising at least 1, 2, 3, 4, 5, 10, 15, or 20 but no more than 30 modifications (e.g., substitutions (e.g., conservative substitutions), insertions, or deletions) relative to the amino acid sequence of SEQ ID NO: 144; and
(b)(i) the second Rep-coding region comprises the nucleotide sequence of SEQ ID NO: 145, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 145; or a nucleotide sequence having at least 10, 20, 50, 100, 150, 200, 250, 300, 350, 400, or 450 but no more than 500 modifications (e.g., substitutions) relative to SEQ ID NO: 145; or
(b)(ii) the second Rep-coding region encodes the amino acid sequence of SEQ ID NO: 146; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 146; or an amino acid sequence comprising at least 1, 2, 3, 4, 5, 10, 15, or 20 but no more than 30 modifications (e.g., substitutions (e.g., conservative substitutions), insertions, or deletions) relative to SEQ ID NO: 146.

5. The AAV expression construct of any one of the preceding claims, wherein the nucleotide sequence of the first Rep-coding region is operably linked to a first promoter, and the nucleotide sequence of the second Rep-coding region is operably linked to a second promoter, optionally, wherein the first promoter, second promoter, or both is selected from a polyhedrin (polh) promoter, a p10 promoter, a conotoxin (ctx) promoter, a gp64 promoter an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.

6. The AAV expression construct of any one of the preceding claims, wherein the first Rep-coding region or the second Rep-coding region comprises an expression-modifier sequence which decreases transcription initiation of the first Rep-coding region, optionally, wherein the expression-modifier sequence comprises a minicistron sequence, for example, from a baculovirus gene, optionally a baculovirus gp64 gene.

7. The AAV expression construct of any one of the preceding claims, wherein the first VP-coding region comprises a nucleotide sequence encoding:
(i) primarily a VP1 protein, e.g., at least 50%, 60%, 70%, 80%, 90% or more VP1 protein relative to a VP2 protein and/or a VP3 protein;
(ii) a VP1 protein only;
(iii) a VP1 protein, but not a VP2 protein or a VP3 protein.

8. The AAV expression construct of any one of the preceding claims, wherein the second VP-coding region comprises a nucleotide sequence encoding:
(i) a VP2 protein and a VP3 protein;
(ii) primarily a VP2 protein, e.g., at least about 50%, 60%, 70%, 80%, 90% or more VP2 protein relative to a VP1 protein and/or a VP3 protein;
(iii) a VP2 protein only;
(iv) a VP2 protein, but not a VP1 protein or a VP3 protein.

9. The AAV expression construct of any one of the preceding claims, comprising a third VP-coding region comprising a nucleotide sequence encoding
(i) primarily a VP3 protein;
(ii) a VP3 protein, but not a VP1 protein or a VP2 protein;
(iii) a VP3 protein only.

10. The AAV expression construct of any one of the preceding claims, wherein
(i) the first VP-coding region encodes a VP1 protein comprising the amino acid sequence of any of SEQ ID NOs: 149, 150, 153, 155, 156, 82, 161, 164, 84, 168, 171, or 174, or an amino acid sequence at least 70%, 75%, 80%, 85%, 90%, 95%, 96%, 97%, 98%, or 99% identical to any of the aforesaid amino acid sequences;
(ii) the second VP-coding region encodes a VP2 protein comprising amino acids 138-736 or SEQ ID NOs: 171, 149, or 150; amino acids 138-743 of SEQ ID NOs: 153, 155, 156, 82, 161, 164, 84; or amino acids 137-724 of SEQ ID NO: 174; and/or
(iii) the third VP-coding region encodes a VP3 protein comprising amino acids 203-736 of SEQ ID NOs: 171, 149, or 150; amino acids 203-743 of SEQ ID NOs: 153, 155, 156, 82, 161, 164, 84; or amino acids 193-724 of SEQ ID NO: 174.

11. The AAV expression construct of any one of the preceding claims, wherein one, two, or all of the first VP-coding region, the second VP-coding region, and the third VP-coding region are operably linked to a promoter, optionally, wherein the promoter for each VP-coding region is independently chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.

12. The AAV expression construct of any one of the preceding claims, wherein the at least one transcriptional regulator element coding region comprises an ORF which comprises a start codon and a nucleotide sequence encoding one or more transcriptional regulator elements, optionally, wherein the at least one regulator element is a Lac repressor (LacR) protein or an engineered Lac repressor protein (eLacr).

13. The AAV expression construct of any one of the preceding claims, wherein, the at least one regulator binding sequence is a Lac Operator (LacO) sequence, optionally, wherein at least one LacO sequence operably linked to the VP1 coding sequence, operably linked to a VP2 coding sequence or both.

14. The AAV expression construct of any one of the preceding claims, wherein the VP1-coding region and/or the VP2-coding region comprises at least one LacO-p10-LacO expression control sequence which comprises the nucleotide sequence of SEQ ID NO: 40 or a nucleotide sequence which is at least 75%, at least 80%, at least 85%, at least 90%, at least 93%, at least 95%, at least 97%, or at least 99% identical to SEQ ID NO: 40, and, optionally, wherein the VP1-coding region comprises a LacO sequence at the 3′ end of the coding sequence.

15. The AAV expression construct of any one of the preceding claims, wherein
(i) the first Rep-coding region is located in a first location of the baculovirus genome, and the second Rep-coding region is located in a second location of the baculovirus genome which is different from the first location of the baculovirus genome;
(ii) the first VP-coding region is located in a third location of the baculovirus genome, and the second VP-coding region is located in a fourth location of the baculovirus genome which is different from the third location of the baculovirus genome, and the third VP-coding region is located in a fifth location of the baculovirus genome which is different from the third location and the fourth location of the baculovirus genome;
(iii) the regulator element coding region is located in a sixth location of the baculovirus; wherein the first location, the second location, the third location, the fourth location, the fifth location, and/or the sixth location of the baculovirus genome are each independently selected from: egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94 gene locus.

16. A recombinant baculovirus genome comprising:
(i) a Rep78-coding region comprising a polh promoter located in the Tn7/polh gene locus of the baculovirus genome;
(ii) a Rep52-coding region comprising a polh promoter located in the egt gene locus of the baculovirus genome;
(iii) a VP1-coding region comprising a p10 promoter and at least one LacO sequence 5′ to the p10 promoter located in the ChiA/v-cath gene locus of the baculovirus genome;
(iv) a VP2-coding region comprising a p10 promoter located in the gta gene locus of the baculovirus genome;
(v) a VP3-coding region comprising a p10 promoter located in the Tn7/polh gene locus of the baculovirus genome; and
(vi) a LacR-coding region comprising a gp64/pol10 promoter located in the p74 gene locus of the baculovirus genome downstream of homologous repeat region hr5.

17. An AAV expression construct comprising:
(i) a Rep78-coding region comprising a polh promoter located in the SOD gene locus of the baculovirus genome;
(ii) a Rep52-coding region comprising a polh promoter, located in the egt gene locus of the baculovirus genome;
(iii) a VP1-coding region comprising a p10 promoter and at least one LacO sequence 5′ to the p10 promoter located in the gta gene locus of the baculovirus genome;
(iv) a VP2-coding region comprising a p10 promoter located in the ChiA/v-cath gene locus of the baculovirus genome;
(v) a VP3-coding region comprising a p10 promoter located in the SOD gene locus of the baculovirus genome; and
(vi) a LacR-coding region comprising a gp64/polh promoter located in the p74 gene locus of the baculovirus genome downstream of homologous repeat region hr5.

18. An AAV expression construct comprising:
(i) a Rep78-coding region comprising a polh promoter located in the SOD gene locus of the baculovirus genome;
(ii) a Rep52-coding region comprising a polh promoter, located in the egt gene locus of the baculovirus genome;
(iii) a first VP-coding region located in the gta gene locus of the baculovirus genome, wherein the first VP-coding region comprises a nucleotide sequence encoding VP1-coding region, a p10 promoter and at least one LacO sequence 5′ to the p10 promoter;
(iv) a second VP-coding region located in the SOD locus of the baculovirus genome, wherein the second VP-coding region comprises a nucleotide sequence encoding VP2 and VP3, and a p10 promoter; and
(vi) a LacR-coding region comprising a gp64/polh promoter located in the p74 gene locus of the baculovirus genome downstream of the homologous repeat region hr5.

19. An AAV viral production system comprising an AAV expression construct of any one of the preceding claims, and an AAV payload construct which comprises a transgene payload, optionally, wherein the comprises an AAV viral production cell, for example, an insect cell such as an Sf9 cell or an Sf21 cell.

20. A method of producing one, two, three, four, or all of a Rep78 protein, a Rep52 protein, a VP1 protein, a VP protein, and/or a VP3 protein, the method comprising:
(i) providing a cell comprising the AAV expression construct of any one of claims 1-18;
(ii) incubating the cell under conditions suitable to produce the one, two, three, four, or all of the Rep78 protein, the Rep52 protein, the VP1 protein, the VP protein, and/or the VP3 protein, optionally, wherein prior to step (i), introducing the AAV expression construct into the cell.

21. A method of producing an AAV particle, the method comprising:
(i) providing a cell comprising the AAV expression construct of any one of claims 1-17 and an AAV payload construct;
(ii) incubating the cell under conditions suitable to produce the AAV particle;
thereby producing the AAV particle.

22. The method of any one of claim 20 or 21, wherein an inducer element (e.g., IPTG) is introduced at a concentration between about 1.0 μM to about 20 μM, between about 1.0 μM to about 5.0 μM, between about 2.0 μM to about 3.0 μM, between about 5.0 μM to about 15.0 μM, or at a concentration of about 10.0 μM.

23. A cell comprising the AAV expression construct of any one of claims 1-17, or the AAV production system of claim 20, optionally, wherein the cell is an insect cell (e.g., an Sf9 cell or an Sf21).

24. A composition comprising the AAV expression construct of any one of claims 1-17, and a carrier.

25. An AAV particle made by the method of any one of claims 20-22.
